(19) United States
(12) Patent Application Publication
(10) Pub. No.: US 2025/0325586 A1
(43) Pub. Date: Oct. 23, 2025

(54) TREATING CANCER

(71) Applicant: Mayo Foundation for Medical Education and Research, Rochester, MN (US)

(72) Inventors: Saad J. Kenderian, Rochester, MN (US); John A. Copland, III, Ponte Vedra Beach, FL (US); Han W. Tun, Jacksonville, FL (US); Kendall J. Schick, Stewartville, MN (US)

(21) Appl. No.: 18/864,688

(22) PCT Filed: May 9, 2023
(86) PCT No.: PCT/US2023/021466

§ 371 Date: Nov. 11, 2024

Related U.S. Application Data
(60) Provisional application No. 63/340,872, filed on May 11, 2022.
(60) Provisional application No. 63/459,839, filed on Apr. 17, 2023.

(51) Int. Cl. A61K 35/17; A61K 40/11; A61K 40/31; A61K 40/42; A61K 45/06; A61P 35/00; C07K 14/705; C07K 14/725; C07K 16/28

(52) U.S. Cl. CPC A61K 35/17; A61K 40/11; A61K 40/31; A61K 40/4202; A61K 45/06; A61P 35/00; C07K 14/7051; C07K 14/70517; C07K 14/70575; C07K 16/2869; A61K 2239/13; A61K 2239/17; A61K 2239/46; C07K 2317/565; C07K 2317/622

(57) ABSTRACT

This document provides methods and materials for treating a mammal having cancer. For example, T cells (e.g., chimeric antigen receptor (CAR) T cells) engineered to express an antigen receptor (e.g., a CAR) that can target a thyroid stimulating hormone receptor (TSHR) polypeptide are provided. In some cases, T cells provided herein can be administered to a mammal having cancer to treat the mammal. For example, one or more T cells expressing (e.g., engineered to express) an antigen receptor (e.g., a CAR) that can target a TSHR polypeptide can be administered (e.g., in an adoptive cell therapy such as a CAR T cell therapy) to a mammal (e.g., a human) having cancer (e.g., thyroid cancer) to treat the mammal.

### CROSS-REFERENCE TO RELATED APPLICATIONS

This application is a National Stage application under 35 U.S.C. § 371 of International Application No. PCT/US2018/063485, having an International Filing Date of Nov. 30, 2018, which claims the benefit of U.S. Patent Application Ser. No. 63/340,872, filed on May 11, 2022, and U.S. Patent Application Ser. No. 63/459,839, filed on Apr. 17, 2023. The disclosures of the prior applications are considered part of, and are incorporated by reference in, the disclosure of this application.

### SEQUENCE LISTING

This application contains a Sequence Listing that has been submitted electronically as an XML file named “07039-2124WO1.xml.” The XML file, created on Apr. 19, 2023, is 175000 bytes in size. The material in the XML file is hereby incorporated by reference in its entirety.

### TECHNICAL FIELD

This document relates to methods and materials for treating a mammal having cancer. For example, this document provides methods and materials for using T cells (e.g., chimeric antigen receptor (CAR) T cells) engineered to express an antigen receptor (e.g., a CAR) that can target a thyroid stimulating hormone receptor (TSHR) polypeptide. In some cases, T cells provided herein can be administered to a mammal having cancer to treat the mammal.

### BACKGROUND INFORMATION

Thyroid cancer represents a significant burden to the healthcare system affecting a total of 893,000 patients in the U.S. alone as of 2018, and an estimated 44,280 new cases in the U.S. alone in 2021. Though rare, poorly differentiated and anaplastic thyroid cancers account for a a large portion of thyroid cancer related deaths while only representing 2-3% of the thyroid cancer diagnoses. While the overall survival of differentiated thyroid cancers (DTC) is around 95%, poorly differentiated thyroid cancers (PDTC) are associated with a 5-year survival of 66%. Furthermore, the overall survival of anaplastic thyroid cancers (ATC) have maintained a median survival of <6 months after diagnosis, with a 1-year survival of 20%, and an estimated 700 deaths per year. Treatment of thyroid cancer include surgery, radiation, and radioactive iodine therapy. Treatment options for PDTC are limited and largely ineffective.

### SUMMARY

This document provides methods and materials for generating T cells (e.g., CAR T cells) expressing (e.g., engineered to express) an antigen receptor (e.g., a CAR) that can target a TSHR polypeptide. For example, a T cell (e.g., a CAR T cell) engineered to express an antigen receptor (e.g., a CAR) that can target a TSHR polypeptide can include (e.g., can be engineered to include) nucleic acid encoding the antigen receptor (e.g., a CAR) that can target the TSHR polypeptide such that the antigen receptor is expressed by the T cell. This document also provides methods and materials for using T cells (e.g., CAR T cells) expressing (e.g., engineered to express) an antigen receptor (e.g., a CAR) that can target a TSHR polypeptide. In some cases, the T cells (e.g., CAR T cells) expressing (e.g., engineered to express) an antigen receptor (e.g., a CAR) that can target a TSHR polypeptide can be administered (e.g., in an adoptive cell therapy) to a mammal having cancer (e.g., thyroid cancer) to treat the mammal.

In general, one aspect of this document features T cells including a heterologous nucleic acid encoding an antigen receptor having the ability to bind to a TSHR polypeptide, where the T cell expresses the antigen receptor. The antigen receptor can be a CAR. The T cell can be a human T cell. The antigen receptor can include a single chain variable fragment (scFv) having the ability to bind to the TSHR polypeptide. The scFv can include a heavy chain variable (VH) domain including a complementarity determining region (CDR) 1 sequence set forth in any one of SEQ ID NOs:1-21, a CDR2 sequence set forth in any one of SEQ ID NOs:22-38, and a CDR3 sequence set forth in any one of SEQ ID NOs:39-50. The scFv can include a VH domain including an amino acid sequence set forth in any one of SEQ ID NOs:51-59. The scFv can include a light chain variable (VL) domain including a CDR1 sequence set forth in any one of SEQ ID NOs:60-68, a CDR2 sequence set forth in any one of SEQ ID NOs:69-77, and a CDR3 sequence set forth in any one of SEQ ID NOs:78-86. The scFv can include a VL domain can include an amino acid sequence set forth in any one of SEQ ID NOs:87-95. The scFv can include a VH domain including a CDR1 sequence set forth in any one of SEQ ID NOs:1-21, a CDR2 sequence set forth in any one of SEQ ID NOs: 22-38, and a CDR3 sequence set forth in any one of SEQ ID NOs:39-50, and a VL domain including a CDR1 sequence set forth in any one of SEQ ID NOs:60-68, a CDR2 sequence set forth in any one of SEQ ID NOs:69-77, and a CDR3 sequence set forth in any one of SEQ ID NOs:78-86. The antigen receptor can include an antigen-binding domain including a VH domain containing a set of CDRs as set forth in Table 4. The antigen receptor can include an antigen-binding domain including a VL domain containing a set of CDRs as set forth in Table 9. The antigen receptor can include an antigen-binding domain including a VH domain containing a set of CDRs as set forth in Table 4 and a VL domain containing a set of CDRs as set forth in Table 9. The antigen receptor can include one or more signaling domains. The one or more signaling domains can be independently selected from the group consisting of a CD3zeta signaling domain, a 4-1BB signaling domain, and a CD28 signaling domain. The antigen receptor can include a hinge domain (e.g., a CD8 hinge domain or a CD28 hinge domain). The antigen receptor can include a transmembrane domain (e.g., a CD3ζ transmembrane domain, a CD4 transmembrane domain, a CD8a transmembrane domain, a CD28 transmembrane domain, or a 4-1BB transmembrane domain).

In another aspect, this document features antibodies having the ability to bind to a TSHR polypeptide, where the antibody includes (a) a VH domain including a CDR1 sequence set forth in any one of SEQ ID NOs:1-21, a CDR2 sequence set forth in any one of SEQ ID NOs: 22-38, and a CDR3 sequence set forth in any one of SEQ ID NOs:39-50, and (b) a VL domain including a CDR1 sequence set forth in any one of SEQ ID NOs:60-68, a CDR2 sequence set forth in any one of SEQ ID NOs:69-77, and a CDR3 sequence set forth in any one of SEQ ID NOs:78-86. The antibody can be an scFv including (a) and (b), a variable domain antibody including (a) and (b), or variable domain antibody including (a) and (b).

In another aspect, this document features CARs having the ability to bind to a TSHR polypeptide, where the CAR includes: a scFv including a VH domain including a CDR1 sequence set forth in any one of SEQ ID NOs:1-21, a CDR2 sequence set forth in any one of SEQ ID NOs: 22-38, and a CDR3 sequence set forth in any one of SEQ ID NOs:39-50, and a VL domain including a CDR1 sequence set forth in any one of SEQ ID NOs:60-68, a CDR2 sequence set forth in any one of SEQ ID NOs:69-77, and a CDR3 sequence set forth in any one of SEQ ID NOs:78-86; a CD8 hinge domain; a 4-1BB signaling domain; and a CD3zeta signaling domain.

In another aspect, this document features nucleic acid constructs encoding CARs having the ability to bind to a TSHR polypeptide, where the CAR includes: a scFv including a VH domain including a CDR1 sequence set forth in any one of SEQ ID NOs:1-21, a CDR2 sequence set forth in any one of SEQ ID NOs: 22-38, and a CDR3 sequence set forth in any one of SEQ ID NOs:39-50, and a VL domain including a CDR1 sequence set forth in any one of SEQ ID NOs:60-68, a CDR2 sequence set forth in any one of SEQ ID NOs:69-77, and a CDR3 sequence set forth in any one of SEQ ID NOs:78-86; a CD8 hinge domain; a 4-1BB signaling domain; and a CD3zeta signaling domain. The nucleic acid construct can be in the form of a viral vector. The viral vector can be a lentiviral vector.

In another aspect, this document features methods for treating a mammal having cancer. The methods can include, or consist essentially of, administering, to a mammal having cancer, T cells including a heterologous nucleic acid encoding an antigen receptor having the ability to bind to a TSHR polypeptide, where the T cell expresses the antigen receptor, where the cancer includes a cancer cell expressing a TSHR polypeptide. The antigen receptor can be a CAR. The T cell can be a human T cell. The antigen receptor can include a scFv having the ability to bind to the TSHR polypeptide. The scFv can include a VH domain including a CDR1 sequence set forth in any one of SEQ ID NOs:1-21, a CDR2 sequence set forth in any one of SEQ ID NOs:22-38, and a CDR3 sequence set forth in any one of SEQ ID NOs:39-50. The scFv can include a VH domain including an amino acid sequence set forth in any one of SEQ ID NOs:51-59. The scFv can include a VL domain including a CDR1 sequence set forth in any one of SEQ ID NOs:60-68, a CDR2 sequence set forth in any one of SEQ ID NOs:69-77, and a CDR3 sequence set forth in any one of SEQ ID NOs:78-86. The scFv can include a VL domain can include an amino acid sequence set forth in any one of SEQ ID NOs:87-95. The scFv can include a VH domain including a CDR1 sequence set forth in any one of SEQ ID NOs:1-21, a CDR2 sequence set forth in any one of SEQ ID NOs: 22-38, and a CDR3 sequence set forth in any one of SEQ ID NOs:39-50, and a VL domain including a CDR1 sequence set forth in any one of SEQ ID NOs:60-68, a CDR2 sequence set forth in any one of SEQ ID NOs:69-77, and a CDR3 sequence set forth in any one of SEQ ID NOs:78-86. The antigen receptor can include an antigen-binding domain including a VH domain containing a set of CDRs as set forth in Table 4. The antigen receptor can include an antigen-binding domain including a VL domain containing a set of CDRs as set forth in Table 9. The antigen receptor can include an antigen-binding domain including a VH domain containing a set of CDRs as set forth in Table 4 and a VL domain containing a set of CDRs as set forth in Table 9. The antigen receptor can include one or more signaling domains. The one or more signaling domains can be independently selected from the group consisting of a CD3zeta signaling domain, a 4-1BB signaling domain, and a CD28 signaling domain. The antigen receptor can include a hinge domain (e.g., a CD8 hinge domain or a CD28 hinge domain). The antigen receptor can include a transmembrane domain (e.g., a CD3ζ transmembrane domain, a CD4 transmembrane domain, a CD8a transmembrane domain, a CD28 transmembrane domain, or a 4-1BB transmembrane domain). The mammal can be a human. The cancer can be a thyroid cancer. The method also can include administering, to the mammal, an agent that increases expression of a TSHR polypeptide on a cancer cell within the mammal. The agent can be a mitogen-activated protein kinase kinase (MEK) inhibitor. The MEK inhibitor can be trametinib, binimetinib, selumetinib, or cobimetinib. The agent can be a v-raf murine sarcoma viral oncogene homolog B1 (BRAF) inhibitor. The BRAF inhibitor can be vemurafenib, dabrafenib, or encorafenib.

In another aspect, this document features methods for treating a mammal having cancer. The methods can include, or consist essentially of, administering, to a mammal having cancer, a population of cells comprising nucleic acid encoding a chimeric antigen receptor having the ability to bind to a TSHR polypeptide. The mammal can be a human. The cancer can be a thyroid cancer. The cells can be T cells. The T cells can be T cells comprising a heterologous nucleic acid encoding an antigen receptor having the ability to bind to a TSHR polypeptide, where the T cell expresses the antigen receptor. The chimeric antigen receptor can be a CAR having the ability to bind to a TSHR polypeptide, where the CAR comprises: a scFv comprising a VH domain comprising a CDR1 sequence set forth in any one of SEQ ID NOs:1-21, a CDR2 sequence set forth in any one of SEQ ID NOs: 22-38, and a CDR3 sequence set forth in any one of SEQ ID NOs:39-50, and a VL domain comprising a CDR1 sequence set forth in any one of SEQ ID NOs:60-68, a CDR2 sequence set forth in any one of SEQ ID NOs:69-77, and a CDR3 sequence set forth in any one of SEQ ID NOs:78-86; a CD8 hinge domain; a 4-1BB signaling domain; and a CD3zeta signaling domain. The method can include administering, to the mammal, one or more agents that increase expression of a TSHR polypeptide on a cancer cell within the mammal. The at least one of the one or more agents can be a MEK inhibitor. The MEK inhibitor can be trametinib, binimetinib, selumetinib, or cobimetinib. The at least one of the one or more agents can be a BRAF inhibitor. The BRAF inhibitor can be vemurafenib, dabrafenib, or encorafenib. The method can include administering, to the mammal, a MEK inhibitor and a BRAF inhibitor. The method can include administering, to the mammal, one or more agents that reduce the number of macrophages within the mammal. The at least one of the one or more agents that reduce the number of macrophages within the mammal can be a CSF-1R specific kinase inhibitor. The CSF-1R specific kinase inhibitor can be Ki20227, pimicotinib (ABSK021), pexidartinib (PLX3397), ARRY-382, PLX7486, BLZ945, or JNJ-40346527. The at least one of the one or more agents that reduce the number of macrophages within the mammal can be a GM-CSF neutralizing antibody, clodronate, emactuzumab, AMG820, IMC-CS4, cabiralizumab, lacnotuzumab (MCS 110), or PD-0360324. The at least one of the one or more agents that reduce the number of macrophages within the mammal can be an immunomodulatory imide drug. The immunomodulatory imide drug can be thalidomide, lenalidomide, pomalidomide, iberdomide, or apremilast.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF THE DRAWINGS

FIGS. 1A-1E. TSHR as a target for CAR T cell therapy in thyroid cancer. FIG. 1A) RIC staining demonstrating characteristic relative TSHR expression across normal thyroid tissues. FIG. 1B) H-scores from benign tumors and various malignant tumors used in FIG. 1A. FIG. 1C) Correlation of NIS. expression with TSHR expression in normal thyroid tissue and in anaplastic thyroid cancer. FIG. 1D) Diagram of the exemplary CAR designs. CAR designs were described in 5′-3′ orientation from left to right. FIG. 1E) A flow cytometry histogram showing a representative comparison between activated non-engineered T cells (UTD) (*) and CAR T cells (+) transduced using lentivirus. As indicated, 92% of CAR T cells (+) had surface CAR expression with a 7.96% false-positive rate as indicated from the UTD (*) group.

FIGS. 2A-2F. TSHR-CAR T cells exhibit potent antitumor activity. FIG. 2A) TSHR-CAR T cells show significant antigen-specific proliferation (*) compared to donor-matched, UTD T cells (+). Statistics were performed using 2-way ANOVA followed by multiple comparisons. FIG. 2B) TSHR-CAR T cells have powerful anti-cancer effects against a TSHR-transduced thyroid cancer cell line (square) as compared to donor matched UTD T cells (circle). The test was performed in technical duplicates of each biological triplicate. Statistics performed were 2-way ANOVA followed by a multiple comparisons test. FIG. 2C) CAR T cells display elevated secretion of IL-2 (†), GM-CSF (§), IFN-γ (±), and express higher levels of MIP1b (¿), and CD107a (») when experiencing CAR-mediated antigen stimulation. Test was performed in technical duplicates of each biological triplicate. Statistics performed were separate T-tests for each comparison. FIG. 2D) NSG mice were engrafted with 1 million K562 leukemic cells transduced to stably express TSHR and luciferase genes. The mice were imaged weekly for tumor burden. Once the mice reached a late-stage cancer at day 30, the mice were randomized by tumor burden and infused with 2 million T cells, either control or CAR T cells, derived from one of two healthy donors. The resulting Kaplan-Meier curve indicates the TSHR-directed CAR T cells delay tumor progression and enhance mouse survival duration. FIG. 2E) NSG mice were injected subcutaneously with FTC-133 thyroid cancer cells, transduced to express TSHR and luciferase genes, suspended in 50% MATRIGEL®. On day 7, mice were randomized based on tumor burden and infused with either 2 million control T cells (square), 2 million CAR T cells (circle), or 5 million CAR T cells (triangle). This study demonstrates that TSHR-directed CAR T cells slow tumor progression and prolong mouse survival in a flank tumor model in a dose dependent manner. FIG. 2F, top). Schematic of patient-derived xenograft (PDX) thyroid cancer experiments. NSG mice were injected subcutaneously with TSHR-transduced THJ529 thyroid cancer cells suspended in MATRIGEL*. On day 3, mice were randomized based on tumor burden and infused with either 5 million control T cells, 4.8 million CAR T cells, or 11 million CAR T cells. FIG. 2F, bottom left). These mice were monitored for tumor volume until endpoint criteria was reached. As indicated, the high dose CAR T cell arm (triangle) had greatly slowed tumor progression vs UTD (circle) and low dose CAR T cell (square) groups. FIG. 2F, bottom right). The Kaplan-Meier curve illustrates that TSHR-directed CAR T cells can elicit survival prolongation in a dose dependent manner in ATC tumor mouse models.

FIGS. 3A-3H. Mitogen-activated protein kinase kinase (MEK) and v-raf murine sarcoma viral oncogene homolog B1 (BRAF) inhibitors upregulated TSHR and increased the therapeutic index of TSHR-CAR T cells. FIG. 3A) Schema of the experiment: NSG mice were engrafted with patient derived tumors through subcutaneous implantation. Mice were then treated with MEK inhibitors, BRAF inhibitors, or the combination of MEK and BRAF inhibitors. Mice were then followed for tumor volume by caliper measurement and the tumor was harvested 7 and 14 days after treatment. FIG. 3B) MEK inhibitors and BRAF inhibitors upregulated TSHR expression in ATC PDX tumors 7 and 14 days post therapy. FIG. 3C) CAR T cell proliferation remained unaffected by MAPKi. Antigen-specific stimulation induced CAR T cell proliferation regardless of the target cell line used. The proliferative capacity of antigen-stimulated CAR T cells was not attenuated by the addition of MEK Inhibitor. FIG. 3D) Antigen stimulated CAR T cell degranulation remained unaffected by MAPKi. The antigen-specific TSHR-CAR T cell release of cytotoxic granules and key cytokines remained unchanged regardless of antigen presenting cells used. FIG. 3E) The antigen-specific anti-tumor response of TSHR-CAR T cells was found to remain unaffected in the presence of MEK inhibitor as tested by 2-way ANOVA. FIG. 3F) Schematic of the experiment: NSG mice were engrafted with ATC tumor by subcutaneous injection. Mice were treated with MEK inhibitors and with BRAF inhibitors for 1-2 weeks. In parallel, mice were engrafted with a tumor one week later. All mice were randomized to receive either TSHR-CAR T cells or control untransduced T cells. Disease burden was monitored using caliper assessment and mice were monitored for survival. FIG. 3G) ATC PDX mice primed with MAPKi responded significantly better to TSHR-CAR T cell therapy compared to control xenografts. FIG. 3H. No difference was observed in CAR T cell expansion in the peripheral blood in mice pretreated with MEK inhibitors compared to control xenografts.

FIGS. 4A-4E. Tumor-associated macrophages (TAM) were abundant in ATC and were associated with poor response to TSHR-CAR T cell therapy. FIG. 4A) The CD14 biomarker demonstrated the presence of macrophages in ATC tumors, CD80 depicted the percentage of M1 (anti-tumor), and CD206 depicted the protumorigenic macrophage populations. Shown here is a globally higher prevalence of M2 macrophage populations in ATC tumors. FIG. 4B) CD3⁺ T cell tumor infiltration was increased in CAR T cell treated mice, and was more significant with higher doses of CAR T cell. FIG. 4C) CAR T cells was associated with polarization to M1 macrophages (pSTAT1) and down-regulation of M2 macrophages as evidenced by attenuated YM-1 positivity in CAR T cell treated tumors. The middle panel exemplifies a cross-section of tumor while the right panel represents a 200 micron close up of protein expression in tumors of (F4/80), M1 (pSTAT1), and M2 (YM-1) macrophages. FIG. 4D) CD3⁺ T cell tumor residence was inversely correlated to YM1 mouse macrophage prevalence in CAR T cell treated mice. This two-tailed Pearson Correlation was statistically significant with a p-value=0.0423. FIG. 4E) CD3⁺ T cell tumor prevalence was directly correlated to subject therapy response. This two-tailed Pearson Correlation was statistically significant with a p-value=0.0498.

FIGS. 5A and 5B. T cell and macrophage infiltration in tumors treated with CAR T in combination with MEK inhibitors and BRAF inhibitors. FIG. 5A) IHC and histology scoring of ATC tumors from the experiment previously described in FIG. 2 demonstrated that T cell tumor infiltration is significant following CAR T cell therapy. FIG. 5B) M2 macrophage infiltration was reduced following CAR T treatment.

FIGS. 6A and 6B. Macrophage attenuation provides survival benefit to TSHR CART against anaplastic thyroid carcinoma (ATC) patient derived xenograft (PDX) mouse model. THJ-560T ATC PDX tumors (5 mm³) were implanted in the right flank s.q. according to IACUC protocols. Tumors grown to ˜75 mm³, were treated daily via oral gavage for one week with 1 mg/kg trametinib (MEK inhibitor) and 0.75 mg/kg dabrafenib (BRAF inhibitor). On day 8, mice were treated with 20 million UDT (control) or TSHR CART cells via tail vein injection (n=6 mice/group). One group of TSHR CART treated mice were administered clodronate or GM-CSF neutralizing antibody to inhibit macrophages, known to promote tumor growth and metastasis. FIG. 6A) Tumor volume (height×length×width×0.523=mm³) was measured twice weekly through day 29. FIG. 6B) Kaplan-Meyer survival curves were plotted using a cutoff of 700 mm³ tumor volume.

FIGS. 7A-7F. TSHR represents an ideal target for CAR-T cell therapy with potent anti-tumor functions. FIG. 7A) IHC staining demonstrating characteristic relative TSHR expression across normal and malignant thyroid tissues. TSHR H-scoring scale of clinical samples from normal and malignant thyroid sections. PTC; Papillary thyroid cancer, FTC; Follicular thyroid cancer and ATC; Anaplastic thyroid cancer. FIG. 7B) H-scoring of various patient thyroid cancer models demonstrating equal expression levels of TSHR amongst various tumor models. FIG. 7C) TSHR CAR-T cells show significant antigen-specific cytotoxicity in comparison to UTD control against TSHR⁺ tumor cells. TSHR CAR-T cells and UTD cells were co-cultured with Luciferase⁺TSHR⁺ tumor cells (FTC133) and cytotoxicity was measured via bioluminescence after 48 hours (Two-way ANOVA; ns=not significant, ****p<0.0001; 2 biological replicates; error bars, SEM). FIG. 7D) TSHR-CART cells presented higher proliferation in comparison to UTD control cells. TSHR CAR-T cells or UTD cells were co-cocultured with either TSHR⁺ cell line FTC133 (CAR stimulation), PMA/ionomycin (Ca⁺ influx stimulation) or media alone (no stimulation) for 5 days. Then, CD3⁺ T cell population was measured via flow cytometry (Two-way ANOVA; ns=not significant, **p<0.01, ***p<0.001, 1 biological replicate; error bars, SEM). FIG. 7E) TSHR CART cells present higher antigen-specific degranulation. TSHR CART cells and UTD cells were-cocultured with TSHR⁺ tumor cells at a 5:1 ratio and 6 hours later, degranulation was measured based on CD107a expression levels via flow cytometry (Unpaired T-test; **p<0.01; 2 biological replicates; error bars, SEM). FIG. 7F) TSHR CART cells display elevated secretion of cytokines via antigen-specific stimulation. TSHR CART cells or UTD cells were co-cultured with TSHR*FTC133 cell line at 1:5 ratio for 6 hours, then, secretion of cytokines was assessed by intracellular staining, followed by flow cytometry (Two-way ANOVA; ns=not significant, *p<0.05, ****p<0.0001; error bars; SEM).

FIGS. 8A-8G. TSHR CAR-T cells exhibit dependent dose-escalation potent anti-tumor activity in vivo. FIG. 8A) Experimental schema of TSHR xenograft model. NSG mice were subcutaneously engrafted with TSHR-transduced THJ529 thyroid cancer cells suspended in Matrigel. On day 7, mice were randomized according to tumor volume to receive CART treatment. Mice were monitored for tumor volume regression, overall survival IHC of CD3⁺ T cell infiltration in tumor sites. FIG. 8B) Treatment with TSHR CART cells resulted in dose-dependent potent anti-tumor activity. Assessment of tumor volume via caliper measurement between UTD cells and high dose or low dose of CART cells. FIG. 8C) TSHR-directed CAR-T cells can elicit prolonged survival in a dose dependent manner. Kaplan-Meier curve illustrating probability of survival between CART and UTD groups (Kaplan-Meier survival curve; *p<0.0.5, **p<0.01; n=5 mice per group). FIGS. 8D and 8E) Assessment of CD3⁺ T cells in TSHR CAR-T and UTD-treated mice. FIG. 8D). TIC section of TSHR⁺ tumor sections showing dose-dependent increase in CD3⁺ T cell population (Kruskal Wallis test; *p=0.0047, **p=0.0305; n=5 mice). FIG. 8E). H-score of CD3⁺ T cell population in TSHR treated tumors (One-way ANOVA; ns=not significant, **p<0.01; n=6 mice; error bars, SEM). FIGS. 8F and 8G) Reduction of TSHR expression on mice treated with high dose of TSHR CAR-T cells. FIG. 8F) Hematoxylin and Eosin (H&E) staining (upper panel) and TSHR expression (lower panel) of representative areas on each mouse group. FIG. 8G) H-score of TSHR-treated tumors analysis.

FIGS. 9A-9J. Treatment of thyroid tumors with MAPK inhibitors results in upregulation of TSHR. FIG. 9A) Dose-escalation of trametinib is proportional to thyroid tumor volume reduction. NSG mice treated with different daily doses of trametinib for 8 days and tumor volume regression was measured. FIG. 9B) TSHR expression was restored with higher dose of trametinib, whereas pERK expression was reduced. Right. IHC section of tumor sections showing dose-dependent increase of TSRH and reduction of pERK. Left. Association between H-score of TSHR and pERK in correlation with trametinib dose-escalation. FIG. 9C) Dose-escalation of R05126766 was proportional to thyroid tumor volume reduction. Assessment of tumor volume on NSG mice treated with different doses of R05126766. FIG. 9D) Higher doses of R05126766 resulted in increased expression of TSRH and lower expression of pERK. Right. ICH tumor sections showing TSRH and pERK expression. Left. H-score of TSRH and pERK in association with increasing doses of R05126766. FIG. 9E) Experimental schema of an ATC PDX model treated with MAPK inhibitors (MEKi and BRAFi). Two different MEK inhibitors (R05126766 or trametinib)+BRAF inhibitor (dabrafenib, 12.5 mg/kg, p.o., daily) were daily dosed orally to NSG mice implanted with ATC THJ-560 PDX tumors. Treatment was begun when tumors were ˜75-100 mm³. FIG. 9F) MEK and BRAF inhibitors upregulated TSHR expression in an ATC-PDX model. Upper panel. H-score of TSHR in ATC-derived treated tumors (One-way ANOVA; **p<0.01; n=4 mice; error bars, SEM). Lower panel. TSHR expression assessment via IHC. FIG. 9G) Combination of MEKi and BRAFi in an ATC PDX model resulted in improved anti-tumor activity. Tumor volume assessment over a period of 7 days of MAPK inhibitors dosage. FIG. 9H) Experimental schema of an ATC PDX model treated with MEK inhibitors. NSG mice were implanted with ATC PDX tumors and treatment with MEK inhibitors (for 14 days) R05126766 and trametinib started (daily dosage) when tumors reached ˜75-100 mm³. FIG. 9I) Addition of MEK inhibitors resulted in increased TSHR expression in ATC PDX mice. Upper panel. IHC score of TSHR in mice treated for 14 days with either placebo, R05126766 or trametinib (One-way ANOVA; *p<0.05, **p<0.01; n=4 mice; error bars, SEM). Lower panel. IHC of TSHR expression. FIG. 9J) Treatment with MEK inhibitor R05126766 resulted in better anti-tumor activity in comparison to trametinib. Tumor volume assessment via caliper measurement in mice treated for 14 days with either placebo, R05126766 or trametinib.

FIGS. 10A-10G. Addition of MAPK inhibitors did not impair CART effector functions and upregulated TSHR expression. FIG. 10A) Addition of MEK inhibitors did not impair TSHR CART cell cytotoxicity. TSHR CAR-T cells and UTD cells were co-cultured with Luciferase⁺TSHR⁺ tumor cells (FTC133) in the presence or absence of 10 nM trametinib and cytotoxicity was measured via bioluminescence after 48 hours (Two-way ANOVA; ns=not significant; 2 biological replicates; error bars, SEM). FIG. 10B) TSHR-CART antigen-specific proliferation was not impaired when MEK inhibitors were included. TSHR CAR-T cells and UTD cells were co-cultured with FTC133 tumor cells in the presence of media or 10 nM trametinib for 5 days, then flow cytometry was performed to assess CD3⁺ T cell expansion (One-way ANOVA; ns=not significant; 2 biological replicates; error bars, SEM). FIG. 10C) Degranulation as determined by CD107⁺ cells was not impaired in the presence of MEK inhibition. FIG. 10D) TSHR CART secretion of cytokines via antigen-specific stimulation was not impaired by addition of MEK inhibitor. TSHR CART cells or UTD cells were co-cultured with TSHR⁺ FTC133 cell line at 1:5 ratio for 6 hours in the presence or absence of trametinib, then, secretion of cytokines was assessed by intracellular staining, followed by flow cytometry (Two-way ANOVA; 2 biological replicates; error bars; SEM). FIG. 10E) Schema for CART in combination with MEK+BRAF inhibitors (Group 1) or single agent CART (Group #2). THJ-560 ATC tumor tissue (5 mm³) was surgically implanted in the in right flank on Day 0 for Group 1. On Day 2 for Group 1, mice were treated daily for 7 days with trametinib (0.25 mg/kg)+dabrafenib (2 mg/kg) to induce TSHR. For Group 1, on day 10, the CART control (untransduced control CART; UTD) or TSHR CART were each injected via tail vein (10⁶ cells). Tumor volume was measured twice weekly until end of experiment. For Group 2 that did not receive the MEK/BRAF inhibitors, tumor tissue was implanted on day 7 with UTD or TSHR CART injected on day 10 and tumor volume measured to end of experiment. FIG. 10F) Combination MEK/BRAF inhibitors+TSHR CART showed statistically significant attenuation of tumor volume compared to all other treatment groups that include control UTD, UTD+trametinib+dabrafenib, and TSHR CART alone. FIG. 10G) Human CD3 immunohistochemistry (IHC) confirms infiltration of the targeted TSHR CART cells into the tumor while UTD control CART cells.

FIGS. 11A-11I. FIG. 11A) TSHR protein expression was lost within 48 hours of MEK+BRAF inhibitor cessation. MC-TH-560 PDX were treated with R05126766+dabrafenib from days 1-7. Tumors were examined for TSHR protein expression via IHC at various time points. TSHR expression was lost by 48 hours of inhibitor cessation. FIG. 111B) Continuous trametinib (0.2 mg/kg)+dabrafenib (2 mg/kg) given daily and throughout experiment in combination with TSHR CART given via tail vein 10 days after THJ-560 tumor implantation demonstrated significant tumor regression with values significantly different compared to TSHR CART or trametinib+dabrafenib alone. Continuous trametinib+dabrafenib showed superiority over the 7-day treatment, discontinuous trametinib+dabrafenib treatment groups, demonstrating that continued TSHR protein expression is critical to overall response. FIGS. 11C-11J) Individual tumor volumes for each mouse and treatment are for UTD control (FIG. 11C), TSHR CART (FIG. 11D), MEK:BRAF inhibitors for 7 days and then TSHR CART (FIG. 11E), and continuous MEK:BRAF inhibitors plus TSHR CART (FIG. 11F). In the following figures, absolute T cell count is shown for FIG. 11G) TSHR CART treatment, FIG. 11H) continuous MEK:BRAF inhibitors plus TSHR CART, and discontinuous MEK:BRAF inhibitors and then TSHR CART (FIG. 11I).

FIGS. 12A-12D. Generation of TSHR CAR construct and manufacturing process. FIG. 12A) TSHR expression across different thyroid tissues with different doses of TSHR antibody. High and low IHC staining of TSRH across different thyroid tissue models. FIG. 12B) Representation of TSHR scFv construct. The CAR design was derived from the TSHR-antagonist autoantibody clone K1-70. Various sections, in particular the ScFvs, underwent manual codon optimization for codons with high human expression frequency and minimized G-C content. FIG. 12C) Procedure timeline to manufacture TSHR CART cells from healthy donor blood. Briefly, human T cells, isolated from healthy donor PBMCs, are stimulated with CD3/CD38 beads. One day later, stimulated T cells are transduced with a lentivirus carrying the TSHR construct and are fed with media for 4 days. On day 6, CART cells are de-beaded and subjected to flow cytometry to confirm % CAR expression. On day 8, CART cells are frozen and cryopreserved. FIG. 12D) Flow cytometry histogram comparing TSHR-CAR expression levels between CART cells and negative control UTD cells.

FIG. 13. Examples of tumor growth in PDX mouse models.

FIGS. 14A-14B. Generation of GM-CSFk/o TSHR CART cells. FIG. 14A) GM-CSF^{k/o} and GM-CSFwt CART cells show similar levels of CAR expression. FIG. 14B) GM-CSF^{k/o} TSHR CART cells exhibit reduced levels of intracellular GM-CSF but maintained levels of IL-2 and CD107α degranulation compared to GM-CSFwt TSHR CART cells.

FIGS. 15A-15B. TSHR CAR T targeted therapy attenuates Hurthle cell thyroid carcinoma XCT.UC1 tumor volume. Hurthle cells XCT.UC1 (10⁶ cells/mouse) were injected into the right flank of NSG mice (n=12) in 50% PBS and 50% Matrigel. After 48 hours, mice were injected via tail vein with either untransduced control T cells (UTD) or TSHR CAR T cells. FIG. 15A) Tumor volume was measured twice weekly. FIG. 15B) Body weight were measured twice weekly. Statistical analysis was determined using unpaired, two-tailed t-test (****p<0.001).

FIGS. 16A-16C. Effect of one or more MEK inhibitors and/or one or more BRAF inhibitors on tumor growth in MC-TH-529 PDX mouse models. FIG. 16A) A graph showing mean tumor volume. FIG. 16B) A graph showing median tumor volume. FIG. 16C) TSHR immunohistochemical staining of MC-TH-529 PDX model at 1 week.

FIGS. 17A-17B. Effect of one or more MEK inhibitors and/or one or more BRAF inhibitors on tumor growth in MC-TH-560 PDX mouse models. FIG. 17A) A graph showing mean tumor volume. FIG. 17B) A graph showing median tumor volume.

### DETAILED DESCRIPTION

This document provides methods and materials for generating T cells (e.g., CAR T cells) engineered to express an antigen receptor (e.g., a CAR) that can target a TSHR polypeptide. For example, a T cell (e.g., a CAR T cell) engineered to express an antigen receptor (e.g., a CAR) that can target a TSHR polypeptide can include (e.g., can be engineered to include) nucleic acid encoding the antigen receptor (e.g., a CAR) that can target the TSHR polypeptide such that the antigen receptor is expressed by the T cell. In some cases, a T cell (e.g., a CAR T cell) engineered to express an antigen receptor (e.g., a CAR) that can target a TSHR polypeptide can target (e.g., target and destroy) cancer cells (e.g., cancer cells such as thyroid cancer cells) within a mammal (e.g., a human).

CAR T cell therapy has emerged as a potentially curative therapy in a subset of patients with hematological malignancies and has been approved by the United States Food and Drug Administration (FDA) in several B cell malignancies. With the advent of adoptively transferred engineered cellular therapies, there is a compelling rationale to apply CAR T cell therapy to the treatment resistant solid tumors. However, the efficacy of CAR T cell therapy in solid tumors has been modest. Due to a lack of unique targets for CAR T cell therapy in solid tumors (most targets are shared between solid tumors and normal tissue), as well as the immunosuppressive tumor microenvironment in solid tumors which has been demonstrated to inhibit CAR T cells.

A T cell (e.g., a CAR T cell) engineered to express an antigen receptor (e.g., a CAR) that can target a TSHR polypeptide provided herein (e.g., a T cell engineered to include nucleic acid encoding a CAR that can target a TSHR polypeptide) can be any appropriate T cell. A T cell can be a naïve T cell. Examples of T cells that can be engineered to express an antigen receptor (e.g., a CAR) that can target a TSHR polypeptide as described herein include, without limitation, cytotoxic T cells (e.g., CD4⁺ CTLs and/or CD8⁺ CTLs), CD3, stem cell memory T cells, natural killer T (NKT) cells, and invariant NKT (iNKT) cells. In some cases, one or more T cells designed to express an antigen receptor (e.g., a CAR) that can target a TSHR polypeptide can be T cells that were obtained from a mammal (e.g., a mammal having cancer) that is to be treated with those T cells designed to express an antigen receptor (e.g., a CAR) that can target a TSHR polypeptide. For example, T cells can be obtained from a mammal to be treated with the materials and method described herein. In some cases, a cell other than a T cell can be designed to include an antigen receptor (e.g., a CAR) provided herein that can target a TSHR polypeptide. For example, macrophages, monocytes, NK cells, and hematopoietic stem cells can be engineered to express an antigen receptor (e.g., a CAR) provided herein that can target a TSHR polypeptide.

A T cell (e.g., a CAR T cell) engineered to express an antigen receptor (e.g., a CAR) that can target a TSHR polypeptide provided herein (e.g., a T cell engineered to include nucleic acid encoding a CAR that can target a TSHR polypeptide) can target any appropriate TSHR polypeptide.

A T cell (e.g., a CAR T cell) engineered to express an antigen receptor (e.g., a CAR) that can target a TSHR polypeptide provided herein (e.g., a T cell engineered to include nucleic acid encoding a CAR that can target a TSHR polypeptide) can express any appropriate type of antigen receptor. In some cases, an antigen receptor can be a heterologous antigen receptor. In some cases, an antigen receptor can be a CAR.

In some cases, an antigen receptor (e.g., a CAR) that can target a TSHR polypeptide can be a CAR that can target a TSHR polypeptide. The CAR that can target a TSHR polypeptide can include an antigen-binding domain that can target a TSHR polypeptide and a signaling domain. An antigen-binding domain that can target a TSHR polypeptide can be any appropriate antigen-binding domain that can target a TSHR polypeptide. In some cases, an antigen-binding domain that can target a TSHR polypeptide can include an antibody or a fragment thereof that binds to a TSHR polypeptide. Examples of antigen-binding domains include, without limitation, an antigen-binding fragment (Fab), a heavy chain variable (VH) domain of an antibody, a light chain variable (VL) domain of an antibody, a single chain variable fragment (scFv), and a protein ligand. In some cases, an antigen-binding domain that can bind to a TSHR polypeptide can include a VH domain of an antibody that binds to a TSHR polypeptide and a VL domain of an antibody that binds to a TSHR polypeptide.

Also provided herein are antigen receptors (e.g., CARs) that can target a TSHR polypeptide described herein and nucleic acid constructs encoding such antigen receptors (e.g., CARs).

An antigen-binding domain in an antigen receptor (e.g., a CAR) that can target a TSHR polypeptide can include any appropriate amino acid sequence. For example, an antigen-binding domain in an antigen receptor (e.g., a CAR) that can bind to a TSHR polypeptide can include complementary-determining regions (CDRs) each having any appropriate amino acid sequence. In some cases, a VH domain of an antigen-binding domain in an antigen receptor (e.g., a CAR) that can target a TSHR polypeptide can have an amino acid sequence that includes: (i) a CDR 1 that comprises, consists essentially of, or consists of the amino acid sequence set forth in any one of SEQ ID NOs:1-21, (ii) a CDR2 that comprises, consists essentially of, or consists of the amino acid sequence set forth in any one of SEQ ID NOs:22-38, and (iii) a CDR3 that comprises, consists essentially of, or consists of the amino acid sequence set forth in any one of SEQ ID NOs:39-50. As used herein, a “CDR1 that consists essentially of the amino acid sequence set forth in anyone of SEQ IDNOs:1-21” is a CDR1 that has zero, one, or two amino acid substitutions within the articulated sequence (e.g., any one of SEQ ID NOs:1-21), that has zero, one, two, three, four, or five amino acid residues directly preceding the articulated sequence (e.g., any one of SEQ ID NOs:1-21), and/or that has zero, one, two, three, four, or five amino acid residues directly following the articulated sequence (e.g., any one of SEQ ID NOs:1-21), provided that the antigen receptor (e.g., a CAR) maintains its basic ability to allow for binding of an antigen-binding domain to a TSHTR polypeptide. Examples of CDR1 amino acid sequences that can be included in a VH domain of an antigen-binding domain of an antigen receptor (e.g., a CAR) that can target a TSHR polypeptide include, without limitation, those set forth in Table 1.

TABLE 1
Exemplary VH domain CDR1s.

| | Sequence | SEQ ID NO |
|---|---|---|
| VH CDR1 | GFSVSGN | 1 |
| VH CDR1 | GNQMT | 2 |
| VH CDR1 | GFSVSGNQMT | 3 |
| VH CDR1 | GFTFTT | 4 |
| VH CDR1 | GFSVGSA | 5 |
| VH CDR1 | SADMS | 6 |
| VH CDR1 | GFSVGSADMS | 7 |
| VH CDR1 | GFTFSNS | 8 |
| VH CDR1 | NSDMA | 9 |
| VH CDR1 | GFTFSNSDMA | 10 |
| VH CDR1 | AIKYS | 11 |
| VH CDR1 | GFSFNDY | 12 |
| VH CDR1 | DYGLH | 13 |
| VH CDR1 | GFSFNDYGLH | 14 |
| VH CDR1 | GFTFSNY | 15 |
| VH CDR1 | NYALS | 16 |
| VH CDR1 | GFTFSNYALS | 17 |
| VH CDR1 | GYSLTDN | 18 |
| VH CDR1 | DNWIG | 19 |
| VH CDR1 | GYSLTDNWIG | 20 |
| VH CDR1 | NYWIG | 21 |

As used herein, a “CDR2 that consists essentially of the amino acid sequence set forth in any one of SEQ ID NOs:22-38” is a CDR2 that has zero, one, or two amino acid substitutions within the articulated sequence (e.g., any one of SEQ ID NOs:22-38), that has zero, one, two, three, four, or five amino acid residues directly preceding the articulated sequence (e.g., any one of SEQ ID NOs:22-38), and/or that has zero, one, two, three, four, or five amino acid residues directly following the articulated sequence (e.g., any one of SEQ ID NOs:22-38), provided that the antigen receptor (e.g., a CAR) maintains its basic ability to allow for binding of an antigen-binding domain to a TSHTR polypeptide. Examples of CDR2 amino acid sequences that can be included in a VH domain of an antigen-binding domain of an antigen receptor (e.g., a CAR) that can target a TSHR polypeptide include, without limitation, those set forth in Table 2.

TABLE 2
Exemplary VH domain CDR2s.

| | Sequence | SEQ ID NO |
|---|---|---|
| VH CDR2 | VKNSDGSTSYADSVKG | 22 |
| VH CDR2 | NSDGS | 23 |
| VH CDR2 | TRNGNGG | 24 |
| VH CDR2 | GNGGR | 25 |
| VH CDR2 | TRNGNGGR | 26 |
| VH CDR2 | ESAGS | 27 |
| VH CDR2 | SKESAGSTFYADSVRG | 28 |
| VH CDR2 | SGSDGT | 29 |
| VH CDR2 | SKSGSDGTTSYADSVRG | 30 |
| VH CDR2 | VKGRFTIARDN | 31 |
| VH CDR2 | LSHGKK | 32 |
| VH CDR2 | SILSHGKKTYYADSVKG | 33 |
| VH CDR2 | YGSVAGRTMT | 34 |
| VH CDR2 | VRQVPGKGLEWVS | 35 |
| VH CDR2 | YPGDSD | 36 |
| VH CDR2 | IYPGDSDTRYSPSFQG | 37 |
| VH CDR2 | IIYPYDSDTRYSPSFEG | 38 |

As used herein, a “CDR3 that consists essentially of the amino acid sequence set forth in any one of SEQ ID NOs:39-50” is a CDR3 that has zero, one, or two amino acid substitutions within the articulated sequence (e.g., any one of SEQ ID NOs:39-50), that has zero, one, two, three, four, or five amino acid residues directly preceding the articulated sequence (e.g., any one of SEQ ID NOs:39-50), and/or that has zero, one, two, three, four, or five amino acid residues directly following the articulated sequence (e.g., any one of SEQ ID NOs:39-50), provided that the antigen receptor (e.g., a CAR) maintains its basic ability to allow for binding of an antigen-binding domain to a TSHR polypeptide. Examples of CDR3 amino acid sequences that can be included in a VH domain of an antigen-binding domain of an antigen receptor (e.g., a CAR) that can target a TSHR polypeptide include, without limitation, those set forth in Table 3.

TABLE 3
Exemplary VH domain CDR3s.

| | | SEQ |
|---|---|---|
| | Sequence | ID NO |
| VH CDR3 | LKNGVFDI | 39 |
| VH CDR3 | DLGPVVRGTFDVW | 40 |
| VH CDR3 | WGQGTMVTVSS | 41 |
| VH CDR3 | DLGPVVRGTFDVWGQGTMVTVSS | 42 |
| VH CDR3 | GSARRSASGWTPYDL | 43 |
| VH CDR3 | GSAFWSGSGFFDS | 44 |
| VH CDR3 | GVNGDYFFD | 45 |
| VH CDR3 | DLVPGAGVEYSGTDV | 46 |
| VH CDR3 | DMVGATWFYGMDV | 47 |
| VH CDR3 | RFTISRDNSKNTLYLEMNGLRVEDTAVYYCAK | 48 |
| VH CDR3 | LDWNYNPLRY | 49 |
| VH CDR3 | PRDGSYPYDAFDI | 50 |

A VH domain that can be included in an antigen receptor (e.g., a CAR) that can target a TSHR polypeptide can any appropriate combination of a CDR1, a CDR2, and a CDR3. For example, a VH domain that can be included in an antigen receptor (e.g., a CAR) that can target a TSHR polypeptide can include a CDR1 having a sequence set forth in any one of SEQ ID NOs:1-21, a CDR2 having a sequence set forth in any one of SEQ ID NOs:22-38, and a CDR3 having a sequence set forth in any one of SEQ ID NOs:39-50. Examples of combinations of CDRs that can be present in a VH domain that can be included in an antigen receptor (e.g., a CAR) that can target a TSHR polypeptide include, without limitation, the combinations set forth in Table 4.

TABLE 4
Exemplary VH domains CDRs.

| | | SEQ ID | | SEQ ID | | SEQ ID |
|---|---|---|---|---|---|---|
| Clone | CDR1 | NO | CDR2 | NO | CDR3 | NO |
| 2-1 | GFSVSGN | 1 | VKNSDGSTSYADSVKG | 22 | LKNGVFDI | 39 |
| 2-1 | GNQMT | 2 | VKNSDGSTSYADSVKG | 22 | LKNGVFDI | 39 |
| 2-1 | GFSVSGNQMT | 3 | VKNSDGSTSYADSVKG | 22 | LKNGVFDI | 39 |
| 2-1 | GFSVSGN | 1 | NSDGS | 23 | LKNGVFDI | 39 |
| 2-1 | GNQMT | 2 | NSDGS | 23 | LKNGVFDI | 39 |
| 2-1 | GFSVSGNQMT | 3 | NSDGS | 23 | LKNGVFDI | 39 |
| 2-2 | GFTFTT | 4 | TRNGNGG | 24 | DLGPVVRGTFDVW | 40 |
| 2-2 | GFTFTT | 4 | GNGGR | 25 | DLGPVVRGTFDVW | 40 |
| 2-2 | GFTFTT | 4 | TRNGNGGR | 26 | DLGPVVRGTFDVW | 40 |
| 2-2 | GFTFTT | 4 | TRNGNGG | 24 | WGQGTMVTVSS | 41 |
| 2-2 | GFTFTT | 4 | GNGGR | 25 | WGQGTMVTVSS | 41 |
| 2-2 | GFTFTT | 4 | TRNGNGGR | 26 | WGQGTMVTVSS | 41 |
| 2-2 | GFTFTT | 4 | TRNGNGG | 24 | DLGPVVRGTFDVWGQGTMVTVSS | 42 |
| 2-2 | GFTFTT | 4 | GNGGR | 25 | DLGPVVRGTFDVWGQGTMVTVSS | 42 |
| 2-2 | GFTFTT | 4 | TRNGNGGR | 26 | DLGPVVRGTFDVWGQGTMVTVSS | 42 |
| 2-3 | GFSVGSA | 5 | ESAGS | 27 | GSARRSASGWTPYDL | 43 |
| 2-3 | SADMS | 6 | ESAGS | 27 | GSARRSASGWTPYDL | 43 |
| 2-3 | GFSVGSADMS | 7 | ESAGS | 27 | GSARRSASGWTPYDL | 43 |
| 2-3 | GFSVGSA | 5 | SKESAGSTFYADSVRG | 28 | GSARRSASGWTPYDL | 43 |
| 2-3 | SADMS | 6 | SKESAGSTFYADSVRG | 28 | GSARRSASGWTPYDL | 43 |
| 2-3 | GFSVGSADMS | 7 | SKESAGSTFYADSVRG | 28 | GSARRSASGWTPYDL | 43 |
| 2-4 | GFTFSNS | 8 | SGSDGT | 29 | GSAFWSGSGFFDS | 44 |
| 2-4 | NSDMA | 9 | SGSDGT | 29 | GSAFWSGSGFFDS | 44 |
| 2-4 | GFTFSNSDMA | 10 | SGSDGT | 29 | GSAFWSGSGFFDS | 44 |
| 2-4 | GFTFSNS | 8 | SKSGSDGTTSYADSVRG | 30 | GSAFWSGSGFFDS | 44 |
| 2-4 | NSDMA | 9 | SKSGSDGTTSYADSVRG | 30 | GSAFWSGSGFFDS | 44 |
| 2-4 | GFTFSNSDMA | 10 | SKSGSDGTTSYADSVRG | 30 | GSAFWSGSGFFDS | 44 |
| 2-5 | AIKYS | 11 | VKGRFTIARDN | 31 | GVNGDYFFD | 45 |
| 2-6 | GFSFNDY | 12 | LSHGKK | 32 | DLVPGAGVEYSGTDV | 46 |
| 2-6 | DYGLH | 13 | LSHGKK | 32 | DLVPGAGVEYSGTDV | 46 |
| 2-6 | GFSFNDYGLH | 14 | LSHGKK | 32 | DLVPGAGVEYSGTDV | 46 |
| 2-6 | GFSFNDY | 12 | SILSHGKKTYYADSVKG | 33 | DLVPGAGVEYSGTDV | 46 |
| 2-6 | DYGLH | 13 | SILSHGKKTYYADSVKG | 33 | DLVPGAGVEYSGTDV | 46 |
| 2-6 | GFSFNDYGLH | 14 | SILSHGKKTYYADSVKG | 33 | DLVPGAGVEYSGTDV | 46 |
| 2-8 | GFTFSNY | 15 | YGSVAGRTMT | 34 | DMVGATWFYGMDV | 47 |
| 2-8 | NYALS | 16 | YGSVAGRTMT | 34 | DMVGATWFYGMDV | 47 |
| 2-8 | GFTFSNYALS | 17 | YGSVAGRTMT | 34 | DMVGATWFYGMDV | 47 |
| 2-8 | GFTFSNY | 15 | VRQVPGKGLEWVS | 35 | DMVGATWFYGMDV | 47 |
| 2-8 | NYALS | 16 | VRQVPGKGLEWVS | 35 | DMVGATWFYGMDV | 47 |
| 2-8 | GFTFSNYALS | 17 | VRQVPGKGLEWVS | 35 | DMVGATWFYGMDV | 47 |
| 2-8 | GFTFSNY | 15 | YGSVAGRTMT | 34 | RFTISRDNSKNTLYLEMNGLRVEDTAVYYCAK | 48 |
| 2-8 | NYALS | 16 | YGSVAGRTMT | 34 | RFTISRDNSKNTLYLEMNGLRVEDTAVYYCAK | 48 |
| 2-8 | GFTFSNYALS | 17 | YGSVAGRTMT | 34 | RFTISRDNSKNTLYLEMNGLRVEDTAVYYCAK | 48 |
| 2-8 | GFTFSNY | 15 | VRQVPGKGLEWVS | 35 | RFTISRDNSKNTLYLEMNGLRVEDTAVYYCAK | 48 |
| 2-8 | NYALS | 16 | VRQVPGKGLEWVS | 35 | RFTISRDNSKNTLYLEMNGLRVEDTAVYYCAK | 48 |
| 2-8 | GFTFSNYALS | 17 | VRQVPGKGLEWVS | 35 | RFTISRDNSKNTLYLEMNGLRVEDTAVYYCAK | 48 |
| K1-70 | GYSLTDN | 18 | YPGDSD | 36 | LDWNYNPLRY | 49 |
| K1-70 | DNWIG | 19 | YPGDSD | 36 | LDWNYNPLRY | 49 |
| K1-70 | GYSLTDNWIG | 20 | YPGDSD | 36 | LDWNYNPLRY | 49 |
| K1-70 | GYSLTDN | 18 | IYPGDSDTRYSPSFQG | 37 | LDWNYNPLRY | 49 |
| K1-70 | DNWIG | 19 | IYPGDSDTRYSPSFQG | 37 | LDWNYNPLRY | 49 |
| K1-70 | GYSLTDNWIG | 20 | IYPGDSDTRYSPSFQG | 37 | LDWNYNPLRY | 49 |
| K1-18 | NYWIG | 21 | IIYPYDSDTRYSPSFEG | 38 | PRDGSYPYDAFDI | 50 |

Examples of VH domains that can be used in an antigen receptor (e.g., a CAR) that can target a TSHR polypeptide and include: (i) a CDR1 that comprises, consists essentially of, or consists of the amino acid sequence set forth in any one of SEQ ID NOs:1-21, (ii) a CDR2 that comprises, consists essentially of, or consists of the amino acid sequence set forth in any one of SEQ ID NOs:22-38, and (iii) a CDR3 that comprises, consists essentially of, or consists of the amino acid sequence set forth in any one of SEQ ID NOs:39-50 include, without limitation, the VH domains set forth in Table 5.

TABLE 5
Exemplary VH domains

| VH Amino Acid Sequence | SEQ ID NO |
|---|---|
| ESKASEVQLLESGGGLVQFRGSRRLSCAVSGFSVSGNQMTWVRQAPGK | 51 |
| GLEWLSVKNSDGSTSYADSVKGRFTIARDEVKNTVFLQMNAVRAEDT | |
| ALYYCARLKNGVFDIWGQGTMVTVSS | |
| ESKASEVQLVESGGGLVQPRGSLRLSCAASGFTFTTFAMSWVRQAPGK | 52 |
| GLEWVATRNGNGGRTYYADSVRGRFTISRDLHLQMNSLRVEDTAVYY | |
| CTKDLGPVVRGTFDVWGQGTMVTVSS | |
| ESKASEVQLLESGGRQVQPRGSLRLSCTASGFSVGSADMSWVRQAPGK | 53 |
| GPEWVSSKESAGSTFYADSVRGRFTIARDNSNNMIFLQLNSLRHEDTAV | |
| YYCVRGSARRSASGWTPYDLWGQGTLVTVSS | |
| ESKASEVQLVESGGTLKQPRGSLRLSCAASGFTFSNSDMAWVRQAPGK | 54 |
| GLEWVSSKSGSDGTTSYADSVRGRFTIARDNSKNTLYLQMNALRVEDT | |
| AVYYCVKGSAFWSGSGFFDSWGQGTLVTVSS | |
| WVRQAPGKGLEWVAIKYSGGHTGYADSVKGRFTIARDNSKNDIYLQM | 55 |
| NALRGEDTAVYYCARGVNGDYFFDYWGQGTLVTVSS | |
| ESKASEVQLVESGGGVVRPAMPLRLSCAASGFSFNDYGLHWVRQAPG | 56 |
| KGLEWVASILSHGKKTYYADSVKGRFTIARDNSENTLYLQMNNLRPGD | |
| TAVYYCAKDLVPGAGVEYSGTDVWGQGTMVTVSS | |
| ESKASEVQLVESGGGSVQPGGSLRLSCAASGFTFSNYALSWVRQVPGK | 57 |
| GLEWVSGIYGSVAGRTMTTFYADFVKGRFTISRDNSKNTLYLEMNGLR | |
| VEDTAVYYCAKDMVGATWFYGMDVWGQGTLVTVSS | |
| LVQSGAEVKKPGQSLKISCKASGYSLTDNWIGWVRQKPGKGLEWMGII | 58 |
| YPGDSDTRYSPSFQGQVTISADKSINTAYLQWSSLKASDTAIYYCVGLD | |
| WNYNPLRYWGPGTLVTVS | |
| EVQLVQSGAEVKKPGESLKISCKGSGYSFTNYWIGWVRQMPGKGLEW | 59 |
| MGIIYPYDSDTRYSPSFEGQVTISADKSIRTAYLHWSSLKASDTAMYYC | |
| VRPRDGSYPYDAFDIWGQGTMVTVSS | |

In some cases, a VL domain of an antigen-binding domain in an antigen receptor (e.g., a CAR) that can target a TSHR polypeptide can have an amino acid sequence that includes: (i) a CDR1 that comprises, consists essentially of, or consists of the amino acid sequence set forth in any one of SEQ ID NOs:60-68, (ii) a CDR2 that comprises, consists essentially of, or consists of the amino acid sequence set forth in any one of SEQ ID NOs:69-77, and (iii) a CDR3 that comprises, consists essentially of, or consists of the amino acid sequence set forth in any one of SEQ ID NOs:78-86. As used herein, a “CDR1 that consists essentially of the amino acid sequence set forth in any one of SEQ ID NOs:60-68” is a CDR1 that has zero, one, or two amino acid substitutions within the articulated sequence (e.g., any one of SEQ ID NOs:60-68), that has zero, one, two, three, four, or five amino acid residues directly preceding the articulated sequence (e.g., any one of SEQ ID NOs:60-68), and/or that has zero, one, two, three, four, or five amino acid residues directly following the articulated sequence (e.g., any one of SEQ ID NOs:60-68), provided that the antigen receptor (e.g., a CAR) maintains its basic ability to allow for binding of an antigen-binding domain to a TSHR polypeptide. Examples of CDR1 amino acid sequences that can be included in a VL domain of an antigen-binding domain of an antigen receptor (e.g., a CAR) that can target a TSHR polypeptide include, without limitation, those set forth in Table 6.

TABLE 6
Exemplary VL domain CDR1s.

| | Sequence | SEQ ID NO |
|---|---|---|
| VL CDR1 | TLRRGINLGAYGIH | 60 |
| VL CDR1 | QGDSLRSYYAT | 61 |
| VL CDR1 | TLRSDINVATQRIY | 62 |
| VL CDR1 | QGNSLRGNSAS | 63 |
| VL CDR1 | SGDALPKKYAY | 64 |
| VL CDR1 | RASQDISRYLN | 65 |
| VL CDR1 | SGSSSNIGSNTVN | 66 |
| VL CDR1 | SCSGSSSDIGSNYVS | 67 |
| VL CDR1 | RASQSVSNNYLA | 68 |

As used herein, a “CDR2 that consists essentially of the amino acid sequence set forth in any one of SEQ ID NOs:69-77” is a CDR2 that has zero, one, or two amino acid substitutions within the articulated sequence (e.g., any one of SEQ ID NOs:69-77), that has zero, one, two, three, four, or five amino acid residues directly preceding the articulated sequence (e.g., any one of SEQ ID NOs:69-77), and/or that has zero, one, two, three, four, or five amino acid residues directly following the articulated sequence (e.g., any one of SEQ ID NOs:69-77), provided that the antigen receptor (e.g., a CAR) maintains its basic ability to allow for binding of an antigen-binding domain to a TSHR polypeptide. Examples of CDR2 amino acid sequences that can be included in a VL domain of an antigen-binding domain of an antigen receptor (e.g., a CAR) that can target a TSHR polypeptide include, without limitation, those set forth in Table 7.

TABLE 7
Exemplary VL domain CDR2s.

| | Sequence | SEQ ID NO |
|---|---|---|
| VL CDR2 | HKSASDKQQGS | 69 |
| VL CDR2 | GKNNRPS | 70 |
| VL CDR2 | RYNSDSDNRLGS | 71 |
| VL CDR2 | HEDRRPS | 72 |
| VL CDR2 | EDNKRPF | 73 |
| VL CDR2 | GASSLES | 74 |
| VL CDR2 | SNNQRPS | 75 |
| VL CDR2 | DNNKRPS | 76 |
| VL CDR2 | GASSRAT | 77 |

As used herein, a “CDR3 that consists essentially of the amino acid sequence set forth in any one of SEQ ID NOs:78-86” is a CDR3 that has zero, one, or two amino acid substitutions within the articulated sequence (e.g., any one of SEQ ID NOs:78-86), that has zero, one, two, three, four, or five amino acid residues directly preceding the articulated sequence (e.g., any one of SEQ ID NOs:78-86), and/or that has zero, one, two, three, four, or five amino acid residues directly following the articulated sequence (e.g., any one of SEQ ID NOs:78-86), provided that the antigen receptor (e.g., a CAR) maintains its basic ability to allow for binding of an antigen-binding domain to a TSHR polypeptide. Examples of CDR3 amino acid sequences that can be included in a VL domain of an antigen-binding domain of an antigen receptor (e.g., a CAR) that can target a TSHR polypeptide include, without limitation, those set forth in Table 8.

TABLE 8
Exemplary VL domain CDR3s.

| | Sequence | SEQ ID NO |
|---|---|---|
| VL CDR3 | MIYYNSAWV | 78 |
| VL CDR3 | GSRDTSDNHLM | 79 |
| VL CDR3 | DYYCVIWH | 80 |
| VL CDR3 | NSRDKSDSVI | 81 |
| VL CDR3 | YSTDSSGNYRV | 82 |
| VL CDR3 | QQSFTTPYT | 83 |
| VL CDR3 | AAWDDSLSGLV | 84 |
| VL CDR3 | GTWDSRLGIAV | 85 |
| VL CDR3 | QHCGSSLRA | 86 |

A VL domain that can be included in an antigen receptor (e.g., a CAR) that can target a TSHR polypeptide can any appropriate combination of a CDR1, a CDR2, and a CDR3. For example, a VL domain that can be included in an antigen receptor (e.g., a CAR) that can target a TSHR polypeptide can include a CDR1 having a sequence set forth in any one of SEQ ID NOs:60-68, a CDR2 having a sequence set forth in any one of SEQ ID NOs:69-77, and a CDR3 having a sequence set forth in any one of SEQ ID NOs:78-86. Examples of combinations of CDRs that can be present in a VL domain that can be included in an antigen receptor (e.g., a CAR) that can target a TSHR polypeptide include, without limitation, the combinations set forth in Table 9.

TABLE 9
Exemplary VL domains CDRs.

| | | SEQ | | SEQ | | SEQ |
|---|---|---|---|---|---|---|
| Clone | CDR1 | ID NO | CDR2 | ID NO | CDR3 | ID NO |
| 2-1 | TLRRGINLGAYGIH | 60 | HKSASDKQQGS | 69 | MIYYNSAWV | 78 |
| 2-2 | QGDSLRSYYAT | 61 | GKNNRPS | 70 | GSRDTSDNHLM | 79 |
| 2-3 | TLRSDINVATQRIY | 62 | RYNSDSDNRLGS | 71 | DYYCVIWH | 80 |
| 2-4 | QGNSLRGNSAS | 63 | HEDRRPS | 72 | NSRDKSDSVI | 81 |
| 2-5 | SGDALPKKYAY | 64 | EDNKRPF | 73 | YSTDSSGNYRV | 82 |
| 2-6 | RASQDISRYLN | 65 | GASSLES | 74 | QQSFTTPYT | 83 |
| 2-8 | SGSSSNIGSNTVN | 66 | SNNQRPS | 75 | AAWDDSLSGLV | 84 |
| K1-70 | SCSGSSSDIGSNYVS | 67 | DNNKRPS | 76 | GTWDSRLGIAV | 85 |
| K1-18 | RASQSVSNNYLA | 68 | GASSRAT | 77 | QHCGSSLRA | 86 |

Examples of VL domains that can be used in an antigen receptor (e.g., a CAR) that can target a TSHR polypeptide and include: (i) a CDR1 that comprises, consists essentially of, or consists of the amino acid sequence set forth in any one of SEQ ID NOs:60-68, (ii) a CDR2 that comprises, consists essentially of, or consists of the amino acid sequence set forth in any one of SEQ ID NOs:69-77, and (iii) a CDR3 that comprises, consists essentially of, or consists of the amino acid sequence set forth in any one of SEQ ID NOs:78-86 include, without limitation, the VL domains set forth in Table 10.

TABLE 10
Exemplary VL domains

| VL Amino Acid Sequences | SEQ ID NO |
|---|---|
| QPVLTQPTSLSASPGASASLTCTLRRGINLGAYGIHWYQQRPGSPPRY | 87 |
| LLRHKSASDKQQGSGVPGRFSGSKDASANAGLLLISGLQSEDEADYY | |
| CMIYYNSAWVFGGGTKLTVLGEGK | |
| SSELTQDPTVSVALGQTVRITCQGDSLRSYYATWYQQKPGQAPILVI | 88 |
| YGKNNRPSGIPDRFSASTSGNTASLTISGAQAEDEADYYCGSRDTSD | |
| NHLMFGGGTKLTVLGEGK | |
| QAVLTQPSSLSAPPGASATLPCTLRSDINVATQRIYWYHQKPGSPLRY | 89 |
| LLRYNSDSDNRLGSGVPSRFSGSKDVSANAASLLISGLQSDDEADYY | |
| CVIWHNSAVVFGGGTKLTVLGEGK | |
| SSELTQDPAVSVALGQTVRITCQGNSLRGNSASWYQQKPGQAPRLV | 90 |
| MYHEDRRPSGVPDRFSGSSSGFISSLTITGAQAADEADYYCNSRDKS | |
| DSVIFGGGTKVTVLGEGK | |
| SYELTQPPSVSVSPGQTARITCSGDALPKKYAYWYQQKSGQAPALVI | 91 |
| YEDNKRPFGIPERFSGSRSGTTATLTISGAQVDDEADYYCYSTDSSGN | |
| YRVFGGGTKLTVLGEGK | |
| DIQMTQSPSSLSASVGARVTLTCRASQDISRYLNWYQQKSGRAPKLL | 92 |
| IYGASSLESGVPSRFSGSASGSTFTLTINSLQPEDFATYYCQQSFTTPY | |
| TFGQGTKVTVLGEGK | |
| LPVLTQPPSASGTPGQRVTISCSGSSSNIGSNTVNWYQQLPGTAPKLLI | 93 |
| YSNNQRPSGVPDRFSGSKSGTSASLAISGLRSEDEADYYCAAWDDSL | |
| SGLVFGGGTKLTVLGEGK | |
| QSVLTQPPSVSAAPGQKVTISCSGSSSDIGSNYVSWYQQFPGTAPKLL | 94 |
| IYDNNKRPSAIPDRFSGSKSGTSATLGITGLQTGDEADYYCGTWDSR | |
| LGIAVFGGGTQLTV | |
| EIVLTQSPGTLSLSPGERATLSCRASQSVSNNYLAWYQQKPGQAPRL | 95 |
| LIYGASSRATGIPDRFSGSGSGTDFTLTISRLEPEDFAVYYCQHCGSSL | |
| RAFGQGTKVEIKR | |

An antigen-binding domain (e.g., a scFv) of an antigen receptor (e.g., a CAR) that can target a TSHR polypeptide can include any one of the VH domains provided herein (e.g., any one of SEQ ID NOs:51-59) and any one of the VL domains provided herein (e.g., any one of SEQ ID NOs:87-95). A VH domain and a VL domain in an antigen-binding domain (e.g., a scFv) of an antigen receptor (e.g., a CAR) that can target a TSHR polypeptide can be present in any order. In some cases, an antigen-binding domain (e.g., a scFv) of an antigen receptor (e.g., a CAR) that can target a TSHR polypeptide can include an amino acid sequence of a VH domain provided herein followed by an amino acid sequence of a VL domain provided herein. In some cases, an antigen-binding domain (e.g., a scFv) of an antigen receptor (e.g., a CAR) that can target a TSHR polypeptide can include an amino acid sequence of a VL domain provided herein followed by an amino acid sequence of a VD domain provided herein.

In some cases, a VH domain and a VL domain in an antigen-binding domain (e.g., a scFv) of an antigen receptor (e.g., a CAR) that can target a TSHR polypeptide can be connected by a linker (e.g., a polypeptide linker). A linker can be any appropriate linker. A linker can be any appropriate length (e.g., can include any number of amino acids). For example, a linker can be from about 3 to about 75 (e.g., from about 3 to about 65, from about 3 to about 50, from about 5 to about 75, from about 10 to about 75, from about 5 to about 50, from about 10 to about 50, from about 10 to about 40, or from about 10 to about 30) amino acid residues in length. Examples of linkers that can be used to connect a VH domain and a VL domain in an antigen-binding domain (e.g., a scFv) of an antigen receptor (e.g., a CAR) that can target a TSHR polypeptide include, without limitation, polypeptides that comprises, consists essentially of, or consists of the amino acid sequences found in Table 11.

TABLE 11
Exemplary Linkers

| Amino Acid Sequence | SEQ ID NO |
|---|---|
| GGGGSGGGGSGGGGSGGGGS | 96 |
| GGGGSGGGGS | 97 |
| GGGGSGGGGSGGGGS | 98 |
| GGGGSGGGGSGGGGSGGGGSGGGGS | 99 |
| SGGGGSGGGG | 100 |
| SGGGGSGGGGSGGGG | 101 |
| SGGGGSGGGGSGGGGSGGGG | 102 |
| SGGGGSGGGGSGGGGSGGGGSGGGG | 103 |

Examples of nucleic acid sequences that can encode an antigen-binding domain (e.g., a ScFv) that can be included in an antigen receptor (e.g., a CAR) that can target a TSHR polypeptide include, without limitation, those nucleic acid sequences set forth in Table 12.

TABLE 12
Exemplary ScFvs

| Amino Acid Sequence | SEQ ID NO |
|---|---|
| ESKASEVQLLESGGGLVQFRGSRRLSCAVSGFSVSGNQMTWVRQAP | 104 |
| GKGLEWLSVKNSDGSTSYADSVKGRFTIARDEVKNTVFLQMNAVR | |
| AEDTALYYCARLKNGVFDIWGQGTMVTVSSGGGGSGGGGSGGGGS | |
| GGGGSQPVLTQPTSLSASPGASASLTCTLRRGINLGAYGIHWYQQRP | |
| GSPPRYLLRHKSASDKQQGSGVPGRFSGSKDASANAGLLLISGLQSE | |
| DEADYYCMIYYNSAWVFGGGTKLTVLGEGK | |
| QPVLTQPTSLSASPGASASLTCTLRRGINLGAYGIHWYQQRPGSPPRY | 105 |
| LLRHKSASDKQQGSGVPGRFSGSKDASANAGLLLISGLQSEDEADYY | |
| CMIYYNSAWVFGGGTKLTVLGEGKGGGGSGGGGSGGGGSGGGGSE | |
| SKASEVQLLESGGGLVQFRGSRRLSCAVSGFSVSGNQMTWVRQAPG | |
| KGLEWLSVKNSDGSTSYADSVKGRFTIARDEVKNTVFLQMNAVRAE | |
| DTALYYCARLKNGVFDIWGQGTMVTVSS | |
| ESKASEVOLVESGGGLVQPRGSLRLSCAASGFTFTTFAMSWVRQAP | 106 |
| GKGLEWVATRNGNGGRTYYADSVRGRFTISRDLHLQMNSLRVEDT | |
| AVYYCTKDLGPVVRGTFDVWGQGTMVTVSSGGGGSGGGGSGGGG | |
| SGGGGSSSELTQDPTVSVALGQTVRITCQGDSLRSYYATWYQQKPG | |
| QAPILVIYGKNNRPSGIPDRFSASTSGNTASLTISGAQAEDEADYYCG | |
| SRDTSDNHLMFGGGTKLTVLGEGK | |
| SSELTQDPTVSVALGQTVRITCQGDSLRSYYATWYQQKPGQAPILVI | 107 |
| YGKNNRPSGIPDRFSASTSGNTASLTISGAQAEDEADYYCGSRDTSD | |
| NHLMFGGGTKLTVLGEGKGGGGSGGGGSGGGGSGGGGSESKASEV | |
| QLVESGGGLVQPRGSLRLSCAASGFTFTTFAMSWVRQAPGKGLEWV | |
| ATRNGNGGRTYYADSVRGRFTISRDLHLQMNSLRVEDTAVYYCTKD | |
| LGPVVRGTFDVWGQGTMVTVSS | |
| ESKASEVQLLESGGRQVQPRGSLRLSCTASGFSVGSADMSWVRQAP | 108 |
| GKGPEWVSSKESAGSTFYADSVRGRFTIARDNSNNMIFLQLNSLRHE | |
| DTAVYYCVRGSARRSASGWTPYDLWGQGTLVTVSSGGGGSGGGGS | |
| GGGGSGGGGSQAVLTQPSSLSAPPGASATLPCTLRSDINVATQRIYW | |
| YHQKPGSPLRYLLRYNSDSDNRLGSGVPSRFSGSKDVSANAASLLIS | |
| GLQSDDEADYYCVIWHNSAVVFGGGTKLTVLGEGK | |
| QAVLTQPSSLSAPPGASATLPCTLRSDINVATQRIYWYHQKPGSPLRY | 109 |
| LLRYNSDSDNRLGSGVPSRFSGSKDVSANAASLLISGLQSDDEADYY | |
| CVIWHNSAVVFGGGTKLTVLGEGKGGGGSGGGGSGGGGSGGGGSE | |
| SKASEVQLLESGGRQVQPRGSLRLSCTASGFSVGSADMSWVRQAPG | |
| KGPEWVSSKESAGSTFYADSVRGRFTIARDNSNNMIFLQLNSLRHED | |
| TAVYYCVRGSARRSASGWTPYDLWGQGTLVTVSS | |
| ESKASEVOLVESGGTLKQPRGSLRLSCAASGFTFSNSDMAWVRQAP | 110 |
| GKGLEWVSSKSGSDGTTSYADSVRGRFTIARDNSKNTLYLQMNALR | |
| VEDTAVYYCVKGSAFWSGSGFFDSWGQGTLVTVSSGGGGSGGGGS | |
| GGGGSGGGGSSSELTQDPAVSVALGQTVRITCQGNSLRGNSASWYQ | |
| QKPGQAPRLVMYHEDRRPSGVPDRFSGSSSGFISSLTITGAQAADEA | |
| DYYCNSRDKSDSVIFGGGTKVTVLGEGK | |
| SSELTQDPAVSVALGQTVRITCQGNSLRGNSASWYQQKPGQAPRLV | 111 |
| MYHEDRRPSGVPDRFSGSSSGFISSLTITGAQAADEADYYCNSRDKS | |
| DSVIFGGGTKVTVLGEGKGGGGSGGGGSGGGGSGGGGSESKASEVQ | |
| LVESGGTLKQPRGSLRLSCAASGFTFSNSDMAWVRQAPGKGLEWVS | |
| SKSGSDGTTSYADSVRGRFTIARDNSKNTLYLQMNALRVEDTAVYY | |
| CVKGSAFWSGSGFFDSWGQGTLVTVSS | |
| WVRQAPGKGLEWVAIKYSGGHTGYADSVKGRFTIARDNSKNDIYLQ | 112 |
| MNALRGEDTAVYYCARGVNGDYFFDYWGQGTLVTVSSGGGGSGG | |
| GGSGGGGSGGGGSSYELTQPPSVSVSPGQTARITCSGDALPKKYAY | |
| WYQQKSGQAPALVIYEDNKRPFGIPERFSGSRSGTTATLTISGAQVD | |
| DEADYYCYSTDSSGNYRVFGGGTKLTVLGEGK | |
| SYELTQPPSVSVSPGQTARITCSGDALPKKYAYWYQQKSGQAPALVI | 113 |
| YEDNKRPFGIPERFSGSRSGTTATLTISGAQVDDEADYYCYSTDSSGN | |
| YRVFGGGTKLTVLGEGKGGGGSGGGGSGGGGSGGGGSWVRQAPG | |
| KGLEWVAIKYSGGHTGYADSVKGRFTIARDNSKNDIYLQMNALRGE | |
| DTAVYYCARGVNGDYFFDYWGQGTLVTVSS | |
| ESKASEVOLVESGGGVVRPAMPLRLSCAASGFSFNDYGLHWVRQAP | 114 |
| GKGLEWVASILSHGKKTYYADSVKGRFTIARDNSENTLYLQMNNLR | |
| PGDTAVYYCAKDLVPGAGVEYSGTDVWGQGTMVTVSSGGGGSGG | |
| GGSGGGGSGGGGSDIQMTQSPSSLSASVGARVTLTCRASQDISRYLN | |
| WYQQKSGRAPKLLIYGASSLESGVPSRFSGSASGSTFTLTINSLQPEDF | |
| ATYYCQQSFTTPYTFGQGTKVTVLGEGK | |
| DIQMTQSPSSLSASVGARVTLTCRASQDISRYLNWYQQKSGRAPKLL | 115 |
| IYGASSLESGVPSRFSGSASGSTFTLTINSLQPEDFATYYCQQSFTTPY | |
| TFGQGTKVTVLGEGKGGGGSGGGGSGGGGSGGGGSESKASEVQLV | |
| ESGGGVVRPAMPLRLSCAASGFSFNDYGLHWVRQAPGKGLEWVASI | |
| LSHGKKTYYADSVKGRFTIARDNSENTLYLQMNNLRPGDTAVYYCA | |
| KDLVPGAGVEYSGTDVWGQGTMVTVSS | |
| ESKASEVOLVESGGGSVQPGGSLRLSCAASGFTFSNYALSWVRQVPG | 116 |
| KGLEWVSGIYGSVAGRTMTTFYADFVKGRFTISRDNSKNTLYLEMN | |
| GLRVEDTAVYYCAKDMVGATWFYGMDVWGQGTLVTVSSGGGGS | |
| GGGGSGGGGSGGGGSLPVLTQPPSASGTPGQRVTISCSGSSSNIGSNT | |
| VNWYQQLPGTAPKLLIYSNNQRPSGVPDRFSGSKSGTSASLAISGLRS | |
| EDEADYYCAAWDDSLSGLVFGGGTKLTVLGEGK | |
| LPVLTQPPSASGTPGQRVTISCSGSSSNIGSNTVNWYQQLPGTAPKLLI | 117 |
| YSNNQRPSGVPDRFSGSKSGTSASLAISGLRSEDEADYYCAAWDDSL | |
| SGLVFGGGTKLTVLGEGKGGGGSGGGGSGGGGSGGGGSESKASEV | |
| QLVESGGGSVQPGGSLRLSCAASGFTFSNYALSWVRQVPGKGLEWV | |
| SGIYGSVAGRTMTTFYADFVKGRFTISRDNSKNTLYLEMNGLRVEDT | |
| AVYYCAKDMVGATWFYGMDVWGQGTLVTVSS | |
| LVQSGAEVKKPGQSLKISCKASGYSLTDNWIGWVRQKPGKGLEWM | 118 |
| GIIYPGDSDTRYSPSFQGQVTISADKSINTAYLQWSSLKASDTAIYYC | |
| VGLDWNYNPLRYWGPGTLVTVSGGGGSGGGGSGGGGSQSVLTQPP | |
| SVSAAPGQKVTISCSGSSSDIGSNYVSWYQQFPGTAPKLLIYDNNKRP | |
| SAIPDRFSGSKSGTSATLGITGLQTGDEADYYCGTWDSRLGIAVFGG | |
| GTQLTV | |
| QSVLTQPPSVSAAPGQKVTISCSGSSSDIGSNYVSWYQQFPGTAPKLL | 119 |
| IYDNNKRPSAIPDRFSGSKSGTSATLGITGLQTGDEADYYCGTWDSR | |
| LGIAVFGGGTQLTVGGGGSGGGGSGGGGSLVQSGAEVKKPGQSLKI | |
| SCKASGYSLTDNWIGWVRQKPGKGLEWMGIIYPGDSDTRYSPSFQG | |
| QVTISADKSINTAYLQWSSLKASDTAIYYCVGLDWNYNPLRYWGPG | |
| TLVTV | |
| EVQLVQSGAEVKKPGESLKISCKGSGYSFTNYWIGWVRQMPGKGLE | 120 |
| WMGIIYPYDSDTRYSPSFEGQVTISADKSIRTAYLHWSSLKASDTAM | |
| YYCVRPRDGSYPYDAFDIWGQGTMVTVSSGGGGSGGGGSGGGGSEI | |
| VLTQSPGTLSLSPGERATLSCRASQSVSNNYLAWYQQKPGQAPRLLI | |
| YGASSRATGIPDRFSGSGSGTDFTLTISRLEPEDFAVYYCQHCGSSLR | |
| AFGQGTKVEIKR | |
| EIVLTQSPGTLSLSPGERATLSCRASQSVSNNYLAWYQQKPGQAPRL | 121 |
| LIYGASSRATGIPDRFSGSGSGTDFTLTISRLEPEDFAVYYCQHCGSSL | |
| RAFGQGTKVEIKRGGGGSGGGGSGGGGSEVQLVQSGAEVKKPGESL | |
| KISCKGSGYSFTNYWIGWVRQMPGKGLEWMGIIYPYDSDTRYSPSFE | |
| GQVTISADKSIRTAYLHWSSLKASDTAMYYCVRPRDGSYPYDAFDI | |
| WGQGTMVTVSS | |

A chimeric antigen receptor provided herein can be designed to include an optional signal peptide, an antigen-binding domain designed to bind to a TSHTR polypeptide (e.g., a human TSHR polypeptide) as described herein, an optional hinge, a transmembrane domain, and one or more intracellular signaling domains. As described herein, the antigen binding-domain of a CAR provided herein can be designed to bind to a TSHTR polypeptide (e.g., a human TSHTR polypeptide). For example, a CAR provided herein can be designed to include the components of an antibody, antigen binding fragment, and/or antibody domain described herein (e.g., a combination of CDRs) as an antigen binding domain provided that that antigen binding domain has the ability to bind to a TSHTR polypeptide (e.g., a human TSHTR polypeptide). In some examples, a CAR provided herein can be designed to include an antigen binding domain that includes two sets of three CDRs (e.g., CDR1, CDR2, and CDR3 of a heavy chain and CDR1, CDR2, and CDR3 of a light chain) of an antigen binding fragment provided herein (e.g., SEQ ID NOs:1-3 and 5-7). In some cases, an antigen binding domain of a CAR targeting a TSHR polypeptide can be designed to include a VH domain described herein or a scFv antibody described herein.

In some cases, a CAR provided herein can be designed to include a signal peptide. Any appropriate signal peptide can be used to design a CAR described herein. Examples of signal peptide that can be used to make a CAR described herein include, without limitation, a human IGKV1-39-, IGKV1-16-, IGKV1-33-, IGKV3-11-, IGKV4-1-, or IGKV6-21-derived signal peptide.

In some cases, a CAR provided herein can be designed to include a leader polypeptide. Any appropriate leader polypeptide can be used to design a CAR described herein. Examples of leader polypeptides that can be used to make a CAR described herein include, without limitation, CD8 leader polypeptides. A CAR provided herein can be designed to include a leader polypeptide of any appropriate length. For example, a CAR provided herein can be designed to include a leader polypeptide that is from about 3 to about 75 (e.g., from about 3 to about 65, from about 3 to about 50, from about 5 to about 75, from about 10 to about 75, from about 5 to about 50, from about 10 to about 50, from about 10 to about 40, or from about 10 to about 30) amino acid residues in length. Examples of leader polypeptides that can be used to make a CAR described herein include, without limitation, a leader polypeptide that comprises, consists essentially of, or consists of the amino acid sequences found in Table 13.

TABLE 13
Exemplary Leader polypeptide

| | | |
|---|---|---|
| CD8 Leader | MALPVTALLLPLALLLHAARP | SEQ ID NO: 122 |
| Polypeptide | | |

In some cases, a CAR provided herein can be designed to include a hinge. Any appropriate hinge can be used to design a CAR described herein. Examples of hinges that can be used to make a CAR described herein include, without limitation, Ig-derived hinges (e.g., an IgG1-derived hinge, an IgG2-derived hinge, or an IgG4-derived hinge), Ig-derived hinges containing a CD2 domain and a CD3 domain, Ig-derived hinges containing a CD2 domain and lacking a CD3 domain, Ig-derived hinges containing a CD3 domain and lacking a CD2 domain, Ig-derived hinges lacking a CD2 domain and lacking a CD3 domain, CD8α-derived hinges, CD28-derived hinges, and CD3ζ-derived hinges. A CAR provided herein can be designed to include a hinge of any appropriate length. For example, a CAR provided herein can be designed to include a hinge that is from about 3 to about 75 (e.g., from about 3 to about 65, from about 3 to about 50, from about 5 to about 75, from about 10 to about 75, from about 5 to about 50, from about 10 to about 50, from about 10 to about 40, or from about 10 to about 30) amino acid residues in length. Examples of hinges that can be used to make a CAR described herein include, without limitation, hinges that comprises, consists essentially of, or consists of the amino acid sequences found in Table 14.

TABLE 14
Exemplary Hinges

| | Amino Acid Sequence | SEQ ID NO |
|---|---|---|
| CD8 Hinge | TTTPAPRPPTPAPTIASQPLSLRPEACRPAAGGAVHTRGLDFACD | 123 |
| CD28 Hinge | LEPKSCDKTHTCPPCPDPK | 124 |

A CAR provided herein can be designed to include any appropriate transmembrane domain. For example, the transmembrane domain of a CAR provided herein can be, without limitation, a CD3ζ transmembrane domain, a CD4 transmembrane domain, a CD8α transmembrane domain, a CD28 transmembrane domain, and a 4-1BB transmembrane domain. Examples of transmembrane domains that can be used to make a CAR described herein include, without limitation, the transmembrane domains that comprises, consists essentially of, or consists of the amino acid sequences found in Table 15.

TABLE 15
Exemplary Transmembrane Domains

| | | SEQ ID |
|---|---|---|
| | Amino Acid Sequence | NO |
| CD8 Transmembrane Domain | IYIWAPLAGTCGVLLLSLVITLYC | 125 |
| CD28 Transmembrane Domain | FWVLVVVGGVLACYSLLVTVAFIIFWV | 126 |

A CAR provided herein can be designed to include one or more intracellular signaling domains. For example, a CAR provided herein can be designed to include one, two, three, or four intracellular signaling domains. Any appropriate intracellular signaling domain or combination of intracellular signaling domains can be used to make a CAR described herein. For example, a CAR provided herein can be designed to include one or more intracellular signaling domains normally found within T cells or NK cells. Examples of intracellular signaling domains that can be used to make a CAR described herein include, without limitation, CD3ζ intracellular signaling domains, CD27 intracellular signaling domains, CD28 intracellular signaling domains, OX40 (CD134) intracellular signaling domains, 4-1BB (CD137) intracellular signaling domains, CD278 intracellular signaling domains, DAP10 intracellular signaling domains, and DAP12 intracellular signaling domains. In some cases, a CAR described herein can be designed to be a first generation CAR having a CD3ζ intracellular signaling domain. In some cases, a CAR described herein can be designed to be a second generation CAR having a CD28 intracellular signaling domain followed by a CD3ζ intracellular signaling domain. In some cases, a CAR described herein can be designed to be a third generation CAR having (a) a CD28 intracellular signaling domain followed by (b) a CD27 intracellular signaling domain, an OX40 intracellular signaling domains, or a 4-1BB intracellular signaling domain followed by (c) a CD3ζ intracellular signaling domain. Examples of intracellular signaling domains that can be used to make a CAR described herein include, without limitation, the intracellular signaling domains that comprises, consists essentially of, or consists of the amino acid sequences found in Table 16.

TABLE 16
Exemplary Intracellular Signaling Domains

| | Amino Acid sequence | SEQ ID NO |
|---|---|---|
| 4-1BB Signaling Domain | KRGRKKLLYIFKQPFMRPVQTTQEEDGCSCRFPE | 127 |
| | EEEGGCEL | |
| CD28 Signaling Domain | RSKRSRLLHSDYMNMTPRRPGPTRKHYQPYAPP | 128 |
| | RDFAAYRS | |
| CD3z Signaling Domain | RVKFSRSADAPAYKQGQNQLYNELNLGRREEY | 129 |
| | DVLDKRRGRDPEMGGKPRRKNPQEGLYNELQK | |
| | DKMAEAYSEIGMKGERRRGKGHDGLYQGLSTA | |
| | TKDTYDALHMQALPPR | |

A nucleic acid construct that can encode an antigen receptor (e.g., a CAR) that can target a TSHR polypeptide described herein can be in the form of a vector (e.g., a viral vector or a non-viral vector). A vector can include any appropriate nucleic acid encoding an antigen receptor (e.g., a CAR) that can target TSHR polypeptide described herein. When a vector including nucleic acid encoding an antigen receptor (e.g., a CAR) that can target a TSHR polypeptide is a viral vector, any appropriate viral vector can be used. A viral vector can be derived from a positive-strand virus or a negative-strand virus. A viral vector can be derived from a virus with a DNA genome or a RNA genome. In some cases, a viral vector can be a chimeric viral vector. In some cases, a viral vector can infect dividing cells. In some cases, a viral vector can infect non-dividing cells. Examples virus-based vectors that can include nucleic acid encoding an antigen receptor (e.g., a CAR) that can target a TSHR polypeptide include, without limitation, lentiviral vectors, retroviral vectors, adenoviral vectors, and adenovirus associated vectors. When a vector including nucleic acid encoding an antigen receptor (e.g., a CAR) that can target a TSHR polypeptide is a non-viral vector, any appropriate non-viral vector can be used. In some cases, a non-viral vector can be an expression plasmid (e.g., a cDNA expression vector).

A nucleic construct that can encode an antigen receptor (e.g., a CAR) that can target a TSHR polypeptide described herein can include a nucleic acid sequence that can encode at least part of an antigen receptor (e.g., a CAR) that can target a TSHR polypeptide. Examples of nucleic acid sequences that can encode a VH domain that can be included in an antigen receptor (e.g., a CAR) that can target a TSHR polypeptide include, without limitation, those nucleic acid sequences set forth in Table 17.

TABLE 17
Exemplary Nucleic Acid Sequences Encoding VH domains

| Nucleic Acid Sequence | SEQ ID NO |
|---|---|
| GAATCAAAAGCCTCTGAAGTCCAGCTGTTGGAAAGCGGCGGTGGTTTG | 130 |
| GTCCAATTTCGCGGCAGCCGACGCCTCTCCTGCGCGGTTTCTGGTTTCT | |
| CAGTCTCCGGTAACCAGATGACATGGGTCCGGCAAGCGCCAGGTAAGG | |
| GCCTTGAATGGCTCTCTGTAAAGAATAGTGATGGCTCCACATCATATG | |
| CAGATTCTGTAAAAGGTAGGTTCACAATCGCTCGCGACGAGGTAAAAA | |
| ACACAGTTTTTCTTCAAATGAACGCTGTACGAGCAGAGGACACCGCGT | |
| TGTATTACTGCGCTAGACTCAAGAATGGCGTGTTCGACATCTGGGGTC | |
| AGGGTACGATGGTAACGGTTAGCTCA | |
| GAAAGTAAGGCTTCCGAGGTACAGTTGGTCGAAAGTGGAGGAGGACT | 131 |
| GGTACAGCCACGAGGTAGCCTCAGACTCTCTTGCGCGGCATCAGGGTT | |
| TACTTTTACAACTTTTGCAATGTCCTGGGTGAGGCAAGCGCCGGGAAA | |
| GGGGCTGGAGTGGGTGGCGACTCGCAATGGAAACGGTGGCCGAACTT | |
| ATTATGCCGACTCAGTACGAGGCAGATTCACAATTTCACGAGACCTGC | |
| ACCTTCAGATGAACTCTTTGCGCGTTGAGGATACGGCAGTTTATTACTG | |
| CACAAAGGACCTTGGGCCAGTCGTAAGAGGCACTTTTGACGTATGGGG | |
| CCAGGGGACGATGGTTACAGTCAGCTCA | |
| GAGTCCAAGGCGAGCGAAGTACAGTTGCTTGAGTCAGGCGGGAGGCA | 132 |
| AGTTCAACCTCGCGGTTCTTTGCGACTGTCCTGTACTGCTTCAGGCTTT | |
| AGTGTGGGGTCAGCCGATATGTCATGGGTACGACAGGCGCCCGGAAA | |
| AGGCCCAGAGTGGGTCTCATCCAAGGAATCTGCAGGTAGCACCTTCTA | |
| CGCAGACAGTGTGAGAGGGAGGTTCACGATAGCGCGAGATAATAGTA | |
| ACAATATGATTTTTTTGCAGCTCAACAGTCTGCGACATGAAGACACTG | |
| CAGTTTATTACTGTGTGAGGGGTTCTGCTAGACGATCAGCATCCGGGT | |
| GGACACCTTATGATCTTTGGGGACAGGGTACTCTGGTAACGGTCAGCT | |
| CA | |
| GAGTCAAAAGCATCCGAGGTTCAACTGGTGGAATCCGGTGGAACATTG | 133 |
| AAACAACCAAGAGGTAGTCTTCGGCTGAGTTGTGCGGCATCTGGTTTC | |
| ACATTCAGTAATAGTGATATGGCATGGGTTAGGCAGGCCCCAGGCAAA | |
| GGCTTGGAATGGGTGAGTTCAAAATCAGGATCTGACGGCACTACGTCA | |
| TACGCCGATAGTGTTAGGGGTCGATTCACCATTGCTCGGGATAACTCT | |
| AAAAACACGCTTTATTTGCAGATGAACGCGCTGCGGGTGGAAGATACC | |
| GCAGTTTACTATTGTGTCAAGGGGAGTGCATTCTGGTCTGGATCTGGAT | |
| TTTTCGACTCATGGGGCCAAGGGACGCTCGTCACTGTGAGCAGT | |
| GAATCCAAAGCTAGCGAGGTGCAGCTGGTGGAGTCTGGAGGAACCTTG | 134 |
| AAACAGTCTGCGGGGTCCCTGAGACTGTCCTGTGCAGCCTCTGGATTC | |
| AGCGTCAGTGATTACCACATGAGCTGGGTCCGCCAGGCTCCAGGGAAG | |
| GGGCTGGAGTGGGTCGCAATAAAATATAGTGGTGGTCACACAGGCTAC | |
| GCAGACTCCGTGAAGGGCCGGTTCACCATCGCCAGAGACAATTCGAAG | |
| AATGACATTTATCTGCAAATGAACGCCCTGAGAGGCGAGGACACGGCC | |
| GTCTATTATTGTGCGAGAGGTGTCAACGGTGACTACTTCTTTGACTATT | |
| GGGGCCAGGGAACCCTGGTCACCGTCTCCTCA | |
| GAATCCAAAGCTAGCGAGGTGCAGCTGGTGGAGTCTGGGGGAGGCGT | 135 |
| GGTCCGGCCTGCGATGCCCCTGAGACTCTCCTGTGCAGCCTCTGGATTC | |
| TCCTTCAATGACTATGGCCTGCACTGGGTCCGTCAGGCTCCGGGCAAG | |
| GGGCTGGAGTGGGTGGCATCTATACTATCTCATGGAAAAAAAACATAC | |
| TATGCAGACTCTGTGAAGGGCCGATTCACCATCGCCAGAGACAATTCC | |
| GAGAACACCCTGTATCTGCAAATGAACAACCTGAGACCTGGGGACACG | |
| GCTGTGTATTATTGTGCGAAAGATCTGGTTCCTGGCGCTGGCGTGGAAT | |
| ACTCTGGGACGGACGTCTGGGGCCAAGGGACAATGGTCACCGTCTCTT | |
| CA | |
| GAATCCAAAGCTAGCGAGGTGCAGCTGGTGGAGTCTGGGGGAGGCTC | 136 |
| GGTTCAGCCGGGGGGGTCCCTGAGACTCTCCTGTGCAGCCTCTGGATT | |
| CACCTTTAGTAACTATGCCCTGAGCTGGGTCCGCCAGGTTCCAGGGAA | |
| GGGGCTGGAGTGGGTCTCGGGTATTTATGGTAGTGTTGCTGGCAGGAC | |
| TATGACAACTTTTTACGCAGACTTCGTGAAGGGCCGGTTCACCATCTCC | |
| AGAGACAATTCCAAGAACACCCTGTACCTGGAAATGAACGGCCTGAG | |
| AGTCGAGGACACGGCCGTATATTACTGTGCGAAAGATATGGTGGGAGC | |
| TACTTGGTTCTACGGTATGGACGTCTGGGGCCAAGGCACCCTGGTCAC | |
| CGTCTCCTCA | |
| CTGGTGCAGTCTGGAGCAGAGGTGAAAAAGCCCGGGCAGTCTCTGAA | 137 |
| GATCTCCTGTAAGGCTTCTGGATACAGCTTAACCGACAACTGGATCGG | |
| CTGGGTGCGCCAGAAGCCCGGGAAAGGCCTGGAGTGGATGGGGATCA | |
| TCTATCCTGGTGACTCTGACACCAGATACAGTCCGTCCTTCCAAGGCCA | |
| GGTCACCATCTCAGCCGACAAGTCCATCAACACCGCCTACCTGCAGTG | |
| GAGCAGCCTGAAGGCCTCGGACACCGCCATATATTACTGTGTGGGACT | |
| CGATTGGAACTACAACCCCCTGCGATACTGGGGACCGGGAACACTGGT | |
| TACCGTTTCA | |
| GAGGTGCAGCTGGTGCAGTCTGGAGCAGAGGTGAAAAAGCCCGGGGA | 138 |
| GTCTCTGAAGATCTCCTGCAAGGGTTCTGGATACAGCTTTACCAACTAC | |
| TGGATCGGCTGGGTGCGCCAGATGCCCGGGAAAGGCCTGGAGTGGAT | |
| GGGGATCATCTATCCTTATGACTCTGATACCAGATATAGCCCGTCCTTC | |
| GAAGGCCAGGTCACCATtTCAGCCGACAAGTCCATCAGGACCGCCTAC | |
| CTGCACTGGAGCAGCCTGAAGGCCTCGGACACCGCCATGTATTACTGT | |
| GTGAGACCCCGCGATGGGAGCTATCCTTATGATGCTTTTGATATCTGGG | |
| GCCAAGGGACAATGGTCACCGTCTCTTCA | |

Examples of nucleic acid sequences that can encode a VL domain that can be included in an antigen receptor (e.g., a CAR) that can target a TSHR polypeptide include, without limitation, those nucleic acid sequences set forth in Table 18.

TABLE 18
Exemplary Nucleic Acid Sequences Encoding VL domains

| Nucleic Acid Sequence | SEQ ID NO |
|---|---|
| CAGCCCGTCCTTACTCAGCCTACATCCCTGTCTGCTAGTCCTGGGGCGT | 139 |
| CCGCGAGTTTGACTTGCACCCTGCGAAGGGGTATTAATTTGGGAGCGT | |
| ATGGCATCCATTGGTACCAGCAACGGCCTGGAAGTCCCCCACGATATC | |
| TGCTCAGACACAAGAGCGCATCAGACAAGCAGCAGGGCAGTGGGGTT | |
| CCAGGGAGGTTTTCCGGGAGCAAGGACGCCAGTGCCAATGCCGGCCTC | |
| CTCCTGATTTCTGGGCTGCAATCAGAAGACGAAGCAGACTACTATTGT | |
| ATGATATACTATAACAGCGCTTGGGTCTTCGGTGGCGGCACAAAACTG | |
| ACCGTTCTGGGCGAGGGTAAA | |
| TCTTCTGAACTGACGCAGGATCCGACTGTGTCAGTCGCTTTGGGACAG | 140 |
| ACGGTACGAATCACCTGCCAAGGAGACAGTCTCCGATCTTACTATGCG | |
| ACGTGGTACCAACAGAAACCTGGGCAGGCACCTATACTGGTCATATAT | |
| GGTAAGAACAATAGACCGTCTGGAATACCTGATAGATTCTCAGCTAGC | |
| ACTAGTGGAAATACAGCAAGCCTCACTATTAGCGGGGCACAAGCCGA | |
| AGACGAGGCCGACTACTATTGTGGCTCCCGAGATACGTCAGACAATCA | |
| CCTGATGTTCGGCGGCGGCACTAAGCTCACGGTCTTGGGGGAAGGGAA | |
| G | |
| CAGGCAGTCCTTACCCAACCCAGTAGCTTGTCAGCTCCTCCAGGAGCG | 141 |
| TCAGCGACGCTCCCATGTACATTGAGGAGCGACATTAACGTGGCTACT | |
| CAAAGGATATACTGGTATCACCAAAAACCTGGTTCACCATTGCGATAC | |
| CTGCTTCGATACAACAGTGACTCTGACAATCGGCTGGGTTCAGGTGTA | |
| CCTAGCCGCTTCAGTGGCAGCAAAGATGTAAGTGCTAATGCGGCCTCA | |
| CTGCTGATCTCCGGACTGCAGAGTGACGACGAGGCCGACTACTACTGT | |
| GTCATCTGGCACAATAGTGCTGTGGTTTTCGGGGGAGGCACTAAACTC | |
| ACAGTACTGGGTGAGGGAAAG | |
| TCTTCTGAGCTCACACAAGACCCAGCCGTGAGTGTTGCGTTGGGCCAG | 142 |
| ACGGTTAGGATAACATGTCAAGGTAACTCTCTTCGAGGGAATAGCGCA | |
| TCATGGTACCAACAGAAGCCTGGACAAGCCCCGAGATTGGTAATGTAC | |
| CATGAAGATCGACGGCCCAGTGGGGTGCCAGATCGCTTCAGCGGTTCC | |
| AGCTCCGGTTTCATCTCTAGCTTGACCATCACGGGAGCCCAGGCTGCA | |
| GACGAGGCCGATTACTACTGCAATAGTCGAGATAAATCCGATTCCGTT | |
| ATCTTTGGCGGCGGTACCAAGGTCACTGTGTTGGGAGAGGGGAAA | |
| TCTTATGAGCTGACACAGCCACCCTCGGTGTCAGTGTCCCCAGGACAA | 143 |
| ACGGCCAGGATCACCTGCTCTGGAGATGCATTGCCAAAAAAATATGCT | |
| TATTGGTACCAGCAGAAGTCAGGCCAGGCCCCTGCACTGGTCATCTAT | |
| GAGGACAACAAACGACCCTTCGGGATCCCTGAGAGATTCTCTGGCTCC | |
| AGGTCAGGGACAACGGCCACCTTGACTATCAGCGGGGCCCAGGTGGA | |
| CGATGAAGCTGACTACTACTGTTACTCAACAGACAGCAGTGGTAATTA | |
| TAGGGTGTTCGGCGGAGGGACCAAGCTCACCGTCCTAGGTGAGGGTAA | |
| A | |
| GACATTCAGATGACCCAGTCTCCATCCTCCCTGTCTGCATCTGTAGGAG | 144 |
| CCAGAGTCACCCTCACTTGTCGGGCAAGTCAGGATATTAGTAGGTACT | |
| TGAATTGGTATCAGCAGAAATCAGGGAGAGCCCCTAAACTCCTGATCT | |
| ATGGTGCATCCAGTTTGGAAAGTGGGGTCCCATCAAGGTTCAGTGGCA | |
| GTGCATCTGGGTCAACTTTCACTCTCACCATCAACAGTCTACAACCTGA | |
| AGATTTTGCAACTTACTACTGTCAACAGAGTTTCACAACCCCGTATACT | |
| TTTGGCCAGGGGACCAAGGTGACCGTCCTAGGTGAGGGTAAA | |
| CTGCCTGTGCTGACTCAGCCACCCTCAGCGTCTGGGACCCCCGGGCAG | 145 |
| AGGGTCACCATCTCTTGTTCTGGAAGCAGCTCCAACATCGGAAGTAAT | |
| ACTGTAAACTGGTACCAGCAGCTCCCAGGAACGGCCCCCAAACTCCTC | |
| ATCTATAGTAATAATCAGCGGCCCTCAGGGGTCCCTGACCGATTCTCTG | |
| GCTCCAAGTCTGGCACCTCAGCCTCCCTGGCCATCAGTGGGCTCCGGTC | |
| CGAGGATGAGGCTGATTATTACTGTGCAGCATGGGATGACAGCCTGAG | |
| TGGTCTGGTATTCGGCGGAGGGACCAAGCTCACCGTCCTAGGTGAGGG | |
| TAAA | |
| CAGTCAGTGTTGACGCAGCCGCCCTCAGTGTCTGCGGCCCCAGGACAG | 146 |
| AAGGTCACCATTTCCTGCTCCGGAAGCAGCTCCGACATTGGGAGTAAT | |
| TATGTATCCTGGTACCAGCAGTTCCCGGGAACAGCCCCCAAACTCCTC | |
| ATTTATGACAATAATAAGCGACCCTCAGCGATTCCTGACCGATTCTCTG | |
| GCTCCAAGTCTGGCACGTCAGCCACCCTGGGCATCACCGGACTCCAGA | |
| CTGGGGACGAGGCCGATTATTACTGCGGAACATGGGATAGCAGACTGG | |
| GTATTGCTGTGTTCGGAGGAGGCACCCAGCTGACCGTC | |
| GAAATTGTGTTGACGCAGTCTCCAGGCACCCTGTCTTTGTCTCCAGGGG | 147 |
| AAAGAGCCACCCTCTCCTGCAGGGCCAGTCAGAGTGTTAGCAACAACT | |
| ACTTAGCCTGGTACCAGCAGAAGCCTGGCCAGGCTCCCAGGCTCCTCA | |
| TCTATGGTGCATCCAGCAGGGCCACTGGCATCCCAGACAGGTTCAGTG | |
| GCAGTGGGTCTGGGACAGATTTCACTTTAACCATCAGCAGACTGGAGC | |
| CTGAAGATTTTGCAGTGTATTACTGTCAGCATTGTGGTAGCTCACTGAG | |
| GGCGTTCGGCCAAGGGACCAAGGTGGAAATCAAACGA | |

Examples of nucleic acid sequences that can encode an antigen-binding domain (e.g., a ScFv) that can be included in an antigen receptor (e.g., a CAR) that can target a TSHR polypeptide include, without limitation, those nucleic acid sequences set forth in Table 19.

TABLE 19
Exemplary Nucleic Acid Sequences Encoding ScFvs

| Nucleic Acid Sequence | SEQ ID NO |
|---|---|
| GAATCAAAAGCCTCTGAAGTCCAGCTGTTGGAAAGCGGCGGTGGTTTG | 148 |
| GTCCAATTTCGCGGCAGCCGACGCCTCTCCTGCGCGGTTTCTGGTTTCT | |
| CAGTCTCCGGTAACCAGATGACATGGGTCCGGCAAGCGCCAGGTAAGG | |
| GCCTTGAATGGCTCTCTGTAAAGAATAGTGATGGCTCCACATCATATG | |
| CAGATTCTGTAAAAGGTAGGTTCACAATCGCTCGCGACGAGGTAAAAA | |
| ACACAGTTTTTCTTCAAATGAACGCTGTACGAGCAGAGGACACCGCGT | |
| TGTATTACTGCGCTAGACTCAAGAATGGCGTGTTCGACATCTGGGGTC | |
| AGGGTACGATGGTAACGGTTAGCTCAGGTGGAGGTGGTTCGGGAGGTG | |
| GAGGTAGCGGAGGTGGTGGATCTCAGCCCGTCCTTACTCAGCCTACAT | |
| CCCTGTCTGCTAGTCCTGGGGCGTCCGCGAGTTTGACTTGCACCCTGCG | |
| AAGGGGTATTAATTTGGGAGCGTATGGCATCCATTGGTACCAGCAACG | |
| GCCTGGAAGTCCCCCACGATATCTGCTCAGACACAAGAGCGCATCAGA | |
| CAAGCAGCAGGGCAGTGGGGTTCCAGGGAGGTTTTCCGGGAGCAAGG | |
| ACGCCAGTGCCAATGCCGGCCTCCTCCTGATTTCTGGGCTGCAATCAG | |
| AAGACGAAGCAGACTACTATTGTATGATATACTATAACAGCGCTTGGG | |
| TCTTCGGTGGCGGCACAAAACTGACCGTTCTGGGCGAGGGTAAA | |
| CAGCCCGTCCTTACTCAGCCTACATCCCTGTCTGCTAGTCCTGGGGCGT | 149 |
| CCGCGAGTTTGACTTGCACCCTGCGAAGGGGTATTAATTTGGGAGCGT | |
| ATGGCATCCATTGGTACCAGCAACGGCCTGGAAGTCCCCCACGATATC | |
| TGCTCAGACACAAGAGCGCATCAGACAAGCAGCAGGGCAGTGGGGTT | |
| CCAGGGAGGTTTTCCGGGAGCAAGGACGCCAGTGCCAATGCCGGCCTC | |
| CTCCTGATTTCTGGGCTGCAATCAGAAGACGAAGCAGACTACTATTGT | |
| ATGATATACTATAACAGCGCTTGGGTCTTCGGTGGCGGCACAAAACTG | |
| ACCGTTCTGGGCGAGGGTAAAGGTGGAGGTGGTTCGGGAGGTGGAGG | |
| TAGCGGAGGTGGTGGATCTGAATCAAAAGCCTCTGAAGTCCAGCTGTT | |
| GGAAAGCGGCGGTGGTTTGGTCCAATTTCGCGGCAGCCGACGCCTCTC | |
| CTGCGCGGTTTCTGGTTTCTCAGTCTCCGGTAACCAGATGACATGGGTC | |
| CGGCAAGCGCCAGGTAAGGGCCTTGAATGGCTCTCTGTAAAGAATAGT | |
| GATGGCTCCACATCATATGCAGATTCTGTAAAAGGTAGGTTCACAATC | |
| GCTCGCGACGAGGTAAAAAACACAGTTTTTCTTCAAATGAACGCTGTA | |
| CGAGCAGAGGACACCGCGTTGTATTACTGCGCTAGACTCAAGAATGGC | |
| GTGTTCGACATCTGGGGTCAGGGTACGATGGTAACGGTTAGCTCA | |
| GAAAGTAAGGCTTCCGAGGTACAGTTGGTCGAAAGTGGAGGAGGACT | 150 |
| GGTACAGCCACGAGGTAGCCTCAGACTCTCTTGCGCGGCATCAGGGTT | |
| TACTTTTACAACTTTTGCAATGTCCTGGGTGAGGCAAGCGCCGGGAAA | |
| GGGGCTGGAGTGGGTGGCGACTCGCAATGGAAACGGTGGCCGAACTT | |
| ATTATGCCGACTCAGTACGAGGCAGATTCACAATTTCACGAGACCTGC | |
| ACCTTCAGATGAACTCTTTGCGCGTTGAGGATACGGCAGTTTATTACTG | |
| CACAAAGGACCTTGGGCCAGTCGTAAGAGGCACTTTTGACGTATGGGG | |
| CCAGGGGACGATGGTTACAGTCAGCTCAGGTGGAGGTGGTTCGGGAG | |
| GTGGAGGTAGCGGAGGTGGTGGATCTTCTTCTGAACTGACGCAGGATC | |
| CGACTGTGTCAGTCGCTTTGGGACAGACGGTACGAATCACCTGCCAAG | |
| GAGACAGTCTCCGATCTTACTATGCGACGTGGTACCAACAGAAACCTG | |
| GGCAGGCACCTATACTGGTCATATATGGTAAGAACAATAGACCGTCTG | |
| GAATACCTGATAGATTCTCAGCTAGCACTAGTGGAAATACAGCAAGCC | |
| TCACTATTAGCGGGGCACAAGCCGAAGACGAGGCCGACTACTATTGTG | |
| GCTCCCGAGATACGTCAGACAATCACCTGATGTTCGGCGGCGGCACTA | |
| AGCTCACGGTCTTGGGGGAAGGGAAG | |
| TCTTCTGAACTGACGCAGGATCCGACTGTGTCAGTCGCTTTGGGACAG | 151 |
| ACGGTACGAATCACCTGCCAAGGAGACAGTCTCCGATCTTACTATGCG | |
| ACGTGGTACCAACAGAAACCTGGGCAGGCACCTATACTGGTCATATAT | |
| GGTAAGAACAATAGACCGTCTGGAATACCTGATAGATTCTCAGCTAGC | |
| ACTAGTGGAAATACAGCAAGCCTCACTATTAGCGGGGCACAAGCCGA | |
| AGACGAGGCCGACTACTATTGTGGCTCCCGAGATACGTCAGACAATCA | |
| CCTGATGTTCGGCGGCGGCACTAAGCTCACGGTCTTGGGGGAAGGGAA | |
| GGGTGGAGGTGGTTCGGGAGGTGGAGGTAGCGGAGGTGGTGGATCTG | |
| AAAGTAAGGCTTCCGAGGTACAGTTGGTCGAAAGTGGAGGAGGACTG | |
| GTACAGCCACGAGGTAGCCTCAGACTCTCTTGCGCGGCATCAGGGTTT | |
| ACTTTTACAACTTTTGCAATGTCCTGGGTGAGGCAAGCGCCGGGAAAG | |
| GGGCTGGAGTGGGTGGCGACTCGCAATGGAAACGGTGGCCGAACTTAT | |
| TATGCCGACTCAGTACGAGGCAGATTCACAATTTCACGAGACCTGCAC | |
| CTTCAGATGAACTCTTTGCGCGTTGAGGATACGGCAGTTTATTACTGCA | |
| CAAAGGACCTTGGGCCAGTCGTAAGAGGCACTTTTGACGTATGGGGCC | |
| AGGGGACGATGGTTACAGTCAGCTCA | |
| GAGTCCAAGGCGAGCGAAGTACAGTTGCTTGAGTCAGGCGGGAGGCA | 152 |
| AGTTCAACCTCGCGGTTCTTTGCGACTGTCCTGTACTGCTTCAGGCTTT | |
| AGTGTGGGGTCAGCCGATATGTCATGGGTACGACAGGCGCCCGGAAA | |
| AGGCCCAGAGTGGGTCTCATCCAAGGAATCTGCAGGTAGCACCTTCTA | |
| CGCAGACAGTGTGAGAGGGAGGTTCACGATAGCGCGAGATAATAGTA | |
| ACAATATGATTTTTTTGCAGCTCAACAGTCTGCGACATGAAGACACTG | |
| CAGTTTATTACTGTGTGAGGGGTTCTGCTAGACGATCAGCATCCGGGT | |
| GGACACCTTATGATCTTTGGGGACAGGGTACTCTGGTAACGGTCAGCT | |
| CAGGTGGAGGTGGTTCGGGAGGTGGAGGTAGCGGAGGTGGTGGATCT | |
| CAGGCAGTCCTTACCCAACCCAGTAGCTTGTCAGCTCCTCCAGGAGCG | |
| TCAGCGACGCTCCCATGTACATTGAGGAGCGACATTAACGTGGCTACT | |
| CAAAGGATATACTGGTATCACCAAAAACCTGGTTCACCATTGCGATAC | |
| CTGCTTCGATACAACAGTGACTCTGACAATCGGCTGGGTTCAGGTGTA | |
| CCTAGCCGCTTCAGTGGCAGCAAAGATGTAAGTGCTAATGCGGCCTCA | |
| CTGCTGATCTCCGGACTGCAGAGTGACGACGAGGCCGACTACTACTGT | |
| GTCATCTGGCACAATAGTGCTGTGGTTTTCGGGGGAGGCACTAAACTC | |
| ACAGTACTGGGTGAGGGAAAG | |
| CAGGCAGTCCTTACCCAACCCAGTAGCTTGTCAGCTCCTCCAGGAGCG | 153 |
| TCAGCGACGCTCCCATGTACATTGAGGAGCGACATTAACGTGGCTACT | |
| CAAAGGATATACTGGTATCACCAAAAACCTGGTTCACCATTGCGATAC | |
| CTGCTTCGATACAACAGTGACTCTGACAATCGGCTGGGTTCAGGTGTA | |
| CCTAGCCGCTTCAGTGGCAGCAAAGATGTAAGTGCTAATGCGGCCTCA | |
| CTGCTGATCTCCGGACTGCAGAGTGACGACGAGGCCGACTACTACTGT | |
| GTCATCTGGCACAATAGTGCTGTGGTTTTCGGGGGAGGCACTAAACTC | |
| ACAGTACTGGGTGAGGGAAAGGGTGGAGGTGGTTCGGGAGGTGGAGG | |
| TAGCGGAGGTGGTGGATCTGAGTCCAAGGCGAGCGAAGTACAGTTGCT | |
| TGAGTCAGGCGGGAGGCAAGTTCAACCTCGCGGTTCTTTGCGACTGTC | |
| CTGTACTGCTTCAGGCTTTAGTGTGGGGTCAGCCGATATGTCATGGGTA | |
| CGACAGGCGCCCGGAAAAGGCCCAGAGTGGGTCTCATCCAAGGAATC | |
| TGCAGGTAGCACCTTCTACGCAGACAGTGTGAGAGGGAGGTTCACGAT | |
| AGCGCGAGATAATAGTAACAATATGATTTTTTTGCAGCTCAACAGTCT | |
| GCGACATGAAGACACTGCAGTTTATTACTGTGTGAGGGGTTCTGCTAG | |
| ACGATCAGCATCCGGGTGGACACCTTATGATCTTTGGGGACAGGGTAC | |
| TCTGGTAACGGTCAGCTCA | |
| GAGTCAAAAGCATCCGAGGTTCAACTGGTGGAATCCGGTGGAACATTG | 154 |
| AAACAACCAAGAGGTAGTCTTCGGCTGAGTTGTGCGGCATCTGGTTTC | |
| ACATTCAGTAATAGTGATATGGCATGGGTTAGGCAGGCCCCAGGCAAA | |
| GGCTTGGAATGGGTGAGTTCAAAATCAGGATCTGACGGCACTACGTCA | |
| TACGCCGATAGTGTTAGGGGTCGATTCACCATTGCTCGGGATAACTCT | |
| AAAAACACGCTTTATTTGCAGATGAACGCGCTGCGGGTGGAAGATACC | |
| GCAGTTTACTATTGTGTCAAGGGGAGTGCATTCTGGTCTGGATCTGGAT | |
| TTTTCGACTCATGGGGCCAAGGGACGCTCGTCACTGTGAGCAGTGGTG | |
| GAGGTGGTTCGGGAGGTGGAGGTAGCGGAGGTGGTGGATCTTCTTCTG | |
| AGCTCACACAAGACCCAGCCGTGAGTGTTGCGTTGGGCCAGACGGTTA | |
| GGATAACATGTCAAGGTAACTCTCTTCGAGGGAATAGCGCATCATGGT | |
| ACCAACAGAAGCCTGGACAAGCCCCGAGATTGGTAATGTACCATGAA | |
| GATCGACGGCCCAGTGGGGTGCCAGATCGCTTCAGCGGTTCCAGCTCC | |
| GGTTTCATCTCTAGCTTGACCATCACGGGAGCCCAGGCTGCAGACGAG | |
| GCCGATTACTACTGCAATAGTCGAGATAAATCCGATTCCGTTATCTTTG | |
| GCGGCGGTACCAAGGTCACTGTGTTGGGAGAGGGGAAA | |
| TCTTCTGAGCTCACACAAGACCCAGCCGTGAGTGTTGCGTTGGGCCAG | 155 |
| ACGGTTAGGATAACATGTCAAGGTAACTCTCTTCGAGGGAATAGCGCA | |
| TCATGGTACCAACAGAAGCCTGGACAAGCCCCGAGATTGGTAATGTAC | |
| CATGAAGATCGACGGCCCAGTGGGGTGCCAGATCGCTTCAGCGGTTCC | |
| AGCTCCGGTTTCATCTCTAGCTTGACCATCACGGGAGCCCAGGCTGCA | |
| GACGAGGCCGATTACTACTGCAATAGTCGAGATAAATCCGATTCCGTT | |
| ATCTTTGGCGGCGGTACCAAGGTCACTGTGTTGGGAGAGGGGAAAGGT | |
| GGAGGTGGTTCGGGAGGTGGAGGTAGCGGAGGTGGTGGATCTGAGTC | |
| AAAAGCATCCGAGGTTCAACTGGTGGAATCCGGTGGAACATTGAAACA | |
| ACCAAGAGGTAGTCTTCGGCTGAGTTGTGCGGCATCTGGTTTCACATTC | |
| AGTAATAGTGATATGGCATGGGTTAGGCAGGCCCCAGGCAAAGGCTTG | |
| GAATGGGTGAGTTCAAAATCAGGATCTGACGGCACTACGTCATACGCC | |
| GATAGTGTTAGGGGTCGATTCACCATTGCTCGGGATAACTCTAAAAAC | |
| ACGCTTTATTTGCAGATGAACGCGCTGCGGGTGGAAGATACCGCAGTT | |
| TACTATTGTGTCAAGGGGAGTGCATTCTGGTCTGGATCTGGATTTTTCG | |
| ACTCATGGGGCCAAGGGACGCTCGTCACTGTGAGCAGT | |
| GAATCCAAAGCTAGCGAGGTGCAGCTGGTGGAGTCTGGAGGAACCTTG | 156 |
| AAACAGTCTGCGGGGTCCCTGAGACTGTCCTGTGCAGCCTCTGGATTC | |
| AGCGTCAGTGATTACCACATGAGCTGGGTCCGCCAGGCTCCAGGGAAG | |
| GGGCTGGAGTGGGTCGCAATAAAATATAGTGGTGGTCACACAGGCTAC | |
| GCAGACTCCGTGAAGGGCCGGTTCACCATCGCCAGAGACAATTCGAAG | |
| AATGACATTTATCTGCAAATGAACGCCCTGAGAGGCGAGGACACGGCC | |
| GTCTATTATTGTGCGAGAGGTGTCAACGGTGACTACTTCTTTGACTATT | |
| GGGGCCAGGGAACCCTGGTCACCGTCTCCTCAGGTGGAGGTGGTTCGG | |
| GAGGTGGAGGTAGCGGAGGTGGTGGATCTTCTTATGAGCTGACACAGC | |
| CACCCTCGGTGTCAGTGTCCCCAGGACAAACGGCCAGGATCACCTGCT | |
| CTGGAGATGCATTGCCAAAAAAATATGCTTATTGGTACCAGCAGAAGT | |
| CAGGCCAGGCCCCTGCACTGGTCATCTATGAGGACAACAAACGACCCT | |
| TCGGGATCCCTGAGAGATTCTCTGGCTCCAGGTCAGGGACAACGGCCA | |
| CCTTGACTATCAGCGGGGCCCAGGTGGACGATGAAGCTGACTACTACT | |
| GTTACTCAACAGACAGCAGTGGTAATTATAGGGTGTTCGGCGGAGGGA | |
| CCAAGCTCACCGTCCTAGGTGAGGGTAAA | |
| TCTTATGAGCTGACACAGCCACCCTCGGTGTCAGTGTCCCCAGGACAA | 157 |
| ACGGCCAGGATCACCTGCTCTGGAGATGCATTGCCAAAAAAATATGCT | |
| TATTGGTACCAGCAGAAGTCAGGCCAGGCCCCTGCACTGGTCATCTAT | |
| GAGGACAACAAACGACCCTTCGGGATCCCTGAGAGATTCTCTGGCTCC | |
| AGGTCAGGGACAACGGCCACCTTGACTATCAGCGGGGCCCAGGTGGA | |
| CGATGAAGCTGACTACTACTGTTACTCAACAGACAGCAGTGGTAATTA | |
| TAGGGTGTTCGGCGGAGGGACCAAGCTCACCGTCCTAGGTGAGGGTAA | |
| AGGTGGAGGTGGTTCGGGAGGTGGAGGTAGCGGAGGTGGTGGATCTG | |
| AATCCAAAGCTAGCGAGGTGCAGCTGGTGGAGTCTGGAGGAACCTTGA | |
| AACAGTCTGCGGGGTCCCTGAGACTGTCCTGTGCAGCCTCTGGATTCA | |
| GCGTCAGTGATTACCACATGAGCTGGGTCCGCCAGGCTCCAGGGAAGG | |
| GGCTGGAGTGGGTCGCAATAAAATATAGTGGTGGTCACACAGGCTACG | |
| CAGACTCCGTGAAGGGCCGGTTCACCATCGCCAGAGACAATTCGAAGA | |
| ATGACATTTATCTGCAAATGAACGCCCTGAGAGGCGAGGACACGGCCG | |
| TCTATTATTGTGCGAGAGGTGTCAACGGTGACTACTTCTTTGACTATTG | |
| GGGCCAGGGAACCCTGGTCACCGTCTCCTCA | |
| GAATCCAAAGCTAGCGAGGTGCAGCTGGTGGAGTCTGGGGGAGGCGT | 158 |
| GGTCCGGCCTGCGATGCCCCTGAGACTCTCCTGTGCAGCCTCTGGATTC | |
| TCCTTCAATGACTATGGCCTGCACTGGGTCCGTCAGGCTCCGGGCAAG | |
| GGGCTGGAGTGGGTGGCATCTATACTATCTCATGGAAAAAAAACATAC | |
| TATGCAGACTCTGTGAAGGGCCGATTCACCATCGCCAGAGACAATTCC | |
| GAGAACACCCTGTATCTGCAAATGAACAACCTGAGACCTGGGGACACG | |
| GCTGTGTATTATTGTGCGAAAGATCTGGTTCCTGGCGCTGGCGTGGAAT | |
| ACTCTGGGACGGACGTCTGGGGCCAAGGGACAATGGTCACCGTCTCTT | |
| CAGGTGGAGGTGGTTCGGGAGGTGGAGGTAGCGGAGGTGGTGGATCT | |
| GACATTCAGATGACCCAGTCTCCATCCTCCCTGTCTGCATCTGTAGGAG | |
| CCAGAGTCACCCTCACTTGTCGGGCAAGTCAGGATATTAGTAGGTACT | |
| TGAATTGGTATCAGCAGAAATCAGGGAGAGCCCCTAAACTCCTGATCT | |
| ATGGTGCATCCAGTTTGGAAAGTGGGGTCCCATCAAGGTTCAGTGGCA | |
| GTGCATCTGGGTCAACTTTCACTCTCACCATCAACAGTCTACAACCTGA | |
| AGATTTTGCAACTTACTACTGTCAACAGAGTTTCACAACCCCGTATACT | |
| TTTGGCCAGGGGACCAAGGTGACCGTCCTAGGTGAGGGTAAA | |
| GACATTCAGATGACCCAGTCTCCATCCTCCCTGTCTGCATCTGTAGGAG | 159 |
| CCAGAGTCACCCTCACTTGTCGGGCAAGTCAGGATATTAGTAGGTACT | |
| TGAATTGGTATCAGCAGAAATCAGGGAGAGCCCCTAAACTCCTGATCT | |
| ATGGTGCATCCAGTTTGGAAAGTGGGGTCCCATCAAGGTTCAGTGGCA | |
| GTGCATCTGGGTCAACTTTCACTCTCACCATCAACAGTCTACAACCTGA | |
| AGATTTTGCAACTTACTACTGTCAACAGAGTTTCACAACCCCGTATACT | |
| TTTGGCCAGGGGACCAAGGTGACCGTCCTAGGTGAGGGTAAAGGTGG | |
| AGGTGGTTCGGGAGGTGGAGGTAGCGGAGGTGGTGGATCTGAATCCA | |
| AAGCTAGCGAGGTGCAGCTGGTGGAGTCTGGGGGAGGCGTGGTCCGG | |
| CCTGCGATGCCCCTGAGACTCTCCTGTGCAGCCTCTGGATTCTCCTTCA | |
| ATGACTATGGCCTGCACTGGGTCCGTCAGGCTCCGGGCAAGGGGCTGG | |
| AGTGGGTGGCATCTATACTATCTCATGGAAAAAAAACATACTATGCAG | |
| ACTCTGTGAAGGGCCGATTCACCATCGCCAGAGACAATTCCGAGAACA | |
| CCCTGTATCTGCAAATGAACAACCTGAGACCTGGGGACACGGCTGTGT | |
| ATTATTGTGCGAAAGATCTGGTTCCTGGCGCTGGCGTGGAATACTCTG | |
| GGACGGACGTCTGGGGCCAAGGGACAATGGTCACCGTCTCTTCA | |
| GAATCCAAAGCTAGCGAGGTGCAGCTGGTGGAGTCTGGGGGAGGCTC | 160 |
| GGTTCAGCCGGGGGGGTCCCTGAGACTCTCCTGTGCAGCCTCTGGATT | |
| CACCTTTAGTAACTATGCCCTGAGCTGGGTCCGCCAGGTTCCAGGGAA | |
| GGGGCTGGAGTGGGTCTCGGGTATTTATGGTAGTGTTGCTGGCAGGAC | |
| TATGACAACTTTTTACGCAGACTTCGTGAAGGGCCGGTTCACCATCTCC | |
| AGAGACAATTCCAAGAACACCCTGTACCTGGAAATGAACGGCCTGAG | |
| AGTCGAGGACACGGCCGTATATTACTGTGCGAAAGATATGGTGGGAGC | |
| TACTTGGTTCTACGGTATGGACGTCTGGGGCCAAGGCACCCTGGTCAC | |
| CGTCTCCTCAGGTGGAGGTGGTTCGGGAGGTGGAGGTAGCGGAGGTGG | |
| TGGATCTCTGCCTGTGCTGACTCAGCCACCCTCAGCGTCTGGGACCCCC | |
| GGGCAGAGGGTCACCATCTCTTGTTCTGGAAGCAGCTCCAACATCGGA | |
| AGTAATACTGTAAACTGGTACCAGCAGCTCCCAGGAACGGCCCCCAAA | |
| CTCCTCATCTATAGTAATAATCAGCGGCCCTCAGGGGTCCCTGACCGAT | |
| TCTCTGGCTCCAAGTCTGGCACCTCAGCCTCCCTGGCCATCAGTGGGCT | |
| CCGGTCCGAGGATGAGGCTGATTATTACTGTGCAGCATGGGATGACAG | |
| CCTGAGTGGTCTGGTATTCGGCGGAGGGACCAAGCTCACCGTCCTAGG | |
| TGAGGGTAAA | |
| CTGCCTGTGCTGACTCAGCCACCCTCAGCGTCTGGGACCCCCGGGCAG | 161 |
| AGGGTCACCATCTCTTGTTCTGGAAGCAGCTCCAACATCGGAAGTAAT | |
| ACTGTAAACTGGTACCAGCAGCTCCCAGGAACGGCCCCCAAACTCCTC | |
| ATCTATAGTAATAATCAGCGGCCCTCAGGGGTCCCTGACCGATTCTCTG | |
| GCTCCAAGTCTGGCACCTCAGCCTCCCTGGCCATCAGTGGGCTCCGGTC | |
| CGAGGATGAGGCTGATTATTACTGTGCAGCATGGGATGACAGCCTGAG | |
| TGGTCTGGTATTCGGCGGAGGGACCAAGCTCACCGTCCTAGGTGAGGG | |
| TAAAGGTGGAGGTGGTTCGGGAGGTGGAGGTAGCGGAGGTGGTGGAT | |
| CTGAATCCAAAGCTAGCGAGGTGCAGCTGGTGGAGTCTGGGGGAGGCT | |
| CGGTTCAGCCGGGGGGGTCCCTGAGACTCTCCTGTGCAGCCTCTGGAT | |
| TCACCTTTAGTAACTATGCCCTGAGCTGGGTCCGCCAGGTTCCAGGGA | |
| AGGGGCTGGAGTGGGTCTCGGGTATTTATGGTAGTGTTGCTGGCAGGA | |
| CTATGACAACTTTTTACGCAGACTTCGTGAAGGGCCGGTTCACCATCTC | |
| CAGAGACAATTCCAAGAACACCCTGTACCTGGAAATGAACGGCCTGAG | |
| AGTCGAGGACACGGCCGTATATTACTGTGCGAAAGATATGGTGGGAGC | |
| TACTTGGTTCTACGGTATGGACGTCTGGGGCCAAGGCACCCTGGTCAC | |
| CGTCTCCTCA | |
| CTGGTGCAGTCTGGAGCAGAGGTGAAAAAGCCCGGGCAGTCTCTGAA | 162 |
| GATCTCCTGTAAGGCTTCTGGATACAGCTTAACCGACAACTGGATCGG | |
| CTGGGTGCGCCAGAAGCCCGGGAAAGGCCTGGAGTGGATGGGGATCA | |
| TCTATCCTGGTGACTCTGACACCAGATACAGTCCGTCCTTCCAAGGCCA | |
| GGTCACCATCTCAGCCGACAAGTCCATCAACACCGCCTACCTGCAGTG | |
| GAGCAGCCTGAAGGCCTCGGACACCGCCATATATTACTGTGTGGGACT | |
| CGATTGGAACTACAACCCCCTGCGATACTGGGGACCGGGAACACTGGT | |
| TACCGTTTCAGGTGGAGGTGGTTCGGGAGGTGGAGGTAGCGGAGGTGG | |
| TGGATCTCAGTCAGTGTTGACGCAGCCGCCCTCAGTGTCTGCGGCCCC | |
| AGGACAGAAGGTCACCATTTCCTGCTCCGGAAGCAGCTCCGACATTGG | |
| GAGTAATTATGTATCCTGGTACCAGCAGTTCCCGGGAACAGCCCCCAA | |
| ACTCCTCATTTATGACAATAATAAGCGACCCTCAGCGATTCCTGACCG | |
| ATTCTCTGGCTCCAAGTCTGGCACGTCAGCCACCCTGGGCATCACCGG | |
| ACTCCAGACTGGGGACGAGGCCGATTATTACTGCGGAACATGGGATAG | |
| CAGACTGGGTATTGCTGTGTTCGGAGGAGGCACCCAGCTGACCGTC | |
| CAGTCAGTGTTGACGCAGCCGCCCTCAGTGTCTGCGGCCCCAGGACAG | 163 |
| AAGGTCACCATTTCCTGCTCCGGAAGCAGCTCCGACATTGGGAGTAAT | |
| TATGTATCCTGGTACCAGCAGTTCCCGGGAACAGCCCCCAAACTCCTC | |
| ATTTATGACAATAATAAGCGACCCTCAGCGATTCCTGACCGATTCTCTG | |
| GCTCCAAGTCTGGCACGTCAGCCACCCTGGGCATCACCGGACTCCAGA | |
| CTGGGGACGAGGCCGATTATTACTGCGGAACATGGGATAGCAGACTGG | |
| GTATTGCTGTGTTCGGAGGAGGCACCCAGCTGACCGTCGGTGGAGGTG | |
| GTTCGGGAGGTGGAGGTAGCGGAGGTGGTGGATCTCTGGTGCAGTCTG | |
| GAGCAGAGGTGAAAAAGCCCGGGCAGTCTCTGAAGATCTCCTGTAAG | |
| GCTTCTGGATACAGCTTAACCGACAACTGGATCGGCTGGGTGCGCCAG | |
| AAGCCCGGGAAAGGCCTGGAGTGGATGGGGATCATCTATCCTGGTGAC | |
| TCTGACACCAGATACAGTCCGTCCTTCCAAGGCCAGGTCACCATCTCA | |
| GCCGACAAGTCCATCAACACCGCCTACCTGCAGTGGAGCAGCCTGAAG | |
| GCCTCGGACACCGCCATATATTACTGTGTGGGACTCGATTGGAACTAC | |
| AACCCCCTGCGATACTGGGGACCGGGAACACTGGTTACCGTTTCA | |
| GAGGTGCAGCTGGTGCAGTCTGGAGCAGAGGTGAAAAAGCCCGGGGA | 164 |
| GTCTCTGAAGATCTCCTGCAAGGGTTCTGGATACAGCTTTACCAACTAC | |
| TGGATCGGCTGGGTGCGCCAGATGCCCGGGAAAGGCCTGGAGTGGAT | |
| GGGGATCATCTATCCTTATGACTCTGATACCAGATATAGCCCGTCCTTC | |
| GAAGGCCAGGTCACCATtTCAGCCGACAAGTCCATCAGGACCGCCTAC | |
| CTGCACTGGAGCAGCCTGAAGGCCTCGGACACCGCCATGTATTACTGT | |
| GTGAGACCCCGCGATGGGAGCTATCCTTATGATGCTTTTGATATCTGGG | |
| GCCAAGGGACAATGGTCACCGTCTCTTCAGGTGGAGGTGGTTCGGGAG | |
| GTGGAGGTAGCGGAGGTGGTGGATCTGAAATTGTGTTGACGCAGTCTC | |
| CAGGCACCCTGTCTTTGTCTCCAGGGGAAAGAGCCACCCTCTCCTGCA | |
| GGGCCAGTCAGAGTGTTAGCAACAACTACTTAGCCTGGTACCAGCAGA | |
| AGCCTGGCCAGGCTCCCAGGCTCCTCATCTATGGTGCATCCAGCAGGG | |
| CCACTGGCATCCCAGACAGGTTCAGTGGCAGTGGGTCTGGGACAGATT | |
| TCACTTTAACCATCAGCAGACTGGAGCCTGAAGATTTTGCAGTGTATTA | |
| CTGTCAGCATTGTGGTAGCTCACTGAGGGCGTTCGGCCAAGGGACCAA | |
| GGTGGAAATCAAACGA | |
| GAAATTGTGTTGACGCAGTCTCCAGGCACCCTGTCTTTGTCTCCAGGGG | 165 |
| AAAGAGCCACCCTCTCCTGCAGGGCCAGTCAGAGTGTTAGCAACAACT | |
| ACTTAGCCTGGTACCAGCAGAAGCCTGGCCAGGCTCCCAGGCTCCTCA | |
| TCTATGGTGCATCCAGCAGGGCCACTGGCATCCCAGACAGGTTCAGTG | |
| GCAGTGGGTCTGGGACAGATTTCACTTTAACCATCAGCAGACTGGAGC | |
| CTGAAGATTTTGCAGTGTATTACTGTCAGCATTGTGGTAGCTCACTGAG | |
| GGCGTTCGGCCAAGGGACCAAGGTGGAAATCAAACGA | |

An example of a nucleic acid sequence that can encode a leader polypeptide (e.g., a CD8 leader polypeptide) that can be included in an antigen receptor (e.g., a CAR) that can target a TSHR polypeptide includes the nucleic acid sequence set forth in SEQ ID NO:166 below.

(SEQ ID NO: 166)
ATGGCCTTACCAGTGACCGCCTTGCTCCTGCCGCTGGCCTTGCTGCTC
CACGCCGCCAGGCCG.

An example of a nucleic acid sequence that can encode a linker polypeptide that can be included in an antigen receptor (e.g., a CAR) that can target a TSHR polypeptide includes the nucleic acid sequence set forth in SEQ ID NO:167 below.

(SEQ ID NO: 167)
GGTGGAGGTGGTTCGGGAGGTGGAGGTAGCGGAGGTGGTGGATCT.

Examples of nucleic acid sequences that can encode a hinge that can be included in an antigen receptor (e.g., a CAR) that can target a TSHR polypeptide include, without limitation, those nucleic acid sequences set forth in Table 20.

TABLE 20
Exemplary Nucleic Acid Sequences Encoding Hinges

| Nucleic Acid Sequence | SEQ ID NO |
|---|---|
| ACCACTACCCCTGCACCGCGACCACCAACACCGGCGCCCACCATTGCG | 168 |
| TCGCAGCCTCTGTCCCTGCGCCCAGAAGCATGCCGTCCAGCAGCAGGT | |
| GGTGCAGTTCATACTCGTGGTCTGGATTTCGCCTGTGAT | |
| CTCGAGCCCAAATCTTGTGACAAAACTCACACATGCCCACCGTGCCCG | 169 |
| GATCCCAAA | |

Examples of nucleic acid sequences that can encode a transmembrane domain that can be included in an antigen receptor (e.g., a CAR) that can target a TSHR polypeptide include, without limitation, those nucleic acid sequences set forth in Table 21.

TABLE 21
Exemplary Nucleic Acid Sequences Encoding Transmembrane Domains

| Nucleic Acid Sequence | SEQ ID NO |
|---|---|
| ATCTACATCTGGGCGCCCTTGGCCGGGACTTGTGGGGTCCTTCTCCTGT | 170 |
| CACTGGTTATCACCCTTTACTGC | |
| TTTTGGGTGCTGGTGGTGGTTGGTGGAGTCCTGGCTTGCTATAGCTTGC | 171 |
| TAGTAACAGTGGCCTTTATTATTTTCTGGGTG | |

Examples of nucleic acid sequences that can encode an intracellular signaling domain that can be included in an antigen receptor (e.g., a CAR) that can target a TSHR polypeptide include, without limitation, those nucleic acid sequences set forth in Table 22.

TABLE 22
Exemplary Nucleic Acid Sequences Encoding Intracellular Signaling Domains

| Nucleic Acid Sequence | SEQ ID NO |
|---|---|
| AAACGGGGCAGAAAGAAACTCCTGTATATATTCAAACAACCATTTATG | 172 |
| AGACCAGTACAAACTACTCAAGAGGAAGATGGCTGTAGCTGCCGATTT | |
| CCAGAAGAAGAAGAAGGAGGATGTGAACTG | |
| AGGAGTAAGAGGAGCAGGCTCCTGCACAGTGACTACATGAACATGAC | 173 |
| TCCCCGCCGCCCCGGGCCCACCCGCAAGCATTACCAGCCCTATGCCCC | |
| ACCACGCGACTTCGCAGCCTATCGCTCC | |
| AGAGTGAAGTTCAGCAGGAGCGCAGACGCCCCCGCGTACAAGCAGGG | 174 |
| CCAGAACCAGCTCTATAACGAGCTCAATCTAGGACGAAGAGAGGAGT | |
| ACGATGTTTTGGACAAGAGACGTGGCCGGGACCCTGAGATGGGGGGA | |
| AAGCCGAGAAGGAAGAACCCTCAGGAAGGCCTGTACAATGAACTGCA | |
| GAAAGATAAGATGGCGGAGGCCTACAGTGAGATTGGGATGAAAGGCG | |
| AGCGCCGGAGGGGCAAGGGGCACGATGGCCTTTACCAGGGTCTCAGT | |
| ACAGCCACCAAGGACACCTACGACGCCCTTCACATGCAGGCCCTGCCC | |
| CCTCGC | |

In some cases, antigen receptors (e.g., CARs) that can target a TSHR polypeptide described herein and nucleic acid constructs encoding such antigen receptors (e.g., CARs) can be generated using gene editing techniques. Examples of gene editing techniques used to construct a an antigen receptor (e.g., a CAR) that can target a TSHR polypeptide include, without limitation, clustered regularly interspaced short palindromic repeats (CRISPR), zinc finger nucleases, and transcription activator-like effector nucleases (TALENs).

Any appropriate method can be used to express an antigen receptor (e.g., a CAR) that can target a TSHR polypeptide on a T cell. For example, nucleic acid encoding an antigen receptor (e.g., a CAR) can be introduced into a T cell. In some cases, nucleic acid encoding an antigen receptor (e.g., a CAR) that can target a TSHR polypeptide can be introduced into a T cell by transduction (e.g., viral transduction using a retroviral vector such as a lentiviral vector) or transfection. In some cases, nucleic acid encoding an antigen receptor (e.g., a CAR) that can target a TSHR polypeptide can be introduced ex vivo into one or more T cells. For example, ex vivo engineering of T cells can include transducing isolated T cells with a lentiviral vector encoding an antigen receptor (e.g., a CAR) that can target a TSHR polypeptide. In cases where T cells were engineered ex vivo to express an antigen receptor (e.g., a CAR) that can target a TSHR polypeptide, the T cells can be obtained from any appropriate source (e.g., a mammal such as the mammal to be treated or a donor mammal, or a cell line).

This document also provides methods and materials involved in treating cancer. For example, one or more T cells expressing (e.g., engineered to express) an antigen receptor (e.g., a CAR) that can target a TSHR polypeptide can be administered (e.g., in an adoptive cell therapy such as a CAR T cell therapy) to a mammal (e.g., a human) having cancer (e.g., thyroid cancer) to treat the mammal.

Any appropriate mammal (e.g., a human) having cancer (e.g., thyroid cancer) can be treated as described herein. Examples of mammals that can be treated as described herein include, without limitation, humans, primates (such as monkeys), dogs, cats, horses, cows, pigs, sheep, mice, and rats. In some cases, a human having cancer (e.g., thyroid cancer) can be treated with one or more T cells (e.g., one or more CAR T cells) expressing (e.g., engineered to express) an antigen receptor (e.g., a CAR) that can target a TSHR polypeptide. For example, a human having cancer (e.g., thyroid cancer) can be administered one or more T cells (e.g., one or more CAR T cells) expressing (e.g., engineered to express) an antigen receptor (e.g., a CAR) that can target a TSHR polypeptide in an adoptive T cell therapy such as a CAR T cell therapy using the methods and materials described herein.

When treating a mammal (e.g., a human) having cancer as described herein, the cancer can be any appropriate type of cancer. In some cases, a cancer that can be treated as described herein can include one or more cancer cells that express a TSHR polypeptide. In some cases, a cancer treated as described herein can include one or more solid tumors. In some cases, a cancer treated as described herein can be a primary cancer. In some cases, a cancer treated as described herein can be a metastatic cancer. In some cases, a cancer treated as described herein can be a refractory cancer. In some cases, a cancer treated as described herein can be a relapsed cancer. In some cases, a cancer treated as described herein can express a TSHR polypeptide. Examples of cancers that can be treated as described herein include, without limitation, thyroid cancers (e.g., anaplastic thyroid cancers, medullary thyroid cancers, and papillary thyroid cancers) and testicular cancers.

In some cases, the methods described herein can include identifying a mammal (e.g., a human) as having cancer (e.g., thyroid cancer). Any appropriate method can be used to identify a mammal having cancer. For example, imaging techniques and biopsy techniques can be used to identify mammals (e.g., humans) having cancer.

Any appropriate amount (e.g., number) of T cells expressing (e.g., engineered to express) an antigen receptor (e.g., a CAR) that can target a TSHR polypeptide can be administered (e.g., in an adoptive cell therapy such as a CAR T cell therapy) to a mammal (e.g., a human) having cancer (e.g., thyroid cancer). In some cases, from about 0.5×10⁶ T cells per kg body weight of the mammal (T cells/kg) to about 10×10⁶ T cells/kg expressing (e.g., engineered to express) an antigen receptor (e.g., a CAR) that can target a TSHR polypeptide can be administered to a mammal having cancer to treat the mammal. For example, a mammal having cancer can be administered a composition including from about 0.5×10⁶ T cells/kg to about 10×10⁶ T cells/kg (e.g., from about 2×10⁶ to about 10×10⁶, from about 3×10⁶ to about 10×10⁶, from about 5×10⁶ to about 10×10⁶, from about 7×10⁶ to about 10×10⁶, from about 0.5×10⁶ to about 8×10⁶, from about 0.5×10⁶ to about 5×10⁶, from about 0.5×10⁶ to about 3×10⁶, from about 1×10⁶ to about 9×10⁶, from about 2×10⁶ to about 8×10⁶, from about 3×10⁶ to about 7×10⁶, from about 4×10⁶ to about 6×10⁶, from about 1×10⁶ to about 3×10⁶, from about 2×10⁶ to about 4×10⁶, from about 3×10⁶ to about 5×10⁶, from about 4×10⁶ to about 6×10⁶, from about 5×10⁶ to about 7×10⁶, from about 6×10⁶ to about 8×10⁶, or from about 7×10⁶ to about 9×10⁶, T cells/kg).

A mammal (e.g., a human) having cancer (e.g., thyroid cancer) can be administered one or more T cells (e.g., one or more CAR T cells) expressing (e.g., engineered to express) an antigen receptor (e.g., a CAR) that can target a TSHR polypeptide using any appropriate method. For example, one or more T cells (e.g., CAR T cells) expressing (e.g., engineered to express) an antigen receptor (e.g., a CAR) that can target a TSHR polypeptide can be used in an adoptive T cell therapy (e.g., a CAR T cell therapy) to treat a mammal having cancer. In some cases, methods of treating a mammal having cancer as described herein can reduce the number of cancer cells (e.g., cancer cells expressing a TSHR polypeptide) within a mammal. In some cases, methods of treating a mammal having cancer as described herein can reduce the size of one or more tumors (e.g., tumors expressing a TSHR polypeptide) within a mammal.

In some cases, one or more T cells (e.g., one or more CAR T cells) expressing (e.g., engineered to express) an antigen receptor (e.g., a CAR) that can target a TSHR polypeptide can be administered to a mammal (e.g., a human) in need thereof (e.g., a human having cancer such as a cancer containing cancer cells that express a TSHR polypeptide) to reduce the size of the cancer present within a mammal. For example, the materials and methods described herein can be used to reduce the number of cancer cells present within a mammal having cancer by, for example, 10, 20, 30, 40, 50, 60, 70, 80, 90, 95, or more percent. For example, the materials and methods described herein can be used to reduce the size (e.g., volume) of one or more tumors present within a mammal having cancer by, for example, 10, 20, 30, 40, 50, 60, 70, 80, 90, 95, or more percent.

In some cases, one or more T cells (e.g., one or more CAR T cells) expressing (e.g., engineered to express) an antigen receptor (e.g., a CAR) that can target a TSHR polypeptide can be administered to a mammal (e.g., a human) in need thereof (e.g., a human having cancer such as a cancer containing cancer cells that express a TSHR polypeptide) to improve survival of the mammal. For example, disease-free survival (e.g., relapse-free survival) can be improved using the materials and methods described herein. For example, progression-free survival can be improved using the materials and methods described herein. In some cases, the materials and methods described herein can be used to improve the survival of a mammal having cancer by, for example, 10, 20, 30, 40, 50, 60, 70, 80, 90, 95, or more percent.

In some cases, one or more T cells (e.g., one or more CAR T cells) expressing (e.g., engineered to express) an antigen receptor (e.g., a CAR) that can target a TSHR polypeptide can be administered to a mammal (e.g., a human) in need thereof (e.g., a human having cancer such as a cancer containing cancer cells that express a TSHR polypeptide) to increase the number of tumor-infiltrating lymphocytes (e.g., T cells present in within the tumor microenvironment of a cancer) within the mammal. For example, the materials and methods described herein can be used to increase the number of tumor-infiltrating lymphocytes within a mammal having cancer by, for example, 10, 20, 30, 40, 50, 60, 70, 80, 90, 95, or more percent.

In some cases, a mammal (e.g., a human) having cancer (e.g., a thyroid cancer) can be administered a single administration of one or more T cells (e.g., one or more CAR T cells) expressing (e.g., engineered to express) an antigen receptor (e.g., a CAR) that can target a TSHR polypeptide. For example, one or more T cells (e.g., one or more CAR T cells) expressing (e.g., engineered to express) an antigen receptor (e.g., a CAR) that can target a TSHR polypeptide can be administered to a mammal having cancer (e.g., a cancer containing one or more cancer cells expressing a TSHR polypeptide) once.

In some cases, a mammal (e.g., a human) having cancer (e.g., a thyroid cancer) can be administered more than one (e.g., two, three, four, five, or more) administrations of one or more T cells (e.g., one or more CAR T cells) expressing (e.g., engineered to express) an antigen receptor (e.g., a CAR) that can target a TSHR polypeptide. For example, one or more T cells (e.g., one or more CAR T cells) expressing (e.g., engineered to express) an antigen receptor (e.g., a CAR) that can target a TSHR polypeptide can be administered to a mammal having cancer (e.g., a cancer containing one or more cancer cells expressing a TSHR polypeptide) multiple times (e.g., over a period of time ranging from days to weeks to months).

In some cases, one or more T cells (e.g., one or more CAR T cells) expressing (e.g., engineered to express) an antigen receptor (e.g., a CAR) that can target a TSHR polypeptide can be packaged for administration to a mammal having a cancer (e.g., a cancer containing one or more cancer cells expressing a TSHR polypeptide). For example, T cells (e.g., one or more CAR T cells) expressing (e.g., engineered to express) an antigen receptor (e.g., a CAR) that can target a TSHR polypeptide can be packaged in a nanoparticle (e.g., a lipid nanoparticle).

In some cases, one or more T cells (e.g., one or more CAR T cells) expressing (e.g., engineered to express) an antigen receptor (e.g., a CAR) that can target a TSHR polypeptide can be formulated into a composition (e.g., a pharmaceutically acceptable composition) for administration to a mammal having a cancer (e.g., a cancer containing one or more cancer cells expressing a TSHR polypeptide). For example, T cells (e.g., one or more CAR T cells) expressing (e.g., engineered to express) an antigen receptor (e.g., a CAR) that can target a TSHR polypeptide can be formulated together with one or more pharmaceutically acceptable carriers (additives), excipients, and/or diluents.

In some cases, a pharmaceutically acceptable carrier, excipient, or diluent can be a naturally occurring pharmaceutically acceptable carrier, excipient, or diluent. In some cases, a pharmaceutically acceptable carrier, excipient, or diluent can be a non-naturally occurring (e.g., an artificial or synthetic) pharmaceutically acceptable carrier, excipient, or diluent. Examples of pharmaceutically acceptable carriers, excipients, and diluents that can be used in a composition described herein include, without limitation, serum proteins (e.g., human serum albumin), water, salts or electrolytes (e.g., saline, protamine sulfate, and DMSO.

A composition containing T cells (e.g., one or more CAR T cells) expressing (e.g., engineered to express) an antigen receptor (e.g., a CAR) that can target a TSHR polypeptide can be designed for parenteral (including subcutaneous, intramuscular, intravenous, and intradermal) or intratumoral administration. Compositions suitable for parenteral administration include aqueous and non-aqueous sterile injection solutions that can contain anti-oxidants, buffers, bacteriostats, and solutes that render the formulation isotonic with the blood of the intended recipient.

A composition containing one or more T cells (e.g., one or more CAR T cells) expressing (e.g., engineered to express) an antigen receptor (e.g., a CAR) that can target a TSHR polypeptide can be administered using any appropriate technique and to any appropriate location. A composition including T cells (e.g., one or more CAR T cells) expressing (e.g., engineered to express) an antigen receptor (e.g., a CAR) that can target a TSHR polypeptide can be administered locally or systemically. For example, a composition provided herein can be administered locally by intratumoral administration (e.g., injection into tumors) or by administration into biological spaces infiltrated by tumors (e.g. intraspinal administration, intracerebellar administration, intraperitoneal administration and/or pleural administration). For example, a composition provided herein can be administered systemically by intravenous administration (e.g., injection or infusion) to a mammal (e.g., a human).

In certain instances, a cancer within a mammal can be monitored to evaluate the effectiveness of the cancer treatment. Any appropriate method can be used to determine whether or not a mammal having cancer is treated. For example, imaging techniques or laboratory assays can be used to assess the number of cancer cells and/or the size of a tumor present within a mammal. For example, imaging techniques or laboratory assays can be used to assess the location of cancer cells and/or a tumor present within a mammal.

In some cases, a mammal (e.g., a human) to be treated as described herein (e.g., by administering one or more T cells (e.g., CAR T cells) expressing (e.g., engineered to express) an antigen receptor (e.g., a CAR) that can target a TSHR polypeptide) also can be administered one or more agents that can increase expression of a TSHR polypeptide on one or more cells (e.g., one or more cancer cells) within the mammal. For example, one or more T cells (e.g., CAR T cells) expressing (e.g., engineered to express) an antigen receptor (e.g., a CAR) that can target a TSHR polypeptide can be administered to a mammal having cancer as a combination therapy with one or more agents that can increase expression of a TSHR polypeptide on one or more cells (e.g., one or more cancer cells) within the mammal.

In some cases, an agent that can increase expression of a TSHR polypeptide on a cell (e.g., a cancer cell) can be a MEK inhibitor. A MEK inhibitor can be an inhibitor of MEK polypeptide activity or an inhibitor of MEK polypeptide expression. Examples of compounds that can reduce or eliminate polypeptide expression of a MEK polypeptide include, without limitation, nucleic acids designed to induce RNA interference of polypeptide expression of a MEK polypeptide (e.g., a small inferring RNA or a short hairpin RNA), and antisense molecules. Examples of compounds that can reduce or eliminate polypeptide activity of a MEK polypeptide include, without limitation, small molecules that target (e.g., target and bind) to a MEK polypeptide, antibodies (e.g., neutralizing antibodies), bispecific antibodies, and Bi-specific T-cell engagers (BiTEs). When a compound that can reduce or eliminate polypeptide activity of a MEK polypeptide is a small molecule that targets (e.g., targets and binds) to a MEK polypeptide, the small molecule can be in the form of a salt (e.g., a pharmaceutically acceptable salt). Examples of MEK inhibitors include, without limitation, trametinib, binimetinib, selumetinib, cobimetinib, and any combinations thereof. When a mammal having cancer is administered both one or more T cells (e.g., CAR T cells) expressing (e.g., engineered to express) an antigen receptor (e.g., a CAR) that can target a TSHR polypeptide and one or more MEK inhibitors, the one or more MEK inhibitors can be administered to the mammal before, concurrent with, and/or after the one or more T cells (e.g., CAR T cells) expressing (e.g., engineered to express) an antigen receptor (e.g., a CAR) that can target a TSHR polypeptide. For example, a mammal (e.g., a human) can be administered one or more T cells (e.g., CAR T cells) expressing (e.g., engineered to express) an antigen receptor (e.g., a CAR) that can target a TSHR polypeptide and can be administered trametinib.

An agent that can increase expression of a TSHR polypeptide on a cell (e.g., a cancer cell) can be a BRAF inhibitor. A BRAF inhibitor can be an inhibitor of BRAF polypeptide activity or an inhibitor of BRAF polypeptide expression. Examples of compounds that can reduce or eliminate polypeptide expression of a BRAF polypeptide include, without limitation, nucleic acids designed to induce RNA interference of polypeptide expression of a BRAF polypeptide (e.g., a small inferring RNA or a short hairpin RNA), and antisense molecules. Examples of compounds that can reduce or eliminate polypeptide activity of a BRAF polypeptide include, without limitation, small molecules that target (e.g., target and bind) to a BRAF polypeptide, antibodies (e.g., neutralizing antibodies), bispecific antibodies, and BiTEs. When a compound that can reduce or eliminate polypeptide activity of a BRAF polypeptide is a small molecule that targets (e.g., targets and binds) to a BRAF polypeptide, the small molecule can be in the form of a salt (e.g., a pharmaceutically acceptable salt). Examples of BRAF inhibitors include, without limitation, vemurafenib, dabrafenib, encorafenib, and any combinations thereof. When a mammal having cancer is administered both one or more T cells (e.g., CAR T cells) expressing (e.g., engineered to express) an antigen receptor (e.g., a CAR) that can target a TSHR polypeptide and one or more BRAF inhibitors, the one or more BRAF inhibitors can be administered to the mammal before, concurrent with, and/or after the one or more T cells (e.g., CAR T cells) expressing (e.g., engineered to express) an antigen receptor (e.g., a CAR) that can target a TSHR polypeptide. For example, a mammal (e.g., a human) can be administered one or more T cells (e.g., CAR T cells) expressing (e.g., engineered to express) an antigen receptor (e.g., a CAR) that can target a TSHR polypeptide and can be administered dabrafenib.

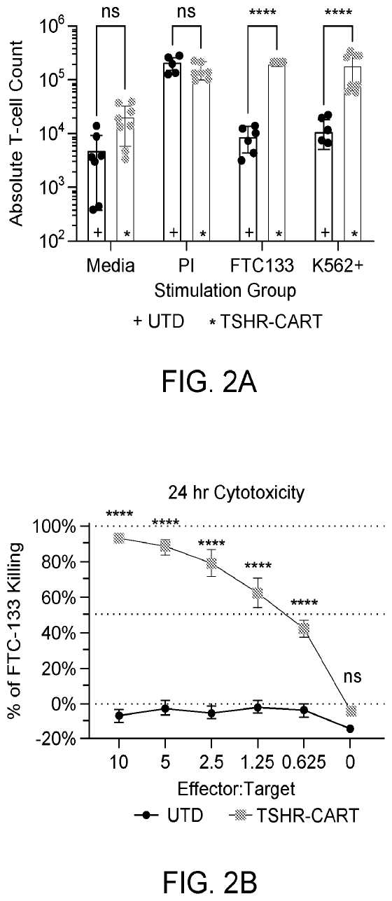

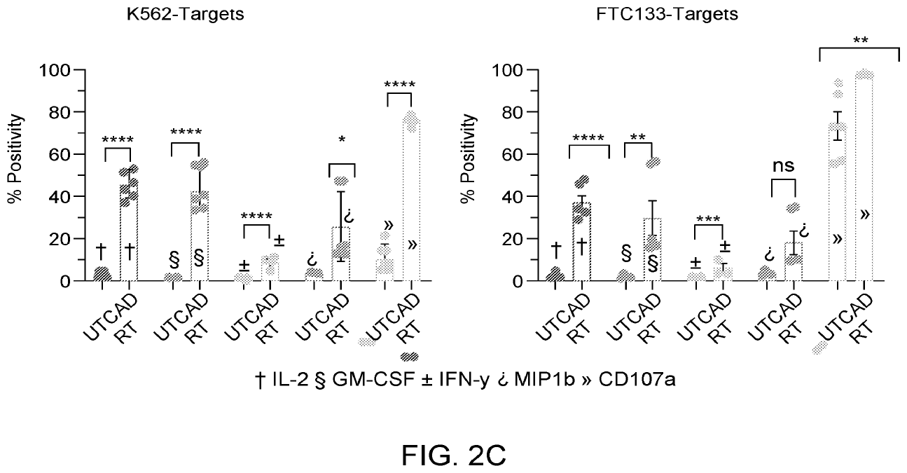

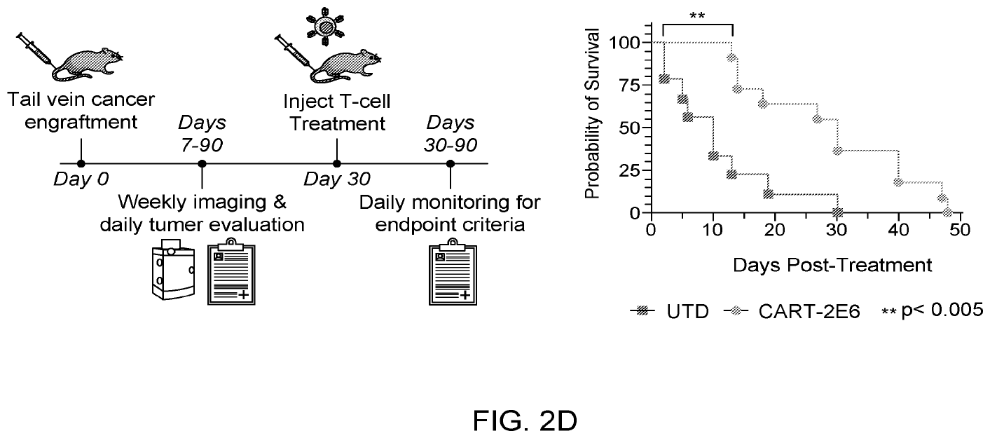

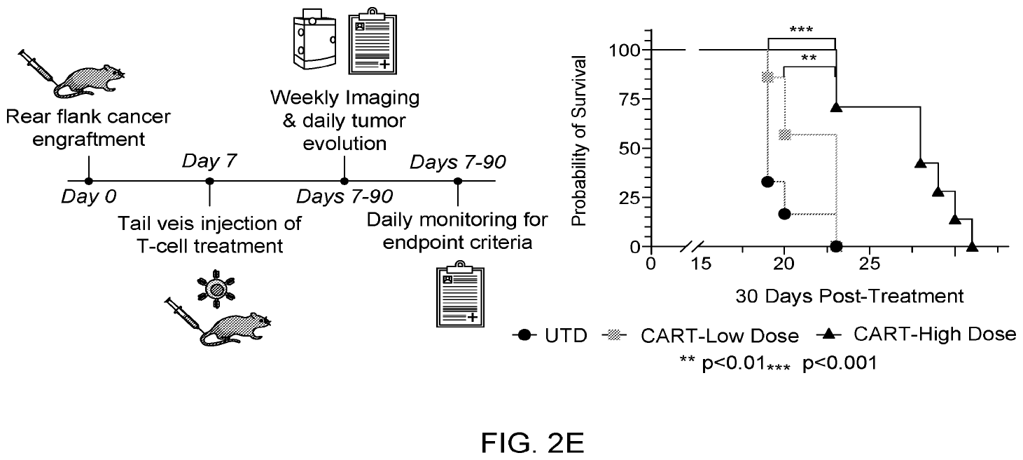

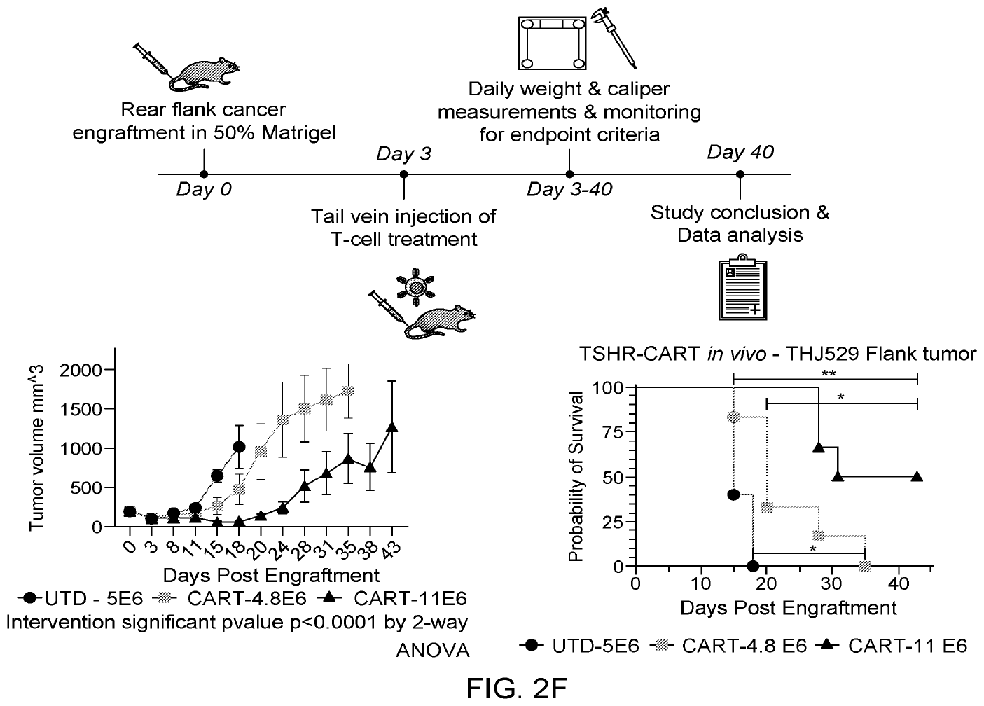

In some cases, a mammal (e.g., a human) to be treated as described herein (e.g., by administering one or more T cells (e.g., CAR T cells) expressing (e.g., engineered to express) an antigen receptor (e.g., a CAR) that can target a TSHR polypeptide) also can be administered one or more agents that can deplete a population of macrophages within the mammal. In some cases, an agent that can deplete a population of macrophages within a mammal can be an anti-CSF-1R antibody. In some cases, an agent that can depleted a population of macrophages within a mammal can be an immunomodulatory imide drug (IMiD). In some cases, an agent that can deplete a population of macrophages within a mammal can convert M2 macrophages to M1 macrophages. In some cases, an agent that can deplete a population of macrophages within a mammal can be a CSF-1R specific kinase inhibitor. Examples of agents that can deplete a population of macrophages include, without limitation, GM-CSF neutralizing antibodies, clodronate, Ki20227, pimicotinib (ABSK021), pexidartinib (PLX3397), ARRY-382, PLX7486, BLZ945, JNJ-40346527, emactuzumab, AMG820, IMC-CS4 (also referred to as LY3022855), cabiralizumab, lacnotuzumab (MCS110), PD-0360324, thalidomide, lenalidomide, pomalidomide, iberdomide, and apremilast (e.g., OTEZLA®). In some cases, an agent that can deplete a population of macrophages within a mammal can be as described elsewhere (see, e.g., Tham et al., Oncotarget, 6(26): 22857-22868 (2015); Wen et al., Eur. J. Med. Chem., 245:114884 (2023) at, for example Table 1; and Jung et al., Front. Neurosci., 15:656921 (2021) at, for example, FIG. 2).

In some cases, one or more T cells (e.g., CAR T cells) expressing (e.g., engineered to express) an antigen receptor (e.g., a CAR) that can target a TSHR polypeptide can be administered to a mammal having cancer as a combination therapy with one or more additional agents and/or therapies used to treat a cancer. In some cases, an anti-cancer agent can be a MEK inhibitor (e.g., an anti-MEK antibody). In some cases, an anti-cancer agent can be a BRAF inhibitor (e.g., an anti-BRAF antibody). In some cases, an anti-cancer agent can be a chemotherapeutic agent. In some cases, and anti-cancer agent can be a radioactive agent (e.g., a radioactive isotope). In some cases, an anti-cancer agent can be an immunotherapeutic agent. Examples of anti-cancer agents include, without limitation, trametinib, dabrafenib, binimetinib, selumetinib, vemurafenib, encorafenib, cobimetinib, busulfan, cisplatin, carboplatin, paclitaxel, docetaxel, nab-paclitaxel, altretamine, capecitabine, cyclophosphamide, etoposide (vp-16), gemcitabine, ifosfamide, irinotecan (cpt-11), liposomal doxorubicin, melphalan, pemetrexed, topotecan, vinorelbine, goserelin, leuprolide, tamoxifen, letrozole, anastrozole, exemestane, bevacizumab, olaparib, rucaparib, niraparib, and any combinations thereof. In cases where one or more T cells (e.g., CAR T cells) expressing (e.g., engineered to express) an antigen receptor (e.g., a CAR) that can target a TSHR polypeptide are used with one or more additional agents treat a cancer, the one or more additional agents can be administered at the same time or independently. In some cases, one or more T cells (e.g., CAR T cells) expressing (e.g., engineered to express) an antigen receptor (e.g., a CAR) that can target a TSHR polypeptide can be administered first, and the one or more additional agents administered second, or vice versa.

Examples of therapies that can be used to treat cancer include, without limitation, surgery, radiation therapies, and cell therapies (e.g., adaptive cell therapies). In cases where one or more T cells (e.g., CAR T cells) expressing (e.g., engineered to express) an antigen receptor (e.g., a CAR) that can target a TSHR polypeptide are used in combination with one or more additional therapies used to treat cancer, the one or more additional therapies can be performed at the same time or independently of the administration of one or more T cells (e.g., CAR T cells) expressing (e.g., engineered to express) an antigen receptor (e.g., a CAR) that can target a TSHR polypeptide. For example, the one or more T cells (e.g., CAR T cells) expressing (e.g., engineered to express) an antigen receptor (e.g., a CAR) that can target a TSHR polypeptide can be administered before, during, and/or after the one or more additional therapies are performed.

The invention will be further described in the following examples, which do not limit the scope of the invention described in the claims.

### EXAMPLES

### Example 1: Sensitization and Targeting of Aggressive Thyroid Cancers with MEK Inhibitors and CAR T Cells

This Example describes the generation of CAR T cells that can target a TSHR polypeptide. This Examples also shows that CAR T cells that can target a TSHR polypeptide demonstrated potent TSHR-specific anti-tumor affects in vitro, in vivo, and in patient-derived xenograft (PDX) mouse models.

### Results

### TSHR is an Ideal Target for CAR T Cell Therapy

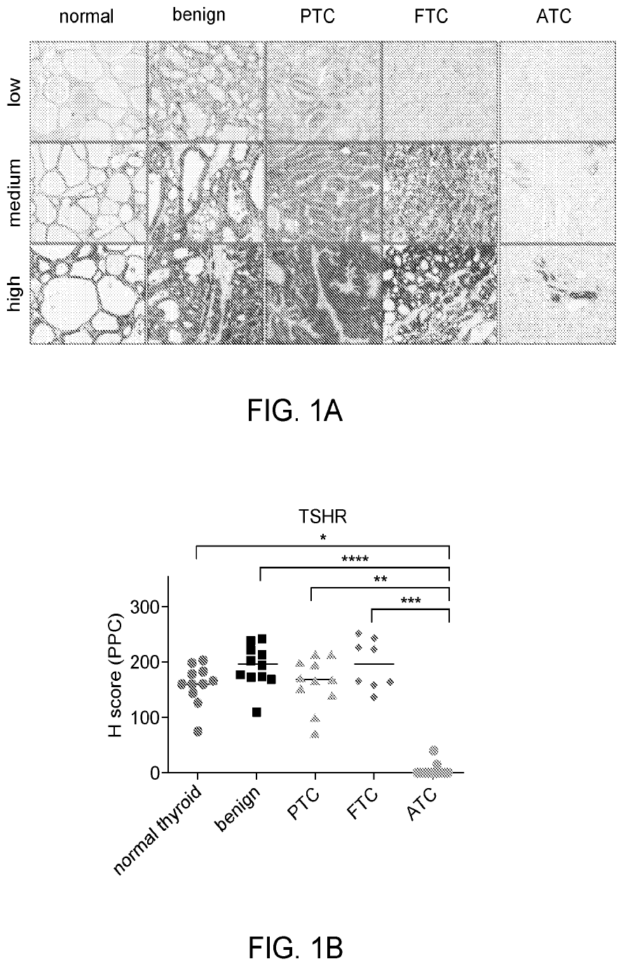

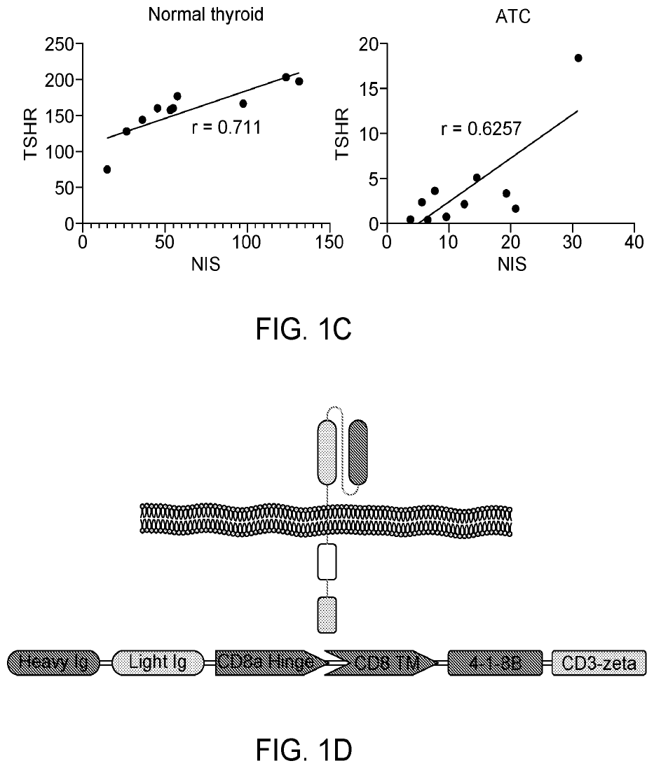

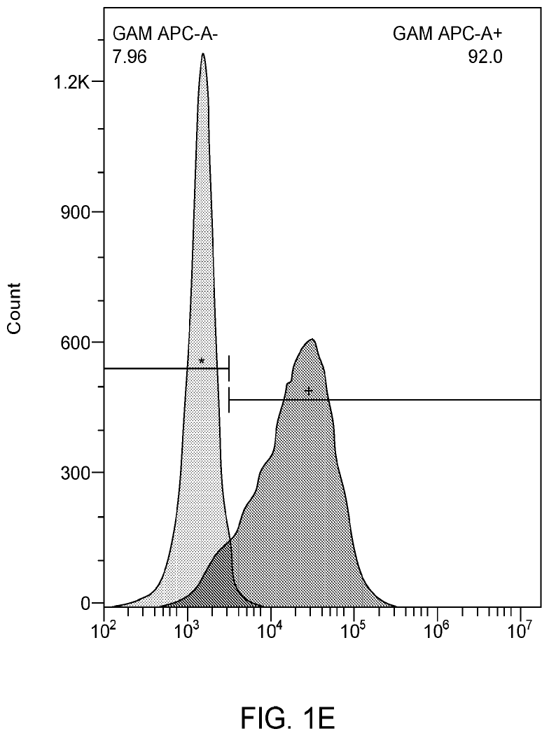

TSHR was identified as a target for CAR T cell therapy in thyroid cancer by examining previously generated gene expression databases including Gene Expression Omnibus Database (accession number GSE65144). TSHR expression was validated by IHC on patient derived samples (FIG. 1A and FIG. 1). Immunohistochemistry (IHC) for TSHR in formalin fixed paraffin embedded (FFPE) benign (N=11), papillary (PTC, N=11), follicular (FTC, N=8), anaplastic (ATC, N=) as well as normal thyroid tissues (N=11) confirmed robust expression of TSHR on thyroid cancer cells (FIG. 1C). Sodium iodide symporter (NIS) is used as a differentiation biomarker in thyroid cancer and for response to radioactive iodine. NIA was used and examined (FIG. 1C). TSHR and NIS protein expression correlated in normal and ATC tissues (FIG. 1D). Having validated TSHR as a potential viable antigen for targeting with CAR, the study designed and generated TSHR directed

### TSHR-CAR T Cells Exhibit Potent and Specific Antitumor Activity Against TSHR Expressing Tumors

Healthy donor T cells were either left as control untransduced T cells (UTD) or transduced with TSHR-CAR lentivirus and incubated with TSHR-expressing FTC133 or TSHR expressing K562 cell lines. TSHR-directed CAR T cells demonstrated profound antigen-specific effector functions against the TSHR+cancer cell lines. Antigen specific T cell proliferation, degranulation, cytotoxicity, and cytokine production was significantly higher when TSHR-CAR T cells were cocultured with the TSHR overexpressing cells lines K562 and FTC133, compared to control UTD cells (FIG. 2A). The study evaluated the antitumor activity of TSHR-CAR T cells in three different xenograft models. In the first model, the TSHR+luciferase+K562 cells lines were used to generate systemic cancer xenografts in NSG mice. Engraftment was confirmed by bioluminescence imaging (BLI) performed every week. To model metastatic disease, mice were allowed to develop disease burden. Mice were then randomized to treatment with UTD or TSHR-CAR T cells intravenously at a dose of 2 million T cells per mouse. Mice were followed by serial BLI as a measure of disease burden. Treatment with TSHR-CAR T cells led to potent antitumor effect and a significantly longer survival than the control mice (FIG. 2D).

To model solid tumor with localized masses, the NSG mice were engrafted subcutaneously with TSHR overexpressing FTC133 cells (FIG. 2E). After 1 week, UTD or CAR T cells were administered intravenously at either a high dose of 5×10⁶ or a low dose of 2×10⁶. Tumor volume and mice were monitored daily for endpoint criteria. Mice treated with the highest dose of TSHR-CAR T cells had significantly prolonged overall survival compared to the mice treated with a low dose of CAR T cell or control T cells indicating an optimal CAR T cell to tumor burden ratio exists. To further validate the antitumor activity of TSHR-CAR T cells, a PDX model was utilized with a very aggressive growing patient derived thyroid cancer cell line ATC THJ-529T cell line overexpressing TSHR (TSHR THJ-529T) (FIG. 2F). Engraftment was confirmed by caliper measurement of tumor masses. When tumor size reached 0.5 cm, mice were randomized to treatment with UTD (5×10⁶cells i.v.), TSHR-CAR T cells (5×10⁶) or TSHR-CAR T cells (10×10⁶ cells per mouse). Treatment with TSHR-CAR T cells led to decreased tumor growth and improved overall survival of the xenografts. (FIG. 2F).

### Downregulation of TSHR in Dedifferentiated Thyroid Cancer

ATC cells have been demonstrated to be associated with dedifferentiation and downregulation of TSHR expression. To examine antigen expression, TSHR expression was measured in ATC patient samples. ATC patient samples had downregulated TSHR expression compared to more differentiated thyroid cancers. (FIG. 1A).

### Meki Impact on CAR T Cell Functionality

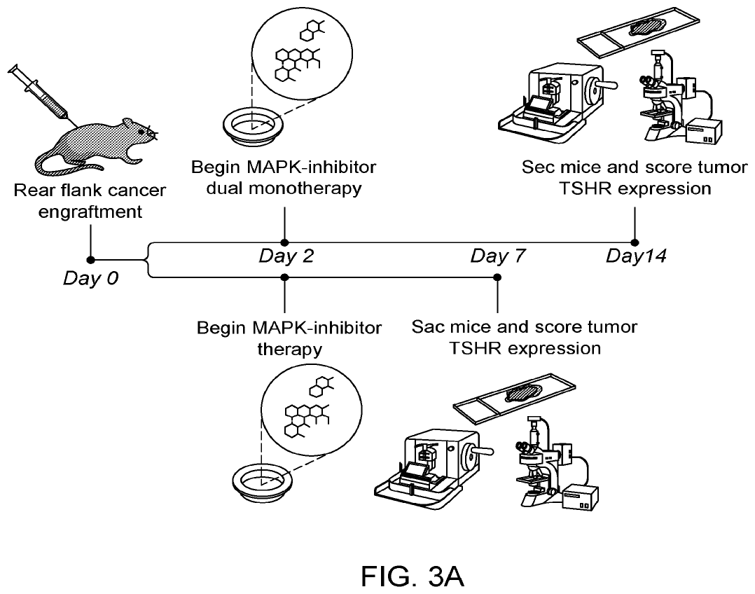

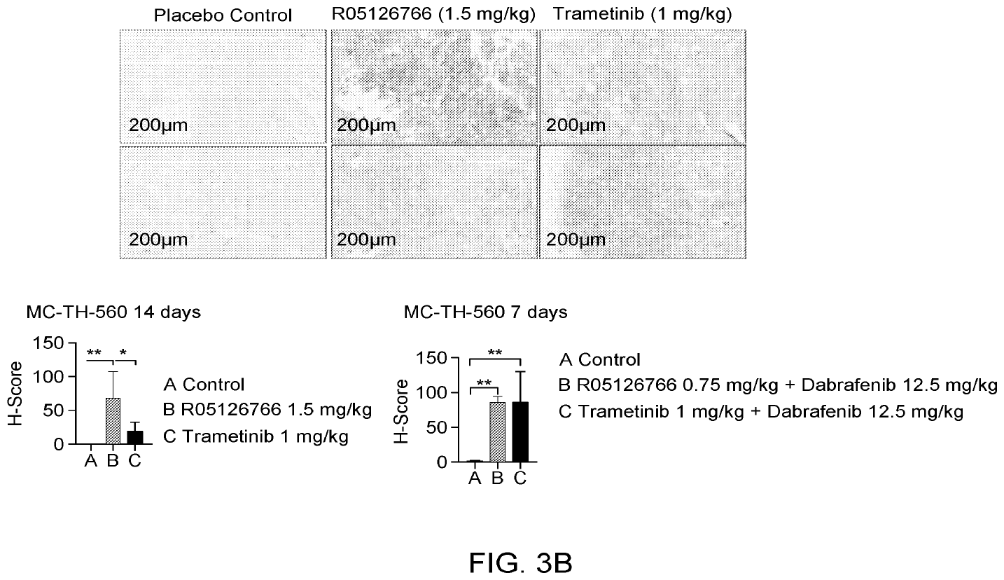

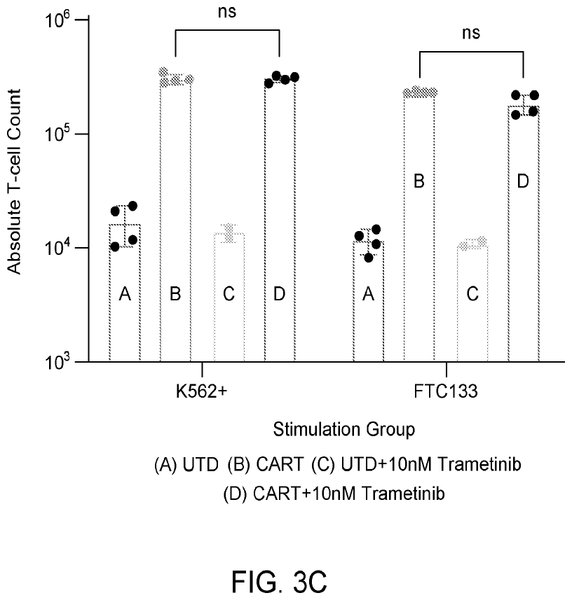

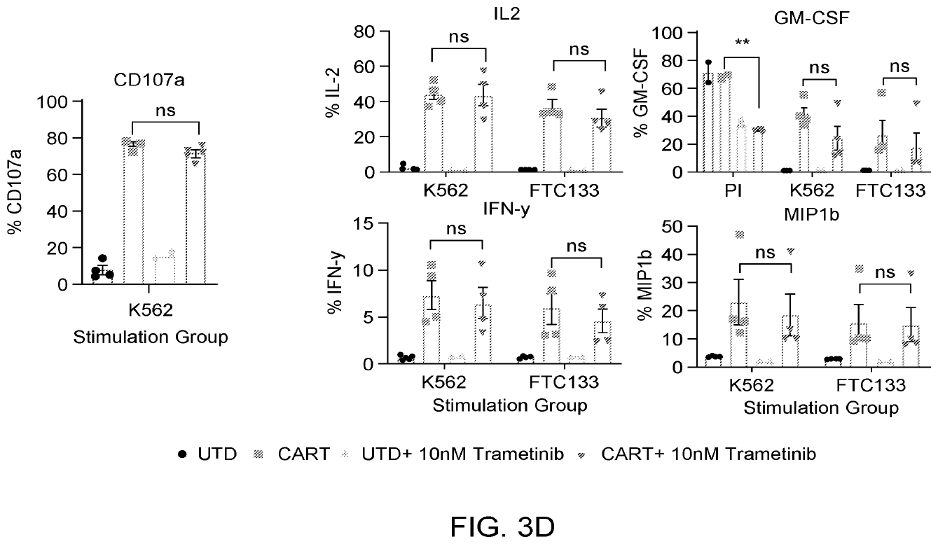

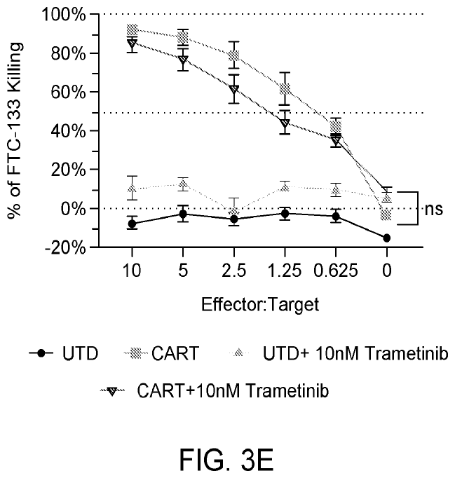

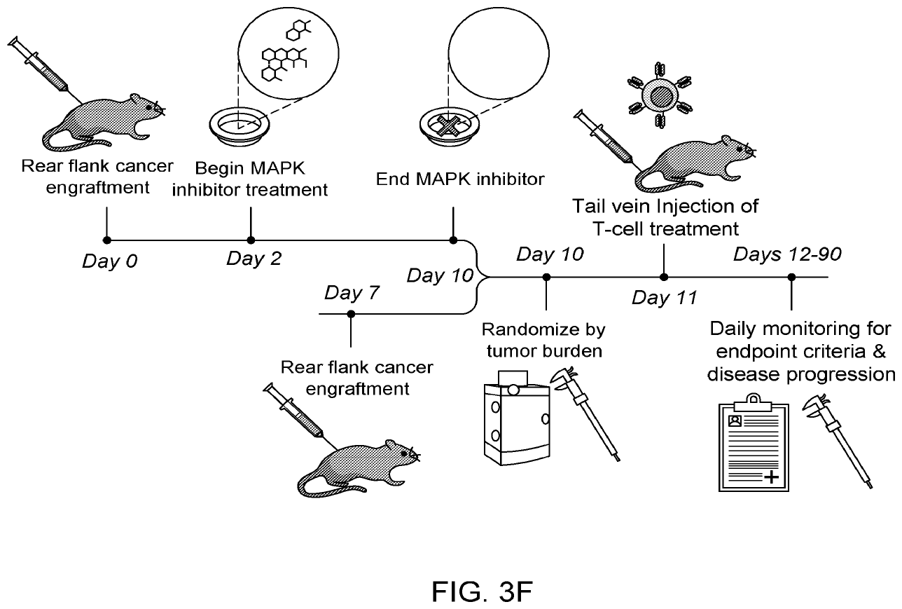

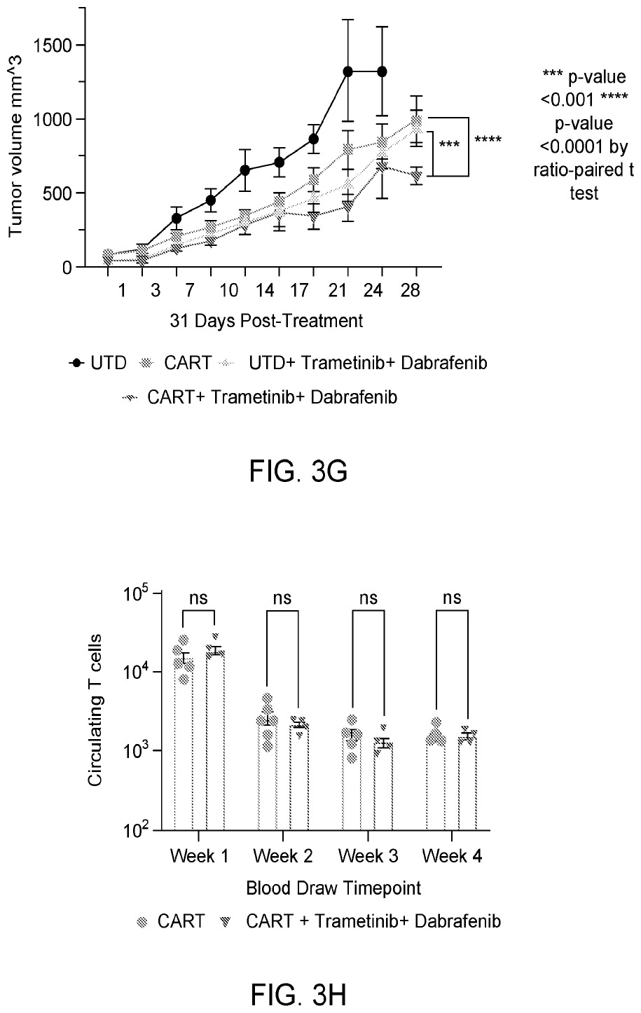

To evaluate MEK inhibition as a strategy to redifferentiate cancer cells, any detrimental effect for MEK inhibition on CAR T cell activity agonists were first examined. A panel of T cell functional assays (FIG. 3D, 3E) demonstrated that MEK inhibition caused a significant reduction in cytotoxicity, proliferation, and secretion of select cytokines of CAR T cell therapy.

### MEK and BRAF Inhibition Redifferentiates Thyroid Cancer, Increases TSHR Expression, and Enhances CAR T Cell Activity

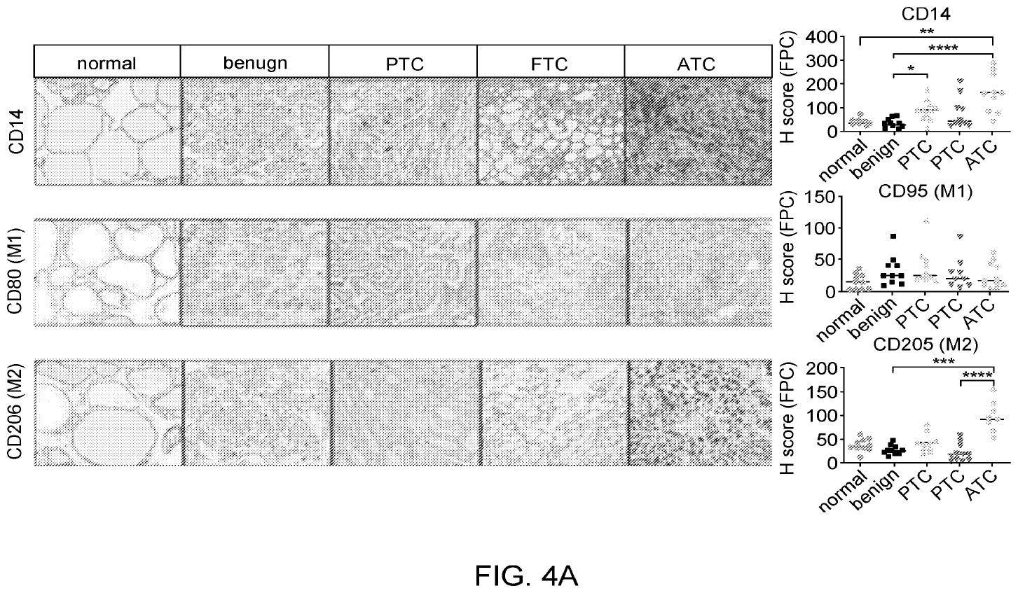

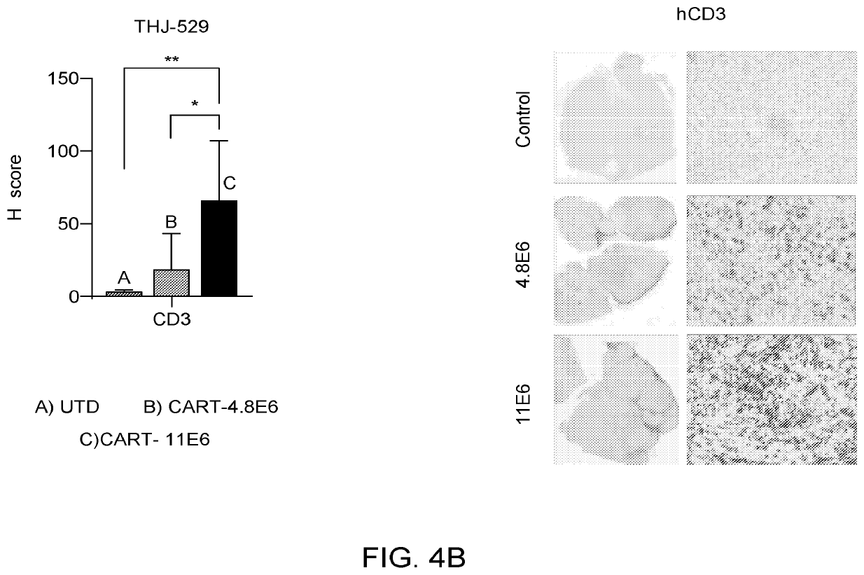

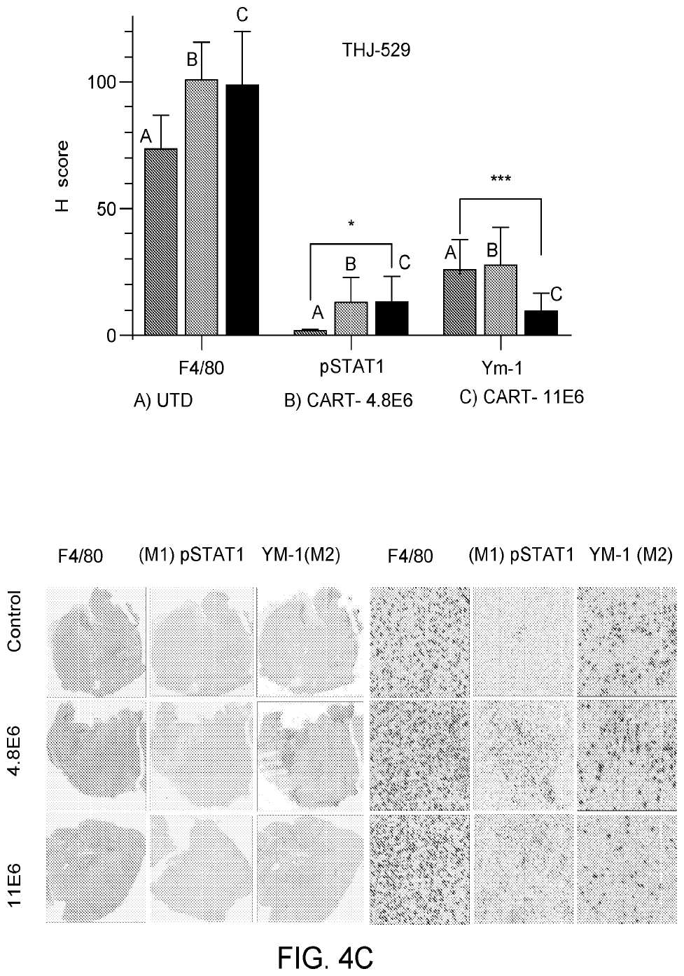

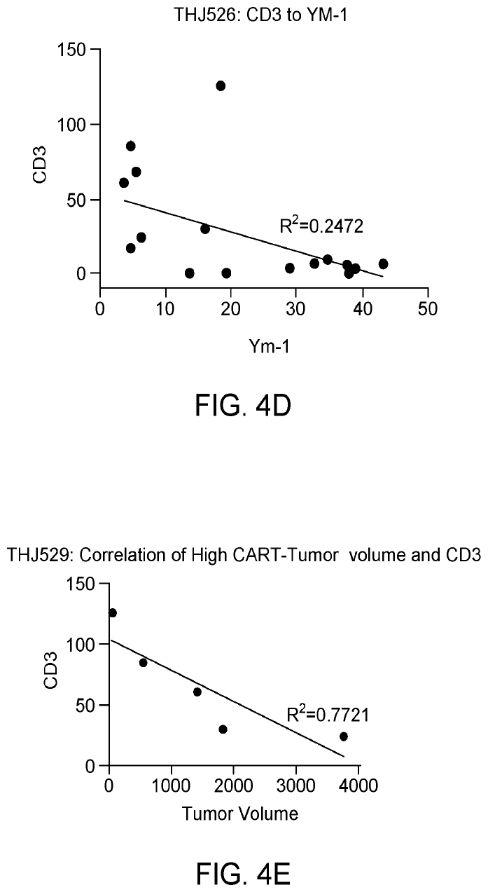

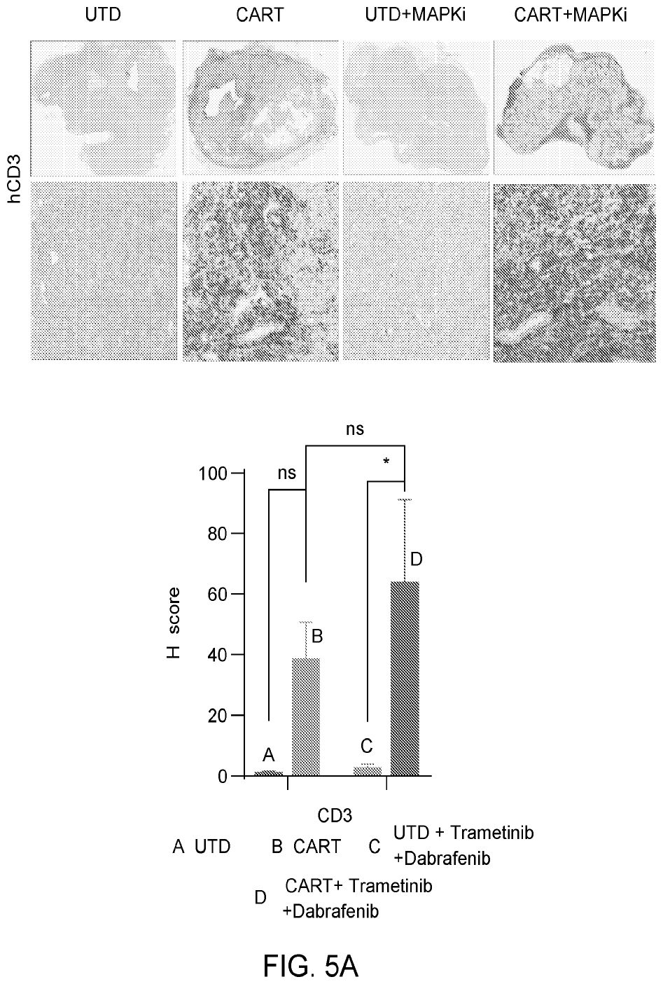

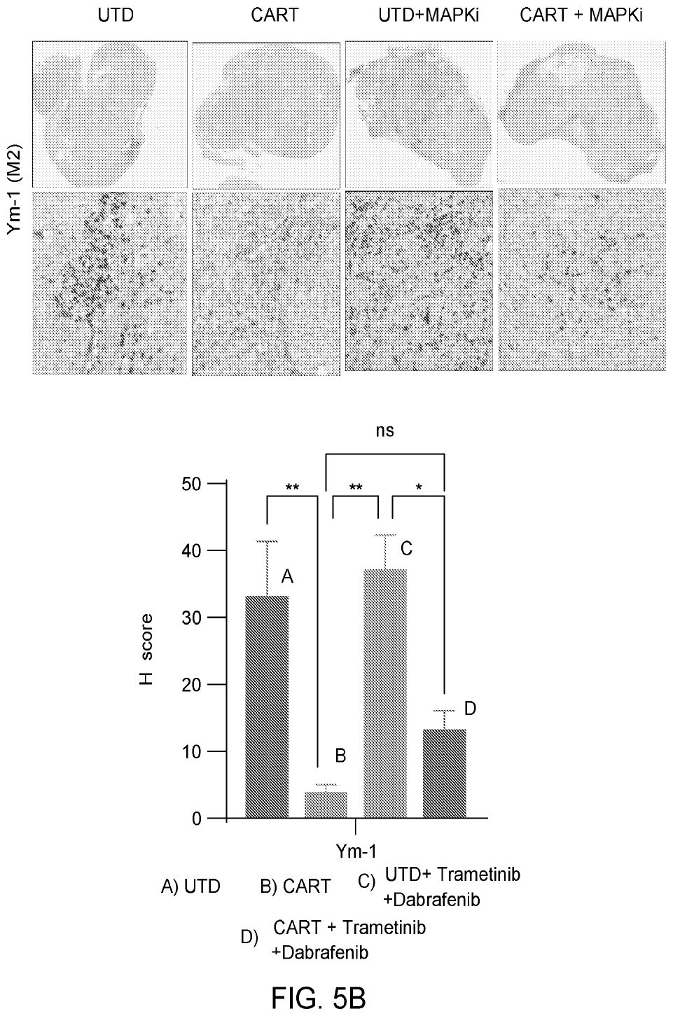

It was examined whether MEK inhibitors can prime thyroid cancer cells to increase the therapeutic index of TSHR-CAR T cells. Anaplastic thyroid cancer PDX mouse models were treated with MEK inhibitors, and the kinetics of TSHR expression in TSHR-CAR T cells were studied. As shown in FIG. 4A-4B, TSHR protein expression by IHC was elevated significantly within 14 days of daily oral treatment with either 1.5 mg/kg R05126766 or 1 mg/kg trametinib over vehicle control demonstrating no TSHR expression (FIG. 3B). Since THJ-529T PDTX is a BRAF mutant ATC tumor, dabrafenib (12.5 mg/kg daily), a BRAF inhibitor, was added to the MEK inhibitor to prevent MEK inhibition escape (FIG. 3B). Similar levels of TSHR upregulation were observed within seven days of daily treatment with the combination therapy.

The sequential treatment of MEK/BRAF inhibition followed by TSHR-CAR T cell therapy was examined (FIG. 3F). On day 0 of the experiment, two treatment groups of NSG mice were engrafted with 5 mm³ ATC PDTX tumors followed a week later by another two treatment groups. The first two treatment groups upon achieving tumor volume of 75-100 mm³ were treated daily with R05126766 at dose of 1.5 mg/kg and dabrafenib 12.5 mg/kg of body weight orally through ad libitum treated diet gel access for 7 days to upregulate TSHR. On Day 8, one group received either 20 million UDT cells or 20 million CAR T cells via intravenous administration. The second two treatment groups were the controls that were implanted a week later and were treated identically except they were not treated with the MEK and BRAF inhibitors. Tumor volume, body weight, and body condition were inspected daily to monitor disease progression and to assay for endpoint criteria. Mice conditioned with R05126766 plus dabrafenib and subsequently treated with THSR-CAR T cells showed the control of tumor (FIG. 3G). Subsequent immunohistochemistry testing displayed a high degree of TSHR upregulation in the tumors treated with R05126766 plus dabrafenib (FIG. 3B). Collectively, the study indicated that MEK/BRAF inhibition of dedifferentiated thyroid cancer upregulated TSHR expression and enhanced the antitumor activity of TSHR-CAR T cell therapy.

Tam Infiltration is Associated with Suboptimal Response to CAR T Cell Therapy

Tumor associated macrophages (TAMs) have been demonstrated to promote tumor growth and inhibit effector functions in thyroid cancer. The study evaluated if macrophages are associated with resistance to CAR T cell therapy in preclinical models. First, the study validated the infiltration of macrophages in thyroid cancer samples and demonstrated significantly higher levels of TAMs in ATC (FIG. 4A). CAR T cell therapy upregulated the M1 and downregulated the M2 macrophage phenotypes indicative an anti-tumor effect of the macrophages.

Additionally, CD3⁺ T cells were upregulated in the ATC tumors by CAR T cell therapy indicative of T cell activation (FIG. 4B). Infiltration of M2 macrophages was associated with reduced T cell infiltration into tumor cells and lower antitumor response (FIGS. 4C, 4D, and 4E). Collectively, data from the study indicate that the TAMs infiltration is associated with reduced T cell infiltration and lack of response to CAR T cell therapy.

Together, these results indicate that T cells (e.g., CAR T cells) engineered to express an antigen receptor (e.g., a CAR) that can target a TSHR polypeptide can be used (e.g., in an adoptive T cell therapy such as a CAR T cell therapy) to treat a mammal having cancer.

### Materials and Methods

### Cell Cultures

The human-derived cell cultures used for the completion of this study include: FTC133, Lam1, Lam136, THJ96N, THJ426N, THJ428N, THJ529T, R082-W-1, and K562. The K562 cells were obtained from American Tissue Culture Collection (ATCC, Manassas, VA, USA). The RO82-W-1 cells were originally obtained from the European Collection of Authenticated Cell Cultures (EACC, London, UK). For some experiments, K562, FTC133, LAM1, LAM136, THJ428N, THJ96N, THJ426N, cells were transduced with a firefly luciferase vector containing a hygromycin resistance gene (Promega, Madison, WI, USA, Cat #E6691) as well as a TSHR—C-Flag-SV40-eGFP-IRES-puromycin vector (Genecopoeia Inc., Rockville, MD, USA) for stable TSHR overexpression in target cell lines. These cells were then cultured in their respective selection media containing either hygromycin (1 μg/mL) or puromycin (2 μg/mL) to obtain pure populations expressing the vectors.

### Designing of Chimeric Antigen Receptor

The CAR design used in this study incorporated 4-1-BB and CD3 zeta costimulation. As previously stated, the study utilized monoclonal autoantibodies from a Graves Disease patient. The clone K1-70 are TSHR stimulating and blocking type antibodies respectively. The hinge and transmembrane domains of the CAR design were incorporated as described elsewhere (see, e.g., Zhao, Z. et al. Cancer Cell 28, 415-428 (2015)). This clone was used as a tool to study the impact of TSHR CAR T cell therapy in preclinical models. Subsequently, eight novel unique clones targeting TSHR were generated and incorporated in the generation of sixteen different CAR T cell constructs Cell lines were cultured in R5 medium made with Roswell Park Memorial Institute (RPMI) 1640 (Gibco, Gaithersburg, MD, USA, Cat #21870), 5% fetal bovine serum (FBS, Sigma, St. Louis, MO, USA, Cat #F8067), 1% penicillin-streptomycin-glutamine (Gibco, Gaithersburg, MD, USA, Cat #10378-016), 1 mM Sodium pyruvate (Sigma, St. Lois, MO, USA, Cat #S8636100 ml), 1X Mem nonessential amino acids (NEAA, Corning, NY, USA, Cat #25025CI), 10 mM 4-(2-hydroxyethyl)-1-piperazineethanesulfonic acid (HEPES, Gibco Gaithersburg, MD, USA Cat #15630080). The cell lines tested negative for mycoplasma (IDEXX, Columbia, MO, USA).

Healthy donor T cells were isolated from de-identified apheresis cones. The peripheral blood mononuclear cell (PBMC) containing fraction was isolated using EasySep™ tubes (STEMCELL Technologies, Vancouver, Canada, Cat #85450), Lymphoprep™ solution (STEMCELL Technologies, Vancouver, Canada, Cat #07851), and a room-temperature tabletop centrifuge. Pan-CD3 T cells were further isolated via magnetic negative selection with the EasySep™ Human T Cell Isolation Kit (STEMCELL Technologies, Vancouver, Canada, Cat #17951) in a Robosep automated separator.

The patient-derived CAR-TSHR plasmid was generated by cloning anti-TSHR ScFvs, a CD8 hinge, a 4-1BB costimulatory domain, and a CD3ζ signaling domain into the lentiviral backbone (FIG. 1D) as described elsewhere (see, e.g., Zhao, Z. et al. Cancer Cell 28, 415-428 (2015)). Healthy donor T cells were stimulated and subsequently expanded in vitro using a bead:cell ratio of 3:1 with anti-CD3/CD28 Dynabeads (Invitrogen, Life Technologies, Grand Island, NY, USA, Cat #11141D, added at day 0 of culture). 24 hours post stimulation, T cells were transduced with lentiviral supernatant from 293T cells transfected with pLV-CAR-TSHR plasmid and two helper plasmids at a multiplicity of infection of three. The anti-CD3/CD28 Dynabeads were removed by magnetic separation on day 6 and flow cytometric analysis for CAR-TSHR surface expression was performed with goat anti-mouse antibody (Invitrogen, Life Technologies, Grand Island, NY, USA Cat #A-21235).

T cells were grown for up to 8 days in T cell media (TCM; X-VIVO™ 15 media (Lonza, Basel, Switzerland, Cat #04-418Q), 10% human AB serum (Corning, NY, USA, Cat #35-060-CI), 1% penicillin-streptomycin-glutamine (Gibco, Gaithersburg, MD, USA, Cat #10378-016) and then cryopreserved (FBS with 10% DMSO) for future use. T cells were thawed and rested overnight at 37° C., 5% CO₂ before experiments.

### Multi-Parametric Flow Cytometry

Anti-human antibodies were purchased from Biolegend (San Diego, CA, USA), eBioscience (San Diego, CA, USA) or BD Biosciences (San Jose, CA, USA). Samples were prepared for flow cytometry as described elsewhere (see, e.g., Sterner, R. M. et al., Blood 133, 697-709 (2019)). Count bright beads (Invitrogen, Carlsbad, CA, USA, Cat #C36950) were used according to the manufacturer's instructions for cell number quantification when sample collection volumetrics were not used. To evaluate, the population of interest was gated based on forward vs side scatter characteristics, followed by singlet gating, and live cells were gated using LIVE/DEAD™ Fixable Aqua Dead Cell Stain Kit (Invitrogen, Carlsbad, CA, USA, Cat #L34966). Flow cytometry was performed on a three-laser CytoFLEX (Beckman Coulter, Chaska, MN, USA). Evaluation was performed using FlowJo X10.0.7r2 software (Ashland, OR, USA).

### Immunohistochemistry (IHC)

Immunostaining was performed on paraffin-embedded tissue. Thyroid tissue microarray (TMA) was made from archival samples. Paraffin blocks were cut into 5 μm thick sections. Slides were deparaffinized, and were rehydrated with decreasing concentrations of ethanol and finally in water. Antigen retrieval was performed using Antigen Retrieval Solution (pH 6 or pH 9) (Dako, Glostrup, Denmark), for at least 15 minutes in an autoclave, and cooling for 30 minutes at room temperature. Washing slides with 3% hydrogen peroxide (Fisher Scientific, Waltham, MA, USA) for 10 minutes blocked the activity of endogenous peroxidases. Primary anti-human antibodies were used for 60 minutes at room temperature and at the following dilutions: TSHR antibody at 1:500 (abcam, ab218108, Cambridge, UK) and NIS antibody at 1:200 (ProteinTech, 24324-1-AP, Rosemont, IL, USA), CD14 antibody at 1:100 (abcam, ab183322, Cambridge, UK), CD80 at 1:1000 (abcam, ab134120, Cambridge, UK), CD206 at 1:5000 (abcam, ab64693, Cambridge, UK), Ki67 at 1:1000 (abcam, ab15580, Cambridge, UK), cleaved caspase 3 (Asp175) CC3 at 1:100 (Cell Signaling 9661, Danvers, MA, USA), and CD31 at (Santa Cruz Biotechnology, sc-1506, Dallas, TX, USA).

The presence of murine macrophages in thyroid carcinoma tumors was determined using anti-mouse F4/80 antibody at 1:50 (BioRad, MCA497R, Hercules, CA, USA), M1 macrophage marker pY701-STAT1 at 1:250 (abcam, ab29045, Cambridge, UK), and M2 macrophage marker Yml at 1:200 (StemCell Technologies, 60130, Vancouver, Canada). The infiltration of human T lymphocytes into the neoplastic tumors was marked using anti-human CD3 at 1:500 (abcam, ab52959, Cambridge, UK). The primary antibody was detected using the Envision Labeled Kit (Dako, Glostrup, Denmark) according to the manufacturer's protocol. After washing, slides were counterstained with Mayer's hematoxylin (Sigma-Aldrich, Burlington, MA, USA). Slides were dehydrated through ethanol and xylene and cover-slipped using a xylene-based mounting medium (Fisher Scientific, Waltham, MA, USA). Samples were examined under bright-field illumination at ×20 objectives, and digital images were obtained using Aperio AT2 (Leica, Wetzlar, Germany). Results were processed using Aperio eSlide Manager and H-Score values were estimated using the Aperio ImageScope Software (both Aperio Technologies, Vista, CA, USA). Normal tissues were used for the positive antibody control and the negative antibody control.

### Immunocytochemistry (ICC—HistoGel)

Cells were washed three times with PBS (Corning, NY, USA), scraped from the bottom of the plate using the cell lifter (Fisherbrand, Waltham, MA, USA), transferred to 50 mL polypropylene the tube, and centrifuged at room temperature for 2 minutes at 500×g. The supernatant was aspirated, and 10% neutral buffered formalin (Fisher Scientific, Waltham, MA, USA) was added for 30 minutes at room temperature. HistoGel (Thermo Scientific, Waltham, MA, USA) was heated in the microwave for 3 seconds at maximal power and converted to a liquid state. Cells were transferred to the HistoScreen Tissue Cassettes (Thermo Scientific, Waltham, MA, USA) and covered with liquid Histogel. Samples were placed at room temperature and allowed to solidify. HistoGel blocks were transferred into tissue embedding cassettes (Thomas Scientific, Swedesboro, NJ, USA), dehydrated in increasing concentrations of ethanol, xylene, and paraffin embedding. Sections were prepared as described in the immunohistochemistry section. Primary anti-human antibody was used for 60 minutes at room temperature: TSHR antibody at 1:1500 (abcam, ab218108, Cambridge, UK). Envision labeled polymer (Dako, Glostrup, Denmark) was used for 30 minutes as a secondary antibody. Slides were stained with diaminobenzidine tetrahydrochloride (DAB) chromogen (Dako, Glostrup, Denmark) for 5 minutes at room temperature and counterstained with Mayer's hematoxylin (Sigma-Aldrich, Burlington, MA, USA). Control staining with hematoxylin and eosin (Sigma-Aldrich, Burlington, MA, USA) was also performed. Aperio AT2 scanner (Leica, Wetzlar, Germany) was used for the digital image, at 20× objective. Images were analyzed using the Aperio ImageScope software (Aperio Technologies, Vista, CA, USA).

Immunocytochemistry (ICC) THJ-529 cells (2×10⁴ cells/chamber) were plated in a 4-well chamber slide (Nunc, Thermo Scientific, Waltham, MA, USA), incubated overnight. After 24 hours, media was aspirated, cells were washed three times with PBS (Corning, NY, USA). Then, 2% paraformaldehyde was added for 20 minutes at room temperature. Again, cells were washed three times with PBS. Next, ice-cold 100% methanol (Fisher Scientific, Waltham, MA, USA) was added for 7 minutes at −20° C. Methanol was aspirated, and samples were allowed to air dry for 10 minutes. Next, serum-free-blocking diluent (Dako, Glostrup, Denmark) was used for 30 minutes at room temperature. Primary anti-human antibody was used for 60 minutes at room temperature: TSHR antibody at 1:1500 (abcam, ab218108, Cambridge, UK). After that, slides were prepared as described in the immunohistochemistry section.

### Tissue Microarray (TMA)

A tissue microarray (TMA) was made from archival formalin fixed paraffin embedded samples. TMA tissues were cut into 5 mm sections, deparaffinized, hydrated, antigen retrieved, and blocked with diluent that contained Background Reducing Components (DAKOCytomation, Glostrup, Denmark). Immunostaining was done with HDAC1 at 1:100 (Santa Cruz) and HDAC6 at 1:100 (Cell Signaling). The Envision Dual Labeled Polymer kit (DAKOCytomation) was used according to the manufacturer's instructions, and then slides were lightly counterstained with Gill I hematoxylin (Sigma-Aldrich) before dehydration and mounting. Images were obtained at 20X using Scanscope XT (Aperio Technologies, Vista, CA, USA), and the staining of the TMA punches were scored using an algorithm in the Imagescope Software (Aperio Technologies) created by a histologist based upon signal intensity (0, 1C, 2C, 3C). H score was then calculated based upon signal intensity and percentage. The: H-score is obtained by the formula: 3×percentage of strongly staining nuclei+2×percentage of moderately staining nuclei+percentage of weakly staining nuclei, giving a range of 0 to 300. Cases were excluded from the study if a section could not be assigned a score due to insufficient quantity of tumor tissue present.

### In Vitro T Cell Function Assays

Proliferation assays: Naïve T cells or TSHR-CAR T cells were re-suspended in T cell media at 2×10⁶/mL, and 50 μL per well were seeded in 96-well plates. Each assay also included cells with media as a blank control, cells with PMA & ionomycin as a positive control. After 120 hours, cells were harvested and stained for APC-H7 anti-human CD3 (eBioscience, San Diego, CA, USA, Cat #560176), BV421-CD4 (BioLegend, San Diego, CA, USA, Cat #304032), and LIVE/DEAD™ Fixable Aqua. Countbright beads were added prior to flow cytometric analysis if sample volumetrics were not used to determine the absolute counts.

Cytotoxicity assays: Cytotoxicity assays were performed as described elsewhere (see, e.g., Sterner, R. M. et al., Blood 133, 697-709 (2019)). In summation, luciferase/TSHR+/+K562 or luciferase/TSHR+/+FTC133 cells was used as a target cell. TSHR-CAR T cells or batch-matched control donor untransduced (non-transduced) T cells (UTD) were co-cultured with target cells at serial effector: target (E:T) ratios in T cell media. Cytotoxic efficiency was calculated by bioluminescence imaging on the Xenogen IVIS-200 Spectrum (PerkinElmer, Hopkinton, MA, USA) or Promega GlowMax Explorer (Promega Corporation, Fitchburg, WI, USA) at 24 hours, 48 hours, and 72 hours, as indicated in the specific experiment.

Degranulation and intracellular cytokine assays: T cells were incubated with various target cells at an effector: target ratio of 1:5.41Antibodies against FITC-CD107α (BD Pharmingen, San Diego, CA, USA, Cat #555800), CD28 (BD Biosciences, San Diego, CA, USA, Cat #348040), CD49d (BD Biosciences, San Diego, CA, USA, Cat #340976), and monensin (Biolegend, San Diego, CA, USA, Cat #420701) were added prior to the incubation. After 6 hours, cells were harvested and stained with LIVE/DEAD™ Fixable Aqua. Cells were then fixed and permeabilized (FIX & PERM Cell Fixation & Cell Permeabilization Kit, Life Technologies, Oslo, Norway, Cat #GAS004) and stained for CD3 (clone UCHT1), APC (Cat #17-0038-42, eBioscience, San Diego, CA, USA), and intracellular cytokines including IL-2 (clone 5344.111) PE-CF594 (BD Pharmingen, San Diego, CA, USA, Cat #562384), GM-CSF (clone BVD2-21C11) BV421 (BD Pharmingen, San Diego, CA, USA, Cat #562930), IFN-7 (clone 4S.B3), APC-eFluor 780 (Invitrogen, Carlsbad, CA, USA, Cat #47-7319-42), and MIP1-P (clone D21-1351) PE-Cy7 (BD Pharmingen, San Diego, CA, USA, Cat #560687).

In vivo Leukemic Mouse Experiments

Non-obese diabetic/severe combined immunodeficient mice (6-8-week-old) bearing a targeted mutation in the interleukin (IL)-2 receptor gamma chain gene (NSG) mice were originally obtained from Jackson Laboratories (Jackson Laboratories, Bar Harbor, ME, USA). Mice were intravenously injected with 1.0×10⁶ TSHR+K562 cells. Once engrafted, mice were imaged weekly with a bioluminescent imager using a Xenogen IVIS-200 Spectrum camera (PerkinElmer, Hopkinton, MA, USA). Mice were bled and subjected to flow cytometry to confirm engraftment. Mice were then randomized based on the flow cytometry indicated tumor burden to receive different treatments as outlined in the specific experiment. Mice were euthanized once endpoint criteria were met or the mice were found dead-in-cage during daily census.

In vivo Flank Tumor Mouse Experiments

In a follow-up experiment, 1e6 TSHR/LUC+/+FTC133 cells were suspended in a 50% MATRIGEL® basement medium and subcutaneously injected into the rear flank of 6-8 week old NSG mice. Imaging to follow tumor burden was performed 10 minutes after intraperitoneal injection of 10 μL/g D-luciferin (15 mg/mL, Gold Biotechnology, St. Louis, MO, USA) on a weekly basis until tumor engraftment was confirmed. Mice were then randomized based on tumor burden to receive different treatments as outlined in the specific experiment. Mice were euthanized once endpoint criteria were met, or the mice were found dead-in-cage during daily census.

In vivo Flank PDX Mouse Experiments

5-10 NSG mice per group were surgically engrafted with 5 mm³ ATC PDX tumors in their rear flank. These mice were then subjected to treatment with a sub-therapeutic dose of 1 mg/kg trametinib as a sensitizing regimen to induce TSHR expression on ATC tumor cells. On Day 7, 5-10 more mice were surgically engrafted with 5 mm³ PDX tumors in the same manner as the first arm, but these did not undergo sensitization with MEK inhibition. On Day 10, the mice from both arms were randomized based on tumor burden and subjected to one of the following therapeutic strategies: UTD, CAR T cell, trametinib, or no treatment. Disease progression was monitored through bi-daily assessment of tumor volume in addition to a daily survival census. Mice were euthanized at endpoints or were found dead-in-cage during daily census.

### Statistical Analysis

Prism Graph Pad (La Jolla, CA, USA) and Microsoft Excel (Microsoft, Redmond, WA, USA) were used to analyze raw experimental data. Statistical specifics are identified in FIG. legends. In summation, normally distributed data were subjected to one- and two-way analysis of variance (ANOVA) followed by Dunnett's multiple comparisons tests. Unpaired and paired two-sample Student's t-test or Mann-Whitney U test were used for two-group comparisons. Finally, survival was estimated using the Kaplan-Meier curve and Log-rank test was used to test the hypotheses for in vivo survival.

### Example 2: Macrophage Attenuation Provides Survival Benefit to TSHR CART Against Anaplastic Thyroid Carcinoma (ATC) Patient Derived Xenograft (PDX) Mouse Model

### Results

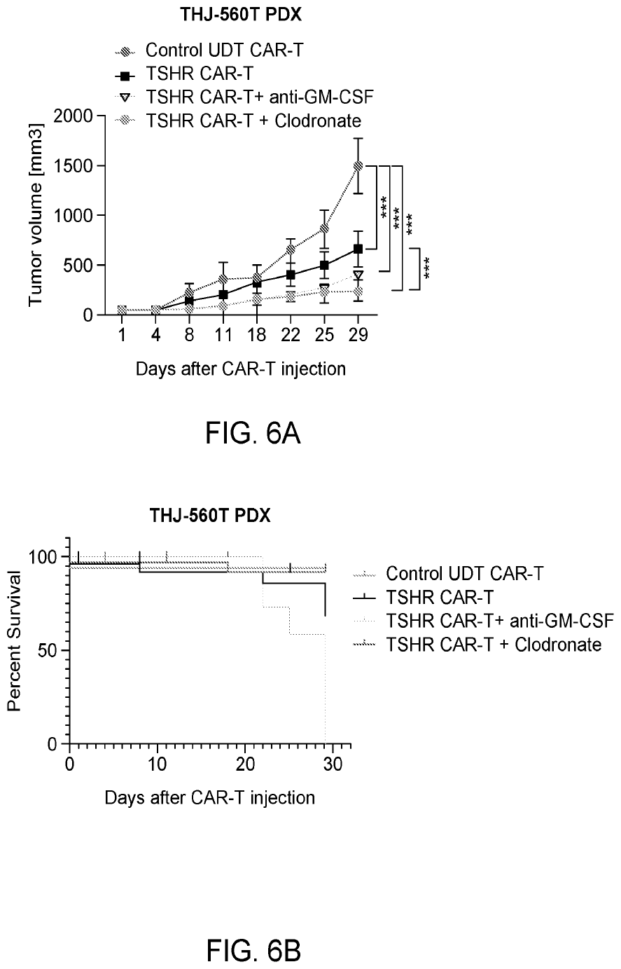

Macrophage depletion combined with TSHR CAR T therapy enhance antitumor activity in a preclinical model of anaplastic thyroid carcinoma (ATC). Empty CAR T cells (UDT) were used as control to be compared to CAR T cells expressing the thyroid stimulating hormone receptor (TSHR). Once tumors reached ˜50-100 mm³ in size, Nod Scid gamma (NSG) mice 4-6 weeks old were treated daily for seven days with a MEK inhibitor (trametinib) and BRAF inhibitor (dabrafenib) to stimulate TSHR protein synthesis. In this same seven day window, two groups of mice were additionally treated daily with macrophage depleting agents (anti-GM-CSF neutralizing antibody and clodronate). At day 7, 20 million CAR T cells were injected via tail vein injection. Tumor volume demonstrated dramatic inhibition of tumor growth when macrophage depletion was combined with TSHR CAR T therapy versus TSHR CAR T alone or control UDT therapy (FIG. 6A). Kaplan-Myer survival curve demonstrated 100% survival at day 29 of treatment compared to 0% in control UDT CAR T treated tumors (FIG. 6B).

### Methods

THJ-560T ATC PDX tumors (5 mm³) were implanted in the right flank s.q. according to IACUC protocols. Tumors grown to ˜75 mm³, were treated daily orally for one week with 1 mg/kg trametinib (MEK inhibitor) and 0.75 mg/kg dabrafenib (BRAF inhibitor). THJ-560T is a BRAF mutant tumor and thus a BRAF inhibitor was added to the MEK inhibitor to promote differentiation by blocking MAPK signaling. On day 8, mice were treated with 20 million UDT (control empty vector CAR T cells) or 20 million TSHR CART cells via tail vein injection (n=6 mice/group). One group of TSHR CART treated mice were administered clodronate liposomes (Clodrosome®+Encapsome®; 200 μlLi.p. every 2 days×4) or GM-CSF neutralizing antibody (10 mg/kg daily, ip, BioXcell, clone MP1-22E9; started on the same first day for MEK/BRAF inhibitor treatment) to inhibit macrophages, and to promote tumor growth and metastasis.

### Example 3: Sensitization of Aggressive Thyroid Cancers with MAPK Inhibitors for More Effective CART Cell Therapy

This Example describes the treatment of thyroid cancer by administering CAR T cells that can target a TSHR polypeptide in combination with one or more MAPK inhibitors.

The results in this Example re-present and expand on at least some of the results provided in other Examples.

### Results

### Tshr is an Ideal Target for CART Cell Therapy

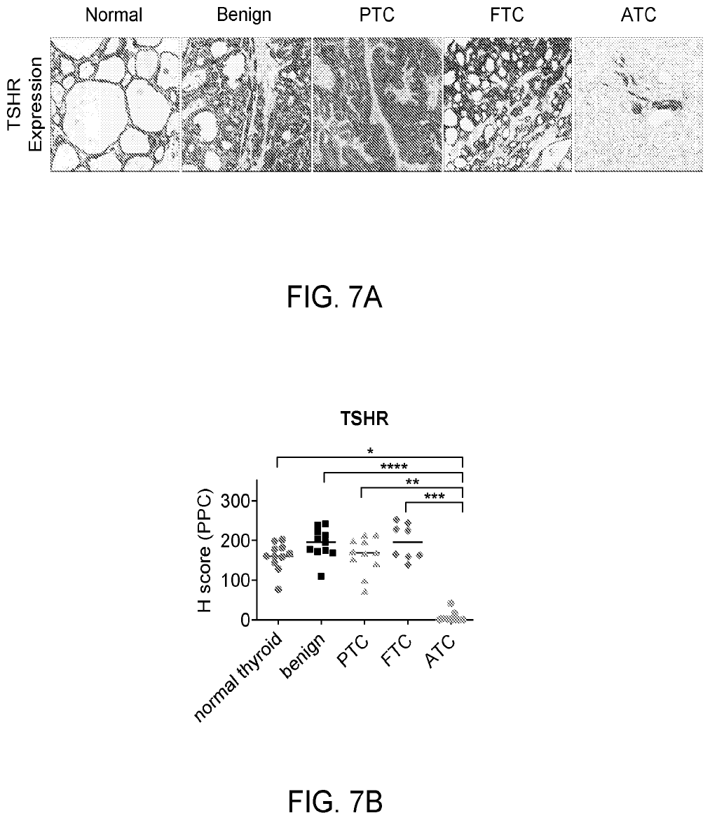

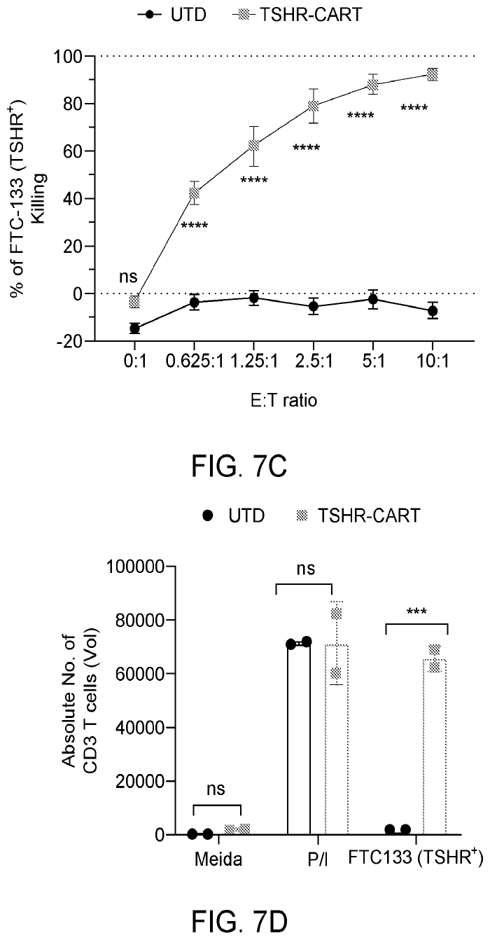

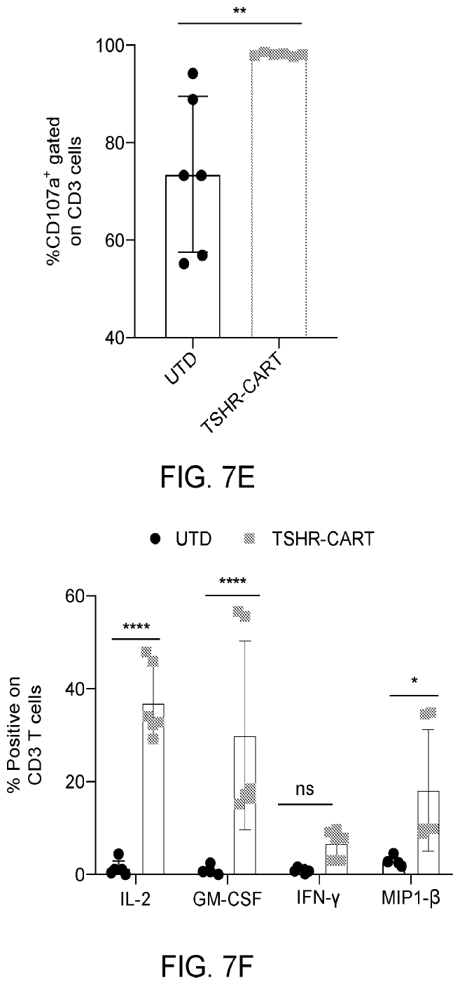

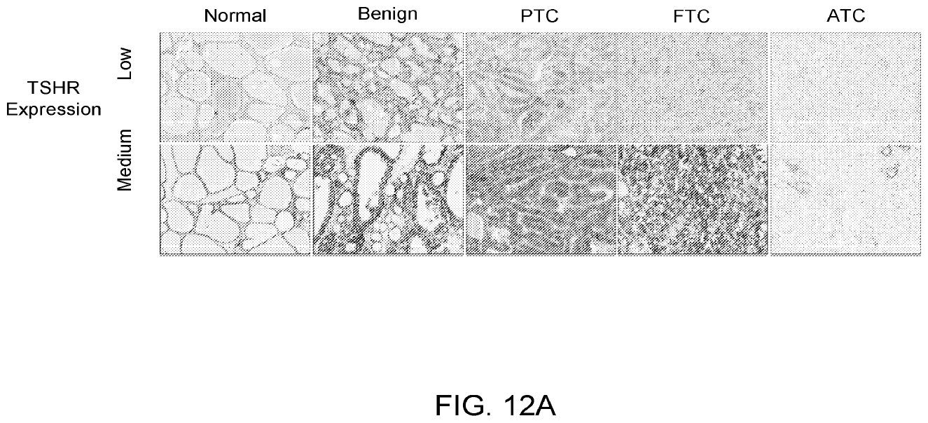

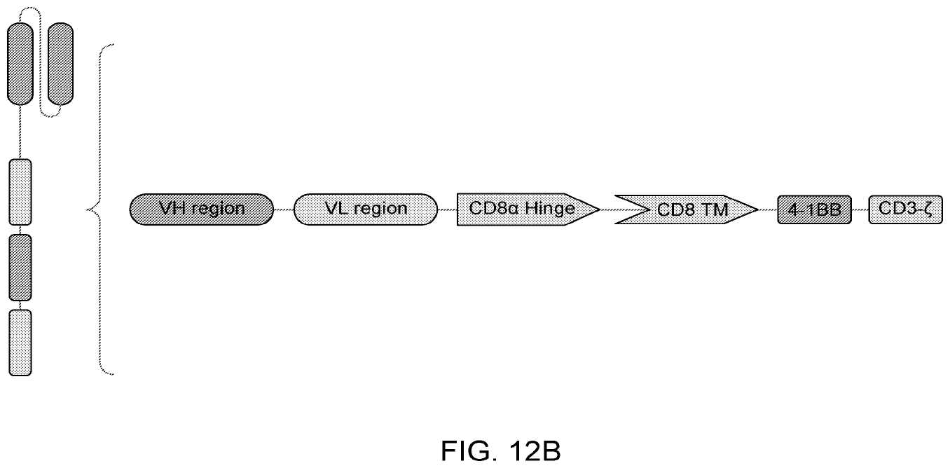

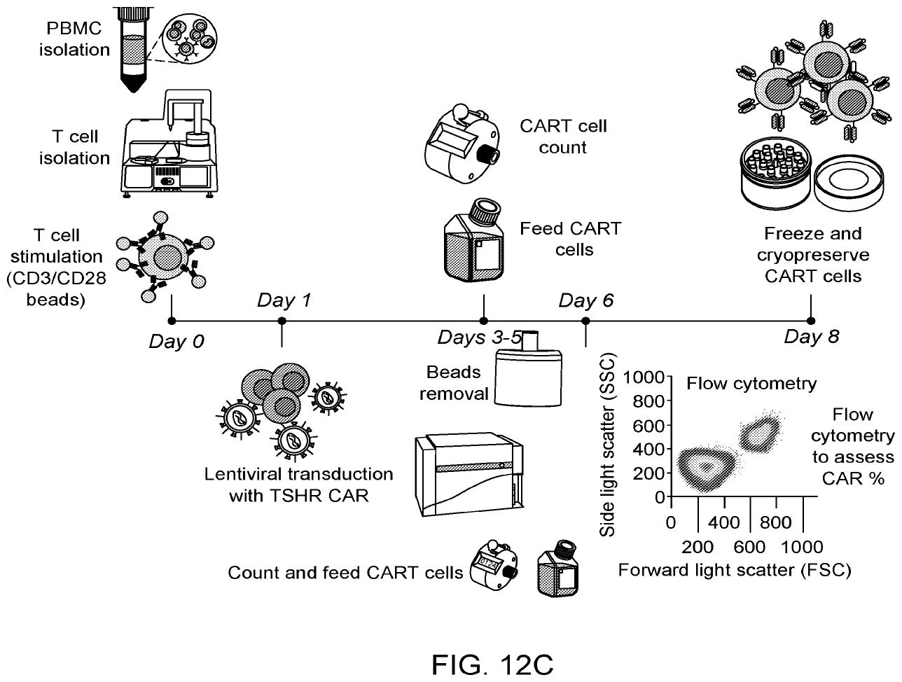

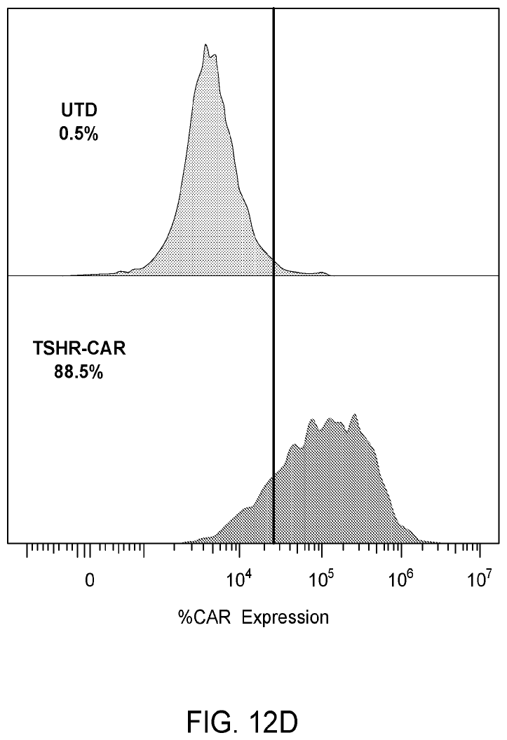

TSHR is a viable antigen to target with CART cell therapy, as it is uniquely and highly expressed in the thyroid gland but is largely absent from normal tissues outside the thyroid. The unique expression of TSHR on thyroid tissue was confirmed using an established thyroid cancer biobank. Immunohistochemistry (IHC) analysis showed that, while TSHR expression was high in normal thyroid tissue as well as multiple thyroid cancers, ATC samples demonstrated attenuated TSHR expression (FIGS. 7A 7B, and FIG. 12A). Having validated TSHR as a potential antigen to target with CART cell TSHR-CART cells therapy, were designed and generated using a single chain variable fragment isolated from the TSHR autoantibody clone K1-71 and cloned into a third-generation lentiviral CAR construct containing 4-1BB and CD3ζ (FIG. 12B). Healthy donor T cells transduced with CAR-encoded lentiviral particles demonstrated high CAR expression as assessed by flow cytometry (FIGS. 12C and 12D).

### TSHR-CART Cells Exhibit Potent and Specific Antitumor Activity Against TSHR-Expressing Tumors

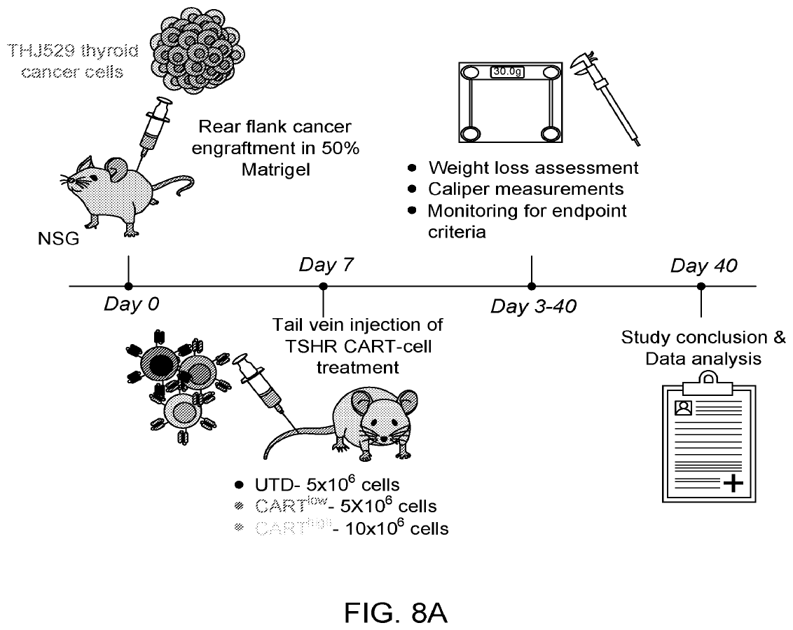

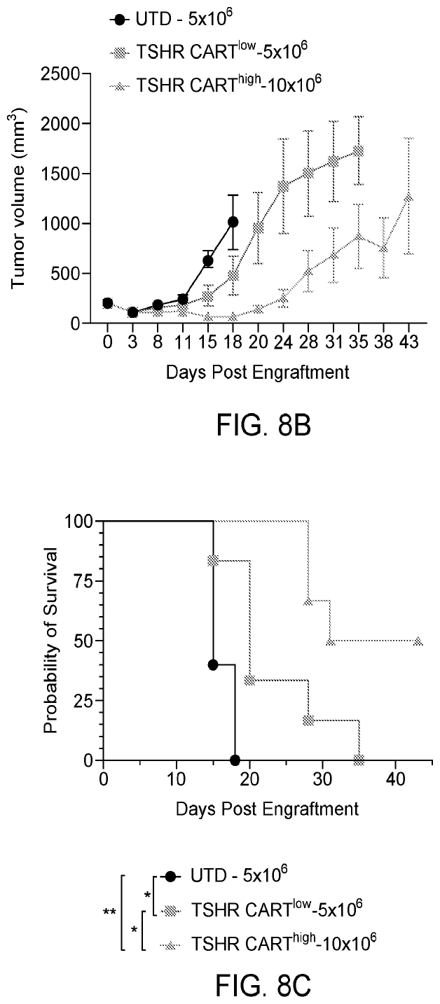

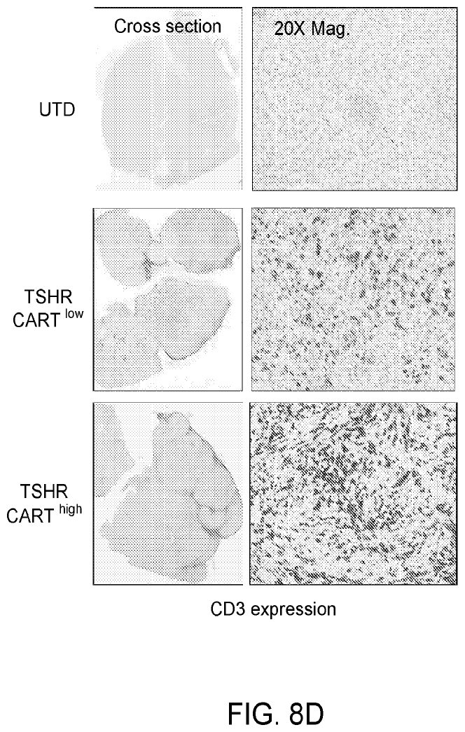

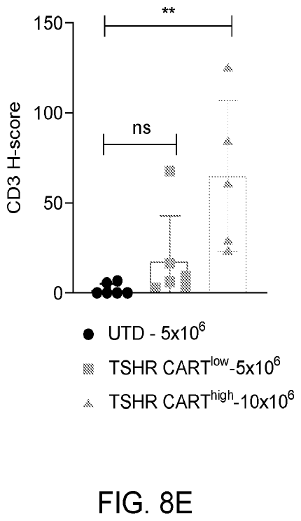

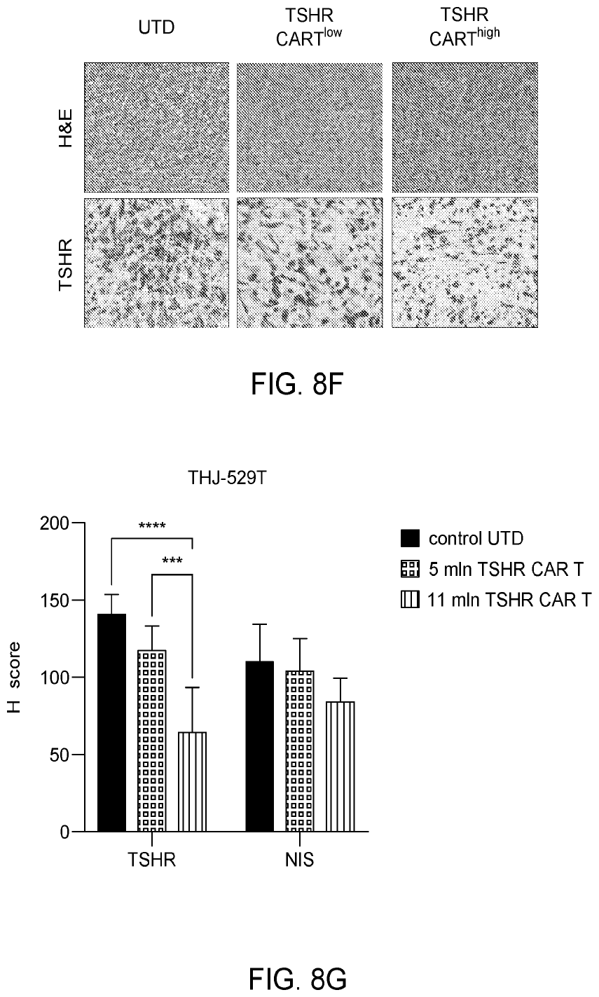

The antigen-specific activation of TSHR-CART cells was examined in vitro and in vivo. TSHR-CART cells or control untransduced T cells (UTD) were incubated with FTC-133 or K562 cell lines which were transduced to overexpress TSHR. Compared to UTD, TSHR-CART cells demonstrated profound antigen-specific effector functions against TSHR⁺ target cells in vitro (FIGS. 7C-7F). Next, the antitumor activity of TSHR-CART cells was evaluated in xenograft models in non-obese diabetic severe combined immunodeficiency IL2rγ^{-/-} (NSG) mice. In one model, NSG mice were engrafted subcutaneously with FTC-133 cells that were transduced to overexpress TSHR. After 1 week, when tumors reached ˜100 mm³ as determined by caliper measurements, mice were randomized to treatment with 5×10⁶ UTD or TSHR-CART cells intravenously through the tail vein. Mice were monitored daily for endpoint criteria. Mice treated with TSHR-CART cells had significantly prolonged survival compared to mice treated with UTD. To further validate the in vivo antitumor activity of TSHR-CART cells, a PDX model using aggressive, patient-derived ATC cells, THJ-529T, which were transduced to overexpress TSHR was employed. Engraftment was confirmed by caliper measurement of tumor masses. Three days after tumor engraftment, mice were randomized to treatment with 5×10⁶ UTD, 5×10⁶ TSHR-CART cells, or 10×10⁶ TSHR-CART cells (FIG. 8A). Treatment with TSHR-CART cells led to a dose-dependent decrease in tumor growth, improved survival outcomes, and increased tumor-infiltrated T cells (FIGS. 8B-8E). However, TSHR-CART treatment also led to a dose-dependent decrease in TSHR expression on tumor tissues (FIGS. 8F and 8G).

TSHR is Downregulated in ATC but is Restored with MEK Inhibition

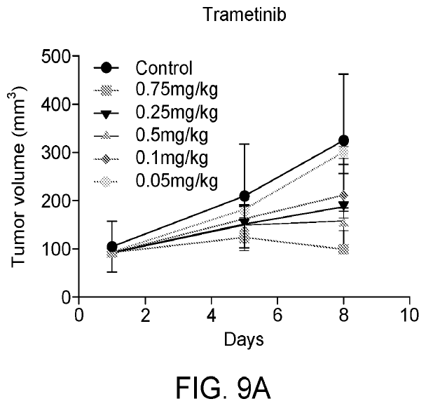

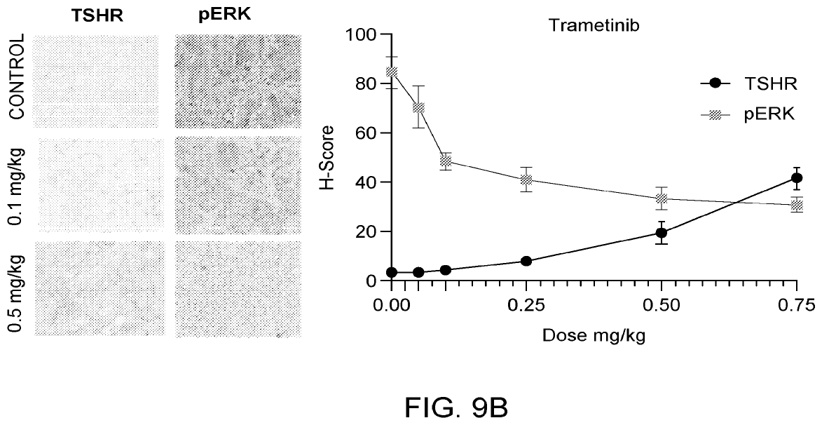

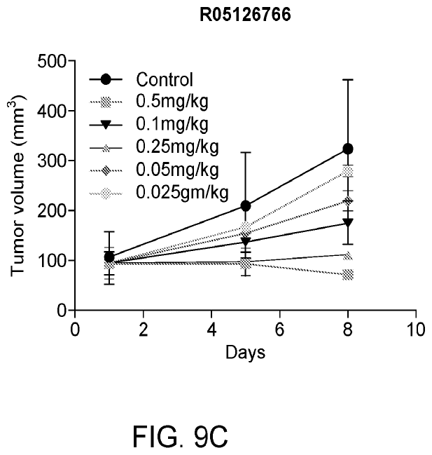

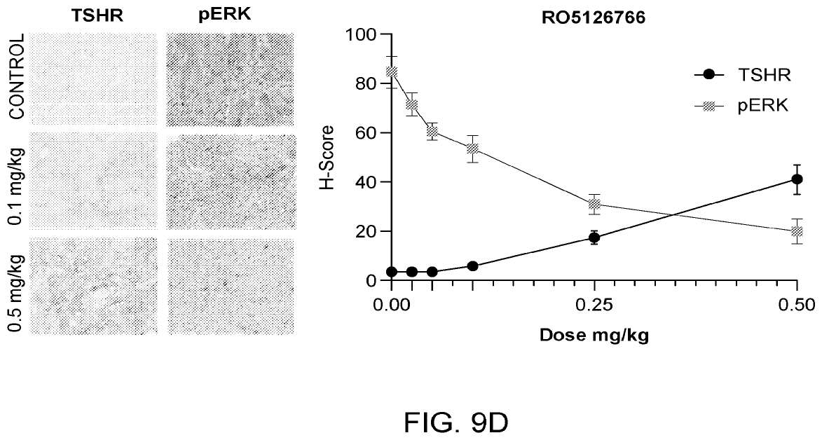

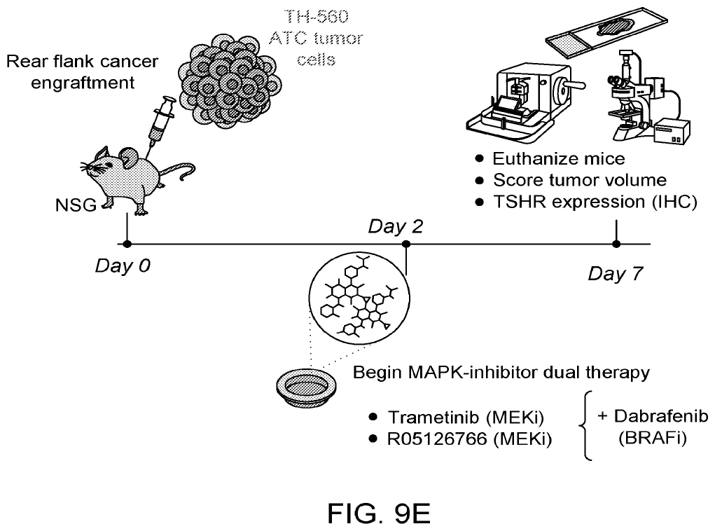

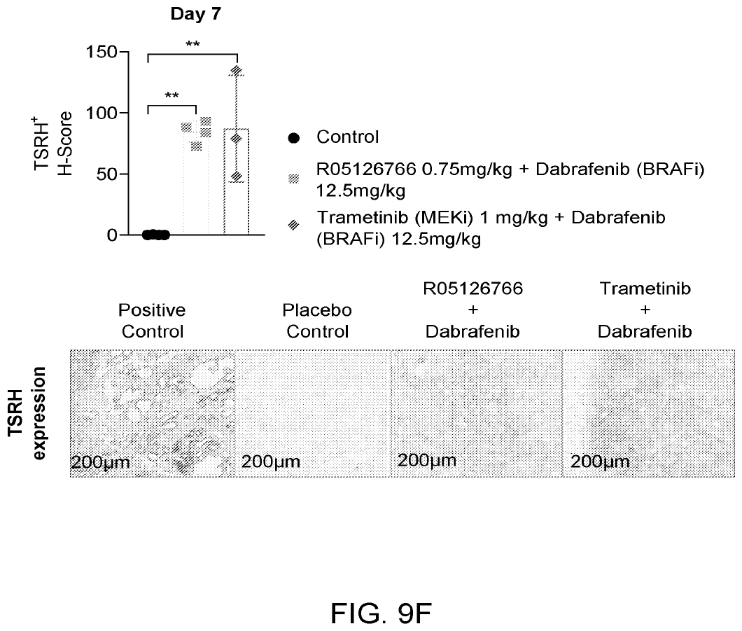

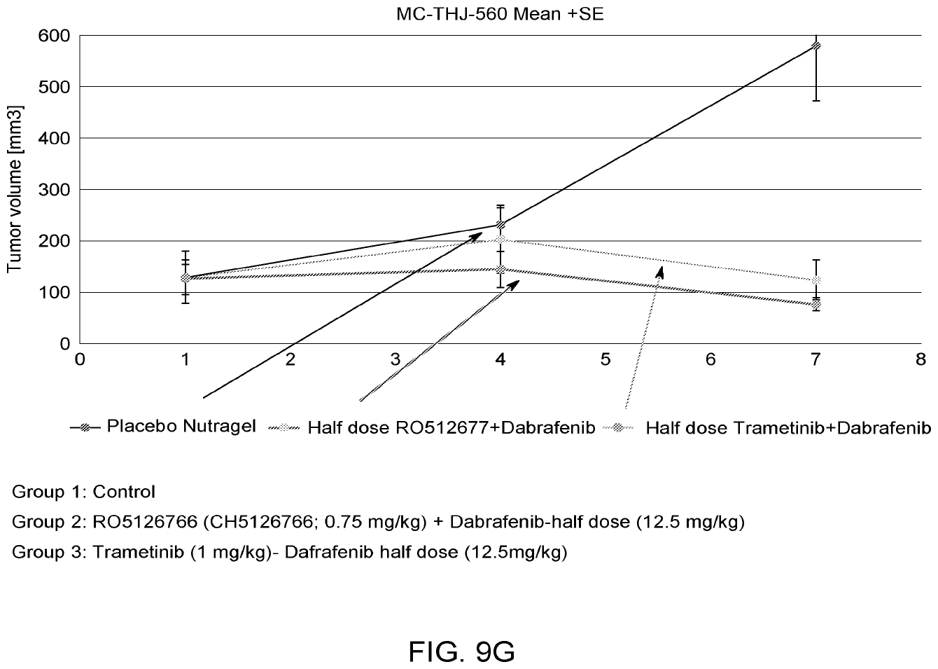

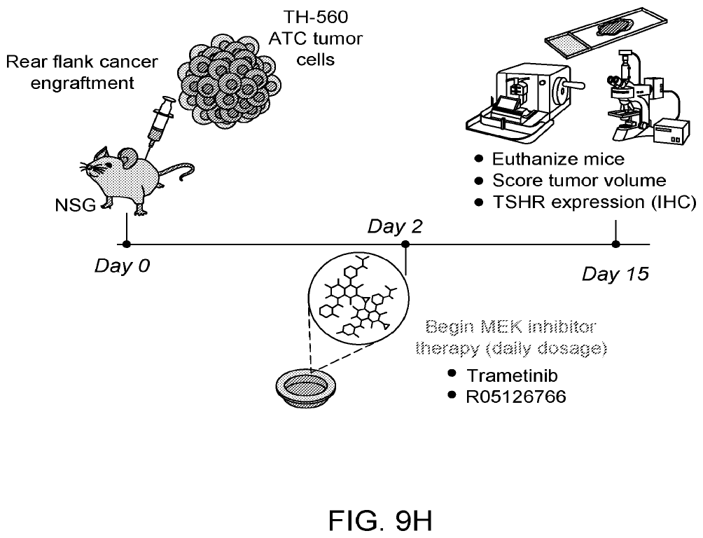

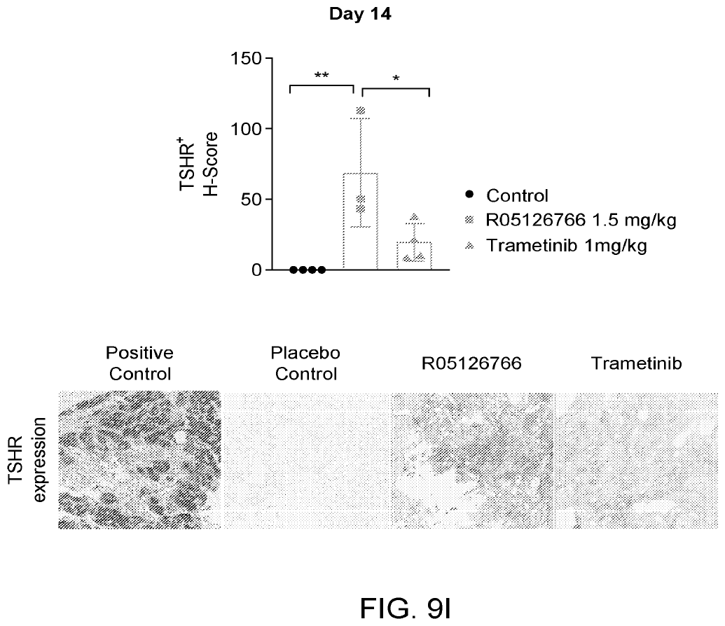

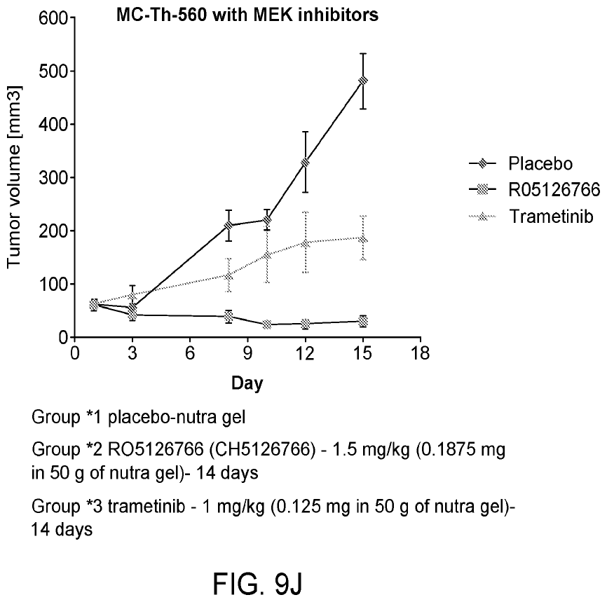

ATC cells have been associated with de-differentiation and downregulation of TSHR expression, and ATC significantly downregulated TSHR expression compared to normal thyroid tissues or more differentiated thyroid cancers (FIGS. 7A and 7B) and after TSHR-CART treatment (FIGS. 8F and 8G). It was verified that MEK and BRAF inhibitors restored TSHR expression in an ATC PDX model. In this model, 5 mm³ MC-Th-560 ATC PDX tissue was surgically implanted into the right lateral flank of NSG mice and grown to ˜100 mm³. Mice then received daily treatment with either vehicle control or trametinib (MEK inhibitor) at the indicated doses. IHC showed significant and dose-dependent TSHR re-expression and pERK reduction after one week of daily MEK inhibitor treatment compared to placebo treatment (FIGS. 9A and 9B). A similar experiment was performed in which mice received either vehicle control or the dual MEK/BRAF inhibitor, R05126766, at the indicated doses. R05126766 treatment resulted in dose-depended tumor reduction (FIG. 9C), increase in TSHR expression, and reduction of pERK (FIG. 9D).

### MEK and BRAF Inhibition Improves TSHR-CART Cell Activity

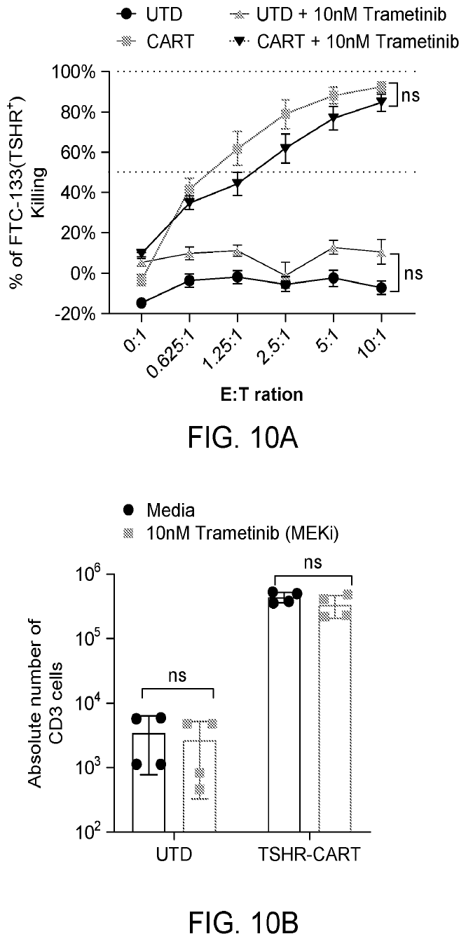

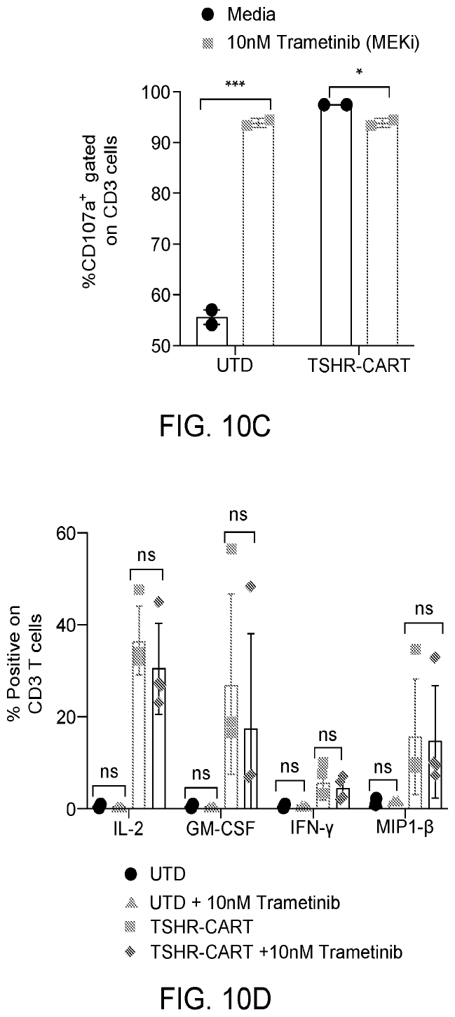

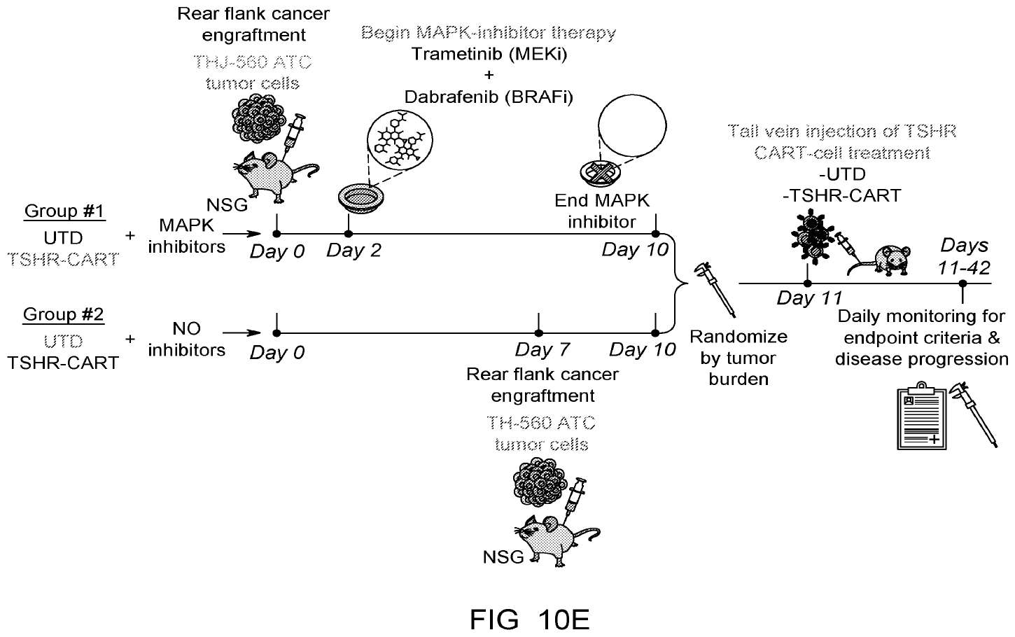

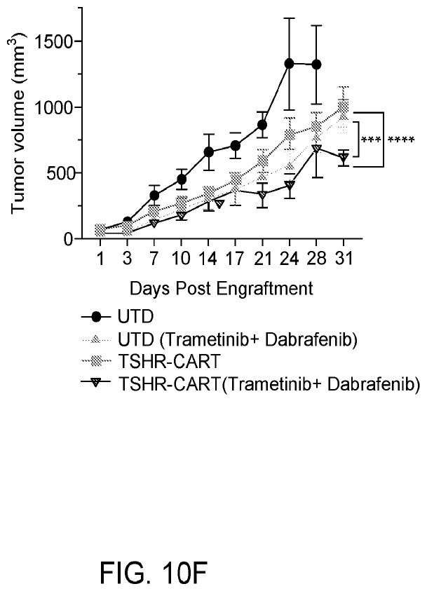

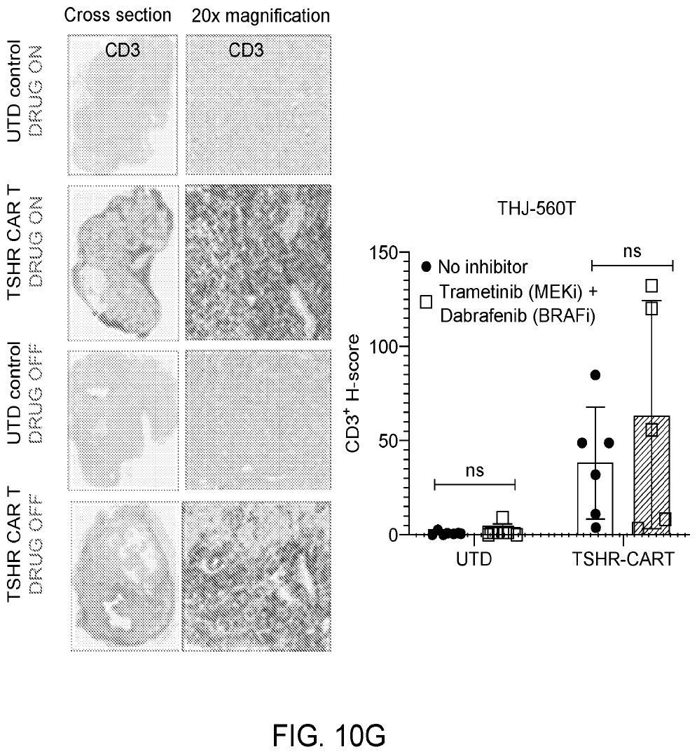

MEK and BRAF inhibition as a strategy to upregulate TSHR expression on thyroid cancer cells and increase the therapeutic index of TSHR-CART cell therapy was evaluated. Prior to developing MEK and BRAF inhibition as a strategy to redifferentiate cancer cells, any detrimental effects of MEK or BRAF inhibition on CART cell activity were ruled out. TSHR-CART cell antigen-specific killing, proliferation, degranulation, and intracellular cytokine production were not affected by the addition of MEK or BRAF inhibitors compared to media control (FIGS. 10A-10D).

The sequential combination of MEK and BRAF inhibition followed by TSHR-CART cell therapy was then tested in an ATC PDX mouse model. NSG mice were engrafted with 5 mm³ ATC BRAF-mutant THJ-560T PDX tumors. When tumor volume reached ˜100 mm³, mice were randomized to daily oral treatment with 1) placebo or 2) 1.5 mg/kg trametinib plus 12.5 mg/kg dabrafenib to upregulate TSHR. One week later, mice received 20×10⁶ of either UTD or TSHR-CART via tail vein injection (FIG. 10E). Because the MEK/BRAF inhibitors alone inhibit tumor growth, the treatment groups receiving placebo were implanted with tumors one week later than groups receiving MEK/BRAF inhibitors to achieve similar tumor volumes at the time of TSHR-CART treatment. Tumor volume, body weight, and body condition were inspected daily to monitor disease progression and to assess for endpoint criteria. Mice conditioned with trametinib plus dabrafenib and subsequently treated with TSHR-CART cells showed the most significant antitumor activity (FIG. 10F). However, statistical significance was only observed at the last treatment point, and there was no significant increase in tumor-infiltrating human CD3⁺ T cells between trametinib plus dabrafenib-treated groups and placebo-treated groups (FIG. 10G). In this model, TSHR expression was minimal in placebo-treated groups and likely explains lack of a more robust response in the placebo plus CART treatment group. Collectively, these findings indicated that MEK/BRAF inhibition of de-differentiated thyroid cancer enhanced TSHR-CART antitumor activity.

### Continuous MEK and BRAF Inhibition Yields Superior TSHR-CART Cell Activity

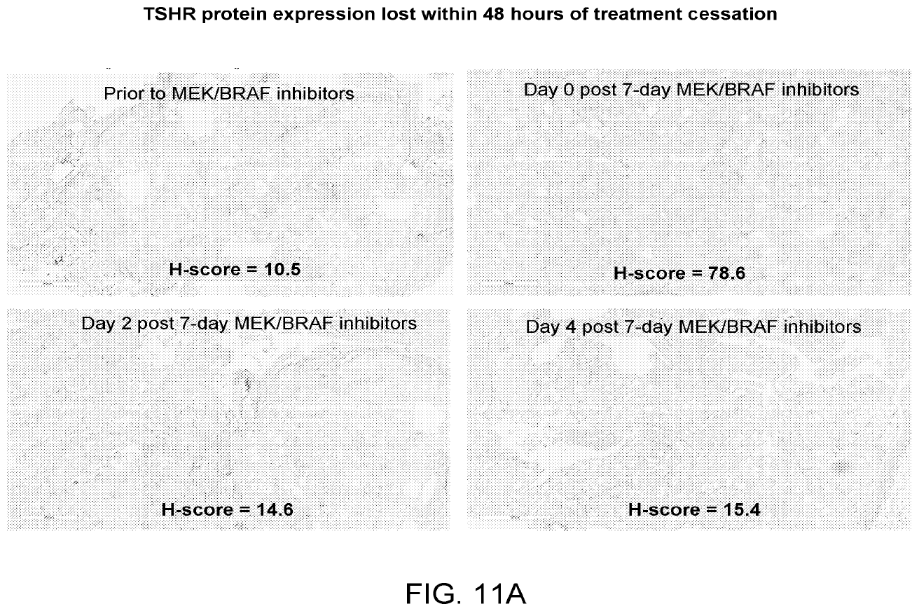

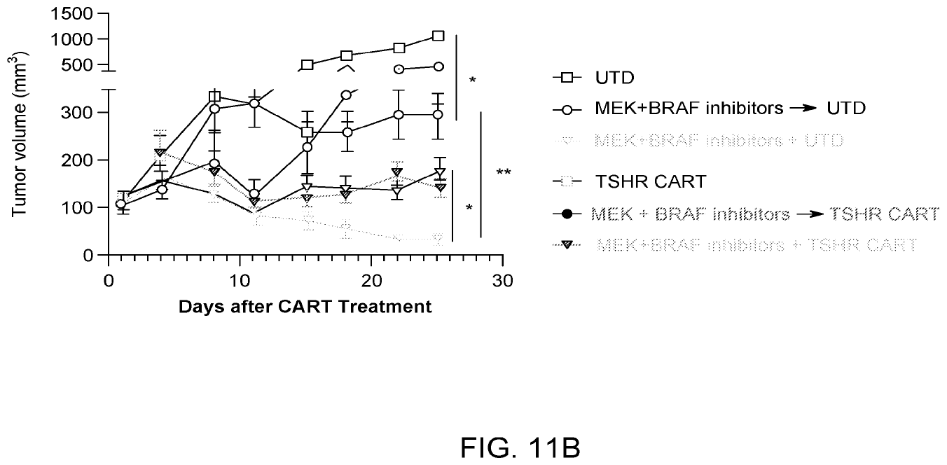

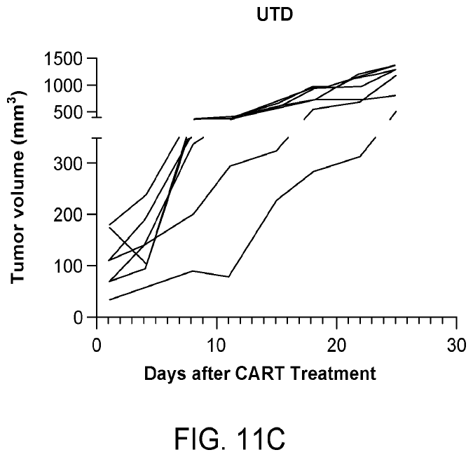

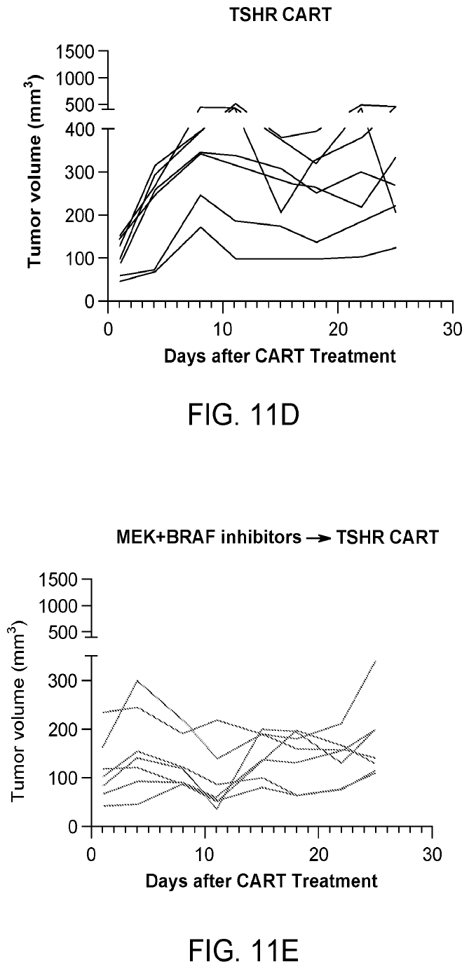

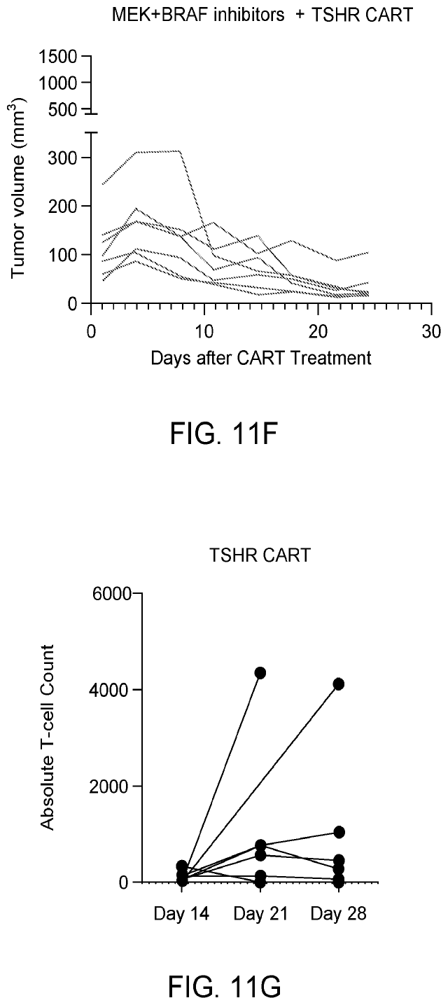

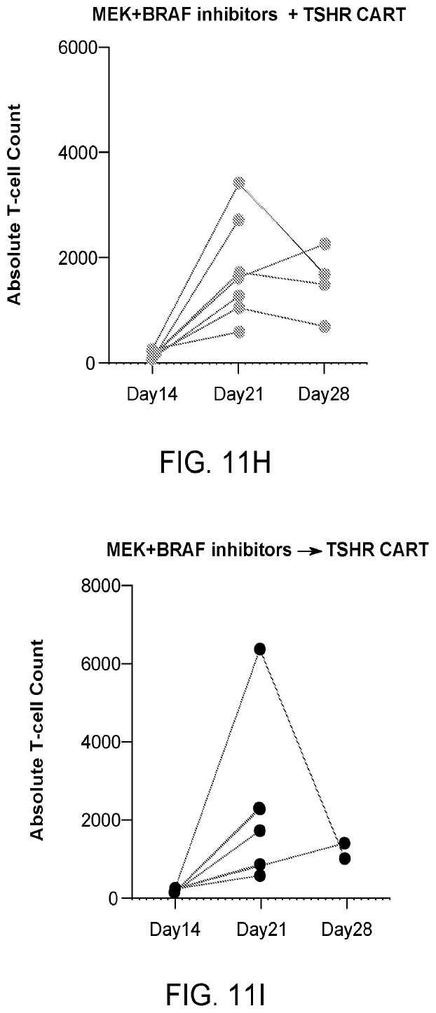

The following was performed to assess the kinetics of TSHR expression after MEK/BRAF inhibition. Mice were implanted with 5 mm³ Th-560 ATC PDX tumors. When tumors reached ˜100 mm³, mice were treated daily with either vehicle control or with 1.5 mg/kg R05126766 plus 1 mg/kg dabrafenib. TSHR protein expression was assessed by IHC and was found to be significantly elevated after 7 days of MEK/BRAF inhibitor treatment compared to vehicle control. However, TSHR expression was subsequently lost within two days after stopping MEK/BRAF inhibitor treatment (FIG. 11).

Different dosing regimens of MEK/BRAF inhibitors in combination with TSHR-CART cell therapy were compared in ATC PDX models. Mice were implanted with 5 mm³ Th-560 ATC PDX tumors. When tumors reached ˜100 mm³, mice were treated daily with vehicle control, 0.25 mg/kg trametinib (continuously administered or stopped after 7 days), or 0.25 mg/kg trametinib plus 2 mg/kg dabrafenib (continuously administered or stopped after 7 days). Mice treated with vehicle control were engrafted with tumor one week later than mice treated with MEK/BRAF inhibitors to ensure equal tumor sizes across all groups when UTD or TSHR-CART cells were administered. Mice were then given 10×10⁶ UTD or TSHR-CART cells via tail vein injection. Tumor volume, body weight, and body condition were inspected daily to monitor disease progression and to assay for endpoint criteria. CART cell expansion was assessed by serial peripheral blood sampling. Mice treated with continuous MEK/BRAF inhibitors and TSHR-CART cells showed superior antitumor efficacy (FIG. 11) and T cell expansion (FIG. 11). These findings indicate that continuous MAPK inhibition of de-differentiated thyroid cancers most effectively upregulates TSHR expression and enhances the antitumor activity of TSHR-CART cell therapy.

### Materials and Methods

### Cell Lines

K562 cells were obtained from ATCC (Manassas, VA, USA). For indicated experiments, K562 and FTC133 cell lines were transduced with a firefly luciferase vector containing a hygromycin resistance gene (Promega, Madison, WI, USA, Cat #E6691) as well as a TSHR vector containing a puromycin resistance gene (Genecopoeia Inc., Rockville, MD, USA). These cells were then cultured in selection media containing either hygromycin (1 μg/mL) or puromycin (2 μg/mL) to obtain a pure population. K562 cell lines were cultured in R10 medium made with RPMI 1640 (Gibco, Gaithersburg, MD, USA), 10% fetal bovine serum (FBS, Sigma, St. Louis, MO, USA), and 1% penicillin-streptomycin-glutamine (Gibco, Gaithersburg, MD, USA). FTC-133 cell lines were cultured in D10 medium made with DMEM (Corning Inc., Corning, NY, USA), 10% Fetal Bovine Serum (FBS, Sigma, St. Louis, MO, USA), and 1% penicillin-streptomycin-glutamine (Gibco, Gaithersburg, MD, USA).

### CAR Construct and CART Cell Production

Peripheral blood mononuclear cells (PBMCs) were isolated from de-identified normal donor blood apheresis cones using SepMate tubes (STEMCELL Technologies, Vancouver, BC, Canada) and Lymphoprep™ solution (STEMCELL Technologies, Vancouver, BC, Canada). T cells were separated with negative selection magnetic beads using EasySep™ Human T Cell Isolation Kit (STEMCELL Technologies, Vancouver, BC, Canada). Second-generation 4-1BB-costimulated TSHR CAR constructs were synthesized de novo (IDT, Coralville, USA) and cloned into a third-generation lentivirus under the control of the EF-1α promotor. The TSHR-targeted single chain variable fragment was derived from an autoantibody from a patient with Graves' disease, clone KI-70. TSHR-CART cells were then generated through the lentiviral transduction of normal donor T cells as described below. Lentiviral particles were generated through the transient transfection of plasmid into 293T virus-producing cells (ATCC, Manassas, VA, USA), in the presence of Lipofectamine 3000 (Invitrogen, Carlsbad, CA, USA), vesicular stomatitis virus G (VSV-G) and packaging plasmids pCMVR8.74 (Addgene, Cambridge, MA, USA). The titers and subsequently multiplicity of infection (MOI) were analyzed and calculated by flow cytometry. T cells isolated from normal donors were stimulated using anti-CD3/CD28 Dynabeads (Life Technologies, Oslo, Norway) at a 3:1 beads-to-cell ratio and then transduced 24 hours after stimulation with lentivirus particles at a MOI of 3. T cells were cultured in T cell medium containing X-VIVO™ 15 media (Lonza, Basel, Switzerland), 10% human AB serum (Corning Inc., Corning, NY, USA), and 1% penicillin-streptomycin-glutamine (Gibco, Gaithersburg, MD, USA). Magnetic bead removal and the evaluation of CAR expression on T cells by flow cytometry were performed on day 6 by staining with a goat anti-mouse F(ab′)2 antibody (Invitrogen, Carlsbad, CA, USA). CART cells were harvested and cryopreserved on day 8 for future experiments. CART cells were thawed and rested in T cell medium overnight at 37° C. and 5% CO₂ prior to experimental use.

### Multi-Parametric Flow Cytometry

Anti-human antibodies were purchased from Biolegend (San Diego, CA, USA), eBioscience (San Diego, CA, USA), or BD Biosciences (San Jose, CA, USA). In all analyses, the population of interest was gated based on forward vs side scatter characteristics, then by singlet gating, and then by live cell gating using LIVE/DEAD™ Fixable Aqua Dead Cell Stain Kit (Invitrogen, Carlsbad, CA, USA). Flow cytometry was performed on a three-laser CytoFLEX (Beckman Coulter, Chaska, MN, USA). All analyses were performed using FlowJo X10.0.7r2 software (Ashland, OR, USA).

### Immunohistochemistry (IHC)

Immunostaining was performed on paraffin-embedded tissue. Thyroid tissue microarray (TMA) was made from archival samples. Paraffin blocks were cut into 5 μm thick sections. Slides were deparaffinized, rehydrated with decreasing concentrations of ethanol, and finally in the water. Antigen retrieval was performed using Antigen Retrieval Solution (pH 6 or pH 9) (Dako, Glostrup, Denmark) for at least 15 minutes in an autoclave, and cooling for 30 minutes at room temperature. Slides were washed with 3% hydrogen peroxide (Fisher Scientific, Waltham, MA, USA) for 10 minutes to block the activity of endogenous peroxidases. Primary anti-human TSHR antibody was used for 60 minutes at room temperature at 1:500 (abcam, ab218108, Cambridge, UK). The infiltration of human T lymphocytes into the neoplastic tumors was marked using anti-human CD3 at 1:500 (abcam, ab52959, Cambridge, UK). The primary antibody was detected using the Envision Labeled Kit (Dako, Glostrup, Denmark) according to the manufacturer's protocol. After washing, slides were counterstained with Mayer's hematoxylin (Sigma-Aldrich, Burlington, MA, USA). Slides were dehydrated through ethanol and xylene and cover-slipped using a xylene-based mounting medium (Fisher Scientific, Waltham, MA, USA). Samples were examined under bright-field illumination at ×20 objectives, and digital images were obtained using Aperio AT2 (Leica, Wetzlar, Germany). Results were processed using Aperio eSlide Manager and H-Score values were estimated using the Aperio ImageScope Software (both Aperio Technologies, Vista, CA, USA). Normal tissues were used as a positive and negative control.

### Immunocytochemistry (ICC—HistoGel)

Cells were washed three times with PBS (Corning, NY, USA), scraped from the bottom of the plate using a cell lifter (Fisherbrand, Waltham, MA, USA), transferred to a 50 mL polypropylene tube, and centrifuged at room temperature for 2 minutes at 500×g. The supernatant was aspirated, and 10% neutral buffered formalin (Fisher Scientific, Waltham, MA, USA) was added for 30 minutes at room temperature. HistoGel (Thermo Scientific, Waltham, MA, USA) was heated in the microwave for 3 seconds at maximal power and converted to a liquid state. Cells were transferred to the HistoScreen Tissue Cassettes (Thermo Scientific, Waltham, MA, USA) and covered with liquid Histogel. Samples were placed at room temperature and allowed to solidify. HistoGel blocks were transferred into tissue embedding cassettes (Thomas Scientific, Swedesboro, NJ, USA), dehydrated in increasing concentrations of ethanol and xylene, and paraffin embedded. Sections were prepared as described in the immunohistochemistry section. Primary anti-human TSHR antibody was used for 60 minutes at room temperature at 1:1500 (abcam, ab218108, Cambridge, UK). Envision labeled polymer (Dako, Glostrup, Denmark) was used for 30 minutes as a secondary antibody. Slides were stained with diaminobenzidine tetrahydrochloride (DAB) chromogen (Dako, Glostrup, Denmark) for 5 minutes at room temperature and counterstained with Mayer's hematoxylin (Sigma-Aldrich, Burlington, MA, USA). Control staining with hematoxylin and eosin (Sigma-Aldrich, Burlington, MA, USA) was also performed. Aperio AT2 scanner (Leica, Wetzlar, Germany) was used for the digital image, at 20× objective. Images were analyzed using the Aperio ImageScope software (Aperio Technologies, Vista, CA, USA).

### Immunocytochemistry (ICC)

THJ-529 cells (2×10⁴ cells/chamber) were plated in a 4-well chamber slide (Nunc, Thermo Scientific, Waltham, MA, USA), incubated overnight. After 24 hours, media was aspirated, cells were washed three times with PBS (Corning, NY, USA). Then, 2% paraformaldehyde was added for 20 minutes at room temperature. Again, cells were washed three times with PBS. Next, ice-cold 100% methanol (Fisher Scientific, Waltham, MA, USA) was added for 7 minutes at −20° C. Methanol was aspirated, and samples were allowed to air dry for 10 minutes. Next, serum-free-blocking diluent (Dako, Glostrup, Denmark) was used for 30 minutes at room temperature. Primary anti-human TSHR antibody was used for 60 minutes at room temperature at 1:1500 (abcam, ab218108, Cambridge, UK). Slides were subsequently prepared as described in the immunohistochemistry section.

### Tissue Mircoarray (TMA)

A tissue microarray (TMA) was made from archival formalin fixed paraffin embedded samples under Mayo Clinic IRB approval. TMA tissues were cut into 5 mm sections, deparaffinized, hydrated, antigen retrieved and blocked with diluent that contained background reducing components (DAKOCytomation, Glostrup, Denmark). Immunostaining was done with HDAC1 at 1:100 (Santa Cruz) and HDAC6 at 1:100 (Cell Signaling). The Envision Dual Labeled Polymer kit (DAKOCytomation) was used according to the manufacturer's instructions and then lightly counterstained with Gill I hematoxylin (Sigma-Aldrich) before dehydration and mounting. Images were obtained at 20X using Scanscope XT (Aperio Technologies, Vista, CA, USA) and the staining of the TMA punches were scored using an algorithm in the Imagescope Software (Aperio Technologies) created by a histologist based upon signal intensity (0, 1C, 2C, 3C). The H-score was determined by adding the results of multiplication of the percentage of cells with staining intensity ordinal value (scored from 0 for “no signal” to 3 for “strong signal”) with 300 possible values. In this system. <0.1% positive cells is considered to be a negative result. Cases were excluded from the study if a section could not be assigned a score due to insufficient quantity of tumor tissue present.

### In Vitro T Cell Function Assays

Proliferation assays were performed as described elsewhere (Sterner, R. M. et al., Blood 133, 697-709 (2019)). UTD or TSHR-CART cells were re-suspended in T cell medium at 1×10⁶/mL, and 100 μL per well were seeded in 96-well plates. Each assay included T cells with media only as a negative control and T cells with PMA and ionomycin as a positive control. After five days, cells were harvested and stained with APC-H7 anti-human CD3 (eBioscience, San Diego, CA, USA), BV421-CD4 (BioLegend, San Diego, CA, USA), and LIVE/DEAD™ Fixable Aqua.

Cytotoxicity assays were performed as described elsewhere (Sterner, R. M. et al., Blood 133, 697-709 (2019)). Luciferase*TSHR*K562 or FTC133 cells were used as target cells. UTD or TSHR-CART cells were co-cultured with target cells at various effector: target (E:T) ratios in T cell medium. Cytotoxic efficiency was calculated by bioluminescence imaging on the Promega GlowMax Explorer (Promega Corporation, Fitchburg, WI, USA) at indicated time points.

Degranulation and intracellular cytokine assays were performed as described elsewhere (Sterner, R. M. et al., Blood 133, 697-709 (2019)). UTD or TSHR-CART cells were incubated with various target cells at an effector:target ratio of 1:5. Antibodies against FITC-CD107α (BD Pharmingen, San Diego, CA, USA, Cat #555800), CD28 (BD Biosciences, San Diego, CA, USA, Cat #348040), CD49d (BD Biosciences, San Diego, CA, USA, Cat #340976) and monensin (Biolegend, San Diego, CA, USA, Cat #420701) were added prior to the incubation. After 6 hours, cells were harvested and stained with LIVE/DEAD™ Fixable Aqua. Cells were then fixed, permeabilized (FIX & PERM Cell Fixation & Cell Permeabilization Kit, Life Technologies, Oslo, Norway, Cat #GAS004), and stained for CD3 (clone UCHT1), APC (Cat #17-0038-42, eBioscience, San Diego, CA, USA), and intracellular cytokines including IL-2 (clone 5344.111; BD Pharmingen, San Diego, CA, USA, Cat #562384), GM-CSF (clone BVD2-21C11; BD Pharmingen, San Diego, CA, USA, Cat #562930), IFN-γ (clone 4S.B3; Invitrogen, Carlsbad, CA, USA, Cat #47-7319-42), and MIP1-β (clone D21-1351; BD Pharmingen, San Diego, CA, USA, Cat #560687).

### In Vivo Models

Male and female 8-12 week old NOD-SCID-IL2^{rγ-/-} (NSG) mice were obtained from Jackson Laboratories (Jackson Laboratories, Bar Harbor, ME, USA) and maintained in an animal barrier space. TSHR/LUC^{+/+} FTC133 cells were suspended in a 50% Matrigel basement medium and subcutaneously injected into the rear flank of 6-8 week-old NSG mice. Imaging to follow tumor burden was performed 10 minutes after intraperitoneal injection of 10 μL/g D-luciferin (15 mg/mL, Gold Biotechnology, St. Louis, MO, USA) on a weekly basis until tumor engraftment was confirmed. Mice were then randomized based on tumor burden to receive different treatments as outlined in the specific experiment. Mice were euthanized once IACUC-approved endpoint criteria were met.

In ATC PDX models, NSG mice were surgically engrafted with 5 mm³ ATC PDX tumors in their rear flank. These mice were then treated with a sub-therapeutic dose of 1 mg/kg trametinib and 1.5 mg/kg dabrafenib as a sensitizing regimen to induce TSHR expression on ATC tumor cells. On day 7, additional mice were surgically engrafted with 5 mm³ PDX tumors in the same manner as the first arm, but were treated with placebo rather than MAPK inhibitors. On day 10, all mice were randomized based on tumor burden and injected with 10×10⁶ UTD or TSHR-CART. Disease progression was monitored through daily assessment of tumor volume with caliper measurements. Weekly tail vein bleeding after injection of CART cells was performed to assess T cell expansion. Mouse peripheral blood was lysed using BD FACS Lyse (BD Biosciences, San Diego, CA, USA) and then analyzed with flow cytometry. Mice were euthanized at IACUC-approved endpoints.

A more detailed protocol for in vivo experiments is shown in Example 4.

### Statistical Analysis

Prism Graph Pad (La Jolla, CA, USA) and Microsoft Excel (Microsoft, Redmond, WA, USA) were used to analyze raw experimental data. Statistical tests are described in figure legends.

### Example 4: Anti-Tumor Activity Against TSHR Expressing Tumor THJ-560T Grown in Mice by TSHR Car T after Dabrafenib and MEK Inhibitor Treatment

### General Protocol

1. Pre-Op: Mice are given 0.2 mg/mL (4.5 mL in water pouch) ibuprofen for 48 hours in the drinking water.
2. Implant: aged 11-12 weeks, implant 25 mm³ tumor tissue PDX THJ-560T in the right flank dipped in Matrigel.
3. Post-Op: Mice are given 0.2 mg/mL (4.5 mL in water pouch) ibuprofen for 48 hours in the drinking water and monitored.
4. Surgery clips are removed 14 days after surgery.

Randomization: Once tumors reach ˜100 mm³ (around 26 days after surgery), mice are treated with Trametinib.

Mice are randomized into nine groups (10 mice in each group) and ear punched for identification.

### GROUPS

1. UTD CAR-T-7 mice
2. 0.25 mg/kg Trametinib (STOP DAY 7)+UDT CAR-T-7 mice
3. 0.25 mg/kg Trametinib+2 mg/kg Dabrafenib (STOP DAY 7)+UDT CAR-T-7 mice
4. 0.25 mg/kg Trametinib (STOP DAY 7)+TSHR CAR-T-7 mice
5. 0.25 mg/kg Trametinib+2 mg/kg Dabrafenib (STOP DAY 7)+TSHR CAR-T-7 mice
6. 0.25 mg/kg Trametinib (CONTINUOUS)+UDT CAR-T-7 mice
7. 0.25 mg/kg Trametinib+2 mg/kg Dabrafenib (CONTINUOUS)+UDT CAR-T-7 mice
8. 0.25 mg/kg Trametinib (CONTINUOUS)+TSHR CAR-T-7 mice
9. 0.25 mg/kg Trametinib+2 mg/kg Dabrafenib (CONTINUOUS)+TSHR CAR-T-7 mice
10. TSHR CAR-T-7 mice
11. 0.25 mg/kg Trametinib (CONTINUOUS)-7 mice
12. 0.25 mg/kg Trametinib+2 mg/kg Dabrafenib (CONTINUOUS)-7 mice

Inject via tail vein 20 million CAR T cells per mouse.

### TREATMENTS

Treatment: Mice are treated with Trametinib 0.25 mg/kg+ and Trametinib 0.25 mg/kg+Dabrafenib 2 mg/kg for one week and continuously.

Trametinib is administered orally on a daily basis supplemented in Nutra-gel. For therapeutic testing, a 10-gram cube of Nutra-gel feeding 25 gram mice is used. For placebo, mice are given Nutra-gel without any additional compounds.

Preparation: Trametinib 0.25 mg/kg
0.00625 mg per mouse
0.03125 mg in 50 g of Nutra-Gel (for 5 mice)
70 g of Nutragel per group per day—0.04375 mg/in 70 g cube
0.04375 mg×5 groups=0.21875 mg per day in 350 g of Nutragel
For two weeks prepare: 3.1 mg of Trametinib in 4900 g of Nutragel (14 days)
   Preparation: Dabrafenib 2 mg/kg
0.05 mg per mouse
0.25 mg in 50 g of Nutra-Gel (for 5 mice)
70 g of Nutra-gel per group per day—0.35 mg/in 70 g cube
0.35 mg×5 groups=1.75 mg per day in 350 g of Nutra-gel
For two weeks prepare: 25 mg of Trametinib in 4900 g of Nutra-gel

### Prepare Nutra-gel

1960 g of Nutra-gel powder+2940 mL of sterile water form DCM (Ratio: 200 g Nutra-gel: 300 mL water)
For 0.25 mg/kg Trametinib add: 3.1 mg of Trametinib
For 0.25 mg Trametinib mix: 3.1 mg of Trametinib+25 mg of Dabrafenib
   Placebo—only two groups Group 1 and 10
70 g of Nutra-gel per day×2 groups=140 g per day
140g×14 days=1960 g
   Prepare Nutra-gel for placebo
784 g of Nutra-gel powder+1176 mL of sterile water form DCM (Ratio:200 g Nutra-gel: 300 ml water)
   Day before treatment—injection of CAR T or UTD cells, prepare the media for the CAR T cells and pull out the cells:
   Media for CAR T cells:
100 mL X-Vivo 15 (Lonza, 04-418Q)
1 mL Pen/Strep/L-Glut (1%)
10 mL human serum albumin (10%)
0.45 μM vacuum filter
0.2 μM vacuum filter

### Media Preparations:

Thaw reagents in the water bath
Prepare 90 mL of X-Vivo 15
Add 1 mL of PSG and 10 mL of Human Serum Albumin
Clarify with 0.45 μM filter
Sterilize with 0.2 μM filter

### Cell's Preparation:

### For UDT and CAR T:

1. Prepare 500 mL TCM media by mixing:
   445 mL X-Vivo 15 (Lonza, 04-418Q) (deli fridge)
   5 mL Pen/Strep/L-Glut (1%) (in −20 rack 6)
   50 mL human serum albumin (10%)
2. Clarify media by using 0.45 μM vacuum filter.
3. Clarify media by using 0.2 μM vacuum filter.
4. Place media in Rock Bath and warm up to 37° C.
5. Prepare 50 mL tube and add 10 mL of TCM media.
7. Thaw the CARTs:
   Remove the CAR Ts from liquid nitrogen and place them in the 37° C. water bath, checking often. Do not leave them in the water bath for more than about 2 minutes.
   In a hood, use a 3 mL transfer pipette to slowly drip approximately 1 mL of the warm TCM from the 50 mL conical into each vial. Use the same transfer pipette to transfer the now liquefied ice crystal into the 50 mL conical.
8. Wash out the diluted cells by spinning at 300×g for 5 minutes.
9. Aspirate the medium, being careful not to disturb the cell pellet.
10. Resuspend the pellet in 5 mL warm TCM
11. Calculate the cells and dilute them up to 2×10⁶/mL and rest overnight at 37° C., 5% CO₂ in the appropriately sized vessel (based on volume).
12. Resuspend 60×10⁶ CAR T cells in 30 mL TCM as a final volume after washing and rested overnight in an upright T75.
13. Count thawed cells and spin at 1500 rpm×2 minutes. Determine the viability of the cells and prepare 20 million or more of live cells for injection for each mouse.
14. Resuspend cells in PBS at 20 million cells/0.2 mL.
15. Inject 20 million/200 μL of cells via tail vein injection into mouse.
16. Body weight and tumor volumes are measured 2-3 times weekly.
   a. Tumor volume (mm³)=0.532 (width×length×height)
   b. Body weight % change (tumor weight corrected)=[(BWₓ−TWₓ)−BW₀]/BW₀
      i. 1000 mm³=1 gram

Mice are in the experiment around 35 days. Placebo group (UTD cells) are sacked when tumor(s) reach 10% of body weight. Blood and tissue are collected.
17. At termination, the following are collected:
   a. Tumor—FFPE blocks in formalin
   b. Blood—½ in sodium heparin tubes (pool n=2-4) for plasma
   c. Blood one week after CAR T injection and every week after treatment, ship samples to Rochester.

As described herein, treatment with TSHR CAR T cells is used to improve survival as compared to the survival levels that can be observed in control animals.

### Example 5: Priming Thyroid Cancer Cells with MAPK Inhibitors to Improve TSHR-CART Therapy

Antitumor efficacy of TSHR CART cell therapy. To evaluate whether the combination of continuous MEK+/−BRAF inhibitor(s) treatment with TSHR CAR T cells results in enhanced (synergistic or additive) antitumor activity due to combined and distinct mechanisms of killing thyroid cancer cells as well as continued elevated expression of TSHR on tumor cells, PDX models representative of metastatic iodine-resistant thyroid cancer were used as described in Example 3. In vivo experiments using THJ-560 (MC-Th-560) PDX models were used to examine TSHR expression based on IHC at different timepoints following different doses and schedules of MEK+/−BRAF inhibitors (FIG. 3B). THJ-529 (MC-Th-529) PDX, representing a poorly differentiated thyroid carcinoma, has been shown to upregulate TSHR in response to MEK inhibition (FIG. 13).

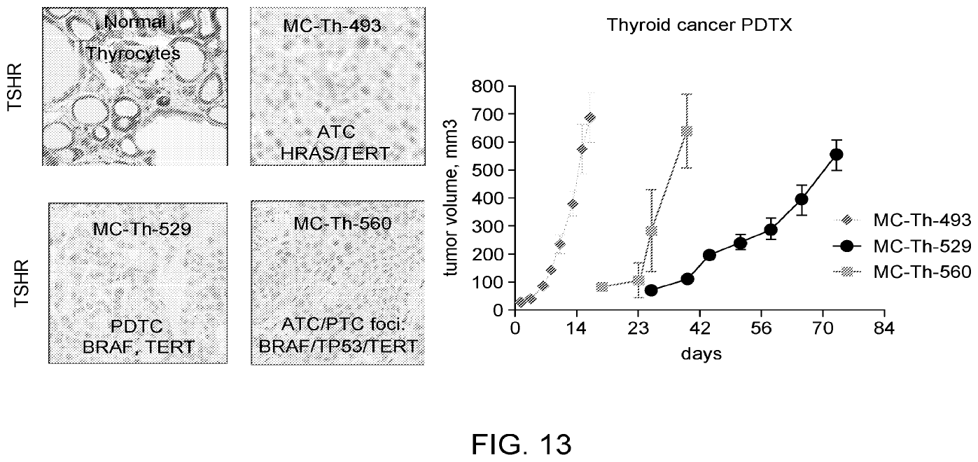

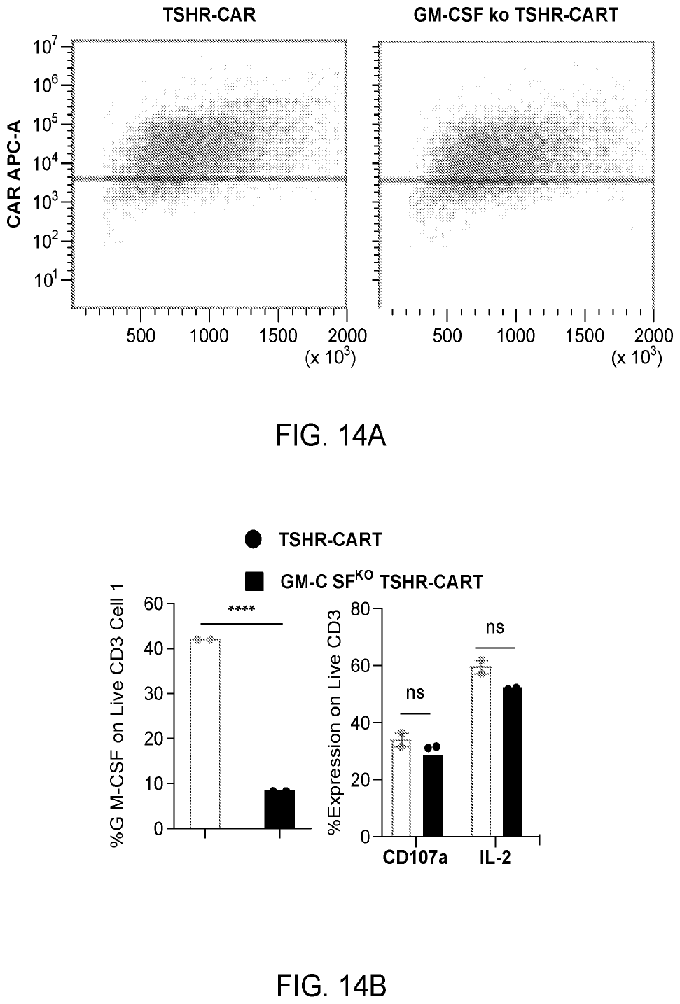

Generation of GM-CSF^{k/o} TSHR CAR T cells. GM-CSF^{k/o} TSHR CAR T cells were generated, in which exon 2 of GM-CSF was disrupted using CRISPR technology during CAR T cell manufacturing. GM-CSF^{k/o} TSHR CAR T cells had similar levels of CAR surface expression to wildtype (GM-CSF^{wt}) TSHR CAR T cells. Upon antigen-specific stimulation with TSHR⁺ cell lines, GM-CSF^{k/o} TSHR CAR T cells exhibited significantly less GM-CSF production but maintained IL-2 and CD107a degranulation (FIG. 14).

### Example 6: TSHR CAR T Targeted Therapy in Hurthle Cell Thyroid Carcinoma XTC.UC1

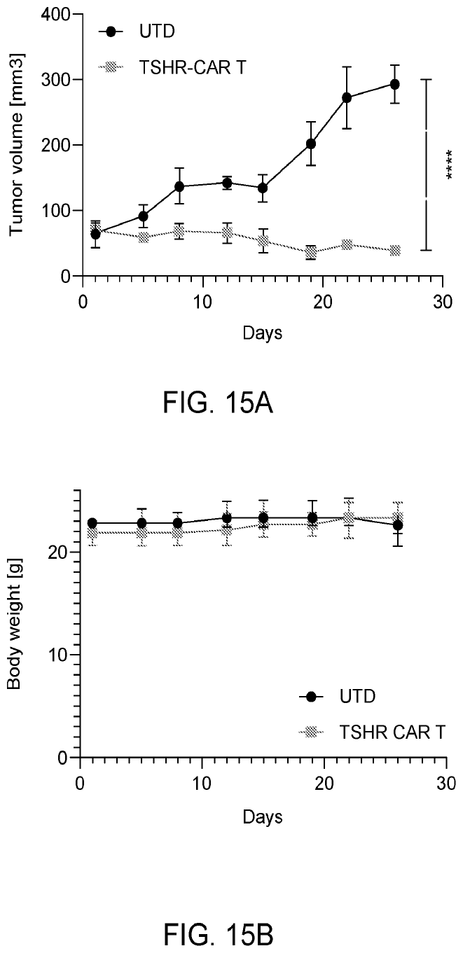

Hurthle cells XCT.UC1 (10⁶ cells/mouse) were injected into the right flank of NSG mice (n=12) in 50% PBS and 50% Matrigel. After 48 hours, mice were randomized into two groups (n=6), with an average tumor size per mouse of around 70 mm³. One group was injected with untransduced control T cells (UTD), and the second group was injected with TSHR CAR T cells. Each group received (10⁶ cells/mouse) via tail vein. Tumor volume and body weight were measured twice weekly. Tumor volume decreased in mice treated with TSHR CAR T cells (FIG. 15A) while the body weight of the mice was unchanged (FIG. 15B). Statistical analysis was determined using unpaired, two-tailed t-test.

These results demonstrate that TSHR CAR T targeted therapy attenuated Hurthle cell thyroid carcinoma XCT.UC1 tumor volume.

### Example 7: TSHR CAR T Targeted Therapy in Hurthle Cell Thyroid Carcinoma XTC. UC1

Previously injected XCT.UC1 cells grown subcutaneously in the right flank of NSG mice treated with TSHR CAR T are monitored for 3-4 months and then mice are rechallenged with new XCT.UC1 tumor cells injected subcutaneously in the left flank. Whether TSHR CAR T cells remain in the mouse and whether these cells expand to destroy the new growing tumor is determined. Blood is drawn on days 7, 14, and 21 post tumor injection and examined for the presence of TSHR CAR T cells. Tumor volume is monitored for 2-3 months dependent upon growth.

As described herein, TSHR CAR T cells can persist within the mice and can expand to target and destroy TSHR-expressing tumor cells in animals.

In vitro assays are performed in cell culture of XCT.UC1 cells treated with UTD or TSHR CAR T cells to examine cytotoxicity of TSHR CAR T cells and killing of XCT.UC1 tumor cells as detected by cytokine secretion. Cytokines that can be examined include interleukin-2 (IL-2), GM-CSF, IFN-gamma, MIP1b, and CD107a.

As described herein, TSHR CAR T cells can exhibit elevated cytokine secretion in animals.

### Example 8: Treatment of Thyroid Tumors with MAPK Inhibitors Improved Anti-Tumor Activity

### Methods

### TSHR Immunohistochemistry Method

Immunostaining was performed on paraffin-embedded tissue. Antigen retrieval was performed using Antigen Retrieval Solution (pH 9) (Dako, S236784-2, Glostrup, Denmark) for at least 30 minutes in an autoclave, and cooling for 10 minutes at room temperature. Washing slides with 3% hydrogen peroxide (Fisher Scientific, Waltham, MA, USA) for 10 minutes blocked the activity of endogenous peroxidases. Primary antibodies were used for 60 minutes at room temperature: anti-human TSHR antibody at 1:500 (Abcam, ab218108, Cambridge, UK). The primary antibody was detected using the HRP Labelled Polymer Anti-Rabbit Envision System Kit (Dako, K4003, Glostrup, Denmark) according to the manufacturer's protocol. After washing, slides were counterstained with Gill I hematoxylin (Epredia, 6765006, Kalamazoo, MI, USA). Slides were dehydrated through ethanol and xylene and cover-slipped using a xylene-based mounting medium (Fisher Scientific, Waltham, MA, USA). Normal thyroid tissues were used as a positive and negative control.

### MEK Inhibitors R05126766 (CH5126766) and Trametinib in Thyroid PDX Models

Subcutaneous thyroid PDX models (based on MC-Th-493, MC-Th-529, MC-Th-560, or MC-THJ374 cells) were established in Nod SCID Gamma (NSG) strain 005557 (6-8 weeks old) from Jackson Laboratory.

Pre-Op: Mice aged 6 −8 weeks were given either 0.035 mg/mL carprofen (315 μl in water pouch) or 0.2 mg/mL (4.5 mL in water pouch) ibuprofen for 24 hours in the drinking water.

Implant: Mice were implanted with 5 mm³ tumor tissue in the right flank and then 100 μL Matrigel was added once the incision was closed. For THJ374, 35 mice (7 groups) were implanted.

Post-Op: Mice were given either 0.035 mg/mL carprofen (315 μL in water pouch) or 0.2 mg/mL (4.5 mL in water pouch) ibuprofen for 48 hours in the drinking water and monitored.

Randomization: Once tumors reach ˜50-75 mm³, mice were randomized into 7 groups (5 mice in each group) and were ear punched for identification.

Therapy: Treatment was started when the tumor reached 145-155 mm³. Treatment groups were as follows:
a. Group *1—placebo (Nutra-gel)-4 mice
b. Group *2—RO5126766-0.75 mg/kg and dabrafenib 12.5 mg/kg-4 mice
c. Group *3—trametinib-1 mg/kg and dabrafenib 12.5 mg/kg-4 mice
   RO5126766 (CH5126766) MEK inhibitor

RO5126766 (CH5126766) was administered orally on a daily basis supplemented in Nutra-gel for 7 days (0.75 mg/kg). For therapeutic testing, a 50-gram cube of Nutra-gel feeding 25 gram mice was supplemented with 0.09375 mg RO5126766 (CH5126766). For placebo, mice were given Nutra-gel without any additional compounds.

Preparation: 0.09375 mg in 50 g of Nutra-gel, 0.9375 mg in 500 g of Nutra-gel (for 10 days), or 0.65625 mg in 250 g Nutra-gel (for 7 days).

### Trametinib MEK Inhibitor

Trametinib was administered orally on a daily basis supplemented in Nutra-gel for 14 days (1 mg/kg). For therapeutic testing, a 50-gram cube of Nutra-gel feeding 25 gram mice was supplemented with 0.125 mg trametinib. For placebo, mice were given Nutra-gel without any additional compounds.

Preparation: 0.125 mg in 50 g of Nutra-gel, 1.25 mg in 500 g of Nutra-gel (for 10 days), or 0.875 mg in 250 g Nutra-gel (for 7 days).

### Dabrafenib-BRAF Inhibitor

Dabrafenib was administered orally on a daily basis supplemented in Nutra-gel for 7 days (12.5 mg/kg). For therapeutic testing, a 50-gram cube of Nutra-gel feeding 25 gram mice was supplemented with 1.5625 mg Dabrafenib.

Preparation: 1.5625 mg in 50 g of Nutra-Gel (for 5 mice), 15.62 5 mg in 500 g of Nutra-gel (for 10 days), or 10.9375 in 250 g of Nutra-gel (for 7 days)

### Tumor Assessment

Mouse body weight and tumor volumes were measured 2-3 times weekly.
Tumor volume (mm³)=0.532 (width×length×height)
Body weight % change (tumor weight corrected)=[(BWₓ−TWₓ)−BW₀]/BW₀
1000 mm³=1 gram

Tumors were collected the after 7 days and were stained for TSHR using IHC.

At termination, tumors and blood were collected from each mouse as follows:
Collect tumor—½ for FFPE blocks+½ in media on ice for Western blot.
Collect blood—½ in sodium heparin tubes (pool n=2-4) for plasma and ½ in 2 mL tubes for clotting, serum (pool n=2). Aliquot serum into 2 tubes and note volume.

### Results

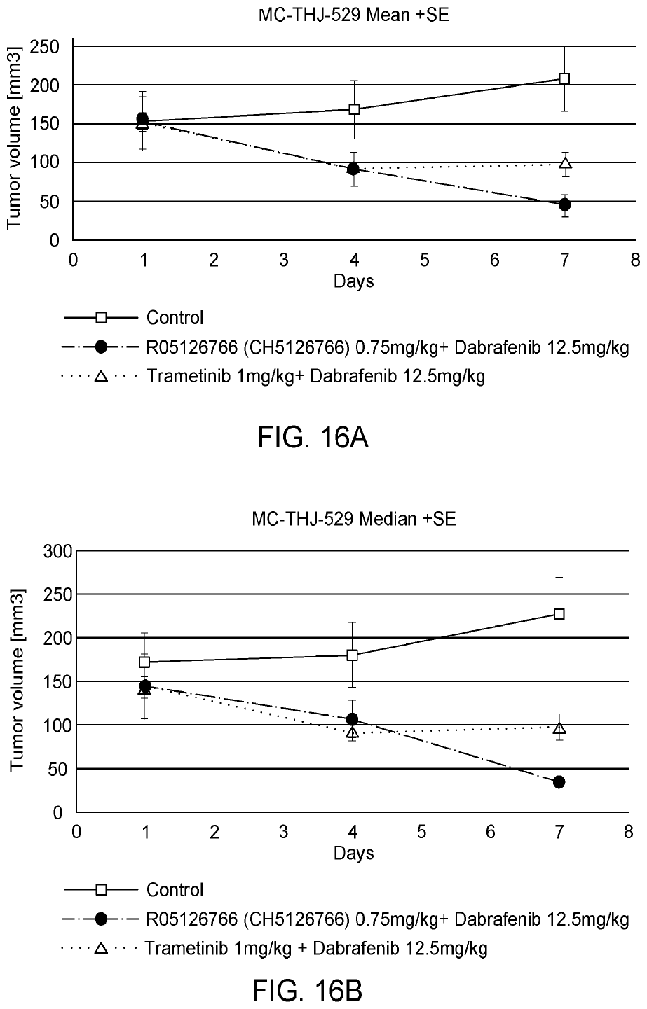

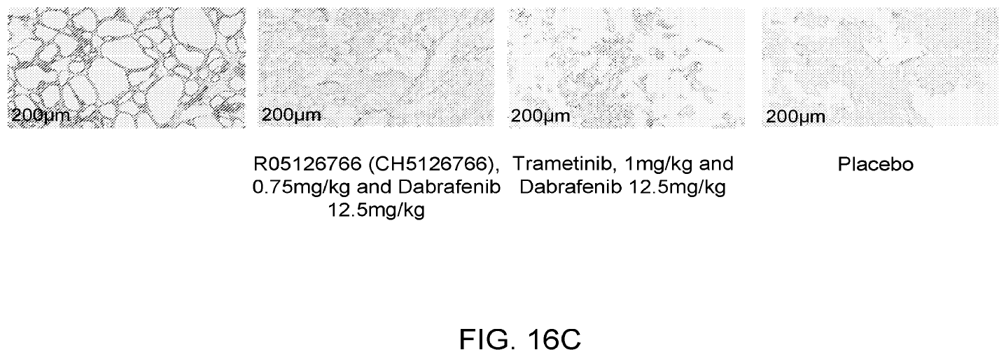

Tumor volumes of MC-THJ-529 tumors treated with one or more MEK inhibitors and/or one or more BRAF inhibitors are shown in Table 23. A MEK inhibitor (trametinib or R05126766) and a BRAF inhibitor together upregulated TSHR on the cell surface membrane as well as inhibited tumor growth of MC-THJ-529 tumors (FIGS. 16A-16C).

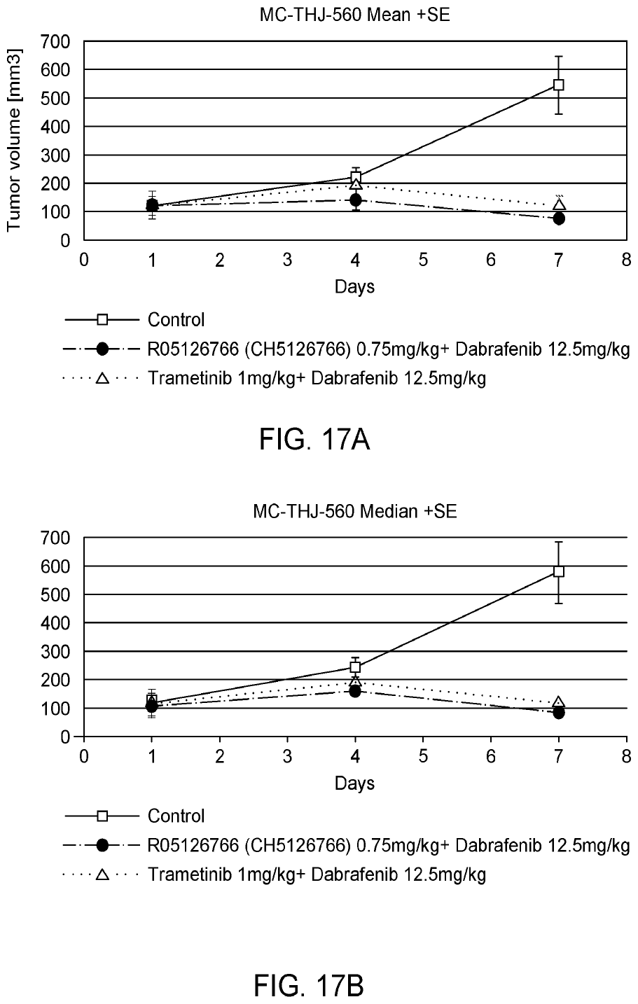

Tumor volumes of MC-THJ-560 tumors treated with one or more MEK inhibitors and/or one or more BRAF inhibitors are shown in Table 24. A MEK inhibitor (trametinib or R05126766) and a BRAF inhibitor together upregulated TSHR on the cell surface membrane as well as inhibited tumor growth of MC-THJ-560 tumors (FIGS. 17A-17B).

TABLE 23
Volume of MC-THJ-529 tumors treated with one or more
MEK inhibitors and/or one or more BRAF inhibitors.

| | | Tumor Volume (mm³) | | |
|---|---|---|---|---|
| | | Day 1 | Day 4 | Day 7 |
| Control | mouse 1 | 191.02 | 246.872 | 271.8073992 |
| (Placebo | mouse 2 | 207.53 | 175.9 | 277.62581 |
| Nutra-Gel ™) | mouse 3 | 49.40 | 67.4581 | 101.0446945 |
| | mouse 4 | 151.46 | 183.7328 | 181.9653425 |
| | avg | 149.8532 | 168.4908 | 208.1108116 |
| | median | 171.24 | 179.82 | 226.89 |
| | sd | 70.97795 | 74.47258 | 83.73834101 |
| | n | 4 | 4 | 4 |
| | SE | 35.48898 | 37.23629 | 41.8691705 |
| R05126766 | mouse 5 | 108.8816 | 92.27 | 32.47820428 |
| (CH5126766; | mouse 6 | 248.5024 | 123.92 | 86.58752256 |
| 0.75 mg/kg) + | mouse 7 | 81.69836 | 29.92 | 35.11309771 |
| dabrafenib | mouse 8 | 179.2806 | 120.58 | 24.05379863 |
| (12.5 mg/kg) | avg | 154.5907 | 91.67199 | 44.5581558 |
| | median | 144.0811 | 106.4267 | 33.795651 |
| | sd | 74.90354 | 43.54859 | 28.41379175 |
| | n | 4 | 4 | 4 |
| | SE | 37.45177 | 21.7743 | 14.20689587 |
| trametinib | mouse 9 | 187.5627 | 119.66 | 128.4804444 |
| (1 mg/kg) + | mouse 10 | 134.666 | 93.16 | 68.65524358 |
| dabrafenib | mouse 11 | 146.8077 | 75.72 | 75.7175342 |
| (12.5 mg/kg) | mouse 12 | 140.1103 | 89.34 | 118.3711526 |
| | avg | 152.29 | 94.47 | 97.81 |
| | median | 143.46 | 91.25 | 97.04 |
| | sd | 24.04 | 18.39 | 30.01 |
| | n | 4 | 4 | 4 |
| | SE | 12.02 | 9.19 | 15.00 |

TABLE 24
Volume of MC-THJ-560 tumors treated with one or more
MEK inhibitors and/or one or more BRAF inhibitors.

| | | Tumor Volume (mm³) | | |
|---|---|---|---|---|
| | | Day 1 | Day 4 | Day 7 |
| Control | mouse 1 | 185.94 | 297.445 | 742.6211993 |
| (Placebo | mouse 2 | 79.70 | 147.298 | 378.4588361 |
| Nutra-Gel ™) | mouse 3 | 165.30 | 263.162 | 785.773021 |
| | mouse 4 | 69.45 | 220.8725 | 412.2729848 |
| | avg | 125.0963 | 232.1944 | 579.7815103 |
| | median | 122.50 | 242.02 | 577.4470921 |
| | sd | 59.09458 | 64.68431 | 214.1177185 |
| | n | 4 | 4 | 4 |
| | SE | 29.54729 | 32.34215 | 107.0588592 |
| R05126766 | mouse 5 | 86.5717 | 54.34 | 41.6141572 |
| (CH5126766; | mouse 6 | 122.4603 | 203.26 | 94.583947 |
| 0.75 mg/kg) + | mouse 7 | 225.6899 | 197.29 | 90.09661122 |
| dabrafenib | mouse 8 | 78.93455 | 121.47 | 80.1483395 |
| (12.5 mg/kg) | avg | 128.4141 | 144.09 | 76.61 |
| | median | 104.516 | 159.3814 | 85.12 |
| | sd | 67.56984 | 70.47163 | 24.09827318 |
| | n | 4 | 4 | 4 |
| | SE | 33.78492 | 35.23582 | 12.04913659 |
| trametinib | mouse 9 | 155.2885 | 186.32 | 155.288534 |
| (1 mg/kg) + | mouse 10 | 76.21813 | 187.15 | 76.21812722 |
| dabrafenib | mouse 11 | 28.76889 | 58.23 | 44.71737732 |
| (12.5 mg/kg) | mouse 12 | 258.0115 | 378.99 | 218.829601 |
| | avg | 129.57 | 202.67 | 123.76 |
| | median | 115.75 | 186.73 | 115.75 |
| | sd | 100.28 | 132.24 | 78.61 |
| | n | 4 | 4 | 4 |
| | SE | 50.14 | 66.12 | 39.31 |

Together these results demonstrate that one or more MEK inhibitors and/or one or more BRAF inhibitors can be used to upregulate TSHR expression. For example, one or more MEK inhibitors and/or one or more BRAF inhibitors can be used improve anti-tumor activity of TSHR CAR T targeted therapy.

### Example 9: Clone 2-1

Heavy chain coding sequence
(SEQ ID NO: 130)
GAATCAAAAGCCTCTGAAGTCCAGCTGTTGGAAAGCGGCGGTGGTTTGGTCCAATTTCGCGGCA
GCCGACGCCTCTCCTGCGCGGTTTCTGGTTTCTCAGTCTCCGGTAACCAGATGACATGGGTCCG
GCAAGCGCCAGGTAAGGGCCTTGAATGGCTCTCTGTAAAGAATAGTGATGGCTCCACATCATAT
GCAGATTCTGTAAAAGGTAGGTTCACAATCGCTCGCGACGAGGTAAAAAACACAGTTTTTCTTC
AAATGAACGCTGTACGAGCAGAGGACACCGCGTTGTATTACTGCGCTAGACTCAAGAATGGCGT
GTTCGACATCTGGGGTCAGGGTACGATGGTAACGGTTAGCTCA
Heavy chain
(SEQ ID NO: 51)

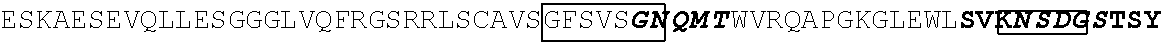

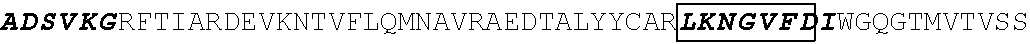

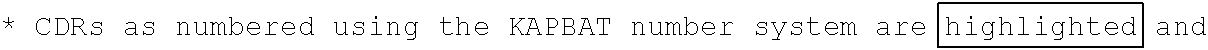

CDRs as numbered using the CHOTIA numbering system are bold and
italicized
Light chain coding sequence
(SEQ ID NO: 139)
CAGCCCGTCCTTACTCAGCCTACATCCCTGTCTGCTAGTCCTGGGGCGTCCGCGAGTTTGACTT
GCACCCTGCGAAGGGGTATTAATTTGGGAGCGTATGGCATCCATTGGTACCAGCAACGGCCTGG
AAGTCCCCCACGATATCTGCTCAGACACAAGAGCGCATCAGACAAGCAGCAGGGCAGTGGGGTT
CCAGGGAGGTTTTCCGGGAGCAAGGACGCCAGTGCCAATGCCGGCCTCCTCCTGATTTCTGGGC
TGCAATCAGAAGACGAAGCAGACTACTATTGTATGATATACTATAACAGCGCTTGGGTCTTCGG
TGGCGGCACAAAACTGACCGTTCTGGGCGAGGGTAAA
Light chain
(SEQ ID NO: 87)

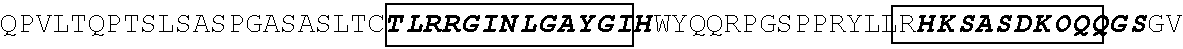

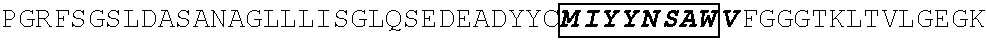

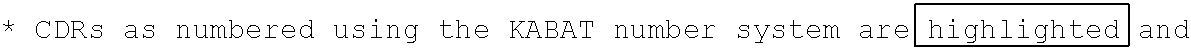

CDRs as numbered using the CHOTIA numbering system are bold and
italicized

### Example 10: Clone 2-2

Heavy chain coding sequence
(SEQ ID NO: 131)
GAAAGTAAGGCTTCCGAGGTACAGTTGGTCGAAAGTGGAGGAGGACTGGTACAGCCACGAGGTA
GCCTCAGACTCTCTTGCGCGGCATCAGGGTTTACTTTTACAACTTTTGCAATGTCCTGGGTGAG
GCAAGCGCCGGGAAAGGGGCTGGAGTGGGTGGCGACTCGCAATGGAAACGGTGGCCGAACTTAT
TATGCCGACTCAGTACGAGGCAGATTCACAATTTCACGAGACCTGCACCTTCAGATGAACTCTT
TGCGCGTTGAGGATACGGCAGTTTATTACTGCACAAAGGACCTTGGGCCAGTCGTAAGAGGCAC
TTTTGACGTATGGGGCCAGGGGACGATGGTTACAGTCAGCTCA
Heavy chain
(SEQ ID NO: 52)

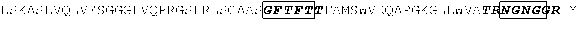

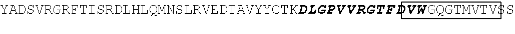

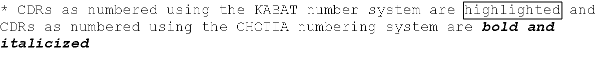

Light chain coding sequence
(SEQ ID NO: 140)
TCTTCTGAACTGACGCAGGATCCGACTGTGTCAGTCGCTTTGGGACAGACGGTACGAATCACCT
GCCAAGGAGACAGTCTCCGATCTTACTATGCGACGTGGTACCAACAGAAACCTGGGCAGGCACC
TATACTGGTCATATATGGTAAGAACAATAGACCGTCTGGAATACCTGATAGATTCTCAGCTAGC
ACTAGTGGAAATACAGCAAGCCTCACTATTAGCGGGGCACAAGCCGAAGACGAGGCCGACTACT
ATTGTGGCTCCCGAGATACGTCAGACAATCACCTGATGTTCGGCGGCGGCACTAAGCTCACGGT
CTTGGGGGAAGGGAAG
Light chain
(SEQ ID NO: 88)

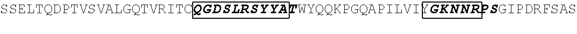

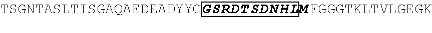

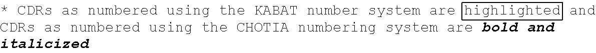

### Example 11: Clone 2-3

Heavy chain coding sequence
(SEQ ID NO: 132)
GAGTCCAAGGCGAGCGAAGTACAGTTGCTTGAGTCAGGCGGGAGGCAAGTTCAACCTCGCGGTT
CTTTGCGACTGTCCTGTACTGCTTCAGGCTTTAGTGTGGGGTCAGCCGATATGTCATGGGTACG
ACAGGCGCCCGGAAAAGGCCCAGAGTGGGTCTCATCCAAGGAATCTGCAGGTAGCACCTTCTAC
GCAGACAGTGTGAGAGGGAGGTTCACGATAGCGCGAGATAATAGTAACAATATGATTTTTTTGC
AGCTCAACAGTCTGCGACATGAAGACACTGCAGTTTATTACTGTGTGAGGGGTTCTGCTAGACG
ATCAGCATCCGGGTGGACACCTTATGATCTTTGGGGACAGGGTACTCTGGTAACGGTCAGCTCA
Heavy chain
(SEQ ID NO: 53)

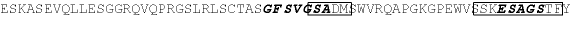

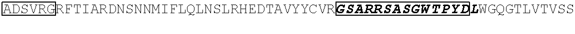

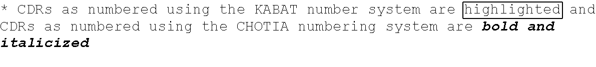

Light chain coding sequence
(SEQ ID NO: 141)
CAGGCAGTCCTTACCCAACCCAGTAGCTTGTCAGCTCCTCCAGGAGCGTCAGCGACGCTCCCAT
GTACATTGAGGAGCGACATTAACGTGGCTACTCAAAGGATATACTGGTATCACCAAAAACCTGG
TTCACCATTGCGATACCTGCTTCGATACAACAGTGACTCTGACAATCGGCTGGGTTCAGGTGTA
CCTAGCCGCTTCAGTGGCAGCAAAGATGTAAGTGCTAATGCGGCCTCACTGCTGATCTCCGGAC
TGCAGAGTGACGACGAGGCCGACTACTACTGTGTCATCTGGCACAATAGTGCTGTGGTTTTCGG
GGGAGGCACTAAACTCACAGTACTGGGTGAGGGAAAG
Light chain
(SEQ ID NO: 89)

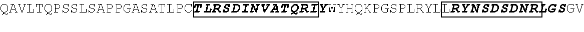

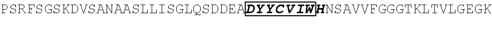

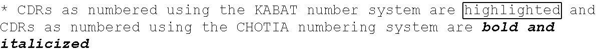

### Example 12: Clone 2-4

Heavy chain coding sequence
(SEQ ID NO: 133)
GAGTCAAAAGCATCCGAGGTTCAACTGGTGGAATCCGGTGGAACATTGAAACAACCAAGAGGTA
GTCTTCGGCTGAGTTGTGCGGCATCTGGTTTCACATTCAGTAATAGTGATATGGCATGGGTTAG
GCAGGCCCCAGGCAAAGGCTTGGAATGGGTGAGTTCAAAATCAGGATCTGACGGCACTACGTCA
TACGCCGATAGTGTTAGGGGTCGATTCACCATTGCTCGGGATAACTCTAAAAACACGCTTTATT
TGCAGATGAACGCGCTGCGGGTGGAAGATACCGCAGTTTACTATTGTGTCAAGGGGAGTGCATT
CTGGTCTGGATCTGGATTTTTCGACTCATGGGGCCAAGGGACGCTCGTCACTGTGAGCAGT
Heavy chain
(SEQ ID NO: 54)

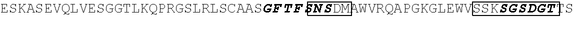

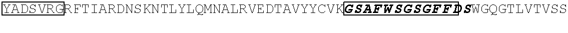

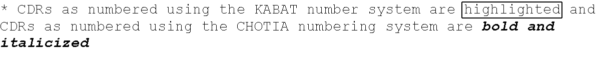

Light chain coding sequence
(SEQ ID NO: 142)
TCTTCTGAGCTCACACAAGACCCAGCCGTGAGTGTTGCGTTGGGCCAGACGGTTAGGATAACAT
GTCAAGGTAACTCTCTTCGAGGGAATAGCGCATCATGGTACCAACAGAAGCCTGGACAAGCCCC
GAGATTGGTAATGTACCATGAAGATCGACGGCCCAGTGGGGTGCCAGATCGCTTCAGCGGTTCC
AGCTCCGGTTTCATCTCTAGCTTGACCATCACGGGAGCCCAGGCTGCAGACGAGGCCGATTACT
ACTGCAATAGTCGAGATAAATCCGATTCCGTTATCTTTGGCGGCGGTACCAAGGTCACTGTGTT
GGGAGAGGGGAAA
Light chain
(SEQ ID NO: 90)

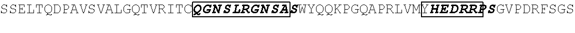

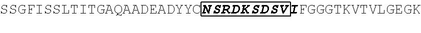

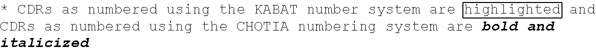

### Example 13: Clone 2-5

Heavy chain coding sequence
(SEQ ID NO: 134)
GAATCCAAAGCTAGCGAGGTGCAGCTGGTGGAGTCTGGAGGAACCTTGAAACAGTCTGCGGGGT
CCCTGAGACTGTCCTGTGCAGCCTCTGGATTCAGCGTCAGTGATTACCACATGAGCTGGGTCCG
CCAGGCTCCAGGGAAGGGGCTGGAGTGGGTCGCAATAAAATATAGTGGTGGTCACACAGGCTAC
GCAGACTCCGTGAAGGGCCGGTTCACCATCGCCAGAGACAATTCGAAGAATGACATTTATCTGC
AAATGAACGCCCTGAGAGGCGAGGACACGGCCGTCTATTATTGTGCGAGAGGTGTCAACGGTGA
CTACTTCTTTGACTATTGGGGCCAGGGAACCCTGGTCACCGTCTCCTCA
Heavy chain
(SEQ ID NO: 55)

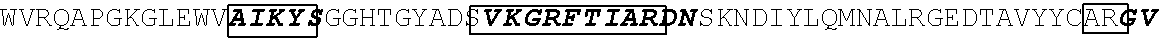

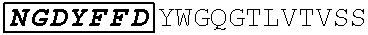

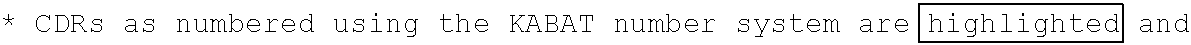

CDRs as numbered using the CHOTIA numbering system are bold and
italicized
Light chain coding sequence
(SEQ ID NO: 143)
TCTTATGAGCTGACACAGCCACCCTCGGTGTCAGTGTCCCCAGGACAAACGGCCAGGATCACCT
GCTCTGGAGATGCATTGCCAAAAAAATATGCTTATTGGTACCAGCAGAAGTCAGGCCAGGCCCC
TGCACTGGTCATCTATGAGGACAACAAACGACCCTTCGGGATCCCTGAGAGATTCTCTGGCTCC
AGGTCAGGGACAACGGCCACCTTGACTATCAGCGGGGCCCAGGTGGACGATGAAGCTGACTACT
ACTGTTACTCAACAGACAGCAGTGGTAATTATAGGGTGTTCGGCGGAGGGACCAAGCTCACCGT
CCTAGGTGAGGGTAAA
Light chain
(SEQ ID NO: 91)

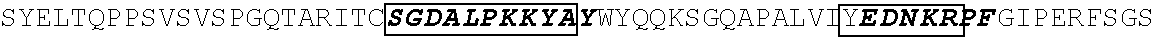

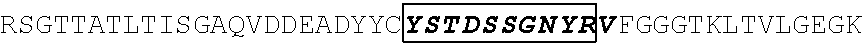

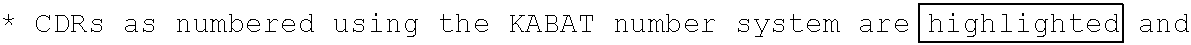

CDRs as numbered using the CHOTIA numbering system are bold and
italicized

### Example 14: Clone 2-6

Heavy chain coding sequence
(SEQ ID NO: 135)
GAATCCAAAGCTAGCGAGGTGCAGCTGGTGGAGTCTGGGGGAGGCGTGGTCCGGCCTGCGATGC
CCCTGAGACTCTCCTGTGCAGCCTCTGGATTCTCCTTCAATGACTATGGCCTGCACTGGGTCCG
TCAGGCTCCGGGCAAGGGGCTGGAGTGGGTGGCATCTATACTATCTCATGGAAAAAAAACATAC
TATGCAGACTCTGTGAAGGGCCGATTCACCATCGCCAGAGACAATTCCGAGAACACCCTGTATC
TGCAAATGAACAACCTGAGACCTGGGGACACGGCTGTGTATTATTGTGCGAAAGATCTGGTTCC
TGGCGCTGGCGTGGAATACTCTGGGACGGACGTCTGGGGCCAAGGGACAATGGTCACCGTCTCT
TCA
Heavy chain
(SEQ ID NO: 56)

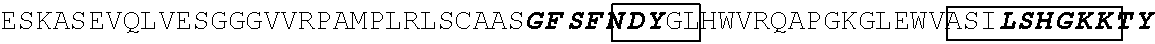

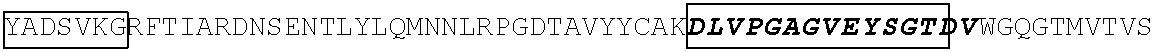

S

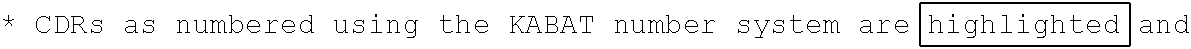

CDRs as numbered using the CHOTIA numbering system are bold and
italicized
Light chain coding sequence
(SEQ ID NO: 144)
GACATTCAGATGACCCAGTCTCCATCCTCCCTGTCTGCATCTGTAGGAGCCAGAGTCACCCTCA
CTTGTCGGGCAAGTCAGGATATTAGTAGGTACTTGAATTGGTATCAGCAGAAATCAGGGAGAGC
CCCTAAACTCCTGATCTATGGTGCATCCAGTTTGGAAAGTGGGGTCCCATCAAGGTTCAGTGGC
AGTGCATCTGGGTCAACTTTCACTCTCACCATCAACAGTCTACAACCTGAAGATTTTGCAACTT
ACTACTGTCAACAGAGTTTCACAACCCCGTATACTTTTGGCCAGGGGACCAAGGTGACCGTCCT
AGGTGAGGGTAAA
Light chain
(SEQ ID NO: 92)

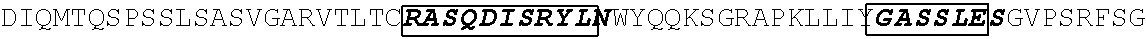

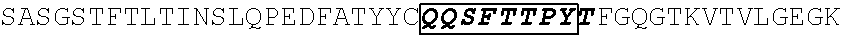

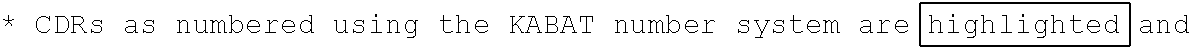

CDRs as numbered using the CHOTIA numbering system are bold and
italicized

### Example 15: Clone 2-8

Heavy chain coding sequence
(SEQ ID NO: 136)
GAATCCAAAGCTAGCGAGGTGCAGCTGGTGGAGTCTGGGGGAGGCTCGGTTCAGCCGGGGGGGT
CCCTGAGACTCTCCTGTGCAGCCTCTGGATTCACCTTTAGTAACTATGCCCTGAGCTGGGTCCG
CCAGGTTCCAGGGAAGGGGCTGGAGTGGGTCTCGGGTATTTATGGTAGTGTTGCTGGCAGGACT
ATGACAACTTTTTACGCAGACTTCGTGAAGGGCCGGTTCACCATCTCCAGAGACAATTCCAAGA
ACACCCTGTACCTGGAAATGAACGGCCTGAGAGTCGAGGACACGGCCGTATATTACTGTGCGAA
AGATATGGTGGGAGCTACTTGGTTCTACGGTATGGACGTCTGGGGCCAAGGCACCCTGGTCACC
GTCTCCTCA
Heavy chain
(SEQ ID NO: 57)

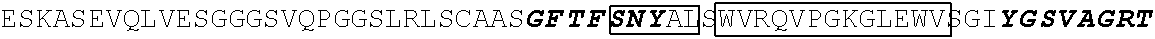

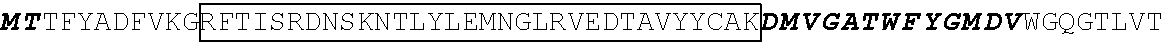

VSS

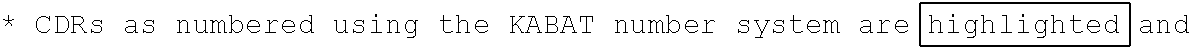

CDRs as numbered using the CHOTIA numbering system are bold and
italicized
Light chain coding sequence
(SEQ ID NO: 145)
CTGCCTGTGCTGACTCAGCCACCCTCAGCGTCTGGGACCCCCGGGCAGAGGGTCACCATCTCTT
GTTCTGGAAGCAGCTCCAACATCGGAAGTAATACTGTAAACTGGTACCAGCAGCTCCCAGGAAC
GGCCCCCAAACTCCTCATCTATAGTAATAATCAGCGGCCCTCAGGGGTCCCTGACCGATTCTCT
GGCTCCAAGTCTGGCACCTCAGCCTCCCTGGCCATCAGTGGGCTCCGGTCCGAGGATGAGGCTG
ATTATTACTGTGCAGCATGGGATGACAGCCTGAGTGGTCTGGTATTCGGCGGAGGGACCAAGCT
CACCGTCCTAGGTGAGGGTAAA
Light chain
(SEQ ID NO: 93)

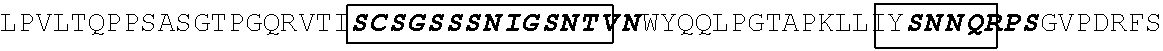

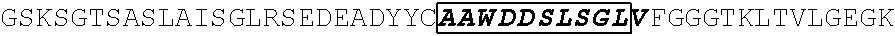

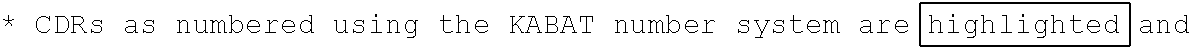

CDRs as numbered using the CHOTIA numbering system are bold and
italicized

### Example 16: Clone K1-70

Heavy chain coding sequence
(SEQ ID NO: 137)
CTGGTGCAGTCTGGAGCAGAGGTGAAAAAGCCCGGGCAGTCTCTGAAGATCTCCTGTAAGGCTT
CTGGATACAGCTTAACCGACAACTGGATCGGCTGGGTGCGCCAGAAGCCCGGGAAAGGCCTGGA
GTGGATGGGGATCATCTATCCTGGTGACTCTGACACCAGATACAGTCCGTCCTTCCAAGGCCAG
GTCACCATCTCAGCCGACAAGTCCATCAACACCGCCTACCTGCAGTGGAGCAGCCTGAAGGCCT
CGGACACCGCCATATATTACTGTGTGGGACTCGATTGGAACTACAACCCCCTGCGATACTGGGG
ACCGGGAACACTGGTTACCGTTTCA
Heavy chain
(SEQ ID NO: 58)

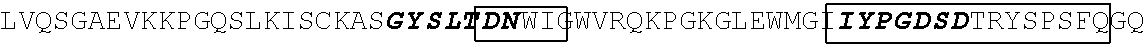

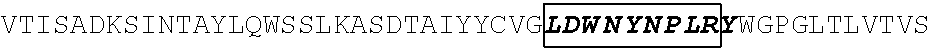

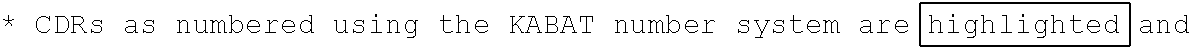

CDRs as numbered using the CHOTIA numbering system are bold and
italicized
Light chain coding sequence
(SEQ ID NO: 146)
CAGTCAGTGTTGACGCAGCCGCCCTCAGTGTCTGCGGCCCCAGGACAGAAGGTCACCATTTCCT
GCTCCGGAAGCAGCTCCGACATTGGGAGTAATTATGTATCCTGGTACCAGCAGTTCCCGGGAAC
AGCCCCCAAACTCCTCATTTATGACAATAATAAGCGACCCTCAGCGATTCCTGACCGATTCTCT
GGCTCCAAGTCTGGCACGTCAGCCACCCTGGGCATCACCGGACTCCAGACTGGGGACGAGGCCG
ATTATTACTGCGGAACATGGGATAGCAGACTGGGTATTGCTGTGTTCGGAGGAGGCACCCAGCT
GACCGTC
Light chain
(SEQ ID NO: 94)

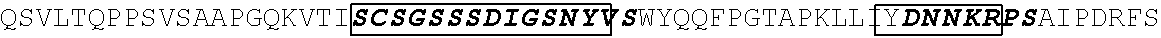

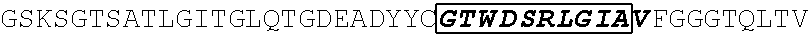

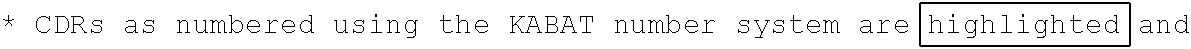

CDRs as numbered using the CHOTIA numbering system are bold and
italicized

### Example 17: Clone KI-18

Heavy chain coding sequence
(SEQ ID NO: 138)
GAGGTGCAGCTGGTGCAGTCTGGAGCAGAGGTGAAAAAGCCCGGGGAGTCTCTGAAGATCTCCT
GCAAGGGTTCTGGATACAGCTTTACCAACTACTGGATCGGCTGGGTGCGCCAGATGCCCGGGAA
AGGCCTGGAGTGGATGGGGATCATCTATCCTTATGACTCTGATACCAGATATAGCCCGTCCTTC
GAAGGCCAGGTCACCATtTCAGCCGACAAGTCCATCAGGACCGCCTACCTGCACTGGAGCAGCC
TGAAGGCCTCGGACACCGCCATGTATTACTGTGTGAGACCCCGCGATGGGAGCTATCCTTATGA
TGCTTTTGATATCTGGGGCCAAGGGACAATGGTCACCGTCTCTTCA
Heavy chain
(SEQ ID NO: 59)

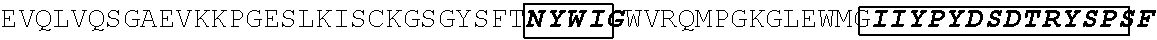

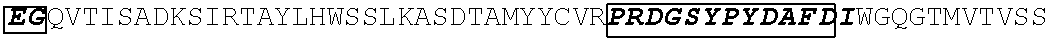

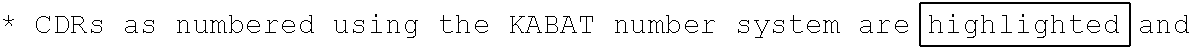

CDRs as numbered using the CHOTIA numbering system are bold and
italicized
Light chain coding sequence
(SEQ ID NO: 147)
GAAATTGTGTTGACGCAGTCTCCAGGCACCCTGTCTTTGTCTCCAGGGGAAAGAGCCACCCTCT
CCTGCAGGGCCAGTCAGAGTGTTAGCAACAACTACTTAGCCTGGTACCAGCAGAAGCCTGGCCA
GGCTCCCAGGCTCCTCATCTATGGTGCATCCAGCAGGGCCACTGGCATCCCAGACAGGTTCAGT
GGCAGTGGGTCTGGGACAGATTTCACTTTAACCATCAGCAGACTGGAGCCTGAAGATTTTGCAG
TGTATTACTGTCAGCATTGTGGTAGCTCACTGAGGGCGTTCGGCCAAGGGACCAAGGTGGAAAT
CAAACGA
Light chain
(SEQ ID NO: 95)

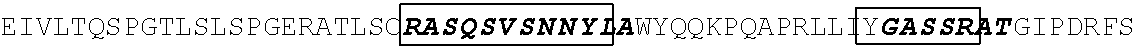

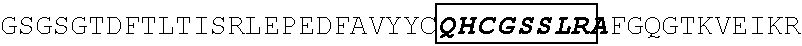

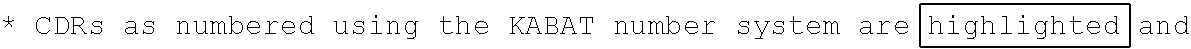

CDRs as numbered using the CHOTIA numbering system are bold and
italicized

### Example 18: Treating Thyroid Cancer

T cells (e.g., CAR T cells) engineered to express an antigen receptor (e.g., a CAR) that can target a TSHR polypeptide are administered to a human identified as having thyroid cancer. The T cells (e.g., CAR T cells) engineered to express an antigen receptor (e.g., a CAR) that can target a TSHR polypeptide are administered using intravenous or intratumoral injection. After the administration of the one or more T cells (e.g., one or more CAR T cells) expressing (e.g., engineered to express) an antigen receptor (e.g., a CAR) that can target a TSHR polypeptide the number of cancer cells (e.g., cancer cells expressing a TSHR polypeptide) within the human is reduced. After the administration of the one or more T cells (e.g., one or more CAR T cells) expressing (e.g., engineered to express) an antigen receptor (e.g., a CAR) that can target a TSHR polypeptide, the size of one or more tumors (e.g., tumors expressing a TSHR polypeptide) within the human is reduced.

### Example 19: Generation of T Cells Expressing an Antigen Receptor that can Target a TSHR Polypeptide

T cells are obtained from a human identified as having thyroid cancer. Nucleic acid encoding an antigen receptor (e.g., a CAR) that can target a TSHR polypeptide is introduced into the T cell by transduction (e.g., viral transduction using a retroviral vector such as a lentiviral vector) or transfection such that the T cell expresses the antigen receptor that can target a TSHR polypeptide. The T cells engineered to express an antigen receptor (e.g., a CAR) that can target a TSHR polypeptide are administered back into the human using intravenous or intratumoral injection. After the administration of the one or more T cells (e.g., one or more CAR T cells) expressing (e.g., engineered to express) an antigen receptor (e.g., a CAR) that can target a TSHR polypeptide the number of cancer cells (e.g., cancer cells expressing a TSHR polypeptide) within the human is reduced. After the administration of the one or more T cells (e.g., one or more CAR T cells) expressing (e.g., engineered to express) an antigen receptor (e.g., a CAR) that can target a TSHR polypeptide, the size of one or more tumors (e.g., tumors expressing a TSHR polypeptide) within the human is reduced.

### Example 20: Treating a Patient with MEK+/−BRAF Inhibitor+Macrophage Depletion Therapy and TSHR CAR T Therapy

A human having anaplastic thyroid carcinoma (ATC) is administered a low dose MEK inhibitor (e.g., trametinib) and a macrophage depletion therapy (e.g., a GM-CSF neutralizing antibody and/or clodronate) daily for one week. The macrophage depletion therapy is stopped at the end of one week while MEK inhibitor treatment is continued.

On day 7, the human is administered a single injection of T cells (e.g., CAR T cells) engineered to express an antigen receptor (e.g., a CAR) that can target a TSHR polypeptide.

Following the administration of the T cells (e.g., CAR T cells) engineered to express an antigen receptor (e.g., a CAR) that can target a TSHR polypeptide, blood draws are performed to determine activity of the CAR T cells and tumor response to therapy.

TSHR polypeptide expression in the thyroid tumor is determined. In some cases, TSHR polypeptide expression is determined using a tissue biopsy obtained from the tumor on day 7, and optionally formalin fixed, and/or flow analysis. In some cases, TSHR polypeptide expression in the thyroid tumor is determined using used for PET/CT imaging and a radiolabeled (e.g., 89-Zr DFO) TSHR antibody.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

The invention claimed is:

1. A T cell comprising a heterologous nucleic acid encoding an antigen receptor having the ability to bind to a thyroid stimulating hormone receptor (TSHR) polypeptide, wherein said T cell expresses said antigen receptor.

2. The T cell of claim 1, wherein said antigen receptor is a chimeric antigen receptor (CAR).

3. The T cell of claim 1, wherein said T cell is a human T cell.

4. The T cell of claim 1, wherein said antigen receptor comprises a single chain variable fragment (scFv) having the ability to bind to said TSHR polypeptide.

5. The T cell of claim 4, wherein said scFv comprises a heavy chain variable (VH) domain comprising a complementarity determining region (CDR) 1 sequence set forth in any one of SEQ ID NOs:1-21, a CDR2 sequence set forth in any one of SEQ ID NOs:22-38, and a CDR3 sequence set forth in any one of SEQ ID NOs:39-50.

6. (canceled)

7. The T cell of claim 4, wherein said scFv comprises a light chain variable (VL) domain comprising a CDR1 sequence set forth in any one of SEQ ID NOs:60-68, a CDR2 sequence set forth in any one of SEQ ID NOs:69-77, and a CDR3 sequence set forth in any one of SEQ ID NOs:78-86.

8-18. (canceled)

19. An antibody having the ability to bind to a TSHR polypeptide, wherein said antibody comprises (a) a VH domain comprising a CDR1 sequence set forth in any one of SEQ ID NOs:1-21, a CDR2 sequence set forth in any one of SEQ ID NOs: 22-38, and a CDR3 sequence set forth in any one of SEQ ID NOs:39-50, and (b) a VL domain comprising a CDR1 sequence set forth in any one of SEQ ID NOs:60-68, a CDR2 sequence set forth in any one of SEQ ID NOs:69-77, and a CDR3 sequence set forth in any one of SEQ ID NOs:78-86.

20. (canceled)

21. A chimeric antigen receptor (CAR) having the ability to bind to a thyroid stimulating hormone receptor (TSHR) polypeptide, wherein said CAR comprises:
a single chain variable fragment (scFv) comprising a VH domain comprising a CDR1 sequence set forth in any one of SEQ ID NOs:1-21, a CDR2 sequence set forth in any one of SEQ ID NOs: 22-38, and a CDR3 sequence set forth in any one of SEQ ID NOs:39-50, and a VL domain comprising a CDR1 sequence set forth in any one of SEQ ID NOs:60-68, a CDR2 sequence set forth in any one of SEQ ID NOs:69-77, and a CDR3 sequence set forth in any one of SEQ ID NOs:78-86;
a CD8 hinge domain;
a 4-1BB signaling domain; and
a CD3zeta signaling domain.

22. A nucleic acid construct encoding a CAR having the ability to bind to a thyroid stimulating hormone receptor (TSHR) polypeptide, wherein said CAR comprises:
a single chain variable fragment (scFv) comprising a VH domain comprising a CDR1 sequence set forth in any one of SEQ ID NOs:1-21, a CDR2 sequence set forth in any one of SEO ID NOs: 22-38, and a CDR3 sequence set forth in any one of SEO ID NOs:39-50, and a VL domain comprising a CDR1 sequence set forth in any one of SEQ ID NOs:60-68, a CDR2 sequence set forth in any one of SEQ ID NOs:69-77, and a CDR3 sequence set forth in any one of SEQ ID NOs:78-86;
a CD8 hinge domain;
a 4-1BB signaling domain; and
a CD3zeta signaling domain.

23-24. (canceled)

25. A method for treating a mammal having cancer, wherein said method comprises administering, to said mammal, a T cell comprising a heterologous nucleic acid encoding an antigen receptor having the ability to bind to a thyroid stimulating hormone receptor (TSHR) polypeptide, wherein said T cell expresses said antigen receptor, wherein said cancer comprises a cancer cell expressing a TSHR polypeptide.

26. The method of claim 25, wherein said mammal is human.

27. The method of claim 25, wherein said cancer is a thyroid cancer.

28-32. (canceled)

33. A method for treating cancer, wherein said method comprises administering, to a mammal having said cancer, a population of cells comprising nucleic acid encoding a chimeric antigen receptor having the ability to bind to a TSHR polypeptide.

34. The method of claim 33, wherein said mammal is human.

35. The method of claim 33, wherein said cancer is a thyroid cancer.

36. The method of claim 33, wherein said cells are T cells.

37-44. (canceled)

45. The method of claim 33, wherein said method comprises administering, to said mammal, one or more agents that reduce the number of macrophages within said mammal.

46. The method of claim 45, wherein at least one of said one or more agents that reduce the number of macrophages within said mammal is a CSF-1R specific kinase inhibitor.

47. (canceled)

48. The method of claim 45, wherein at least one of said one or more agents that reduce the number of macrophages within said mammal is selected from the group consisting of GM-CSF neutralizing antibodies, clodronate, emactuzumab, AMG820, IMC-CS4, cabiralizumab, lacnotuzumab, and PD-0360324.

49. The method of claim 45, wherein at least one of said one or more agents that reduce the number of macrophages within said mammal is an immunomodulatory imide drug.

50. (canceled)


SEQUENCE LISTING (174 sequences)

SEQ ID NO: 1 - protein, 7 aa, synthetic construct
GFSVSGN

SEQ ID NO: 2 - protein, 5 aa, synthetic construct
GNQMT

SEQ ID NO: 3 - protein, 10 aa, synthetic construct
GFSVSGNQMT

SEQ ID NO: 4 - protein, 6 aa, synthetic construct
GFTFTT

SEQ ID NO: 5 - protein, 7 aa, synthetic construct
GFSVGSA

SEQ ID NO: 6 - protein, 5 aa, synthetic construct
SADMS

SEQ ID NO: 7 - protein, 10 aa, synthetic construct
GFSVGSADMS

SEQ ID NO: 8 - protein, 7 aa, synthetic construct
GFTFSNS

SEQ ID NO: 9 - protein, 5 aa, synthetic construct
NSDMA

SEQ ID NO: 10 - protein, 10 aa, synthetic construct
GFTFSNSDMA

SEQ ID NO: 11 - protein, 5 aa, synthetic construct
AIKYS

SEQ ID NO: 12 - protein, 7 aa, synthetic construct
GFSFNDY

SEQ ID NO: 13 - protein, 5 aa, synthetic construct
DYGLH

SEQ ID NO: 14 - protein, 10 aa, synthetic construct
GFSFNDYGLH

SEQ ID NO: 15 - protein, 7 aa, synthetic construct
GFTFSNY

SEQ ID NO: 16 - protein, 5 aa, synthetic construct
NYALS

SEQ ID NO: 17 - protein, 10 aa, synthetic construct
GFTFSNYALS

SEQ ID NO: 18 - protein, 7 aa, synthetic construct
GYSLTDN

SEQ ID NO: 19 - protein, 5 aa, synthetic construct
DNWIG

SEQ ID NO: 20 - protein, 10 aa, synthetic construct
GYSLTDNWIG

SEQ ID NO: 21 - protein, 5 aa, synthetic construct
NYWIG

SEQ ID NO: 22 - protein, 16 aa, synthetic construct
VKNSDGSTSYADSVKG

SEQ ID NO: 23 - protein, 5 aa, synthetic construct
NSDGS

SEQ ID NO: 24 - protein, 7 aa, synthetic construct
TRNGNGG

SEQ ID NO: 25 - protein, 5 aa, synthetic construct
GNGGR

SEQ ID NO: 26 - protein, 8 aa, synthetic construct
TRNGNGGR

SEQ ID NO: 27 - protein, 5 aa, synthetic construct
ESAGS

SEQ ID NO: 28 - protein, 16 aa, synthetic construct
SKESAGSTFYADSVRG

SEQ ID NO: 29 - protein, 6 aa, synthetic construct
SGSDGT

SEQ ID NO: 30 - protein, 17 aa, synthetic construct
SKSGSDGTTSYADSVRG

SEQ ID NO: 31 - protein, 11 aa, synthetic construct
VKGRFTIARDN

SEQ ID NO: 32 - protein, 6 aa, synthetic construct
LSHGKK

SEQ ID NO: 33 - protein, 17 aa, synthetic construct
SILSHGKKTYYADSVKG

SEQ ID NO: 34 - protein, 10 aa, synthetic construct
YGSVAGRTMT

SEQ ID NO: 35 - protein, 13 aa, synthetic construct
VRQVPGKGLEWVS

SEQ ID NO: 36 - protein, 6 aa, synthetic construct
YPGDSD

SEQ ID NO: 37 - protein, 16 aa, synthetic construct
IYPGDSDTRYSPSFQG

SEQ ID NO: 38 - protein, 17 aa, synthetic construct
IIYPYDSDTRYSPSFEG

SEQ ID NO: 39 - protein, 8 aa, synthetic construct
LKNGVFDI

SEQ ID NO: 40 - protein, 13 aa, synthetic construct
DLGPVVRGTFDVW

SEQ ID NO: 41 - protein, 11 aa, synthetic construct
WGQGTMVTVSS

SEQ ID NO: 42 - protein, 23 aa, synthetic construct
DLGPVVRGTFDVWGQGTMVTVSS

SEQ ID NO: 43 - protein, 15 aa, synthetic construct
GSARRSASGWTPYDL

SEQ ID NO: 44 - protein, 13 aa, synthetic construct
GSAFWSGSGFFDS

SEQ ID NO: 45 - protein, 9 aa, synthetic construct
GVNGDYFFD

SEQ ID NO: 46 - protein, 15 aa, synthetic construct
DLVPGAGVEYSGTDV

SEQ ID NO: 47 - protein, 13 aa, synthetic construct
DMVGATWFYGMDV

SEQ ID NO: 48 - protein, 32 aa, synthetic construct
RFTISRDNSKNTLYLEMNGLRVEDTAVYYCAK

SEQ ID NO: 49 - protein, 10 aa, synthetic construct
LDWNYNPLRY

SEQ ID NO: 50 - protein, 13 aa, synthetic construct
PRDGSYPYDAFDI

SEQ ID NO: 51 - protein, 121 aa, synthetic construct
ESKASEVQLLESGGGLVQFRGSRRLSCAVSGFSVSGNQMTWVRQAPGKGLEWLSVKNSDGSTSYADSVKGRFTIARDEVKNTVFLQMNAVRAEDTALYYCARLKNGVFDIWGQGTMVTVSS

SEQ ID NO: 52 - protein, 121 aa, synthetic construct
ESKASEVQLVESGGGLVQPRGSLRLSCAASGFTFTTFAMSWVRQAPGKGLEWVATRNGNGGRTYYADSVRGRFTISRDLHLQMNSLRVEDTAVYYCTKDLGPVVRGTFDVWGQGTMVTVSS

SEQ ID NO: 53 - protein, 128 aa, synthetic construct
ESKASEVQLLESGGRQVQPRGSLRLSCTASGFSVGSADMSWVRQAPGKGPEWVSSKESAGSTFYADSVRGRFTIARDNSNNMIFLQLNSLRHEDTAVYYCVRGSARRSASGWTPYDLWGQGTLVTVSS

SEQ ID NO: 54 - protein, 127 aa, synthetic construct
ESKASEVQLVESGGTLKQPRGSLRLSCAASGFTFSNSDMAWVRQAPGKGLEWVSSKSGSDGTTSYADSVRGRFTIARDNSKNTLYLQMNALRVEDTAVYYCVKGSAFWSGSGFFDSWGQGTLVTVSS

SEQ ID NO: 55 - protein, 83 aa, synthetic construct
WVRQAPGKGLEWVAIKYSGGHTGYADSVKGRFTIARDNSKNDIYLQMNALRGEDTAVYYCARGVNGDYFFDYWGQGTLVTVSS

SEQ ID NO: 56 - protein, 129 aa, synthetic construct
ESKASEVQLVESGGGVVRPAMPLRLSCAASGFSFNDYGLHWVRQAPGKGLEWVASILSHGKKTYYADSVKGRFTIARDNSENTLYLQMNNLRPGDTAVYYCAKDLVPGAGVEYSGTDVWGQGTMVTVSS

SEQ ID NO: 57 - protein, 131 aa, synthetic construct
ESKASEVQLVESGGGSVQPGGSLRLSCAASGFTFSNYALSWVRQVPGKGLEWVSGIYGSVAGRTMTTFYADFVKGRFTISRDNSKNTLYLEMNGLRVEDTAVYYCAKDMVGATWFYGMDVWGQGTLVTVSS

SEQ ID NO: 58 - protein, 115 aa, synthetic construct
LVQSGAEVKKPGQSLKISCKASGYSLTDNWIGWVRQKPGKGLEWMGIIYPGDSDTRYSPSFQGQVTISADKSINTAYLQWSSLKASDTAIYYCVGLDWNYNPLRYWGPGTLVTVS

SEQ ID NO: 59 - protein, 122 aa, synthetic construct
EVQLVQSGAEVKKPGESLKISCKGSGYSFTNYWIGWVRQMPGKGLEWMGIIYPYDSDTRYSPSFEGQVTISADKSIRTAYLHWSSLKASDTAMYYCVRPRDGSYPYDAFDIWGQGTMVTVSS

SEQ ID NO: 60 - protein, 14 aa, synthetic construct
TLRRGINLGAYGIH

SEQ ID NO: 61 - protein, 11 aa, synthetic construct
QGDSLRSYYAT

SEQ ID NO: 62 - protein, 14 aa, synthetic construct
TLRSDINVATQRIY

SEQ ID NO: 63 - protein, 11 aa, synthetic construct
QGNSLRGNSAS

SEQ ID NO: 64 - protein, 11 aa, synthetic construct
SGDALPKKYAY

SEQ ID NO: 65 - protein, 11 aa, synthetic construct
RASQDISRYLN

SEQ ID NO: 66 - protein, 13 aa, synthetic construct
SGSSSNIGSNTVN

SEQ ID NO: 67 - protein, 15 aa, synthetic construct
SCSGSSSDIGSNYVS

SEQ ID NO: 68 - protein, 12 aa, synthetic construct
RASQSVSNNYLA

SEQ ID NO: 69 - protein, 11 aa, synthetic construct
HKSASDKQQGS

SEQ ID NO: 70 - protein, 7 aa, synthetic construct
GKNNRPS

SEQ ID NO: 71 - protein, 12 aa, synthetic construct
RYNSDSDNRLGS

SEQ ID NO: 72 - protein, 7 aa, synthetic construct
HEDRRPS

SEQ ID NO: 73 - protein, 7 aa, synthetic construct
EDNKRPF

SEQ ID NO: 74 - protein, 7 aa, synthetic construct
GASSLES

SEQ ID NO: 75 - protein, 7 aa, synthetic construct
SNNQRPS

SEQ ID NO: 76 - protein, 7 aa, synthetic construct
DNNKRPS

SEQ ID NO: 77 - protein, 7 aa, synthetic construct
GASSRAT

SEQ ID NO: 78 - protein, 9 aa, synthetic construct
MIYYNSAWV

SEQ ID NO: 79 - protein, 11 aa, synthetic construct
GSRDTSDNHLM

SEQ ID NO: 80 - protein, 8 aa, synthetic construct
DYYCVIWH

SEQ ID NO: 81 - protein, 10 aa, synthetic construct
NSRDKSDSVI

SEQ ID NO: 82 - protein, 11 aa, synthetic construct
YSTDSSGNYRV

SEQ ID NO: 83 - protein, 9 aa, synthetic construct
QQSFTTPYT

SEQ ID NO: 84 - protein, 11 aa, synthetic construct
AAWDDSLSGLV

SEQ ID NO: 85 - protein, 11 aa, synthetic construct
GTWDSRLGIAV

SEQ ID NO: 86 - protein, 9 aa, synthetic construct
QHCGSSLRA

SEQ ID NO: 87 - protein, 119 aa, synthetic construct
QPVLTQPTSLSASPGASASLTCTLRRGINLGAYGIHWYQQRPGSPPRYLLRHKSASDKQQGSGVPGRFSGSKDASANAGLLLISGLQSEDEADYYCMIYYNSAWVFGGGTKLTVLGEGK

SEQ ID NO: 88 - protein, 112 aa, synthetic construct
SSELTQDPTVSVALGQTVRITCQGDSLRSYYATWYQQKPGQAPILVIYGKNNRPSGIPDRFSASTSGNTASLTISGAQAEDEADYYCGSRDTSDNHLMFGGGTKLTVLGEGK

SEQ ID NO: 89 - protein, 119 aa, synthetic construct
QAVLTQPSSLSAPPGASATLPCTLRSDINVATQRIYWYHQKPGSPLRYLLRYNSDSDNRLGSGVPSRFSGSKDVSANAASLLISGLQSDDEADYYCVIWHNSAVVFGGGTKLTVLGEGK

SEQ ID NO: 90 - protein, 111 aa, synthetic construct
SSELTQDPAVSVALGQTVRITCQGNSLRGNSASWYQQKPGQAPRLVMYHEDRRPSGVPDRFSGSSSGFISSLTITGAQAADEADYYCNSRDKSDSVIFGGGTKVTVLGEGK

SEQ ID NO: 91 - protein, 112 aa, synthetic construct
SYELTQPPSVSVSPGQTARITCSGDALPKKYAYWYQQKSGQAPALVIYEDNKRPFGIPERFSGSRSGTTATLTISGAQVDDEADYYCYSTDSSGNYRVFGGGTKLTVLGEGK

SEQ ID NO: 92 - protein, 111 aa, synthetic construct
DIQMTQSPSSLSASVGARVTLTCRASQDISRYLNWYQQKSGRAPKLLIYGASSLESGVPSRFSGSASGSTFTLTINSLQPEDFATYYCQQSFTTPYTFGQGTKVTVLGEGK

SEQ ID NO: 93 - protein, 114 aa, synthetic construct
LPVLTQPPSASGTPGQRVTISCSGSSSNIGSNTVNWYQQLPGTAPKLLIYSNNQRPSGVPDRFSGSKSGTSASLAISGLRSEDEADYYCAAWDDSLSGLVFGGGTKLTVLGEGK

SEQ ID NO: 94 - protein, 109 aa, synthetic construct
QSVLTQPPSVSAAPGQKVTISCSGSSSDIGSNYVSWYQQFPGTAPKLLIYDNNKRPSAIPDRFSGSKSGTSATLGITGLQTGDEADYYCGTWDSRLGIAVFGGGTQLTV

SEQ ID NO: 95 - protein, 109 aa, synthetic construct
EIVLTQSPGTLSLSPGERATLSCRASQSVSNNYLAWYQQKPGQAPRLLIYGASSRATGIPDRFSGSGSGTDFTLTISRLEPEDFAVYYCQHCGSSLRAFGQGTKVEIKR

SEQ ID NO: 96 - protein, 20 aa, synthetic construct
GGGGSGGGGSGGGGSGGGGS

SEQ ID NO: 97 - protein, 10 aa, synthetic construct
GGGGSGGGGS

SEQ ID NO: 98 - protein, 15 aa, synthetic construct
GGGGSGGGGSGGGGS

SEQ ID NO: 99 - protein, 25 aa, synthetic construct
GGGGSGGGGSGGGGSGGGGSGGGGS

SEQ ID NO: 100 - protein, 10 aa, synthetic construct
SGGGGSGGGG

SEQ ID NO: 101 - protein, 15 aa, synthetic construct
SGGGGSGGGGSGGGG

SEQ ID NO: 102 - protein, 20 aa, synthetic construct
SGGGGSGGGGSGGGGSGGGG

SEQ ID NO: 103 - protein, 25 aa, synthetic construct
SGGGGSGGGGSGGGGSGGGGSGGGG

SEQ ID NO: 104 - protein, 260 aa, synthetic construct
ESKASEVQLLESGGGLVQFRGSRRLSCAVSGFSVSGNQMTWVRQAPGKGLEWLSVKNSDGSTSYADSVKGRFTIARDEVKNTVFLQMNAVRAEDTALYYCARLKNGVFDIWGQGTMVTVSSGGGGSGGGGSGGGGSGGGGSQPVLTQPTSLSASPGASASLTCTLRRGINLGAYGIHWYQQRPGSPPRYLLRHKSASDKQQGSGVPGRFSGSKDASANAGLLLISGLQSEDEADYYCMIYYNSAWVFGGGTKLTVLGEGK

SEQ ID NO: 105 - protein, 260 aa, synthetic construct
QPVLTQPTSLSASPGASASLTCTLRRGINLGAYGIHWYQQRPGSPPRYLLRHKSASDKQQGSGVPGRFSGSKDASANAGLLLISGLQSEDEADYYCMIYYNSAWVFGGGTKLTVLGEGKGGGGSGGGGSGGGGSGGGGSESKASEVQLLESGGGLVQFRGSRRLSCAVSGFSVSGNQMTWVRQAPGKGLEWLSVKNSDGSTSYADSVKGRFTIARDEVKNTVFLQMNAVRAEDTALYYCARLKNGVFDIWGQGTMVTVSS

SEQ ID NO: 106 - protein, 253 aa, synthetic construct
ESKASEVQLVESGGGLVQPRGSLRLSCAASGFTFTTFAMSWVRQAPGKGLEWVATRNGNGGRTYYADSVRGRFTISRDLHLQMNSLRVEDTAVYYCTKDLGPVVRGTFDVWGQGTMVTVSSGGGGSGGGGSGGGGSGGGGSSSELTQDPTVSVALGQTVRITCQGDSLRSYYATWYQQKPGQAPILVIYGKNNRPSGIPDRFSASTSGNTASLTISGAQAEDEADYYCGSRDTSDNHLMFGGGTKLTVLGEGK

SEQ ID NO: 107 - protein, 253 aa, synthetic construct
SSELTQDPTVSVALGQTVRITCQGDSLRSYYATWYQQKPGQAPILVIYGKNNRPSGIPDRFSASTSGNTASLTISGAQAEDEADYYCGSRDTSDNHLMFGGGTKLTVLGEGKGGGGSGGGGSGGGGSGGGGSESKASEVQLVESGGGLVQPRGSLRLSCAASGFTFTTFAMSWVRQAPGKGLEWVATRNGNGGRTYYADSVRGRFTISRDLHLQMNSLRVEDTAVYYCTKDLGPVVRGTFDVWGQGTMVTVSS

SEQ ID NO: 108 - protein, 267 aa, synthetic construct
ESKASEVQLLESGGRQVQPRGSLRLSCTASGFSVGSADMSWVRQAPGKGPEWVSSKESAGSTFYADSVRGRFTIARDNSNNMIFLQLNSLRHEDTAVYYCVRGSARRSASGWTPYDLWGQGTLVTVSSGGGGSGGGGSGGGGSGGGGSQAVLTQPSSLSAPPGASATLPCTLRSDINVATQRIYWYHQKPGSPLRYLLRYNSDSDNRLGSGVPSRFSGSKDVSANAASLLISGLQSDDEADYYCVIWHNSAVVFGGGTKLTVLGEGK

SEQ ID NO: 109 - protein, 267 aa, synthetic construct
QAVLTQPSSLSAPPGASATLPCTLRSDINVATQRIYWYHQKPGSPLRYLLRYNSDSDNRLGSGVPSRFSGSKDVSANAASLLISGLQSDDEADYYCVIWHNSAVVFGGGTKLTVLGEGKGGGGSGGGGSGGGGSGGGGSESKASEVQLLESGGRQVQPRGSLRLSCTASGFSVGSADMSWVRQAPGKGPEWVSSKESAGSTFYADSVRGRFTIARDNSNNMIFLQLNSLRHEDTAVYYCVRGSARRSASGWTPYDLWGQGTLVTVSS

SEQ ID NO: 110 - protein, 258 aa, synthetic construct
ESKASEVQLVESGGTLKQPRGSLRLSCAASGFTFSNSDMAWVRQAPGKGLEWVSSKSGSDGTTSYADSVRGRFTIARDNSKNTLYLQMNALRVEDTAVYYCVKGSAFWSGSGFFDSWGQGTLVTVSSGGGGSGGGGSGGGGSGGGGSSSELTQDPAVSVALGQTVRITCQGNSLRGNSASWYQQKPGQAPRLVMYHEDRRPSGVPDRFSGSSSGFISSLTITGAQAADEADYYCNSRDKSDSVIFGGGTKVTVLGEGK

SEQ ID NO: 111 - protein, 258 aa, synthetic construct
SSELTQDPAVSVALGQTVRITCQGNSLRGNSASWYQQKPGQAPRLVMYHEDRRPSGVPDRFSGSSSGFISSLTITGAQAADEADYYCNSRDKSDSVIFGGGTKVTVLGEGKGGGGSGGGGSGGGGSGGGGSESKASEVQLVESGGTLKQPRGSLRLSCAASGFTFSNSDMAWVRQAPGKGLEWVSSKSGSDGTTSYADSVRGRFTIARDNSKNTLYLQMNALRVEDTAVYYCVKGSAFWSGSGFFDSWGQGTLVTVSS

SEQ ID NO: 112 - protein, 215 aa, synthetic construct
WVRQAPGKGLEWVAIKYSGGHTGYADSVKGRFTIARDNSKNDIYLQMNALRGEDTAVYYCARGVNGDYFFDYWGQGTLVTVSSGGGGSGGGGSGGGGSGGGGSSYELTQPPSVSVSPGQTARITCSGDALPKKYAYWYQQKSGQAPALVIYEDNKRPFGIPERFSGSRSGTTATLTISGAQVDDEADYYCYSTDSSGNYRVFGGGTKLTVLGEGK

SEQ ID NO: 113 - protein, 215 aa, synthetic construct
SYELTQPPSVSVSPGQTARITCSGDALPKKYAYWYQQKSGQAPALVIYEDNKRPFGIPERFSGSRSGTTATLTISGAQVDDEADYYCYSTDSSGNYRVFGGGTKLTVLGEGKGGGGSGGGGSGGGGSGGGGSWVRQAPGKGLEWVAIKYSGGHTGYADSVKGRFTIARDNSKNDIYLQMNALRGEDTAVYYCARGVNGDYFFDYWGQGTLVTVSS

SEQ ID NO: 114 - protein, 260 aa, synthetic construct
ESKASEVQLVESGGGVVRPAMPLRLSCAASGFSFNDYGLHWVRQAPGKGLEWVASILSHGKKTYYADSVKGRFTIARDNSENTLYLQMNNLRPGDTAVYYCAKDLVPGAGVEYSGTDVWGQGTMVTVSSGGGGSGGGGSGGGGSGGGGSDIQMTQSPSSLSASVGARVTLTCRASQDISRYLNWYQQKSGRAPKLLIYGASSLESGVPSRFSGSASGSTFTLTINSLQPEDFATYYCQQSFTTPYTFGQGTKVTVLGEGK

SEQ ID NO: 115 - protein, 260 aa, synthetic construct
DIQMTQSPSSLSASVGARVTLTCRASQDISRYLNWYQQKSGRAPKLLIYGASSLESGVPSRFSGSASGSTFTLTINSLQPEDFATYYCQQSFTTPYTFGQGTKVTVLGEGKGGGGSGGGGSGGGGSGGGGSESKASEVQLVESGGGVVRPAMPLRLSCAASGFSFNDYGLHWVRQAPGKGLEWVASILSHGKKTYYADSVKGRFTIARDNSENTLYLQMNNLRPGDTAVYYCAKDLVPGAGVEYSGTDVWGQGTMVTVSS

SEQ ID NO: 116 - protein, 265 aa, synthetic construct
ESKASEVQLVESGGGSVQPGGSLRLSCAASGFTFSNYALSWVRQVPGKGLEWVSGIYGSVAGRTMTTFYADFVKGRFTISRDNSKNTLYLEMNGLRVEDTAVYYCAKDMVGATWFYGMDVWGQGTLVTVSSGGGGSGGGGSGGGGSGGGGSLPVLTQPPSASGTPGQRVTISCSGSSSNIGSNTVNWYQQLPGTAPKLLIYSNNQRPSGVPDRFSGSKSGTSASLAISGLRSEDEADYYCAAWDDSLSGLVFGGGTKLTVLGEGK

SEQ ID NO: 117 - protein, 265 aa, synthetic construct
LPVLTQPPSASGTPGQRVTISCSGSSSNIGSNTVNWYQQLPGTAPKLLIYSNNQRPSGVPDRFSGSKSGTSASLAISGLRSEDEADYYCAAWDDSLSGLVFGGGTKLTVLGEGKGGGGSGGGGSGGGGSGGGGSESKASEVQLVESGGGSVQPGGSLRLSCAASGFTFSNYALSWVRQVPGKGLEWVSGIYGSVAGRTMTTFYADFVKGRFTISRDNSKNTLYLEMNGLRVEDTAVYYCAKDMVGATWFYGMDVWGQGTLVTVSS

SEQ ID NO: 118 - protein, 239 aa, synthetic construct
LVQSGAEVKKPGQSLKISCKASGYSLTDNWIGWVRQKPGKGLEWMGIIYPGDSDTRYSPSFQGQVTISADKSINTAYLQWSSLKASDTAIYYCVGLDWNYNPLRYWGPGTLVTVSGGGGSGGGGSGGGGSQSVLTQPPSVSAAPGQKVTISCSGSSSDIGSNYVSWYQQFPGTAPKLLIYDNNKRPSAIPDRFSGSKSGTSATLGITGLQTGDEADYYCGTWDSRLGIAVFGGGTQLTV

SEQ ID NO: 119 - protein, 238 aa, synthetic construct
QSVLTQPPSVSAAPGQKVTISCSGSSSDIGSNYVSWYQQFPGTAPKLLIYDNNKRPSAIPDRFSGSKSGTSATLGITGLQTGDEADYYCGTWDSRLGIAVFGGGTQLTVGGGGSGGGGSGGGGSLVQSGAEVKKPGQSLKISCKASGYSLTDNWIGWVRQKPGKGLEWMGIIYPGDSDTRYSPSFQGQVTISADKSINTAYLQWSSLKASDTAIYYCVGLDWNYNPLRYWGPGTLVTV

SEQ ID NO: 120 - protein, 246 aa, synthetic construct
EVQLVQSGAEVKKPGESLKISCKGSGYSFTNYWIGWVRQMPGKGLEWMGIIYPYDSDTRYSPSFEGQVTISADKSIRTAYLHWSSLKASDTAMYYCVRPRDGSYPYDAFDIWGQGTMVTVSSGGGGSGGGGSGGGGSEIVLTQSPGTLSLSPGERATLSCRASQSVSNNYLAWYQQKPGQAPRLLIYGASSRATGIPDRFSGSGSGTDFTLTISRLEPEDFAVYYCQHCGSSLRAFGQGTKVEIKR

SEQ ID NO: 121 - protein, 246 aa, synthetic construct
EIVLTQSPGTLSLSPGERATLSCRASQSVSNNYLAWYQQKPGQAPRLLIYGASSRATGIPDRFSGSGSGTDFTLTISRLEPEDFAVYYCQHCGSSLRAFGQGTKVEIKRGGGGSGGGGSGGGGSEVQLVQSGAEVKKPGESLKISCKGSGYSFTNYWIGWVRQMPGKGLEWMGIIYPYDSDTRYSPSFEGQVTISADKSIRTAYLHWSSLKASDTAMYYCVRPRDGSYPYDAFDIWGQGTMVTVSS

SEQ ID NO: 122 - protein, 21 aa, synthetic construct
MALPVTALLLPLALLLHAARP

SEQ ID NO: 123 - protein, 45 aa, synthetic construct
TTTPAPRPPTPAPTIASQPLSLRPEACRPAAGGAVHTRGLDFACD

SEQ ID NO: 124 - protein, 19 aa, synthetic construct
LEPKSCDKTHTCPPCPDPK

SEQ ID NO: 125 - protein, 24 aa, synthetic construct
IYIWAPLAGTCGVLLLSLVITLYC

SEQ ID NO: 126 - protein, 27 aa, synthetic construct
FWVLVVVGGVLACYSLLVTVAFIIFWV

SEQ ID NO: 127 - protein, 42 aa, synthetic construct
KRGRKKLLYIFKQPFMRPVQTTQEEDGCSCRFPEEEEGGCEL

SEQ ID NO: 128 - protein, 41 aa, synthetic construct
RSKRSRLLHSDYMNMTPRRPGPTRKHYQPYAPPRDFAAYRS

SEQ ID NO: 129 - protein, 112 aa, synthetic construct
RVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 130 - DNA, 363 bp, synthetic construct
  source 1..363: mol_type = other DNA
gaatcaaaagcctctgaagtccagctgttggaaagcggcggtggtttggtccaatttcgcggcagccgacgcctctcctgcgcggtttctggtttctcagtctccggtaaccagatgacatgggtccggcaagcgccaggtaagggccttgaatggctctctgtaaagaatagtgatggctccacatcatatgcagattctgtaaaaggtaggttcacaatcgctcgcgacgaggtaaaaaacacagtttttcttcaaatgaacgctgtacgagcagaggacaccgcgttgtattactgcgctagactcaagaatggcgtgttcgacatctggggtcagggtacgatggtaacggttagctca

SEQ ID NO: 131 - DNA, 363 bp, synthetic construct
  source 1..363: mol_type = other DNA
gaaagtaaggcttccgaggtacagttggtcgaaagtggaggaggactggtacagccacgaggtagcctcagactctcttgcgcggcatcagggtttacttttacaacttttgcaatgtcctgggtgaggcaagcgccgggaaaggggctggagtgggtggcgactcgcaatggaaacggtggccgaacttattatgccgactcagtacgaggcagattcacaatttcacgagacctgcaccttcagatgaactctttgcgcgttgaggatacggcagtttattactgcacaaaggaccttgggccagtcgtaagaggcacttttgacgtatggggccaggggacgatggttacagtcagctca

SEQ ID NO: 132 - DNA, 384 bp, synthetic construct
  source 1..384: mol_type = other DNA
gagtccaaggcgagcgaagtacagttgcttgagtcaggcgggaggcaagttcaacctcgcggttctttgcgactgtcctgtactgcttcaggctttagtgtggggtcagccgatatgtcatgggtacgacaggcgcccggaaaaggcccagagtgggtctcatccaaggaatctgcaggtagcaccttctacgcagacagtgtgagagggaggttcacgatagcgcgagataatagtaacaatatgatttttttgcagctcaacagtctgcgacatgaagacactgcagtttattactgtgtgaggggttctgctagacgatcagcatccgggtggacaccttatgatctttggggacagggtactctggtaacggtcagctca

SEQ ID NO: 133 - DNA, 381 bp, synthetic construct
  source 1..381: mol_type = other DNA
gagtcaaaagcatccgaggttcaactggtggaatccggtggaacattgaaacaaccaagaggtagtcttcggctgagttgtgcggcatctggtttcacattcagtaatagtgatatggcatgggttaggcaggccccaggcaaaggcttggaatgggtgagttcaaaatcaggatctgacggcactacgtcatacgccgatagtgttaggggtcgattcaccattgctcgggataactctaaaaacacgctttatttgcagatgaacgcgctgcgggtggaagataccgcagtttactattgtgtcaaggggagtgcattctggtctggatctggatttttcgactcatggggccaagggacgctcgtcactgtgagcagt

SEQ ID NO: 134 - DNA, 369 bp, synthetic construct
  source 1..369: mol_type = other DNA
gaatccaaagctagcgaggtgcagctggtggagtctggaggaaccttgaaacagtctgcggggtccctgagactgtcctgtgcagcctctggattcagcgtcagtgattaccacatgagctgggtccgccaggctccagggaaggggctggagtgggtcgcaataaaatatagtggtggtcacacaggctacgcagactccgtgaagggccggttcaccatcgccagagacaattcgaagaatgacatttatctgcaaatgaacgccctgagaggcgaggacacggccgtctattattgtgcgagaggtgtcaacggtgactacttctttgactattggggccagggaaccctggtcaccgtctcctca

SEQ ID NO: 135 - DNA, 387 bp, synthetic construct
  source 1..387: mol_type = other DNA
gaatccaaagctagcgaggtgcagctggtggagtctgggggaggcgtggtccggcctgcgatgcccctgagactctcctgtgcagcctctggattctccttcaatgactatggcctgcactgggtccgtcaggctccgggcaaggggctggagtgggtggcatctatactatctcatggaaaaaaaacatactatgcagactctgtgaagggccgattcaccatcgccagagacaattccgagaacaccctgtatctgcaaatgaacaacctgagacctggggacacggctgtgtattattgtgcgaaagatctggttcctggcgctggcgtggaatactctgggacggacgtctggggccaagggacaatggtcaccgtctcttca

SEQ ID NO: 136 - DNA, 393 bp, synthetic construct
  source 1..393: mol_type = other DNA
gaatccaaagctagcgaggtgcagctggtggagtctgggggaggctcggttcagccgggggggtccctgagactctcctgtgcagcctctggattcacctttagtaactatgccctgagctgggtccgccaggttccagggaaggggctggagtgggtctcgggtatttatggtagtgttgctggcaggactatgacaactttttacgcagacttcgtgaagggccggttcaccatctccagagacaattccaagaacaccctgtacctggaaatgaacggcctgagagtcgaggacacggccgtatattactgtgcgaaagatatggtgggagctacttggttctacggtatggacgtctggggccaaggcaccctggtcaccgtctcctca

SEQ ID NO: 137 - DNA, 345 bp, synthetic construct
  source 1..345: mol_type = other DNA
ctggtgcagtctggagcagaggtgaaaaagcccgggcagtctctgaagatctcctgtaaggcttctggatacagcttaaccgacaactggatcggctgggtgcgccagaagcccgggaaaggcctggagtggatggggatcatctatcctggtgactctgacaccagatacagtccgtccttccaaggccaggtcaccatctcagccgacaagtccatcaacaccgcctacctgcagtggagcagcctgaaggcctcggacaccgccatatattactgtgtgggactcgattggaactacaaccccctgcgatactggggaccgggaacactggttaccgtttca

SEQ ID NO: 138 - DNA, 366 bp, synthetic construct
  source 1..366: mol_type = other DNA
gaggtgcagctggtgcagtctggagcagaggtgaaaaagcccggggagtctctgaagatctcctgcaagggttctggatacagctttaccaactactggatcggctgggtgcgccagatgcccgggaaaggcctggagtggatggggatcatctatccttatgactctgataccagatatagcccgtccttcgaaggccaggtcaccatttcagccgacaagtccatcaggaccgcctacctgcactggagcagcctgaaggcctcggacaccgccatgtattactgtgtgagaccccgcgatgggagctatccttatgatgcttttgatatctggggccaagggacaatggtcaccgtctcttca

SEQ ID NO: 139 - DNA, 357 bp, synthetic construct
  source 1..357: mol_type = other DNA
cagcccgtccttactcagcctacatccctgtctgctagtcctggggcgtccgcgagtttgacttgcaccctgcgaaggggtattaatttgggagcgtatggcatccattggtaccagcaacggcctggaagtcccccacgatatctgctcagacacaagagcgcatcagacaagcagcagggcagtggggttccagggaggttttccgggagcaaggacgccagtgccaatgccggcctcctcctgatttctgggctgcaatcagaagacgaagcagactactattgtatgatatactataacagcgcttgggtcttcggtggcggcacaaaactgaccgttctgggcgagggtaaa

SEQ ID NO: 140 - DNA, 336 bp, synthetic construct
  source 1..336: mol_type = other DNA
tcttctgaactgacgcaggatccgactgtgtcagtcgctttgggacagacggtacgaatcacctgccaaggagacagtctccgatcttactatgcgacgtggtaccaacagaaacctgggcaggcacctatactggtcatatatggtaagaacaatagaccgtctggaatacctgatagattctcagctagcactagtggaaatacagcaagcctcactattagcggggcacaagccgaagacgaggccgactactattgtggctcccgagatacgtcagacaatcacctgatgttcggcggcggcactaagctcacggtcttgggggaagggaag

SEQ ID NO: 141 - DNA, 357 bp, synthetic construct
  source 1..357: mol_type = other DNA
caggcagtccttacccaacccagtagcttgtcagctcctccaggagcgtcagcgacgctcccatgtacattgaggagcgacattaacgtggctactcaaaggatatactggtatcaccaaaaacctggttcaccattgcgatacctgcttcgatacaacagtgactctgacaatcggctgggttcaggtgtacctagccgcttcagtggcagcaaagatgtaagtgctaatgcggcctcactgctgatctccggactgcagagtgacgacgaggccgactactactgtgtcatctggcacaatagtgctgtggttttcgggggaggcactaaactcacagtactgggtgagggaaag

SEQ ID NO: 142 - DNA, 333 bp, synthetic construct
  source 1..333: mol_type = other DNA
tcttctgagctcacacaagacccagccgtgagtgttgcgttgggccagacggttaggataacatgtcaaggtaactctcttcgagggaatagcgcatcatggtaccaacagaagcctggacaagccccgagattggtaatgtaccatgaagatcgacggcccagtggggtgccagatcgcttcagcggttccagctccggtttcatctctagcttgaccatcacgggagcccaggctgcagacgaggccgattactactgcaatagtcgagataaatccgattccgttatctttggcggcggtaccaaggtcactgtgttgggagaggggaaa

SEQ ID NO: 143 - DNA, 336 bp, synthetic construct
  source 1..336: mol_type = other DNA
tcttatgagctgacacagccaccctcggtgtcagtgtccccaggacaaacggccaggatcacctgctctggagatgcattgccaaaaaaatatgcttattggtaccagcagaagtcaggccaggcccctgcactggtcatctatgaggacaacaaacgacccttcgggatccctgagagattctctggctccaggtcagggacaacggccaccttgactatcagcggggcccaggtggacgatgaagctgactactactgttactcaacagacagcagtggtaattatagggtgttcggcggagggaccaagctcaccgtcctaggtgagggtaaa

SEQ ID NO: 144 - DNA, 333 bp, synthetic construct
  source 1..333: mol_type = other DNA
gacattcagatgacccagtctccatcctccctgtctgcatctgtaggagccagagtcaccctcacttgtcgggcaagtcaggatattagtaggtacttgaattggtatcagcagaaatcagggagagcccctaaactcctgatctatggtgcatccagtttggaaagtggggtcccatcaaggttcagtggcagtgcatctgggtcaactttcactctcaccatcaacagtctacaacctgaagattttgcaacttactactgtcaacagagtttcacaaccccgtatacttttggccaggggaccaaggtgaccgtcctaggtgagggtaaa

SEQ ID NO: 145 - DNA, 342 bp, synthetic construct
  source 1..342: mol_type = other DNA
ctgcctgtgctgactcagccaccctcagcgtctgggacccccgggcagagggtcaccatctcttgttctggaagcagctccaacatcggaagtaatactgtaaactggtaccagcagctcccaggaacggcccccaaactcctcatctatagtaataatcagcggccctcaggggtccctgaccgattctctggctccaagtctggcacctcagcctccctggccatcagtgggctccggtccgaggatgaggctgattattactgtgcagcatgggatgacagcctgagtggtctggtattcggcggagggaccaagctcaccgtcctaggtgagggtaaa

SEQ ID NO: 146 - DNA, 327 bp, synthetic construct
  source 1..327: mol_type = other DNA
cagtcagtgttgacgcagccgccctcagtgtctgcggccccaggacagaaggtcaccatttcctgctccggaagcagctccgacattgggagtaattatgtatcctggtaccagcagttcccgggaacagcccccaaactcctcatttatgacaataataagcgaccctcagcgattcctgaccgattctctggctccaagtctggcacgtcagccaccctgggcatcaccggactccagactggggacgaggccgattattactgcggaacatgggatagcagactgggtattgctgtgttcggaggaggcacccagctgaccgtc

SEQ ID NO: 147 - DNA, 327 bp, synthetic construct
  source 1..327: mol_type = other DNA
gaaattgtgttgacgcagtctccaggcaccctgtctttgtctccaggggaaagagccaccctctcctgcagggccagtcagagtgttagcaacaactacttagcctggtaccagcagaagcctggccaggctcccaggctcctcatctatggtgcatccagcagggccactggcatcccagacaggttcagtggcagtgggtctgggacagatttcactttaaccatcagcagactggagcctgaagattttgcagtgtattactgtcagcattgtggtagctcactgagggcgttcggccaagggaccaaggtggaaatcaaacga

SEQ ID NO: 148 - DNA, 765 bp, synthetic construct
  source 1..765: mol_type = other DNA
gaatcaaaagcctctgaagtccagctgttggaaagcggcggtggtttggtccaatttcgcggcagccgacgcctctcctgcgcggtttctggtttctcagtctccggtaaccagatgacatgggtccggcaagcgccaggtaagggccttgaatggctctctgtaaagaatagtgatggctccacatcatatgcagattctgtaaaaggtaggttcacaatcgctcgcgacgaggtaaaaaacacagtttttcttcaaatgaacgctgtacgagcagaggacaccgcgttgtattactgcgctagactcaagaatggcgtgttcgacatctggggtcagggtacgatggtaacggttagctcaggtggaggtggttcgggaggtggaggtagcggaggtggtggatctcagcccgtccttactcagcctacatccctgtctgctagtcctggggcgtccgcgagtttgacttgcaccctgcgaaggggtattaatttgggagcgtatggcatccattggtaccagcaacggcctggaagtcccccacgatatctgctcagacacaagagcgcatcagacaagcagcagggcagtggggttccagggaggttttccgggagcaaggacgccagtgccaatgccggcctcctcctgatttctgggctgcaatcagaagacgaagcagactactattgtatgatatactataacagcgcttgggtcttcggtggcggcacaaaactgaccgttctgggcgagggtaaa

SEQ ID NO: 149 - DNA, 765 bp, synthetic construct
  source 1..765: mol_type = other DNA
cagcccgtccttactcagcctacatccctgtctgctagtcctggggcgtccgcgagtttgacttgcaccctgcgaaggggtattaatttgggagcgtatggcatccattggtaccagcaacggcctggaagtcccccacgatatctgctcagacacaagagcgcatcagacaagcagcagggcagtggggttccagggaggttttccgggagcaaggacgccagtgccaatgccggcctcctcctgatttctgggctgcaatcagaagacgaagcagactactattgtatgatatactataacagcgcttgggtcttcggtggcggcacaaaactgaccgttctgggcgagggtaaaggtggaggtggttcgggaggtggaggtagcggaggtggtggatctgaatcaaaagcctctgaagtccagctgttggaaagcggcggtggtttggtccaatttcgcggcagccgacgcctctcctgcgcggtttctggtttctcagtctccggtaaccagatgacatgggtccggcaagcgccaggtaagggccttgaatggctctctgtaaagaatagtgatggctccacatcatatgcagattctgtaaaaggtaggttcacaatcgctcgcgacgaggtaaaaaacacagtttttcttcaaatgaacgctgtacgagcagaggacaccgcgttgtattactgcgctagactcaagaatggcgtgttcgacatctggggtcagggtacgatggtaacggttagctca

SEQ ID NO: 150 - DNA, 744 bp, synthetic construct
  source 1..744: mol_type = other DNA
gaaagtaaggcttccgaggtacagttggtcgaaagtggaggaggactggtacagccacgaggtagcctcagactctcttgcgcggcatcagggtttacttttacaacttttgcaatgtcctgggtgaggcaagcgccgggaaaggggctggagtgggtggcgactcgcaatggaaacggtggccgaacttattatgccgactcagtacgaggcagattcacaatttcacgagacctgcaccttcagatgaactctttgcgcgttgaggatacggcagtttattactgcacaaaggaccttgggccagtcgtaagaggcacttttgacgtatggggccaggggacgatggttacagtcagctcaggtggaggtggttcgggaggtggaggtagcggaggtggtggatcttcttctgaactgacgcaggatccgactgtgtcagtcgctttgggacagacggtacgaatcacctgccaaggagacagtctccgatcttactatgcgacgtggtaccaacagaaacctgggcaggcacctatactggtcatatatggtaagaacaatagaccgtctggaatacctgatagattctcagctagcactagtggaaatacagcaagcctcactattagcggggcacaagccgaagacgaggccgactactattgtggctcccgagatacgtcagacaatcacctgatgttcggcggcggcactaagctcacggtcttgggggaagggaag

SEQ ID NO: 151 - DNA, 744 bp, synthetic construct
  source 1..744: mol_type = other DNA
tcttctgaactgacgcaggatccgactgtgtcagtcgctttgggacagacggtacgaatcacctgccaaggagacagtctccgatcttactatgcgacgtggtaccaacagaaacctgggcaggcacctatactggtcatatatggtaagaacaatagaccgtctggaatacctgatagattctcagctagcactagtggaaatacagcaagcctcactattagcggggcacaagccgaagacgaggccgactactattgtggctcccgagatacgtcagacaatcacctgatgttcggcggcggcactaagctcacggtcttgggggaagggaagggtggaggtggttcgggaggtggaggtagcggaggtggtggatctgaaagtaaggcttccgaggtacagttggtcgaaagtggaggaggactggtacagccacgaggtagcctcagactctcttgcgcggcatcagggtttacttttacaacttttgcaatgtcctgggtgaggcaagcgccgggaaaggggctggagtgggtggcgactcgcaatggaaacggtggccgaacttattatgccgactcagtacgaggcagattcacaatttcacgagacctgcaccttcagatgaactctttgcgcgttgaggatacggcagtttattactgcacaaaggaccttgggccagtcgtaagaggcacttttgacgtatggggccaggggacgatggttacagtcagctca

SEQ ID NO: 152 - DNA, 786 bp, synthetic construct
  source 1..786: mol_type = other DNA
gagtccaaggcgagcgaagtacagttgcttgagtcaggcgggaggcaagttcaacctcgcggttctttgcgactgtcctgtactgcttcaggctttagtgtggggtcagccgatatgtcatgggtacgacaggcgcccggaaaaggcccagagtgggtctcatccaaggaatctgcaggtagcaccttctacgcagacagtgtgagagggaggttcacgatagcgcgagataatagtaacaatatgatttttttgcagctcaacagtctgcgacatgaagacactgcagtttattactgtgtgaggggttctgctagacgatcagcatccgggtggacaccttatgatctttggggacagggtactctggtaacggtcagctcaggtggaggtggttcgggaggtggaggtagcggaggtggtggatctcaggcagtccttacccaacccagtagcttgtcagctcctccaggagcgtcagcgacgctcccatgtacattgaggagcgacattaacgtggctactcaaaggatatactggtatcaccaaaaacctggttcaccattgcgatacctgcttcgatacaacagtgactctgacaatcggctgggttcaggtgtacctagccgcttcagtggcagcaaagatgtaagtgctaatgcggcctcactgctgatctccggactgcagagtgacgacgaggccgactactactgtgtcatctggcacaatagtgctgtggttttcgggggaggcactaaactcacagtactgggtgagggaaag

SEQ ID NO: 153 - DNA, 786 bp, synthetic construct
  source 1..786: mol_type = other DNA
caggcagtccttacccaacccagtagcttgtcagctcctccaggagcgtcagcgacgctcccatgtacattgaggagcgacattaacgtggctactcaaaggatatactggtatcaccaaaaacctggttcaccattgcgatacctgcttcgatacaacagtgactctgacaatcggctgggttcaggtgtacctagccgcttcagtggcagcaaagatgtaagtgctaatgcggcctcactgctgatctccggactgcagagtgacgacgaggccgactactactgtgtcatctggcacaatagtgctgtggttttcgggggaggcactaaactcacagtactgggtgagggaaagggtggaggtggttcgggaggtggaggtagcggaggtggtggatctgagtccaaggcgagcgaagtacagttgcttgagtcaggcgggaggcaagttcaacctcgcggttctttgcgactgtcctgtactgcttcaggctttagtgtggggtcagccgatatgtcatgggtacgacaggcgcccggaaaaggcccagagtgggtctcatccaaggaatctgcaggtagcaccttctacgcagacagtgtgagagggaggttcacgatagcgcgagataatagtaacaatatgatttttttgcagctcaacagtctgcgacatgaagacactgcagtttattactgtgtgaggggttctgctagacgatcagcatccgggtggacaccttatgatctttggggacagggtactctggtaacggtcagctca

SEQ ID NO: 154 - DNA, 759 bp, synthetic construct
  source 1..759: mol_type = other DNA
gagtcaaaagcatccgaggttcaactggtggaatccggtggaacattgaaacaaccaagaggtagtcttcggctgagttgtgcggcatctggtttcacattcagtaatagtgatatggcatgggttaggcaggccccaggcaaaggcttggaatgggtgagttcaaaatcaggatctgacggcactacgtcatacgccgatagtgttaggggtcgattcaccattgctcgggataactctaaaaacacgctttatttgcagatgaacgcgctgcgggtggaagataccgcagtttactattgtgtcaaggggagtgcattctggtctggatctggatttttcgactcatggggccaagggacgctcgtcactgtgagcagtggtggaggtggttcgggaggtggaggtagcggaggtggtggatcttcttctgagctcacacaagacccagccgtgagtgttgcgttgggccagacggttaggataacatgtcaaggtaactctcttcgagggaatagcgcatcatggtaccaacagaagcctggacaagccccgagattggtaatgtaccatgaagatcgacggcccagtggggtgccagatcgcttcagcggttccagctccggtttcatctctagcttgaccatcacgggagcccaggctgcagacgaggccgattactactgcaatagtcgagataaatccgattccgttatctttggcggcggtaccaaggtcactgtgttgggagaggggaaa

SEQ ID NO: 155 - DNA, 759 bp, synthetic construct
  source 1..759: mol_type = other DNA
tcttctgagctcacacaagacccagccgtgagtgttgcgttgggccagacggttaggataacatgtcaaggtaactctcttcgagggaatagcgcatcatggtaccaacagaagcctggacaagccccgagattggtaatgtaccatgaagatcgacggcccagtggggtgccagatcgcttcagcggttccagctccggtttcatctctagcttgaccatcacgggagcccaggctgcagacgaggccgattactactgcaatagtcgagataaatccgattccgttatctttggcggcggtaccaaggtcactgtgttgggagaggggaaaggtggaggtggttcgggaggtggaggtagcggaggtggtggatctgagtcaaaagcatccgaggttcaactggtggaatccggtggaacattgaaacaaccaagaggtagtcttcggctgagttgtgcggcatctggtttcacattcagtaatagtgatatggcatgggttaggcaggccccaggcaaaggcttggaatgggtgagttcaaaatcaggatctgacggcactacgtcatacgccgatagtgttaggggtcgattcaccattgctcgggataactctaaaaacacgctttatttgcagatgaacgcgctgcgggtggaagataccgcagtttactattgtgtcaaggggagtgcattctggtctggatctggatttttcgactcatggggccaagggacgctcgtcactgtgagcagt

SEQ ID NO: 156 - DNA, 750 bp, synthetic construct
  source 1..750: mol_type = other DNA
gaatccaaagctagcgaggtgcagctggtggagtctggaggaaccttgaaacagtctgcggggtccctgagactgtcctgtgcagcctctggattcagcgtcagtgattaccacatgagctgggtccgccaggctccagggaaggggctggagtgggtcgcaataaaatatagtggtggtcacacaggctacgcagactccgtgaagggccggttcaccatcgccagagacaattcgaagaatgacatttatctgcaaatgaacgccctgagaggcgaggacacggccgtctattattgtgcgagaggtgtcaacggtgactacttctttgactattggggccagggaaccctggtcaccgtctcctcaggtggaggtggttcgggaggtggaggtagcggaggtggtggatcttcttatgagctgacacagccaccctcggtgtcagtgtccccaggacaaacggccaggatcacctgctctggagatgcattgccaaaaaaatatgcttattggtaccagcagaagtcaggccaggcccctgcactggtcatctatgaggacaacaaacgacccttcgggatccctgagagattctctggctccaggtcagggacaacggccaccttgactatcagcggggcccaggtggacgatgaagctgactactactgttactcaacagacagcagtggtaattatagggtgttcggcggagggaccaagctcaccgtcctaggtgagggtaaa

SEQ ID NO: 157 - DNA, 750 bp, synthetic construct
  source 1..750: mol_type = other DNA
tcttatgagctgacacagccaccctcggtgtcagtgtccccaggacaaacggccaggatcacctgctctggagatgcattgccaaaaaaatatgcttattggtaccagcagaagtcaggccaggcccctgcactggtcatctatgaggacaacaaacgacccttcgggatccctgagagattctctggctccaggtcagggacaacggccaccttgactatcagcggggcccaggtggacgatgaagctgactactactgttactcaacagacagcagtggtaattatagggtgttcggcggagggaccaagctcaccgtcctaggtgagggtaaaggtggaggtggttcgggaggtggaggtagcggaggtggtggatctgaatccaaagctagcgaggtgcagctggtggagtctggaggaaccttgaaacagtctgcggggtccctgagactgtcctgtgcagcctctggattcagcgtcagtgattaccacatgagctgggtccgccaggctccagggaaggggctggagtgggtcgcaataaaatatagtggtggtcacacaggctacgcagactccgtgaagggccggttcaccatcgccagagacaattcgaagaatgacatttatctgcaaatgaacgccctgagaggcgaggacacggccgtctattattgtgcgagaggtgtcaacggtgactacttctttgactattggggccagggaaccctggtcaccgtctcctca

SEQ ID NO: 158 - DNA, 765 bp, synthetic construct
  source 1..765: mol_type = other DNA
gaatccaaagctagcgaggtgcagctggtggagtctgggggaggcgtggtccggcctgcgatgcccctgagactctcctgtgcagcctctggattctccttcaatgactatggcctgcactgggtccgtcaggctccgggcaaggggctggagtgggtggcatctatactatctcatggaaaaaaaacatactatgcagactctgtgaagggccgattcaccatcgccagagacaattccgagaacaccctgtatctgcaaatgaacaacctgagacctggggacacggctgtgtattattgtgcgaaagatctggttcctggcgctggcgtggaatactctgggacggacgtctggggccaagggacaatggtcaccgtctcttcaggtggaggtggttcgggaggtggaggtagcggaggtggtggatctgacattcagatgacccagtctccatcctccctgtctgcatctgtaggagccagagtcaccctcacttgtcgggcaagtcaggatattagtaggtacttgaattggtatcagcagaaatcagggagagcccctaaactcctgatctatggtgcatccagtttggaaagtggggtcccatcaaggttcagtggcagtgcatctgggtcaactttcactctcaccatcaacagtctacaacctgaagattttgcaacttactactgtcaacagagtttcacaaccccgtatacttttggccaggggaccaaggtgaccgtcctaggtgagggtaaa

SEQ ID NO: 159 - DNA, 765 bp, synthetic construct
  source 1..765: mol_type = other DNA
gacattcagatgacccagtctccatcctccctgtctgcatctgtaggagccagagtcaccctcacttgtcgggcaagtcaggatattagtaggtacttgaattggtatcagcagaaatcagggagagcccctaaactcctgatctatggtgcatccagtttggaaagtggggtcccatcaaggttcagtggcagtgcatctgggtcaactttcactctcaccatcaacagtctacaacctgaagattttgcaacttactactgtcaacagagtttcacaaccccgtatacttttggccaggggaccaaggtgaccgtcctaggtgagggtaaaggtggaggtggttcgggaggtggaggtagcggaggtggtggatctgaatccaaagctagcgaggtgcagctggtggagtctgggggaggcgtggtccggcctgcgatgcccctgagactctcctgtgcagcctctggattctccttcaatgactatggcctgcactgggtccgtcaggctccgggcaaggggctggagtgggtggcatctatactatctcatggaaaaaaaacatactatgcagactctgtgaagggccgattcaccatcgccagagacaattccgagaacaccctgtatctgcaaatgaacaacctgagacctggggacacggctgtgtattattgtgcgaaagatctggttcctggcgctggcgtggaatactctgggacggacgtctggggccaagggacaatggtcaccgtctcttca

SEQ ID NO: 160 - DNA, 780 bp, synthetic construct
  source 1..780: mol_type = other DNA
gaatccaaagctagcgaggtgcagctggtggagtctgggggaggctcggttcagccgggggggtccctgagactctcctgtgcagcctctggattcacctttagtaactatgccctgagctgggtccgccaggttccagggaaggggctggagtgggtctcgggtatttatggtagtgttgctggcaggactatgacaactttttacgcagacttcgtgaagggccggttcaccatctccagagacaattccaagaacaccctgtacctggaaatgaacggcctgagagtcgaggacacggccgtatattactgtgcgaaagatatggtgggagctacttggttctacggtatggacgtctggggccaaggcaccctggtcaccgtctcctcaggtggaggtggttcgggaggtggaggtagcggaggtggtggatctctgcctgtgctgactcagccaccctcagcgtctgggacccccgggcagagggtcaccatctcttgttctggaagcagctccaacatcggaagtaatactgtaaactggtaccagcagctcccaggaacggcccccaaactcctcatctatagtaataatcagcggccctcaggggtccctgaccgattctctggctccaagtctggcacctcagcctccctggccatcagtgggctccggtccgaggatgaggctgattattactgtgcagcatgggatgacagcctgagtggtctggtattcggcggagggaccaagctcaccgtcctaggtgagggtaaa

SEQ ID NO: 161 - DNA, 780 bp, synthetic construct
  source 1..780: mol_type = other DNA
ctgcctgtgctgactcagccaccctcagcgtctgggacccccgggcagagggtcaccatctcttgttctggaagcagctccaacatcggaagtaatactgtaaactggtaccagcagctcccaggaacggcccccaaactcctcatctatagtaataatcagcggccctcaggggtccctgaccgattctctggctccaagtctggcacctcagcctccctggccatcagtgggctccggtccgaggatgaggctgattattactgtgcagcatgggatgacagcctgagtggtctggtattcggcggagggaccaagctcaccgtcctaggtgagggtaaaggtggaggtggttcgggaggtggaggtagcggaggtggtggatctgaatccaaagctagcgaggtgcagctggtggagtctgggggaggctcggttcagccgggggggtccctgagactctcctgtgcagcctctggattcacctttagtaactatgccctgagctgggtccgccaggttccagggaaggggctggagtgggtctcgggtatttatggtagtgttgctggcaggactatgacaactttttacgcagacttcgtgaagggccggttcaccatctccagagacaattccaagaacaccctgtacctggaaatgaacggcctgagagtcgaggacacggccgtatattactgtgcgaaagatatggtgggagctacttggttctacggtatggacgtctggggccaaggcaccctggtcaccgtctcctca

SEQ ID NO: 162 - DNA, 717 bp, synthetic construct
  source 1..717: mol_type = other DNA
ctggtgcagtctggagcagaggtgaaaaagcccgggcagtctctgaagatctcctgtaaggcttctggatacagcttaaccgacaactggatcggctgggtgcgccagaagcccgggaaaggcctggagtggatggggatcatctatcctggtgactctgacaccagatacagtccgtccttccaaggccaggtcaccatctcagccgacaagtccatcaacaccgcctacctgcagtggagcagcctgaaggcctcggacaccgccatatattactgtgtgggactcgattggaactacaaccccctgcgatactggggaccgggaacactggttaccgtttcaggtggaggtggttcgggaggtggaggtagcggaggtggtggatctcagtcagtgttgacgcagccgccctcagtgtctgcggccccaggacagaaggtcaccatttcctgctccggaagcagctccgacattgggagtaattatgtatcctggtaccagcagttcccgggaacagcccccaaactcctcatttatgacaataataagcgaccctcagcgattcctgaccgattctctggctccaagtctggcacgtcagccaccctgggcatcaccggactccagactggggacgaggccgattattactgcggaacatgggatagcagactgggtattgctgtgttcggaggaggcacccagctgaccgtc

SEQ ID NO: 163 - DNA, 717 bp, synthetic construct
  source 1..717: mol_type = other DNA
cagtcagtgttgacgcagccgccctcagtgtctgcggccccaggacagaaggtcaccatttcctgctccggaagcagctccgacattgggagtaattatgtatcctggtaccagcagttcccgggaacagcccccaaactcctcatttatgacaataataagcgaccctcagcgattcctgaccgattctctggctccaagtctggcacgtcagccaccctgggcatcaccggactccagactggggacgaggccgattattactgcggaacatgggatagcagactgggtattgctgtgttcggaggaggcacccagctgaccgtcggtggaggtggttcgggaggtggaggtagcggaggtggtggatctctggtgcagtctggagcagaggtgaaaaagcccgggcagtctctgaagatctcctgtaaggcttctggatacagcttaaccgacaactggatcggctgggtgcgccagaagcccgggaaaggcctggagtggatggggatcatctatcctggtgactctgacaccagatacagtccgtccttccaaggccaggtcaccatctcagccgacaagtccatcaacaccgcctacctgcagtggagcagcctgaaggcctcggacaccgccatatattactgtgtgggactcgattggaactacaaccccctgcgatactggggaccgggaacactggttaccgtttca

SEQ ID NO: 164 - DNA, 738 bp, synthetic construct
  source 1..738: mol_type = other DNA
gaggtgcagctggtgcagtctggagcagaggtgaaaaagcccggggagtctctgaagatctcctgcaagggttctggatacagctttaccaactactggatcggctgggtgcgccagatgcccgggaaaggcctggagtggatggggatcatctatccttatgactctgataccagatatagcccgtccttcgaaggccaggtcaccatttcagccgacaagtccatcaggaccgcctacctgcactggagcagcctgaaggcctcggacaccgccatgtattactgtgtgagaccccgcgatgggagctatccttatgatgcttttgatatctggggccaagggacaatggtcaccgtctcttcaggtggaggtggttcgggaggtggaggtagcggaggtggtggatctgaaattgtgttgacgcagtctccaggcaccctgtctttgtctccaggggaaagagccaccctctcctgcagggccagtcagagtgttagcaacaactacttagcctggtaccagcagaagcctggccaggctcccaggctcctcatctatggtgcatccagcagggccactggcatcccagacaggttcagtggcagtgggtctgggacagatttcactttaaccatcagcagactggagcctgaagattttgcagtgtattactgtcagcattgtggtagctcactgagggcgttcggccaagggaccaaggtggaaatcaaacga

SEQ ID NO: 165 - DNA, 327 bp, synthetic construct; identical to SEQ ID NO: 147
  source 1..327: mol_type = other DNA

SEQ ID NO: 166 - DNA, 63 bp, synthetic construct
  source 1..63: mol_type = other DNA
atggccttaccagtgaccgccttgctcctgccgctggccttgctgctccacgccgccaggccg

SEQ ID NO: 167 - DNA, 45 bp, synthetic construct
  source 1..45: mol_type = other DNA
ggtggaggtggttcgggaggtggaggtagcggaggtggtggatct

SEQ ID NO: 168 - DNA, 135 bp, synthetic construct
  source 1..135: mol_type = other DNA
accactacccctgcaccgcgaccaccaacaccggcgcccaccattgcgtcgcagcctctgtccctgcgcccagaagcatgccgtccagcagcaggtggtgcagttcatactcgtggtctggatttcgcctgtgat

SEQ ID NO: 169 - DNA, 57 bp, synthetic construct
  source 1..57: mol_type = other DNA
ctcgagcccaaatcttgtgacaaaactcacacatgcccaccgtgcccggatcccaaa

SEQ ID NO: 170 - DNA, 72 bp, synthetic construct
  source 1..72: mol_type = other DNA
atctacatctgggcgcccttggccgggacttgtggggtccttctcctgtcactggttatcaccctttactgc

SEQ ID NO: 171 - DNA, 81 bp, synthetic construct
  source 1..81: mol_type = other DNA
ttttgggtgctggtggtggttggtggagtcctggcttgctatagcttgctagtaacagtggcctttattattttctgggtg

SEQ ID NO: 172 - DNA, 126 bp, synthetic construct
  source 1..126: mol_type = other DNA
aaacggggcagaaagaaactcctgtatatattcaaacaaccatttatgagaccagtacaaactactcaagaggaagatggctgtagctgccgatttccagaagaagaagaaggaggatgtgaactg

SEQ ID NO: 173 - DNA, 123 bp, synthetic construct
  source 1..123: mol_type = other DNA
aggagtaagaggagcaggctcctgcacagtgactacatgaacatgactccccgccgccccgggcccacccgcaagcattaccagccctatgccccaccacgcgacttcgcagcctatcgctcc

SEQ ID NO: 174 - DNA, 336 bp, synthetic construct
  source 1..336: mol_type = other DNA
agagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc